(19) United States
(12) Patent Application Publication
(10) Pub. No.: US 2026/0070929 A1
(43) Pub. Date: Mar. 12, 2026

(54) METHODS AND COMPOSITIONS FOR MODULATING KRAS(G12D)

(71) Applicant: Ranok Therapeutics (Hangzhou) Co. LTD., Zhejiang (CN)

(72) Inventors: Weiwen Ying, Lexington, MA (US); Kevin P. Foley, Waltham, MA (US); Chenghao Ying, Shanghai (CN); Yaya Wang, Hangzhou (CN); Yan Dai, Hangzhou (CN); Guoqiang Wang, Lexington, MA (US); Wei Yin, Lianyungang (CN); Jinhua Li, Hangzhou (CN); Thomas Prince, Danville, PA (US); Zhiyong Wang, Hangzhou (CN)

(21) Appl. No.: 19/106,175

(22) PCT Filed: Aug. 24, 2023
(86) PCT No.: PCT/US2023/031083

§ 371 Date: Feb. 24, 2025

(30) Foreign Application Priority Data
Aug. 24, 2022 (WO) PCT/CN2022/114635

(51) Int. Cl. C07D 519/00; A61K 31/519; A61K 31/551; A61K 47/55; A61P 35/00

(52) U.S. Cl. CPC C07D 519/00; A61K 31/519; A61K 31/551; A61K 47/55; A61P 35/00

(57) ABSTRACT

Provided are tumor-targeted protein degradation chimeras, termed chaperone-mediated protein degraders (CHAMPs) comprising a first moiety that is capable of binding to a target protein (e.g., KRAS(G12D) or proteins and a second moiety that is capable of binding a chaperone protein or proteins or protein component of chaperone complexes (e.g., HSP90). Pharmaceutical compositions comprising the disclosed CHAMPs and their uses for treating thereof, which are useful for treating cancers and related conditions are also provided.

### RELATED APPLICATIONS

This application claims the benefit of priority to International Application No. PCT/CN2022/114635, filed Aug. 24, 2022, the entire contents of which are incorporated herein by reference.

### BACKGROUND

Protein homeostasis, or proteostasis, refers to the ability of cells to regulate the synthesis, folding, trafficking and degradation of proteins. In particular, properly regulated protein degradation is required for the normal functioning of cells, including their proliferation, differentiation and death, and is often dysregulated in cancers and other diseases (Van Die, Chin J Cancer, 2011, 30:124-137).

The ubiquitin-proteasome system (UPS) is one of the major pathways in cells that mediates the disposal and metabolic recycling of proteins (Yu and Matouschek, Annu Rev Biophys, 2017, 46:149-173; Navon and Ciechanover, J Biol Chem, 2009, 284:33713-33718). Ubiquitin is a 76 amino acid-residue protein that is ubiquitously expressed. With respect to protein degradation by the UPS, the process of ubiquitination occurs when a ubiquitin is attached to a lysine amino acid residue in a substrate protein, which involves a series of enzymatic steps. First, ubiquitin is transferred to an E1 ubiquitin-activating enzyme. Second, activated ubiquitin is transferred from the E1 to an E2 ubiquitin-conjugating enzyme. And third, one of the several hundred different E3 ubiquitin ligase enzymes links the ubiquitin to a lysine residue in a substrate protein. Repetition of this enzymatic process results in tagging substrate proteins with polyubiquitin chains. Such ubiquitin-tagged proteins can then be delivered to the proteasome, a large multi-subunit complex that degrades proteins. The ability of some cellular chaperone proteins and chaperone complexes to direct proteins towards the UPS is facilitated by their direct interaction with E3 ubiquitin ligases (Amm et al., Biochim Biophys Acta, 2014, 1843:182-196; Taipale et al., Cell, 2012, 150:987-1001).

Chemically induced, targeted protein degradation (TPD) has emerged as an important for drug development. A small molecule can be used to promote the interaction of a target protein or proteins with a component or components of various cellular protein degradation pathways, thereby inducing the degradation of the targeted protein or proteins as a way to treat disease.

In particular, proteolysis-targeting chimeras (PROTACs) are an example of such small molecules that induce protein degradation of specific proteins by coopting the UPS (Burslem and Crews, Cell, 2020, 181:102-114; Pettersson and Crews, Drug Discov Today Technol, 2019, 31:15-27). PROTAC molecules are bifunctional small molecules that simultaneously bind to a target protein or proteins and an E3 ubiquitin ligase, creating ternary complexes in cells between the target protein(s), the PROTAC molecule and an E3 ligase protein. The induced proximity of the target protein(s) and the E3 ligase causes the ubiquitination of the target protein(s) and subsequent degradation of the target protein(s) by the proteasome. Although PROTACs that incorporate target protein binders that promiscuously bind to multiple proteins can often degrade multiple proteins, in some cases protein-protein interactions between individual targets and an E3 ligase can increase or decrease the observed potency and selectivity of degradation, for example by inhibiting formation of some ternary complexes due to charge repulsion and steric clashing between a given target protein and E3 ligase pair (Pettersson and Crews, Drug Discov Today Technol, 2019, 31:15-27; Bondeson et al., Cell Chem Biol, 2018, 25:78-87; Gadd et al., Nat Chem Biol, 2017, 13:514-521; Zengerle et al., ACS Chem Biol, 2015, 10:1770-1777).

Other methods to chemically induce TPD have also been described, such as molecular glues (Che et al., Bioog Med Chem Lett, 2018, 28:2585-2592), AUTACs, ATTECs and LYTACs (Ding et al., Trends Pharmacol Sci, 2020, 41:464-474). For example, AUTAC technology follows a similar principle of induced proximity, but targets proteins for degradation via autophagy (Daiki et al., Mol Cell, 2019, 76:797-810).

Collectively, TPD technologies have a number of advantages over conventional biochemical inhibitors (Pettersson and Crews, Drug Discov Today Technol, 2019, 31:15-27; Ding et al., Trends Pharmacol Sci, 2020, 41:464-474). For example, unlike conventional inhibitors, TPD agents work sub-stoichiometrically and can typically mediate the sequential degradation of multiple molecules of the target protein(s), often leading to greater potency than the isolated target binding moiety that they incorporate and other biochemical inhibitors. Also, since inhibition of target protein(s) function by TPD agents is principally due to degradation rather than solely biochemical inhibition, recovery of the function of target protein(s) is typically slower than is observed for biochemical inhibitors. TPD agents may also have improved target selectivity over biochemical inhibitors. Finally, TPD agents can target proteins that are not amenable to biochemical inhibition by interacting with binding pockets that do not affect the biochemical activity of the target but still permit its degradation.

However, some disadvantages are associated with current TPD technologies. These include the promiscuous degradation of the target protein(s) in many tissues and organs, not just the tissue(s) and organ(s) where the target protein(s) is involved in a disease process, which is expected to result in unwanted side effects of treatment. Also, resistance to these technologies can develop through mutations or alterations in expression of components of the UPS such as E3 ligases (Ottis et al., ACS Chem Biol, 2019, 14:2215-2223; Zhang et al., Mol Cancer Ther, 2019, 18:1302-1311), resulting in loss of therapeutic efficacy. As such, a need exists for improved/alternative methods and compositions for TPD.

It is therefore desirable to develop improved/alternative TPD agents that mediate the degradation of proteins involved in cancer and other diseases. HSP90 (Heat Shock Protein 90-kDa) is a molecular chaperone that interacts with a significant portion of signaling proteins expressed in cells. These proteins include kinases, transcription factors, and numerous E3 ligases that direct protein degradation (Taipale et al., Cell, 2012, 150:987-100). Moreover, HSP90 folds and maintains the structure of many mutated or overexpressed oncoproteins that drive tumor growth (Schwartz et al., Cell Stress Chaperones. 2015, 20:729-41). Remarkably, molecular inhibitors that bind to the N-terminus of HSP90 are observed to accumulate in tumor cells (Kamal et al., Nature, 2003, 425:407-102003; Moulick et al., Nat Chem Biol. 2011, 7:818-26). HSP90 inhibitor binding induces HSP90-dependent oncoprotein depletion through a process involving E3-ligases and the UPS (Li et al., Cell Rep. 2017, 20; 19:2515-2528; Xu et al., Proc Natl Acad Sci USA. 2002, 99:12847-52). Consequently, targeted inhibition of HSP90 results in tumor cell cytoxicity (Wang et al., Curr Opin Investig Drugs. 2010, 11:1466-76; Trepel et al., Nat Rev Cancer. 2010, 10:537-49).

The Kirsten rat sarcoma virus homolog (KRAS) is a monomeric small 21 kDa GTPase that has long been an elusive cancer drug target (Chang et al., PNAS, 1982, 79:4848-52; McCoy et al., Nature, 1983, 302:79-8). KRAS functions as a molecular switch for promoting cell growth by cycling between GTP- and GDP-bound states. In the GTP-bound state, KRAS signals for growth through the RAF-MAPK and PI3K-AKT-MTOR pathways. KRAS subsequently hydrolyzes GTP-to-GDP with the aid of GTPase activating proteins (GAPs). This GDP-bound state switches “off” KRAS pro-growth signaling. KRAS can then be switched back “on” by GDP-to-GTP exchange through the aid of guanine nucleotide exchange factors, such as SOS1 (Cox and Der, Small GTPases, 2010, 1:2-27; Kerk et al., Nat Rev Cancer, 2021, 21:510-525). Preventing this exchange by locking KRAS in the GDP-bound state is a practical method for inhibiting its oncogenic activity.

The human KRAS gene is encoded on Chromosome 12p12.1 and is among the most frequently mutated genes in human cancers (Pylayeva-Gupta et al., Nat Rev Cancer, 2011, 11:761-774). Mutations that prevent GTP-hydrolysis lock KRAS in the active GTP-bound state and reprogram cells for perpetual proliferation. KRAS mutated from glycine (G) at the 12^{t} codon to aspartate (D) creates a chronically active KRAS(G12D) oncogene observed in 6.8% of cancers cases as analyzed by next-generation sequencing (Zhou et al., Pathol Oncol Res, 2020, 26:2835-2837). KRAS(G12D) is associated with poor clinical outcomes and observed in 4.9% of lung adenocarcinomas, 12.5% of colorectal carcinomas, and 37% of pancreatic ductal adenocarcinomas (Hofman et al., Cancer Discov. 2022, 12:924-937). HSP90 notably is not known to chaperone/associate with KRAS or its mutants.

Historically, oncogenic KRAS mutants have been considered undruggable (McCormick F., Biochem J, 2019, 476:356-74), however the discovery of an allosteric pocket in GDP-bound KRAS has allowed for the search for small molecule ihibitors (Ostrem et al., Nature, 2013, 503: 548-51). The G12D mutation moreover provides a unique moiety binding space due to the encoding of an acidic amino acid residue (D) in place of a small flexible amino acid residue possessing only a hydrogen side-chain (G). This alteration of the KRAS protein structure provides a unique space that may be targeted with small molecules drugs that specifically bind and inhibit KRAS(G12D) oncogenic activity.

It is therefore desirable to design and develop agents that modulate KRAS(G12D). It is also desirable to design and develop agents that bind both HSP90 and KRAS(G12D) to induce novel protein-protein interactions that promotes TPD of KRAS(G12D) as a treatment for cancer and other diseases. It is likewise desirable to develop agents that inhibit and induce degradation of both KRAS(G12D) and one or more other proteins, particularly those involved in mediating resistance to KRAS(G12D) inhibitors. Additionally, it is desirable to develop agents that accumulate in tumor cells.

### SUMMARY

The present disclosure provides tumor-targeted protein degradation chimeras, termed chaperone-mediated protein degraders (CHAMPs) comprising a first moiety that is capable of binding to a target protein (e.g., KRAS(G12D)) or proteins and a second moiety that is capable of binding a chaperone protein or proteins or protein component of chaperone complexes (e.g., HSP90).

Such CHAMP compounds include those having the Formula K-L-Hs, wherein K is a chemical entity that binds to KRAS(G12D), L is a linker, and Hs is chemical entity that binds HSP90 protein.

Compositions comprising the disclosed CHAMP compounds as well as methods for their manufacture are also provided. In one aspect, the disclosed CHAMP compounds induce targeted oncogenic protein degradation in a tumor-selective fashion and are useful in the treatment of cancer and related condition.

### DETAILED DESCRIPTION

### 1. General Description of Compounds

In a first embodiment, the CHAMP compounds described herein are of the Formula I or II:

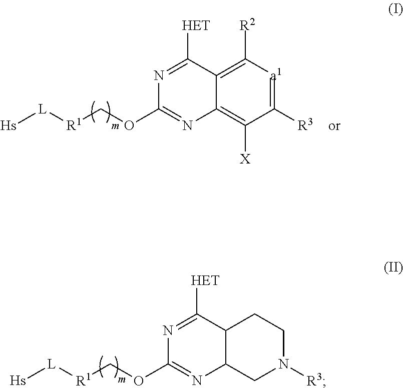

or a pharmaceutically acceptable salt thereof, wherein
   HET is an optionally substituted heterocyclyl;
   Hs is a chemical moiety that binds HSP90;
   X is hydrogen or halo;
   L is a linker;
   m is 0, 1, 2, or 3;
   R¹ is selected from (C₁-C₄)alkylO(C₁-C₄)alkyl, —(C₁-C₄)alkylNH(C₁-C₄)alkyl, —(C₁-C₄)alkylN[(C₁-C₄)alkyl]2, heterocyclyl, and cycloalkyl, wherein said heterocyclyl and cycloalkyl are each optionally and independently substituted;
   R² is selected from hydrogen, (C₁-C₄)alkyl, (C₁-C₄)haloalkyl, (C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]2, —P(O)[(C₁-C₄)alkyl]2, and —S(C₁-C₄)alkyl;
   R³ is selected from

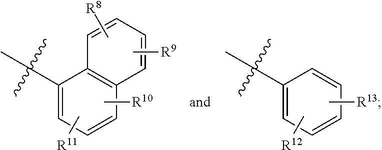

a¹ is N or CHZ¹;
Z¹ is selected from hydrogen, halo, (C₁-C₄)alkyl, (C₂-C₄)alkenyl, cyano, cyano(C₁-C₄)alkyl, —S[halo(C₁-C₄)alkyl], and (C₃-C₆)cycloalkyl;
R⁸, R⁹, R¹⁰, R¹¹, R¹², and R¹³ are each independently selected from hydrogen, (C₁-C₄)alkyl, (C₁-C₄)haloalkyl, (C₁-C₄)cyanoalkyl, (C₁-C₄)hydroxyalkyl, —(C₁-C₄)alkylNR^{a}R^{b}, —(C₁-C₄)alkylC(O)NR^{a}R^{b}, (C₁-C₄)alkylO(C₁-C₄)alkyl, —(C₁-C₄)alkylC(O)OR^{a}, —(C₁-C₄)alkylNR^{a}C(O)OR^{b}, —(C₁-C₄)alkylC(O)R^{a}, —(C₁-C₄)alkylheterocyclyl, —(C₁-C₄)alkylaryl, —(C₁-C₄)alkylheteroaryl, (C₂-C₄)alkenyl, (C₂-C₄)haloalkenyl, (C₂-C₄)cyanoalkenyl, (C₂-C₄)hydroxyalkenyl, —(C₂-C₄)alkenylNR^{a}R^{b}, (C₂-C₄)alkynyl, (C₂-C₄)haloalkynyl, (C₂-C₄)cyanoalkynyl, (C₂-C₄)hydroxyalkynyl, —(C₂-C₄)alkynylNR^{a}R^{b}, (C₁-C₄)alkoxy, (C₁-C₄)haloalkoxy, halo, cyano, oxo, hydroxy, —S(C₁-C₄)alkyl, —S(C₁-C₄)haloalkyl, —NR^{a}R^{b}, —NR^{a}C(O)R^{b}, —C(O)R^{a}, —C(O)OR^{a}, —SO₂R^{a}, —S(O)R^{a}, —SO₂NR^{a}R^{b}, —NR^{a}SO₂R^{b}, (C₃-C₆)cycloalkyl, 5- or 6-membered heteroaryl, and 4- to 6-membered heterocyclyl, wherein said heterocyclyl, aryl, and heteroaryl of —(C₁-C₄)alkylheterocyclyl, —(C₁-C₄)alkylaryl, and —(C₁-C₄)alkylheteroaryl, and said (C₃-C₆)cycloalkyl, 5- or 6-membered heteroaryl, and 4-to 6-membered heterocyclyl are each optionally and independently substituted with 1 to 3 groups selected from R^{c};
R^{a} and R^{b} are each independently selected from hydrogen, (C₁-C₄)alkyl, and (C₁-C₄)haloalkyl, or R^{a} and R^{b}, when on the same nitrogen atom, may be taken together to form a heterocyclyl; and
R^{c} is selected from halo, (C₁-C₄)alkyl, (C₁-C₄)haloalkyl, (C₁-C₄)alkoxy, (C₁-C₄)haloalkoxy, cyano, hydroxyl, oxo, —C(O)OR^{a}, —C(O)R^{a}, —SO₂R^{a}, —S(O)R^{a}, —SO₂NR^{a}R^{b}, —NRaC(O)R^{b}, —NRaSO₂R^{b}, —NR^{a}R^{b}, and NO₂.

### 2. Definitions

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “IC₅₀” or “EC₅₀” refers to an amount, concentration, or dosage of a compound that is required for 50% inhibition of a maximal response in an assay that measures such a response.

The term “compound”, as used herein, unless otherwise indicated, refers to any CHAMP compound disclosed herein. In some aspects one or more hydrogen atoms on a disclosed compound may be replaced with deuterium. Such deuterated compounds may have one or more improved pharmacokinetic or pharmacodynamic properties (e.g., longer half-life) compared to the equivalent “un-deuterated” compound.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkenyl” means a saturated straight chain or branched non-cyclic hydrocarbon having, unless specified otherwise, from 2 to 10 carbon atoms (e.g., (C₂-C₆)alkenyl or (C₂-C₄)alkenyl) and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

The term “aryl” used alone or as part of a larger moiety as in “—(C₁-C₄)alkylaryl”, refers to monocyclic and bicyclic carbon ring systems having a total of six to 10 ring members, wherein at least one ring in the system is aromatic. Examples include, but are not limited to phenyl, naphthyl, anthracyl and the like. It will be understood that when specified, optional substituents on an aryl group may be present on any substitutable position.

As used herein, the term “cycloalkyl” means a saturated, monocyclic alkyl radical having from e.g., 3 to 10 carbon atoms (e.g., from 3 to 6 carbon atoms).

Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

As used herein, the term “halogen” or “halo” means F, Cl, Br or I.

As used herein, the term “heterocyclyl” means a 4- to 12-membered monocyclic or polycyclic saturated or partially unsaturated heterocyclic ring containing 1 to 4 heteroatoms independently selected from N, O, and S. The heterocycle may be attached via any heteroatom or carbon atom, as valency permits.

Representative heterocycles include morpholinyl, thiomorpholinyl, pyrrolidinonyl, pyrrolidinyl, piperidinyl, piperazinyl, oxiranyl, dioxanyl, oxetanyl, dihydrofuranyl, dihydropyranyl, isoindolinyl, dihydropyridinyl, tetrahydrofuranyl, tetrahydropyranyl, tetrahydropyrindinyl, tetrahydropyrimidinyl, diazabicyclooctanyl, hexahydropyrrolizinyl, 2-azaspiro[3.3]heptanyl, 2,7-diazaspiro[3.5]nonanyl, 2-azaspiro[3.5]nonanyl, 3-azabicyclo[3.1.0]hexanyl, 8-azabicyclo[3.2.1]octanyl, 3,8-diazabicyclo[3.2.1]octanyl, 3,6-diazabicyclo[3.1.1]heptanyl, octahydro-1H-pyrrolo[2,3-c]pyridinyl, and the like. Optional substituents on a heterocyclyl group may be present on any substitutable position and, include, e.g., the position at which the heterocyclyl is attached, valence permitting.

As used herein, the term “heteroaryl” means a 5- to 12-membered aromatic radical containing 1-4 heteroatoms selected from N, O, and S. A heteroaryl group may be mono- or bicyclic. The heteroaryl may be attached via any heteroatom or carbon atom, as valency permits. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. Optional substituents on a heteroaryl group may be present on any substitutable position and, include, e.g., the position at which the heteroaryl is attached, valence permitting.

When used in connection to describe a chemical group that may have multiple points of attachment, a hyphen (-) designates the point of attachment of that group to the variable to which it is defined. For example, —(C₁-C₄)alkylaryl and means that the point of attachment for these groups occurs on the alkyl group.

The term “alkylene” refers to a linear or branched saturated divalent hydrocarbon radical. For example, C₁₋₆ alkanediyl refers to a linear saturated divalent hydrocarbon radical of 1 to 6 carbon atoms or a branched saturated divalent hydrocarbon radical of 3 to 6 carbon atoms.

The term “aralkyl” or “arylalkyl” refers to a monovalent alkyl group substituted with one or more aryl groups. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl).

A hash bond as in “ [symbol] ” represents the point at which the depicted group is [symbol] attached to the defined variable.

Unless otherwise indicated, an “optionally substituted” group may have a suitable substituent at each substitutable position of the group, and when more than one position in any given structure may be substituted with more than one substituent selected from a specified group, the substituent may be either the same or different at each position. Optional substituents include, but are not limited to, one or more groups selected from cyano (—CN), halo, imino (═NH), nitro (—NO₂), oxo (═O), —C(O)Rⁱ, —C(O)ORⁱ, —C(O)NRⁱⁱRⁱⁱⁱ, —C(O)SRⁱ, —C(NRⁱ)NRⁱⁱRⁱⁱⁱ, —C(S)Rⁱ, —C(S)ORⁱ, —C(S)NRⁱⁱRⁱⁱⁱ, —ORⁱ, —OC(O)Rⁱ, —OC(O)ORⁱ, —OC(O)NRⁱⁱRⁱⁱⁱ, —OC(O)SRⁱ, —OC(NRⁱ)NRⁱⁱRⁱⁱⁱ, —OC(S)Rⁱ, —OC(S)ORⁱ, —OC(S)NRⁱⁱRⁱⁱⁱ, —OP(O)(ORⁱⁱ)ORⁱⁱⁱ, —OS(O)Rⁱ, —OS(O)₂Rⁱ, —OS(O)NRⁱⁱRⁱⁱⁱ, —OS(O)₂NRⁱⁱRⁱⁱⁱ, —NRⁱⁱRⁱⁱⁱ, —NRⁱC(O)R^{iv}, —NRⁱC(O)OR^{iv}, —NRⁱC(O)NRⁱⁱRⁱⁱⁱ, —NRⁱC(O)SR^{iv}, —NRⁱC(NR^{iv})NRⁱⁱRⁱⁱⁱ, —NRⁱC(S)R^{iv}, —NRⁱC(S)OR^{iv}, —NRWC(S)NRⁱⁱRⁱⁱⁱ, —NRS(O)R^{iv}, —NR'S(O)₂R^{iv}, —NRⁱS(O)NRⁱⁱRⁱⁱⁱ, —NRⁱS(O)₂NRⁱⁱR^{iv}, —SRⁱ, —S(O)Rⁱ, —S(O)₂Rⁱ, —S(O)NRⁱⁱR^{iv}, —S(O)₂NRⁱⁱR^{iv}, alkyl, heteroalkyl, alkenyl, alkynyl, cycloalkyl, aryl, heteroaryl, and heterocyclyl, wherein said alkyl, heteroalkyl, alkenyl, alkynyl, cycloalkyl, aryl, heteroaryl, and heterocyclyl are each further optionally substituted with one or more, in one embodiment, one, two, three, or four, substituents Q^{a}, wherein each Rⁱ, Rⁱⁱ, Rⁱⁱⁱ, and R^{iv} is independently alkyl, heteroalkyl, alkenyl, alkynyl, cycloalkyl, aryl, heteroaryl, or heterocyclyl, each of which is optionally substituted with one or more, in one embodiment, one, two, three, or four, substituents Q^{a} or Rⁱⁱ and Rⁱⁱⁱ together with the N atom to which they are attached form heterocyclyl optionally substituted with one or more, in one embodiment, one, two, three, or four, substituents Q^{a}, wherein each Q^{a} is independently selected from cyano, halo, imino, nitro, oxo, alkyl, heteroalkyl, alkenyl, alkynyl, cycloalkyl, C₆₋₁₄ aryl, heteroaryl, heterocyclyl, —C(O)R^{v}, —C(O)OR^{v}, —C(O)NR^{vi}R^{vii}, —C(O)SR^{v}, —C(NR^{v})NR^{vi}R^{vii}, —C(S)R^{v}, —C(S)OR^{v}, —C(S)NR^{vi}R^{vii}, —OR^{v}, —OC(O)R^{v}, —OC(O)OR^{v}, —OC(O)NR^{vi}R^{vii}, —OC(O)SR^{v}, —OC(NR)NR^{vi}R^{vii}, —OC(S)R^{v}, —OC(S)OR^{v}, —OC(S)NR^{vi}R^{vii}, —OP(O)(OR^{v})OR^{vi}, —OS(O)R^{v}, —OS(O)₂R^{v}, —OS(O)NR^{vi}R^{vii}, —OS(O)₂NR^{v}R^{vii},
NR^{vi}R^{vii}, —NR^{v}C(O)R^{viii}, —NR^{e}C(O)OR^{vi}, —NR^{v}C(O)NR^{vi}R^{vii}, —NR^{v}C(O)SR^{vi}, —NR^{v}C(NR^{viii})NR^{vi}R^{vii}, —NR^{v}C(S)R^{viii}, —NR^{v}C(S)OR^{vi}, —NR^{v}C(S)NR^{vi}R^{vii}, —NR^{v}S(O)R^{viii}, —NR^{v}(O)₂R^{viii}, —NR^{v}S(O)NR^{vi}R^{vii}, —NR^{v}S(O)₂NR^{vi}R^{vii}, —SR^{v}, —S(O)R^{v}, —S(O)₂R^{v}, —S(O)NR^{vi}R^{vii}, and —S(O)₂NR^{vi}R^{vii}; wherein each R^{v}, R^{vi}, R^{vii}, and R^{viii} is independently alkyl, alkenyl, alkynyl, cycloalkyl, aryl, aralkyl, heteroaryl, or heterocyclyl; or (iii) R^{vi} and R^{vii} together with the N atom to which they are attached form heterocyclyl.

The term “linker” or “tether,” used interchangeably, refers to a chemical moiety that joins two other moieties (e.g., a first binding moiety and a second binding moiety). A linker can covalently join a first binding moiety and a second binding moiety. In one aspect, the linker is uncleavable in vivo. In one aspect, the linker comprises one or more cyclic ring systems. In another aspect, the linker comprises an alkyl chain optionally substituted by and/or interrupted with one or more chemical groups. In one aspect, the linker comprises optimal spatial and chemical properties to effectuate optimal therapeutic activity. In one aspect, the linker does not interfere with the ability of the first binding moiety and/or the second binding moiety to bind their respective targets (e.g., HSP90 and KRAS(G12D)). In one aspect, the linker alters the ability of the first binding moiety and/or the second binding moiety to bind their respective targets (e.g., HSP90 and KRAS(G12D)).

The term “KRAS” refers to the protein product of the KRAS proto-oncogene, GTPase gene.

The term “HSP90” refers collectively, individually or in various combinations to the protein products of members of the heat shock protein 90 (90 kDa) gene family, including: HSP90AA1 (HSP90-alpha or HSP90(a), HSP90AB1 (HSP90-beta or HSP90p), HSP90B1 (GRP94) and TRAP1.

The term “KRAS(G12D)” refers to the protein product of the KRAS gene carrying a mutation that results in the glycine amino acid at position 12 of KRAS being replaced by an aspartate.

A “chemical entity which binds KRAS(G12D)” refers to a small molecule or a distinct portion of a larger molecule which binds to a portion of KRAS(G12D). In some aspects, the chemical entity which binds KRAS(G12D) is a small molecule. In some aspects, the chemical entity which binds KRAS(G12D) is a small molecule having a molecular weight of less than 2,000 g/mol. In some aspects, the chemical entity which binds KRAS(G12D) induces a confirmation change in KRAS(G12D).

The term “SOS1” refers to the protein product of the SOS1 gene that functions as a guanine nucleotide exchange factor for RAS proteins.

When used in connection to describe a chemical group that may have multiple points of attachment, a hyphen (-) designates the point of attachment of that group to the variable to which it is defined. For example, —NR^{a}R^{b} and —C(O)NR^{a}(C₁₋₄alkylene)NR^{a}R mean that the point of attachment for these groups occur on the nitrogen atom and carbon atom respectively.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. For example, when a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. In another preferred embodiment, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

In a second embodiment, the CHAMP compounds described herein are of the Formula I:

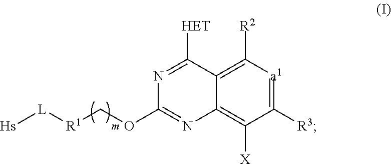

or a pharmaceutically acceptable salt thereof, wherein the remaining variables are as described above.

In a third embodiment, HET in the compounds of Formula I or II, or a pharmaceutically acceptable salt thereof, is optionally substituted 3,8-diazabicyclo[3.2.1]octanyl, wherein the remaining variables are as described above for Formula I and II. Alternatively, as part of the third embodiment, HET in the compounds of Formula I or II, or a pharmaceutically acceptable salt thereof, is optionally substituted 3,8-diazabicyclo[3.2.1]octanyl or 3,6-diazabicyclo[3.2.1]octanyl, wherein the remaining variables are as described above for Formula I and II.

In a fourth embodiment, the CHAMP compounds described herein are of the Formula Ia or IIa:

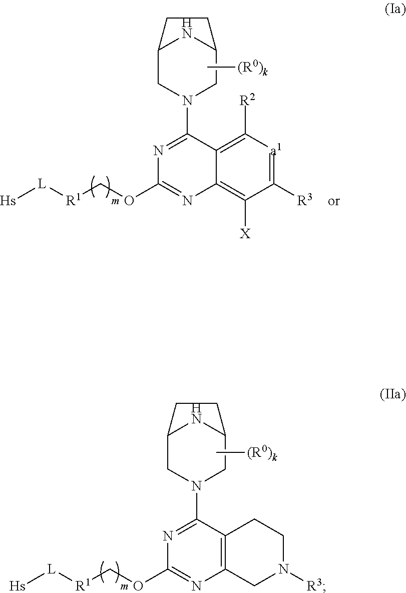

or a pharmaceutically acceptable salt thereof, wherein R⁰ is halo, (C₁-C₄)alkyl, hydroxy(C₁-C₄)alkyl, cyano(C₁-C₄)alkyl, —C(O)H, —C(O)₂H, —C(O)₂(C₁-C₄)alkyl, —C(O)(C₁-C₄)alkyl, —C(O)(C₁-C₄)haloalkyl, —C(O)₂(C₁-C₄)haloalkyl, C(O)₂NH₂, —C(O)₂NH(C₁-C₄)alkyl, —C(O)₂N[(C₁-C₄)alkyl]₂, —S(O)₂(C₁-C₄)alkyl and —S(O)₂(C₁-C₄)haloalkyl, or a 5- to 6-membered optionally substituted heteroaryl; and k is 0, 1, 2, or 3 and wherein the remaining variables are as described above for Formula I and II.

In a fifth embodiment, the CHAMP compounds described herein are of the Formula Ib or IIb:

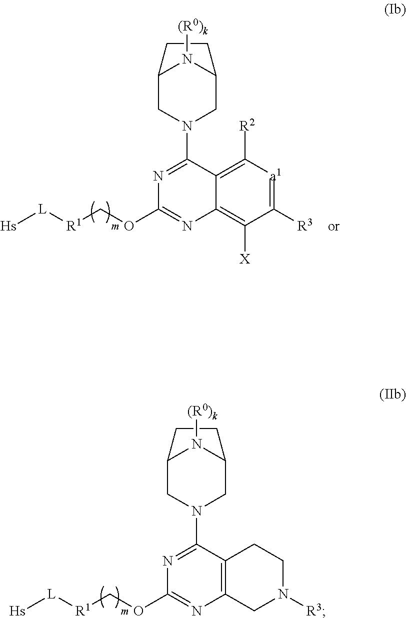

or a pharmaceutically acceptable salt thereof, wherein k is 0 or 1 and wherein the remaining variables are as described above for Formula I and II or the fourth embodiment.

In a sixth embodiment, R⁰ in the compound of Formula Ia, IIa, Ib, or IIb, or a pharmaceutically acceptable salt thereof, is —C(O)(C₁-C₄)alkyl or —C(O)(C₁-C₄)haloalkyl, wherein the remaining variables are as described above for Formula I and II or the fourth embodiment. Alternatively, as part of the sixth embodiment, R⁰ in the compound of Formula Ia, IIa, Ib, or IIb, or a pharmaceutically acceptable salt thereof, is cyano(C₁-C₄)alkyl, —C(O)(C₁-C₄)alkyl, or —C(O)(C₁-C₄)haloalkyl, wherein the remaining variables are as described above for Formula I and II or the fourth embodiment.

In a seventh embodiment, k in the compound of Formula Ia, IIa, Ib, or IIb, or a pharmaceutically acceptable salt thereof, is 0, wherein the remaining variables are as described above for Formula I and II or any one of the fourth or sixth embodiment.

In an eighth embodiment, a¹ in the compound of Formula I, Ia, or Ib, is N, wherein the remaining variables are as described above for Formula I, Ia, or Ib or any one of the fourth, sixth, or seventh embodiments.

In a ninth embodiment, X in the compound of Formula I, Ia, or Ib, or a pharmaceutically acceptable salt thereof, is halo, wherein the remaining variables are as described above for Formula I, Ia, or Ib or any one of the fourth or sixth to eighth embodiments. Alternatively, as part of a ninth embodiment, X in the compound of Formula I, Ia, or Ib, or a pharmaceutically acceptable salt thereof, is fluoro, wherein the remaining variables are as described above for Formula I, Ia, or Ib or any one of the fourth or sixth to eighth embodiments.

In a tenth embodiment, R² in the compound of Formula Ia, IIa, Ib, or IIb, or a pharmaceutically acceptable salt thereof, is hydrogen or (C₁-C₄)alkoxy, wherein the remaining variables are as described above for Formula Ia, IIa, Ib, or IIb or any one of the fourth or sixth to ninth embodiments. Alternatively, as part of a tenth embodiment, R² in the compound of Formula Ia, IIa, Ib, or IIb, or a pharmaceutically acceptable salt thereof, is hydrogen, wherein the remaining variables are as described above for Formula Ia, IIa, Ib, or IIb or any one of the fourth or sixth to ninth embodiments.

In an eleventh embodiment, R⁸, R⁹, R¹⁰, R¹¹, R¹², and R¹³ in the compound of Formula Ia, IIa, Ib, or IIb, or a pharmaceutically acceptable salt thereof, are each independently selected from hydrogen, halo, (C₂-C₄)alkynyl, halo(C₁-C₄)alkyl, (C₁-C₄)alkyl, cyano, (C₁-C₄)alkoxy, and hydroxyl, wherein the remaining variables are as described above for Formula Ia, IIa, Ib, or IIb or any one of the fourth or sixth to tenth embodiments. Alternatively, as part of an eleventh embodiment, R⁸, R⁹, R¹⁰, and R¹¹ in the compound of Formula Ia, IIa, Ib, or IIb, or a pharmaceutically acceptable salt thereof, are each hydrogen; or R⁹, R¹⁰ and R¹¹ are each hydrogen and R⁸ is selected from halo, cyano, hydroxy, (C₁-C₄)alkoxy, and (C₂-C₄)alkynyl; or R⁸, R⁹ and R¹⁰ are each hydrogen and R¹¹ is hydroxy; or R¹⁰ is hydrogen and R⁸, R⁹, and Rⁱⁱ are each independently selected from halo, (C₂-C₄)alkynyl, (C₁-C₄)alkyl, cyano, (C₁-C₄)alkoxy, and hydroxy; or R¹² and R¹³ are each hydrogen; or R¹² is halo(C₁-C₄)alkyl and R¹³ is hydrogen; or R¹² and R¹³ are each independently selected from halo(C₁-C₄)alkyl, (C₁-C₄)alkyl, and halo, wherein the remaining variables are as described above for Formula Ia, IIa, Ib, or IIb or any one of the fourth or sixth to tenth embodiments.

In a twelfth embodiment, R³ in the compound of Formula Ia, IIa, Ib, or IIb, or a pharmaceutically acceptable salt thereof, is selected from

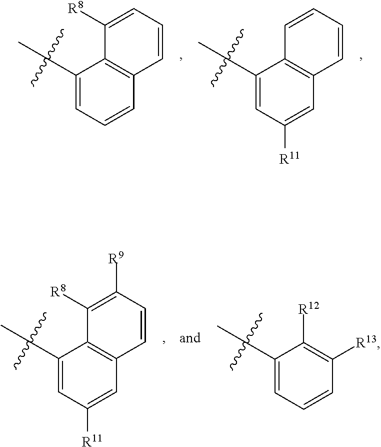

wherein the remaining variables are as described above for Formula Ia, IIa, Ib, or IIb or any one of the fourth or sixth to eleventh embodiments. Alternatively, as part of a twelfth embodiment, R³ in the compound of Formula Ia, IIa, Ib, or IIb, or a pharmaceutically acceptable salt thereof, is selected

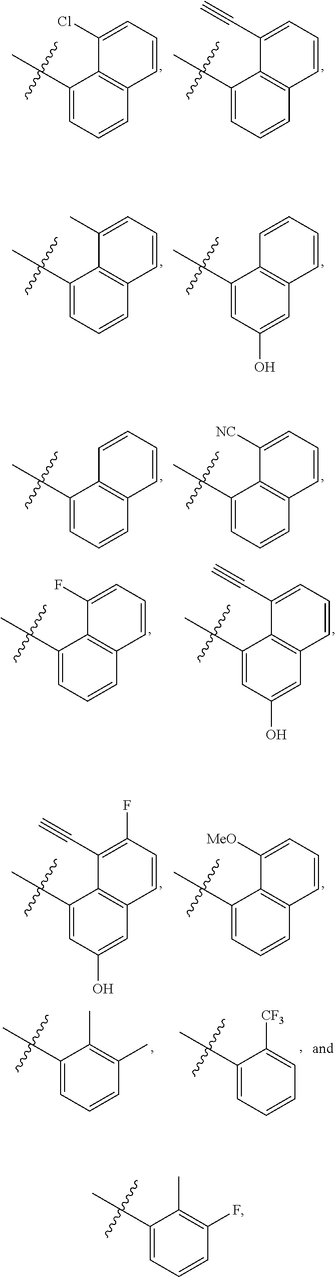

wherein the remaining variables are as described above for Formula Ia, IIa, Ib, or IIb or any one of the fourth or sixth to eleventh embodiments.

In a thirteenth embodiment, R¹ in the compound of Formula Ia, IIa, Ib, or IIb, or a pharmaceutically acceptable salt thereof, is selected from an optionally substituted heterocyclyl and an optionally substituted cycloalkyl, wherein the remaining variables are as described above for Formula Ia, IIa, Ib, or IIb or any one of the fourth or sixth to twelfth embodiments. Alternatively, as part of a thirteenth embodiment, R¹ in the compound of Formula Ia, IIa, Ib, or IIb, or a pharmaceutically acceptable salt thereof, is selected from an optionally substituted 4- to 6-membered nitrogen containing heterocyclyl, an optionally substituted 8- to 10-membered fused bicyclic heterocyclyl, and an optionally substituted (C₃-C₄)cycloalkyl, wherein the remaining variables are as described above for Formula Ia, IIa, Ib, or IIb or any one of the fourth or sixth to twelfth embodiments. In another alternative, as part of a thirteenth embodiment, R¹ in the compound of Formula Ia, IIa, Ib, or IIb, or a pharmaceutically acceptable salt thereof, is selected from azetidinyl, pyrrolidinyl, cyclopropyl, piperazinyl, and hexahydro-1H-pyrrolizinyl, each of which are optionally substituted with 1 to 3 groups selected from (C₁-C₄)alkyl, (C₁-C₄)haloalkyl, (C₁-C₄)alkoxy, C₁-C₄)haloalkoxy, and halo, wherein the remaining variables are as described above for Formula Ia, IIa, Ib, or IIb or any one of the fourth or sixth to twelfth embodiments. In another alternative, as part of a thirteenth embodiment, R¹ in the compound of Formula Ia, IIa, Ib, or IIb, or a pharmaceutically acceptable salt thereof, is selected from pyrrolidinyl, cyclopropyl, piperazinyl, and hexahydro-1H-pyrrolizinyl, each of which are optionally substituted with 1 to 3 groups selected from (C₁-C₄)alkyl, (C₁-C₄)haloalkyl, (C₁-C₄)alkoxy, C₁-C₄)haloalkoxy, and halo, wherein the remaining variables are as described above for Formula Ia, IIa, Ib, or IIb or any one of the fourth or sixth to twelfth embodiments.

In yet another alternative, as part of a thirteenth embodiment, R¹ in the compound of Formula Ia, IIa, Ib, or IIb, or a pharmaceutically acceptable salt thereof, is selected from azetidinyl, pyrrolidinyl, cyclopropyl, piperazinyl, and hexahydro-1H-pyrrolizinyl, each of which are optionally substituted with (C₁-C₄)alkyl, wherein the remaining variables are as described above for Formula Ia, IIa, Ib, or IIb or any one of the fourth or sixth to twelfth embodiments. In yet another alternative, as part of a thirteenth embodiment, R¹ in the compound of Formula Ia, IIa, Ib, or IIb, or a pharmaceutically acceptable salt thereof, is selected from pyrrolidinyl, cyclopropyl, piperazinyl, and hexahydro-1H-pyrrolizinyl, each of which are optionally substituted with (C₁-C₄)alkyl, wherein the remaining variables are as described above for Formula Ia, IIa, Ib, or IIb or any one of the fourth or sixth to twelfth embodiments. In yet another alternative, as part of a thirteenth embodiment, R¹ in the compound of Formula Ia, IIa, Ib, or IIb, or a pharmaceutically acceptable salt thereof, is selected from

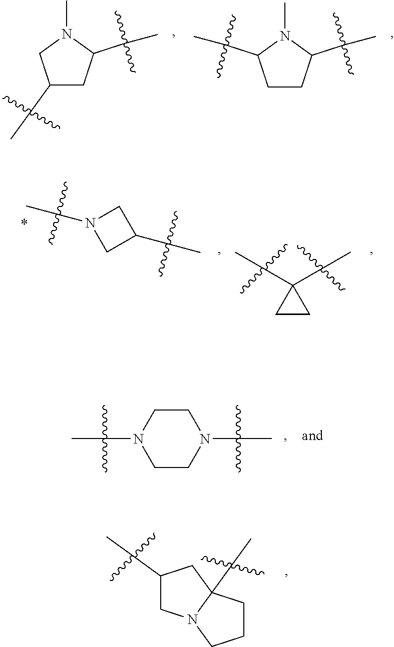

wherein the remaining variables are as described above for Formula Ia, IIa, Ib, or IIb or any one of the fourth or sixth to twelfth embodiments. In yet another alternative, as part of a thirteenth embodiment, R¹ in the compound of Formula Ia, IIa, Ib, or IIb, or a pharmaceutically acceptable salt thereof, is selected from

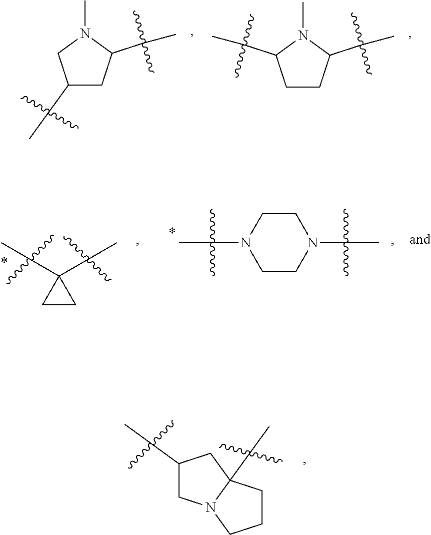

wherein the remaining variables are as described above for Formula Ia, IIa, Ib, or IIb or any one of the fourth or sixth to twelfth embodiments. In yet another alternative, as part of a thirteenth embodiment, R¹ in the compound of Formula Ia, IIa, Ib, or IIb, or a pharmaceutically acceptable salt thereof, is selected from

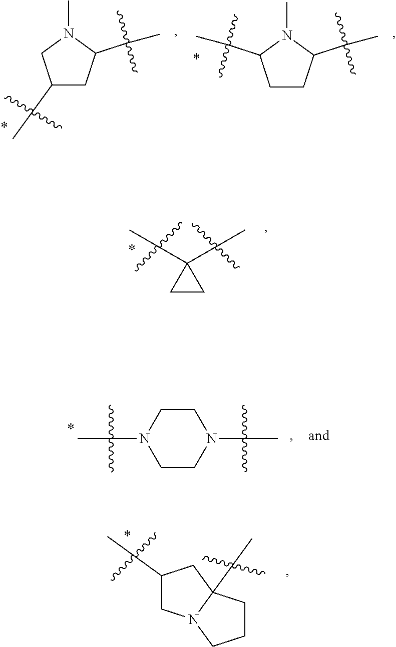

wherein the * indicates the point of attachment to L, wherein the remaining variables are as described above for Formula Ia, IIa, Ib, or IIb or any one of the fourth or sixth to twelfth embodiments.

In a fourteenth embodiment, m in the compound of Formula Ia, IIa, Ib, or IIb, or a pharmaceutically acceptable salt thereof, is 0, 1, or 2, wherein the remaining variables are as described above for Formula Ia, IIa, Ib, or IIb or any one of the fourth or sixth to thirteenth embodiments.

In a fifteenth embodiment, Hs in the compound of Formula Ia, IIa, Ib, or IIb, or a pharmaceutically acceptable salt thereof, is selected from

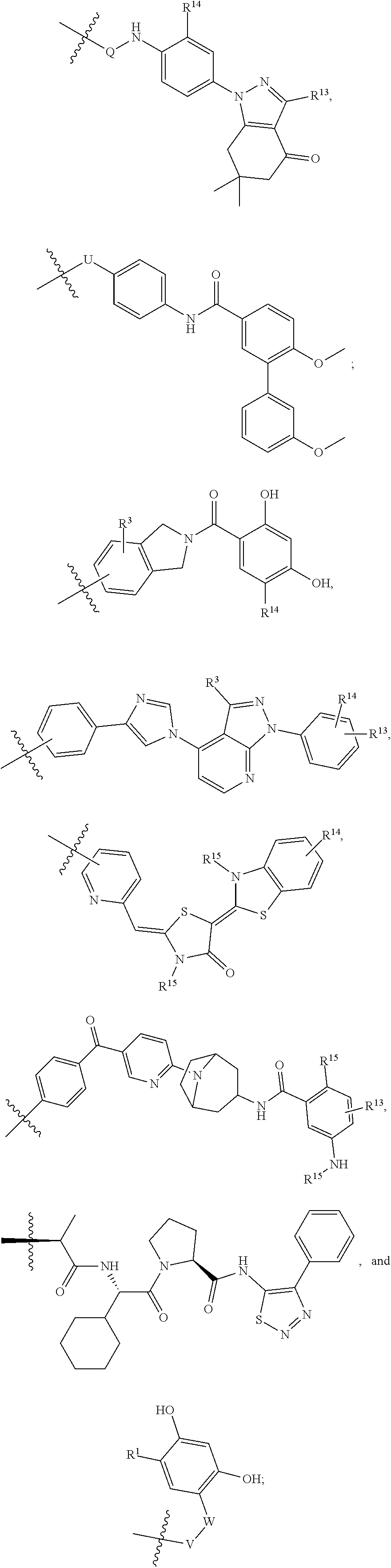

wherein
Q and U are each independently selected from phenyl, heteroaryl, heterocyclyl, and cycloalkyl, each of which being optionally substituted with 1 to 3 groups selected from R¹⁴;
R¹⁷, R¹⁸, R¹⁹ and R²⁰ are each independently selected from hydrogen, halo, CN, (C₁-C₄)alkyl, halo(C₁-C₄)alkyl, and —C(O)NR^{a}R^{b};
R²¹, R²², and R²³ are each independently hydrogen, (C₁-C₄)alkyl, or halo(C₁-C₄)alkyl;
W is 5- or 6-membered heteroaryl optionally substituted with 1 to 3 groups selected from R¹⁴;
V is phenyl or 5- to 9-membered heteroaryl optionally substituted with 1 to 3 groups selected from R¹⁵;
R⁵ is halo, (C₁-C₄)alkyl, halo(C₁-C₄)alkyl, (C₁-C₄)alkoxy, or halo(C₁-C₄)alkoxy;
R¹⁴ is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{e}, —OR^{e}, —C(O)R^{e}, —C(O)OR^{e}, —C(O)NR^{e}R^{f}, —C(O)NR^{e}(C₁₋₄alkylene)OR^{e}, —C(O)NR^{e}(C₁-₄alkylene)NR^{e}R^{f}, —C(O)NR^{e}(C₁₋₄alkylene)OR, —NR^{e}R^{f}, —O(C₁₋₄alkylene)NR^{e}R^{f}, —SH, —S(C₁₋₄alkyl), —C₁₋₄alkylNR^{e}R^{f}, —SR^{e}, —S(O)R^{e}, —S(O)₂R^{e}, —S(O)NR^{e}R^{f}, —SO₂NR^{e}R^{f}, —NR^{e}(C₁₋₄alkyl)OR^{e}, —NR^{e}(C₁₋₄alkyl)NR^{e}R^{f}, —C₁₋₆alkylC(O)NR^{e}R^{f}, phenyl or 5- to 7-membered heteroaryl, wherein said phenyl and 5- to 7-membered heteroaryl are each optionally and independently substituted with 1 to 3 groups selected from R¹⁶;
R^{e} and R^{f} are each independently selected from hydrogen and (C₁-C₄)alkyl, wherein said (C₁-C₄)alkyl is optionally substituted with one or more halo or a 3- to 7-membered heterocyclyl, or both; and
R¹⁵ and R¹⁶ are each independently halo, —NR^{e}R^{f}, (C₁-C₄)alkyl, halo(C₁-C₄)alkyl, (C₁-C₄)alkoxy, or halo(C₁-C₄)alkoxy, wherein the remaining variables are as described above for Formula Ia, IIa, Ib, or IIb or any one of the fourth or sixth to fourteenth embodiments.

Alternatively, as part of the fifteenth embodiment, Hs in the compound of Formula Ia, IIa, Ib, or IIb, or a pharmaceutically acceptable salt thereof, is selected from

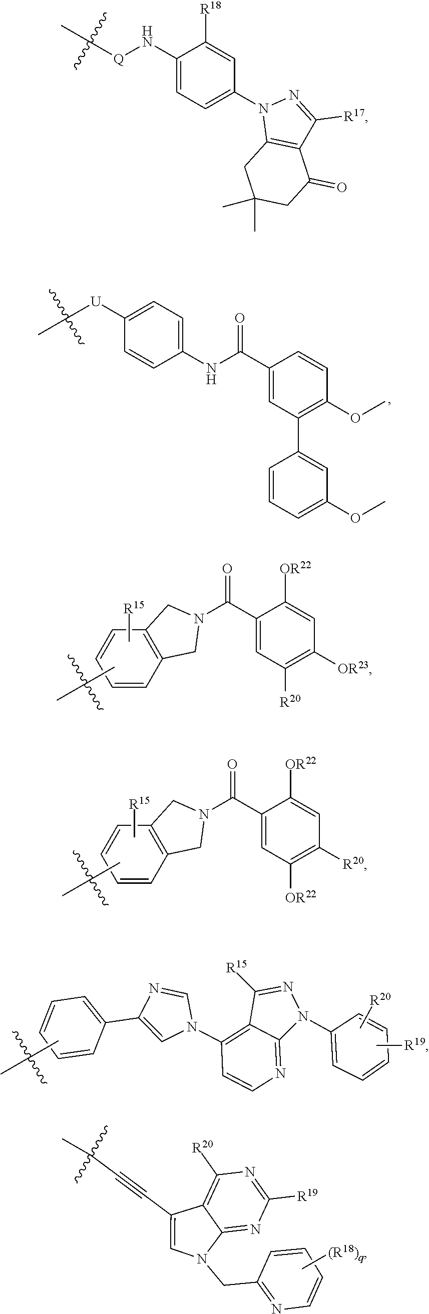

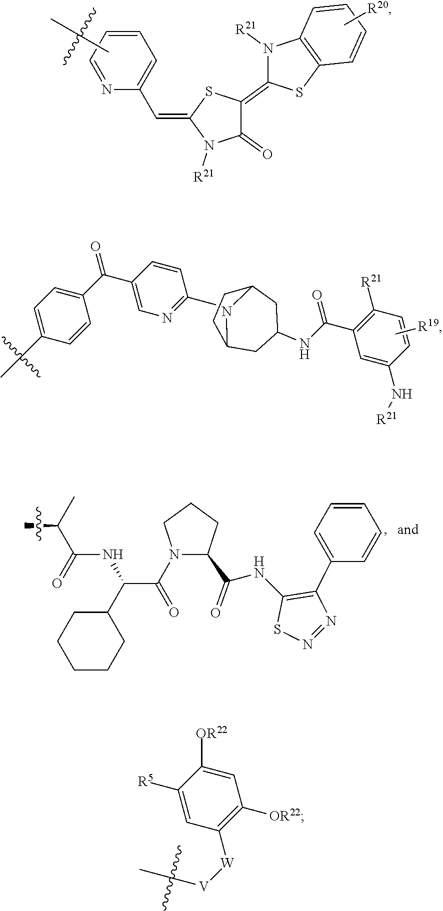

Q and U are each independently selected from phenyl, heteroaryl, heterocyclyl, and cycloalkyl, each of which being optionally substituted with 1 to 3 groups selected from R¹⁴;
R¹⁷, R¹⁸, R¹⁹ and R²⁰ are each independently selected from hydrogen, halo, CN, (C₁-C₄)alkyl, halo(C₁-C₄)alkyl, —NR^{a}R^{b}, —OR^{e}, and —C(O)NR^{a}R^{b};
q is 0, 1, 2, or 3;
R²¹, R²², and R²³ are each independently hydrogen, (C₁-C₄)alkyl, or halo(C₁-C₄)alkyl;
W is 5- or 6-membered heteroaryl optionally substituted with 1 to 3 groups selected from R¹⁴;
V is phenyl or 5- to 9-membered heteroaryl optionally substituted with 1 to 3 groups selected from R¹⁵;
R⁵ is halo, (C₁-C₄)alkyl, halo(C₁-C₄)alkyl, (C₁-C₄)alkoxy, or halo(C₁-C₄)alkoxy;
R¹⁴ is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{e}, —OR^{e}, —C(O)R^{e}, —C(O)OR^{e}, —C(O)NR^{e}R^{f}, —C(O)NR^{e}(C₁₋₄alkylene)OR^{e}, —C(O)NR^{e}(C₁₋₄alkylene)NR^{e}R^{f}, —C(O)NR^{e}(C₁₋₄alkylene)OR, —NR^{e}R^{f}, —O(C₁₋₄alkylene)NR^{e}R^{f}, —SH, —S(C₁₋₄alkyl), —C₁₋₄alkylNR^{e}R^{f}, —SR^{e}, —S(O)R^{e}, —S(O)₂R^{e}, —S(O)NR^{e}R^{f}, —SO₂NR^{e}R^{f}, —NR^{e}(C₁₋₄alkyl)OR^{e}, —NR^{e}(C₁₋₄alkyl)NR^{e}R^{f}, —C₁₋₆alkylC(O)NR^{e}R^{f}, phenyl or 5- to 7-membered heteroaryl, wherein said phenyl and 5- to 7-membered heteroaryl are each optionally and independently substituted with 1 to 3 groups selected from R¹⁶;
   R^{e} and R^{f} are each independently selected from hydrogen and (C₁-C₄)alkyl, wherein said (C₁-C₄)alkyl is optionally substituted with one or more halo or a 3- to 7-membered heterocyclyl, or both; and
   R¹⁵ and R¹⁶ are each independently halo, —NR^{e}R^{f}, (C₁-C₄)alkyl, halo(C₁-C₄)alkyl, (C₁-C₄)alkoxy, or halo(C₁-C₄)alkoxy, wherein the remaining variables are as described above for Formula Ia, IIa, Ib, or IIb or any one of the fourth or sixth to fourteenth embodiments.

In a sixteenth embodiment, Hs in the compound of Formula Ia, IIa, Ib, or IIb, or a pharmaceutically acceptable salt thereof, is

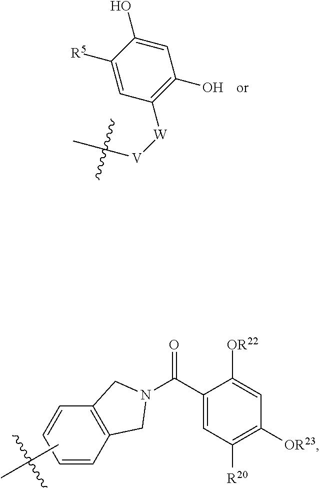

wherein the remaining variables are as described above for Formula Ia, IIa, Ib, or IIb or any one of the fourth or sixth to fifteenth embodiments.

In a seventeenth embodiment, R²⁰ as described in the preceding embodiments is (C₁-C₄)alkyl.

In an eighteenth embodiment, R²² and R²³ as described in the preceding embodiments are each hydrogen, R²² is hydrogen and R²³ is (C₁-C₄)alkyl, or R²³ is hydrogen and R²² is (C₁-C₄)alkyl. Alternatively, as part of the eighteenth embodiment, R²² and R²³ as described in the preceding embodiments are each hydrogen, R²² and R²³ are each (C₁-C₄)alkyl, R²² is hydrogen and R²³ is (C₁-C₄)alkyl, or R²³ is hydrogen and R²² is (C₁-C₄)alkyl.

In a nineteenth embodiment, Hs in the compound of Formula Ia, IIa, Ib, or IIb, or a pharmaceutically acceptable salt thereof, is selected from

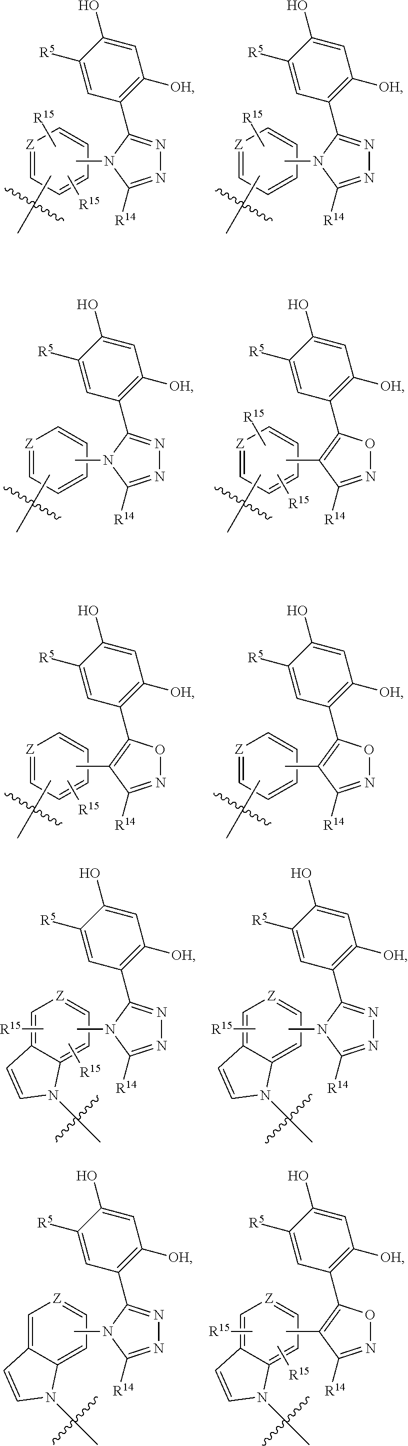

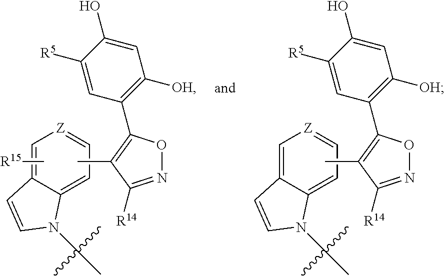

Z is N or CH (preferably CH), wherein the remaining variables are as described above for Formula Ia, IIa, Ib, or IIb or any one of the fourth or sixth to fifteenth embodiments.

In a twentieth embodiment, R¹⁵ as described in the fifteenth through nineteenth embodiments, is independently (C₁-C₄)alkyl or halo.

In a twenty-first embodiment, Hs in the compound of Formula Ia, IIa, Ib, or IIb, or a pharmaceutically acceptable salt thereof, is

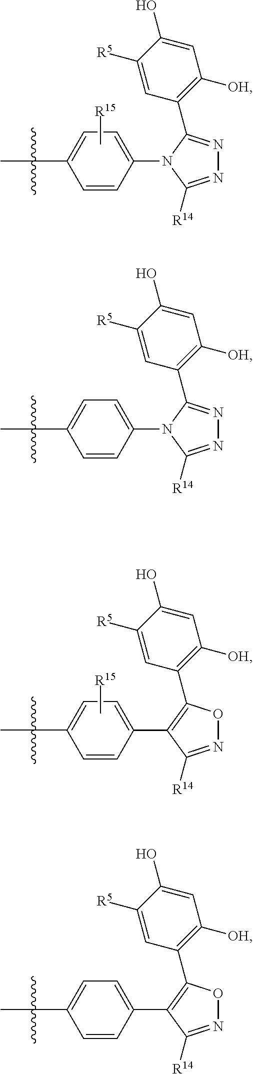

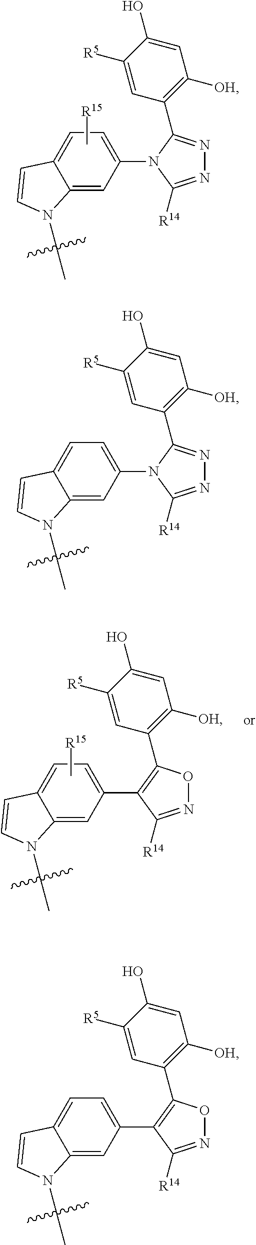

wherein the remaining variables are as described above for Formula Ia, IIa, Ib, or IIb or any one of the fourth or sixth to twentieth embodiments. Alternatively, as part of a twenty-first embodiment, Hs in the compound of Formula Ia, IIa, Ib, or IIb, or a pharmaceutically acceptable salt thereof, is

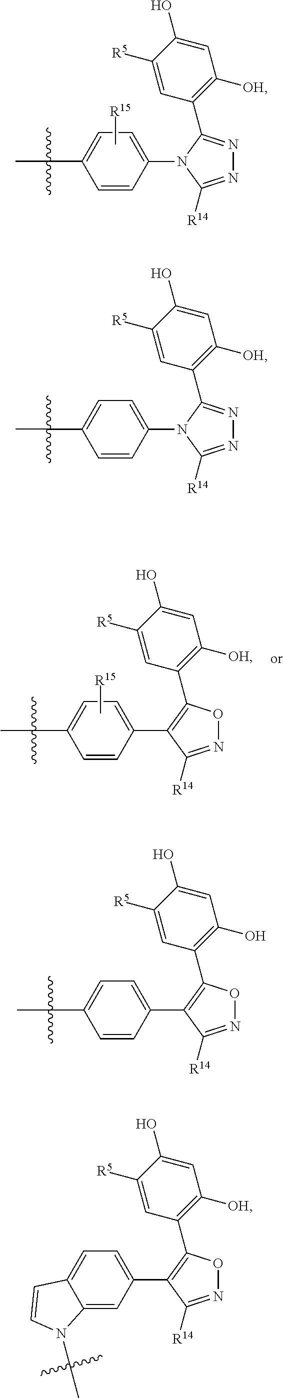

wherein the remaining variables are as described above for Formula Ia, IIa, Ib, or IIb or any one of the fourth or sixth to twentieth embodiments. In another alternative, as part of a twenty-first embodiment, Hs in the compound of Formula Ia, IIa, Ib, or IIb, or a pharmaceutically acceptable salt thereof, is

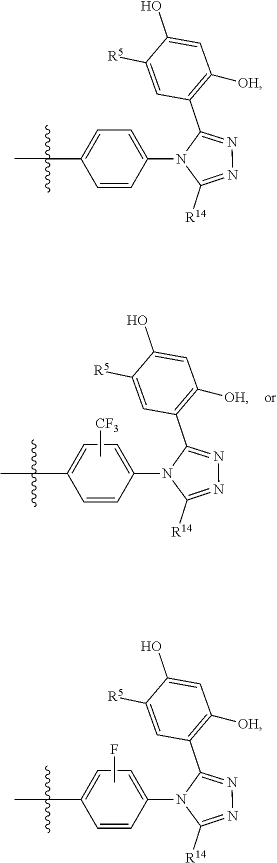

wherein the remaining variables are as described above for Formula Ia, IIa, Ib, or IIb or any one of the fourth or sixth to twentieth embodiments. In yet another alternative, as part of a twenty-first embodiment, Hs in the compound of Formula Ia, IIa, Ib, or IIb, or a pharmaceutically acceptable salt thereof, is

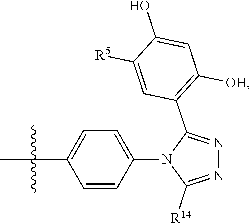

wherein the remaining variables are as described above for Formula Ia, IIa, Ib, or IIb or any one of the fourth or sixth to twentieth embodiments.

In a twenty-second embodiment, R⁵ in any one of the fifteenth to twenty-first embodiments, is halo or (C₁-C₄)alkyl. Alternatively, as part of a twenty-second embodiment, R⁵ in any one of the fifteenth to twenty-first embodiments, is chloro, isopropyl, methyl, propyl, or ethyl. In another alternative, as part of a twenty-second embodiment, R⁵ in any one of the fifteenth to twenty-first embodiments, is isopropyl or ethyl.

In a twenty-third embodiment, R¹⁴ in any one of the fifteenth to twenty-second embodiments, is —OR^{e}, —SR^{e}, —C(O)NR^{e}R^{f}, or —C(O)NR^{e}(C₁₋₄alkylene)NR^{e}R^{f}.

In a twenty-fourth embodiment, R^{e} and R^{f} in any one of the fifteenth to twenty-third embodiments, are each independently selected from hydrogen and (C₁-C₄)alkyl, wherein said (C₁-C₄)alkyl is optionally substituted with 1 to 3 halo or a 6-membered heterocyclyl.

In a twenty-fifth embodiment, R¹⁴ in any one of the fifteenth to twenty-fourth embodiments, is OH, —C(O)NHCH₂CF₃, —C(O)NHCH₂CH₃, —C(O)NHCH(CH₃)₂, —C(O)NH(CH₂CH₃)₂, —C(O)NHCH(CH₃)CF₃, —C(O)NHcyclopropyl, —C(O)NHmethylcyclopropyl, C(O)NH₂, or —C(O)NH(CH₂)₂piperidinyl. Alternatively, as part of a twenty-fifth embodiment, R¹⁴ in any one of the fifteenth to twenty-fourth embodiments, is —C(O)NHCH₂CF₃ or OH. In another alternative, as part of a twenty-fifth embodiment, R¹⁴ in any one of the fifteenth to twenty-fourth embodiments, is OH.

In a twenty-sixth embodiment, Hs in the compound of Formula Ia, IIa, Ib, or IIb, or a pharmaceutically acceptable salt thereof, is selected from

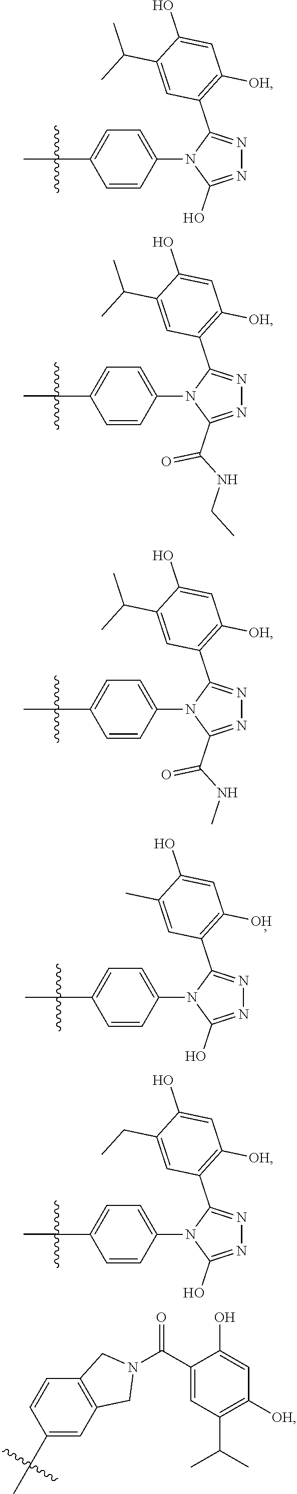

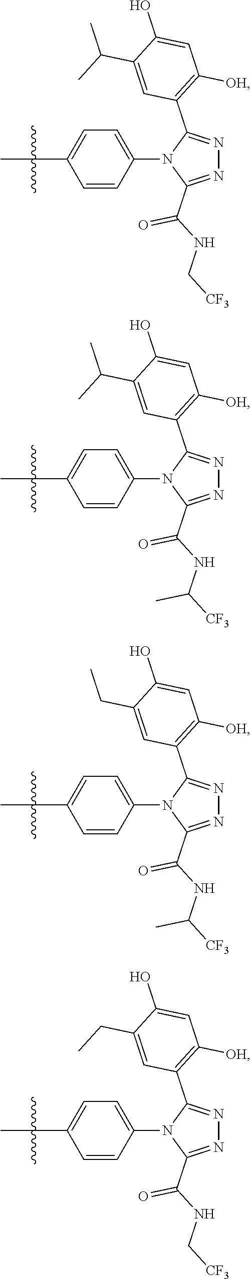 [SMILES: *c1ccc(-n2c(C(=O)NCC(F)(F)F)nnc2-c2cc(C(C)C)c(O)cc2O)cc1 ; *c1ccc(-n2c(C(=O)NC(C)C(F)(F)F)nnc2-c2cc(C(C)C)c(O)cc2O)cc1 ; *c1ccc(-n2c(C(=O)NC(C)C(F)(F)F)nnc2-c2cc(CC)c(O)cc2O)cc1 ; *c1ccc(-n2c(C(=O)NCC(F)(F)F)nnc2-c2cc(CC)c(O)cc2O)cc1]

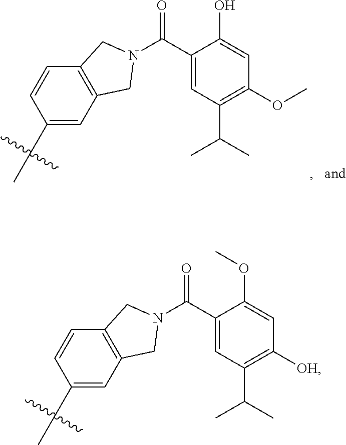 [SMILES: *c1ccc2c(c1)CN(C(=O)c1cc(C(C)C)c(OC)cc1O)C2 ; *c1ccc2c(c1)CN(C(=O)c1cc(C(C)C)c(O)cc1OC)C2]

wherein the remaining variables are as described above for Formula Ia, IIa, Ib, or IIb or any one of the fourth or sixth to fourteenth embodiments. Alternatively, as part of the twenty-sixth embodiment, Hs in the compound of Formula Ia, IIa, Ib, or IIb, or a pharmaceutically acceptable salt thereof, is selected from

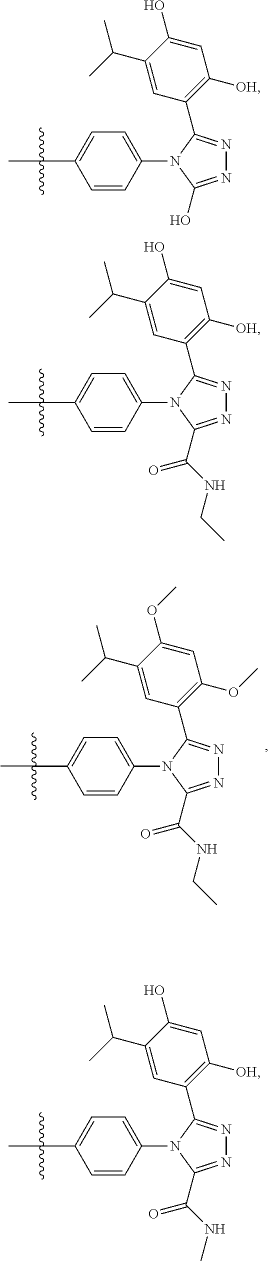

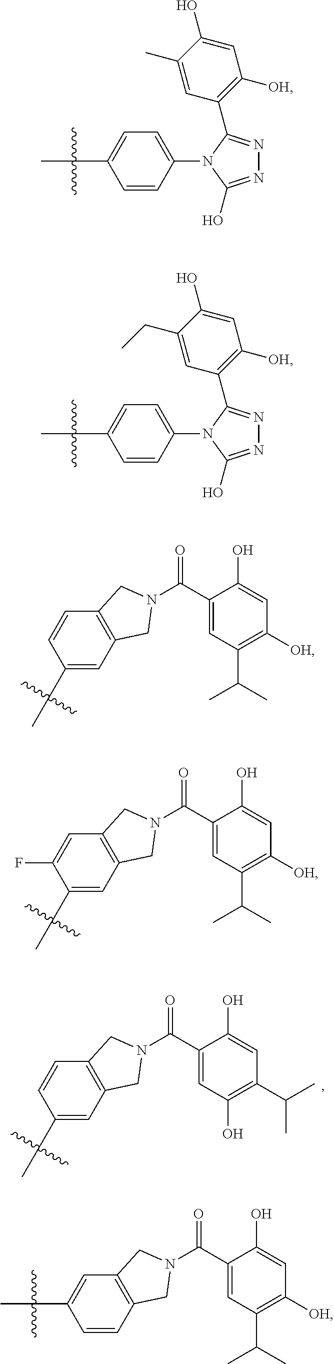 [SMILES: *c1ccc(-n2c(O)nnc2-c2cc(C)c(O)cc2O)cc1 ; *c1ccc(-n2c(O)nnc2-c2cc(CC)c(O)cc2O)cc1 ; *c1ccc2c(c1)CN(C(=O)c1cc(C(C)C)c(O)cc1O)C2 ; *c1cc2c(cc1F)CN(C(=O)c1cc(C(C)C)c(O)cc1O)C2 ; *c1ccc2c(c1)CN(C(=O)c1cc(O)c(C(C)C)cc1O)C2 ; *c1ccc2c(c1)CN(C(=O)c1cc(C(C)C)c(O)cc1O)C2]

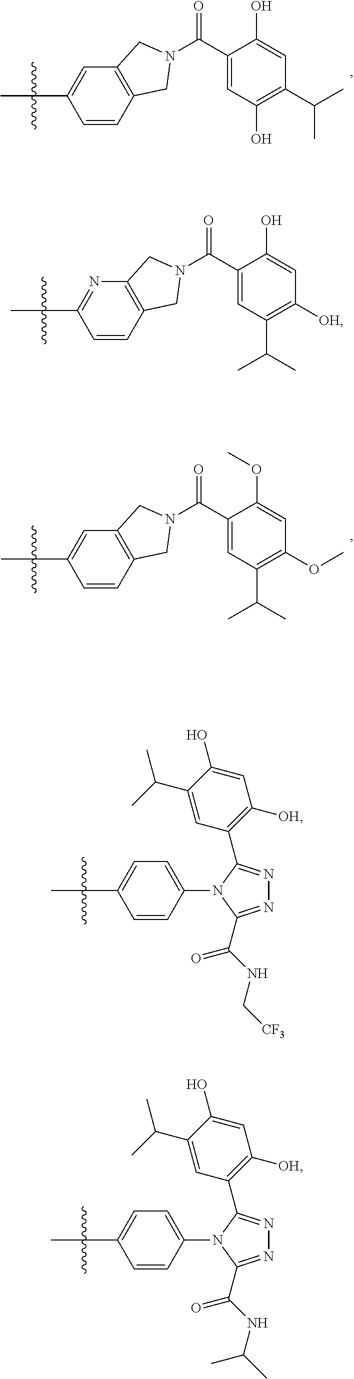 [SMILES: *c1ccc2c(c1)CN(C(=O)c1cc(O)c(C(C)C)cc1O)C2 ; *c1ccc2c(n1)CN(C(=O)c1cc(C(C)C)c(O)cc1O)C2 ; *c1ccc2c(c1)CN(C(=O)c1cc(C(C)C)c(OC)cc1OC)C2 ; *c1ccc(-n2c(C(=O)NCC(F)(F)F)nnc2-c2cc(C(C)C)c(O)cc2O)cc1 ; *c1ccc(-n2c(C(=O)NC(C)C)nnc2-c2cc(C(C)C)c(O)cc2O)cc1]

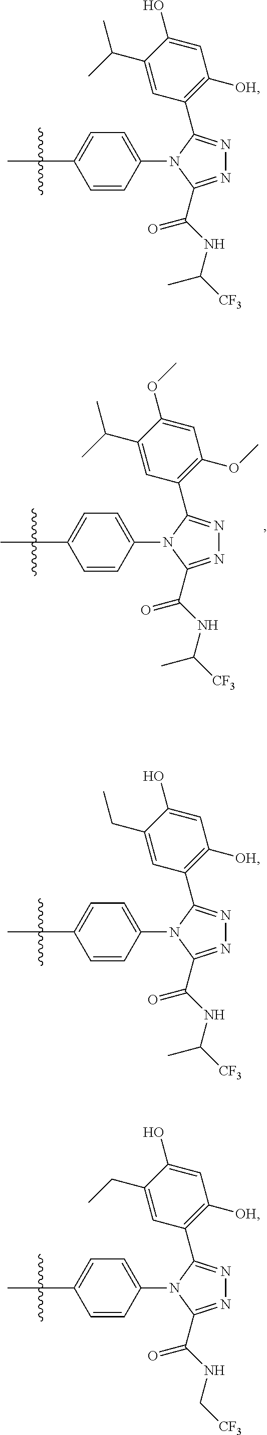 [SMILES: *c1ccc(-n2c(C(=O)NC(C)C(F)(F)F)nnc2-c2cc(C(C)C)c(O)cc2O)cc1 ; *c1ccc(-n2c(C(=O)NC(C)C(F)(F)F)nnc2-c2cc(C(C)C)c(OC)cc2OC)cc1 ; *c1ccc(-n2c(C(=O)NC(C)C(F)(F)F)nnc2-c2cc(CC)c(O)cc2O)cc1 ; *c1ccc(-n2c(C(=O)NCC(F)(F)F)nnc2-c2cc(CC)c(O)cc2O)cc1]

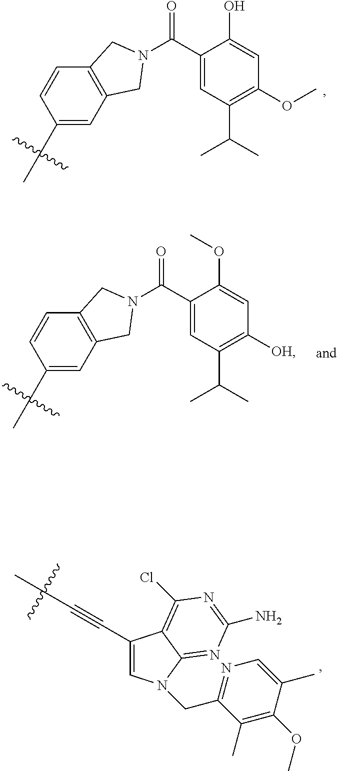 [SMILES: *c1ccc2c(c1)CN(C(=O)c1cc(C(C)C)c(OC)cc1O)C2 ; *c1ccc2c(c1)CN(C(=O)c1cc(C(C)C)c(O)cc1OC)C2 ; *C#Cc1cn(Cc2ncc(C)c(OC)c2C)c2nc(N)nc(Cl)c12]

wherein the remaining variables are as described above for Formula Ia, IIa, Ib, or IIb or any one of the fourth or sixth to fourteenth embodiments.

In a twenty-seventh embodiment, L in the compound of Formula Ia, IIa, Ib, or IIb, or a pharmaceutically acceptable salt thereof, is selected from (C₁-C₆)alkyl, (CH₂CH₂O)ᵥ, (C₁-C₆)alkylNR^{q}, (C₁-C₆)alkylC(O), (C₁-C₆)alkylC(O)O, (C₁-C₆)alkylOC(O), (C₁-C₆)alkylC(O)NR^{q}, (C₁-C₆)alkylNR^{q}C(O), X¹-Het¹-X², X¹-Het¹-X²—X³, X¹-Het¹-X²-Het²-X³—, X¹-Het¹-X²-Het²-X³-Het³-X⁴—, (CH₂CH₂O)ᵥ—X¹-Het¹-X², (CH₂CH₂O)ᵥ—X¹-Het¹-X²—X³, (CH₂CH₂O)ᵥ—X¹-Het¹-X²-Het²-X³—, (CH₂CH₂O)ᵥ—X¹-Het¹-X²-Het²-X³-Het³-X⁴—, (C₁-C₆)alkylNR^{q}—X¹-Het¹-X², (C₁-C₆)alkylNR^{q}—X¹-Het¹-X²—X³, (C₁-C₆)alkylNR^{q}—X¹-Het¹-X²-Het²-X³—, (C₁-C₆)alkylNR^{q}—X¹-Het¹-X²-Het²-X³-Het³-X⁴—, (C₁-C₆)alkylC(O)—X¹-Het¹-X², (C₁-C₆)alkylC(O)—X¹-Het¹-X²—X³, (C₁-C₆)alkylC(O)—X¹-Het¹-X²-Het²-X³—, (C₁-C₆)alkylC(O)—X¹-Het¹-X²-Het²-X³-Het³-X⁴—, (C₁-C₆)alkylC(O)NR^{q}—X¹-Het¹-X², (C₁-C₆)alkylC(O)NR^{q}—X¹-Het¹-X²—X³, (C₁—C)alkylC(O)NR^{q}—X¹-Het¹-X²-Het²-X³—, and (C₁—C)alkylC(O)NR^{q}—X¹-Het¹-X²-Het²-X³-Het³-X⁴—;
each X¹, X², X³, and X⁴ are independently absent or selected from (C₁-C₄)alkylene, O, NR^{q}, and C(O);
v is 1, 2, 3, 4, 5, or 6
R^{q} is hydrogen or (C₁-C₄)alkyl; and
each Het¹, Het², and Het³ are independently selected from a 4- to 6-membered heterocyclyl and (C₃-C₆)cycloalkyl, each optionally substituted with 1 to 3 groups selected from halo, (C₁-C₄)alkyl, (C₁-C₄)haloalkyl, (C₁-C₄)alkoxy, and (C₁-C₄)haloalkoxy, wherein the remaining variables are as described above for Formula Ia, IIa, Ib, or IIb or any one of the fourth or sixth to twenty-sixth embodiments.

Alternatively, as part of the twenty-seventh embodiment, L in the compound of Formula Ia, IIa, Ib, or IIb, or a pharmaceutically acceptable salt thereof, is selected from (C₁-C₆)alkyl, (CH₂CH₂O)ᵥ, (C₁-C₆)alkylNR^{q}, (C₁-C₆)alkylC(O), (C₁-C₆)alkylC(O)O, (C₁-C₆)alkylOC(O), (C₁-C₆)alkylC(O)NR^{q}, (C₁-C₆)alkylNR^{q}C(O), X¹-Het¹-X², X¹-Het¹-X²—X³, X¹-Het¹-X²—X³—X⁴, X¹-Het¹-X²—X³—X⁴—X⁵, X¹-Het¹-X²-Het²-X³—, X¹-Het¹-X²-Het²-X³-Het³-X⁴—, (CH₂CH₂O)ᵥ—X¹-Het¹-X², (CH₂CH₂O)ᵥ—X¹-Het¹-X²—X³, (CH₂CH₂O)ᵥ—X¹-Het¹-X²-Het²-X³—, (CH₂CH₂O)ᵥ—X¹-Het¹-X²-Het²-X³-Het³-X⁴—, (C₁-C₆)alkylNR^{q}—X¹-Het¹-X², (C₁-C₆)alkylNR^{q}—X¹-Het¹-X²—X³, (C₁-C₆)alkylNR^{q}—X¹-Het¹-X²-Het²-X³—, (C₁-C₆)alkylNR^{q}—X¹-Het¹-X²-Het²-X³-Het³-X⁴—, (C₁-C₆)alkylC(O)—X¹-Het¹-X², (C₁-C₆)alkylC(O)—X¹-Het¹-X²—X³, (C₁-C₆)alkylC(O)—X¹-Het¹-X²-Het²-X³—, (C₁-C₆)alkylC(O)—X¹-Het¹-X²-Het²-X³-Het³-X⁴—, (C₁-C₆)alkylC(O)NR^{q}—X¹-Het¹-X², (C₁-C₆)alkylC(O)NR^{q}—X¹-Het¹-X²—X³, (C₁-C₆)alkylC(O)NR^{q}—X¹-Het¹-X²-Het²-X³—, and (C₁-C₆)alkylC(O)NR^{q}—X¹-Het¹-X²-Het²-X³-Het³-X⁴—;
each X¹, X², X³, X⁴, and X⁵ are independently absent or selected from (C₁-C₄)alkylene, O, NR^{q}, and C(O);
v is 1, 2, 3, 4, 5, or 6
R^{q} is hydrogen or (C₁-C₄)alkyl; and
each Het¹, Het², and Het³ are independently selected from a 4- to 6-membered heterocyclyl and (C₃-C₆)cycloalkyl, each optionally substituted with 1 to 3 groups selected from halo, (C₁-C₄)alkyl, (C₁-C₄)haloalkyl, (C₁-C₄)alkoxy, and (C₁-C₄)haloalkoxy, wherein the remaining variables are as described above for Formula Ia, IIa, Ib, or IIb or any one of the fourth or sixth to twenty-sixth embodiments.

In a twenty-eighth embodiment, L in the compound of Formula Ia, IIa, Ib, or IIb, or a pharmaceutically acceptable salt thereof, is selected from (C₁-C₆)alkyl, (C₁-C₆)alkylNR^{q}, X¹-Het²-X², X¹-Het¹-X²—X³, X¹-Het¹-X²-Het²-X³—, and X¹-Het¹-X²-Het²-X³-Het³-X⁴—, wherein the remaining variables are as described above for Formula Ia, IIa, Ib, or IIb or any one of the fourth or sixth to twenty-sixth embodiments. Alternatively, as part of the twenty-eighth embodiment, L in the compound of Formula Ia, IIa, Ib, or IIb, or a pharmaceutically acceptable salt thereof, is selected from (C₁-C₆)alkyl, (C₁-C₆)alkylNR^{q}, X¹-Het²-X², X¹-Het²-X²—X³, X¹-Het¹-X²—X³—X⁴, X¹-Het¹-X²—X³—X⁴—X⁵, X¹-Het¹-X²-Het²-X³—, and X¹-Het¹-X²- Het²-X³-Het³-X⁴—, wherein the remaining variables are as described above for Formula Ia, IIa, Ib, or IIb or any one of the fourth or sixth to twenty-sixth embodiments.

In a twenty-ninth embodiment, each Het¹, Het², and Het³ as described in the twenty-seventh or twenty-eighth embodiments are independently absent or selected from piperidinyl, piperazinyl, cyclohexyl, cyclobutyl, azetidinyl, and pyrrolidinyl, each optionally substituted with 1 to 3 groups selected from halo, (C₁-C₄)alkyl, (C₁-C₄)haloalkyl, (C₁-C₄)alkoxy, and (C₁-C₄)haloalkoxy. Alternatively, as part of a twenty-ninth embodiment, each Het¹, Het², and Het³ are independently absent or selected from piperidinyl, piperazinyl, and pyrrolidinyl. In yet another alternative, as part of the twenty-ninth embodiment, each Het¹, Het², and Het³ as described in the twenty-seventh or twenty-eighth embodiments are independently absent or selected from piperidinyl, piperazinyl, cyclohexyl, cyclopropyl, cyclobutyl, azetidinyl, and pyrrolidinyl, each optionally substituted with 1 to 3 groups selected from halo, (C₁-C₄)alkyl, (C₁-C₄)haloalkyl, (C₁-C₄)alkoxy, and (C₁-C₄)haloalkoxy.

In a thirtieth embodiment, L in the compound of Formula Ia, IIa, Ib, or IIb, or a pharmaceutically acceptable salt thereof, is selected from —CH₂—, CH₂N(CH₃),

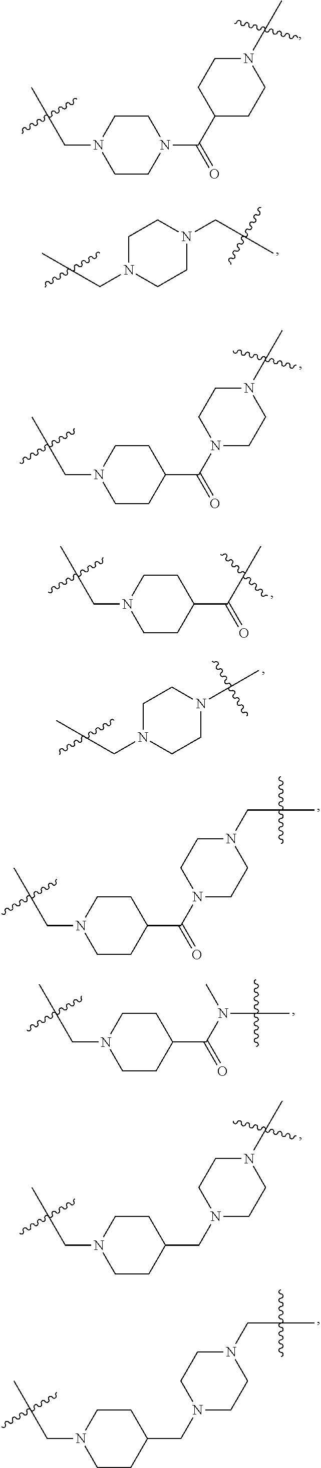

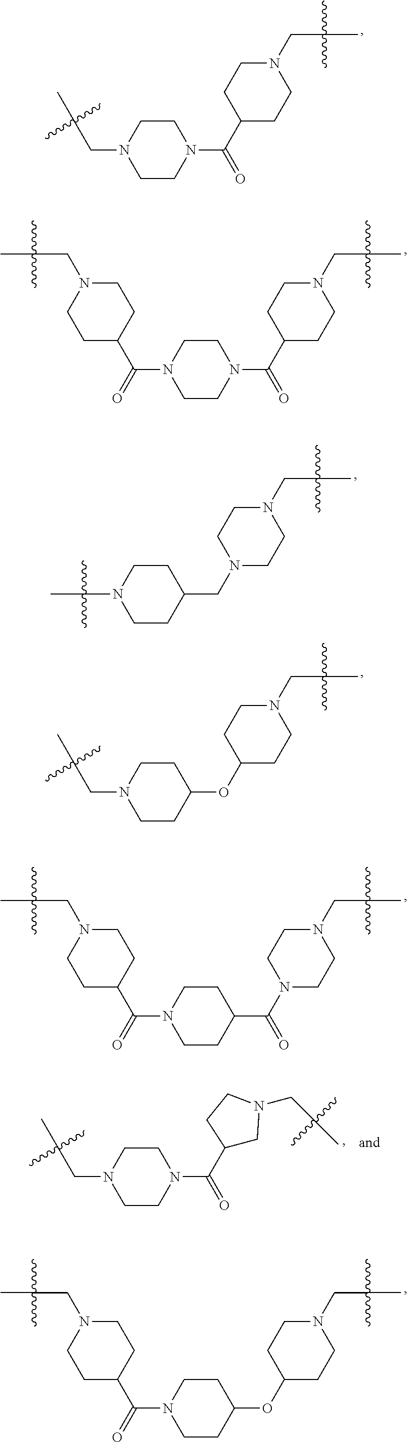

wherein the remaining variables are as described above for Formula Ia, IIa, Ib, or IIb or any one of the fourth or sixth to twenty-sixth embodiments. Alternatively, as part of a thirtieth embodiment, L in the compound of Formula Ia, IIa, Ib, or IIb, or a pharmaceutically acceptable salt thereof, is selected from —CH₂—, *CH₂N(CH₃),

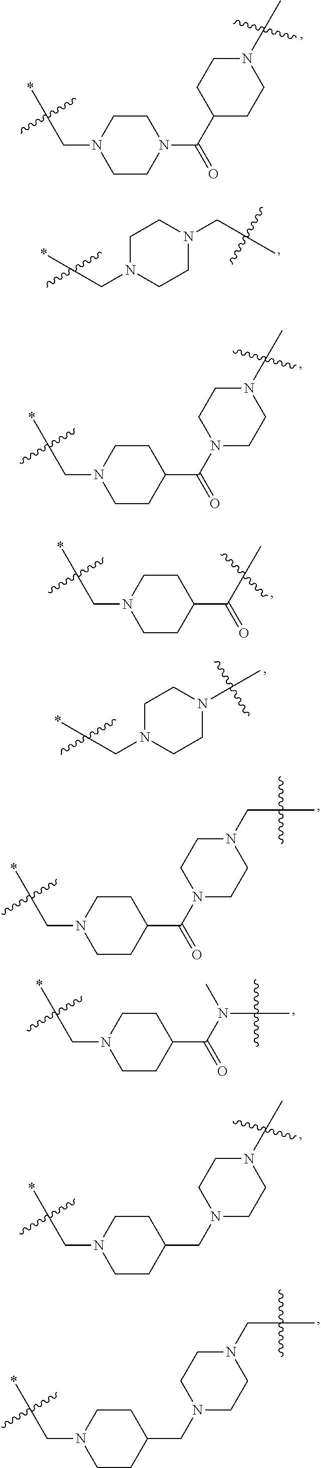

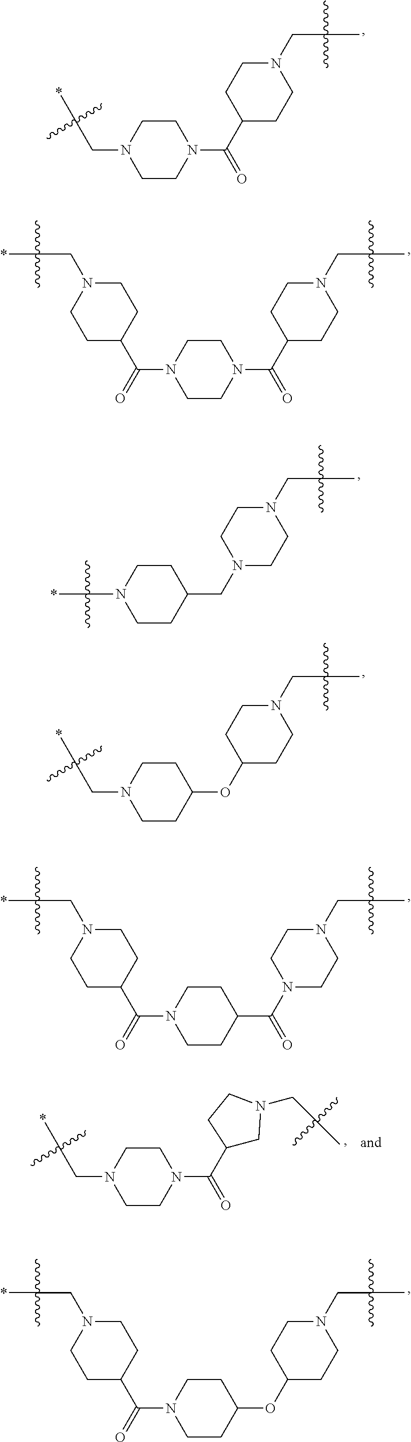

wherein the * indicates the point of attachment to Hs, wherein the remaining variables are as described above for Formula Ia, IIa, Ib, or IIb or any one of the fourth or sixth to twenty-sixth embodiments. In yet another alternative, as part of a thirtieth embodiment, L in the compound of Formula Ia, IIa, Ib, or IIb, or a pharmaceutically acceptable salt thereof, is selected from —CH₂—, *CH₂N(CH₃),

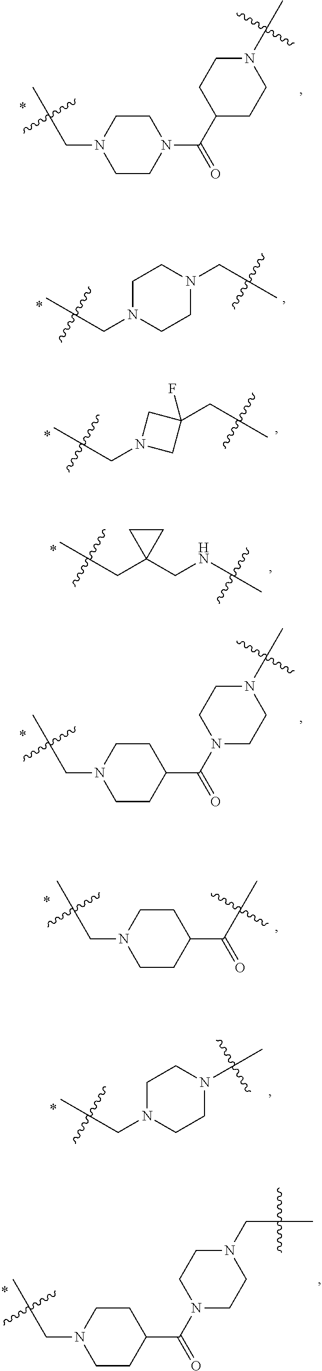

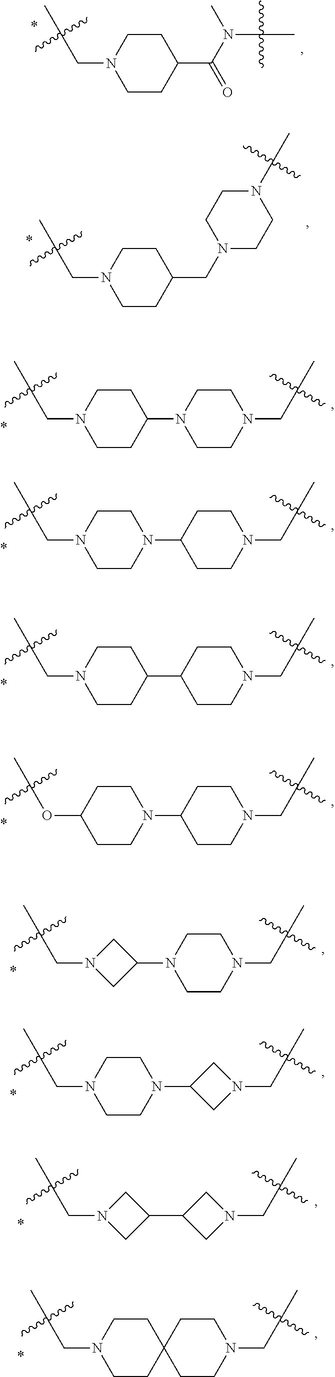

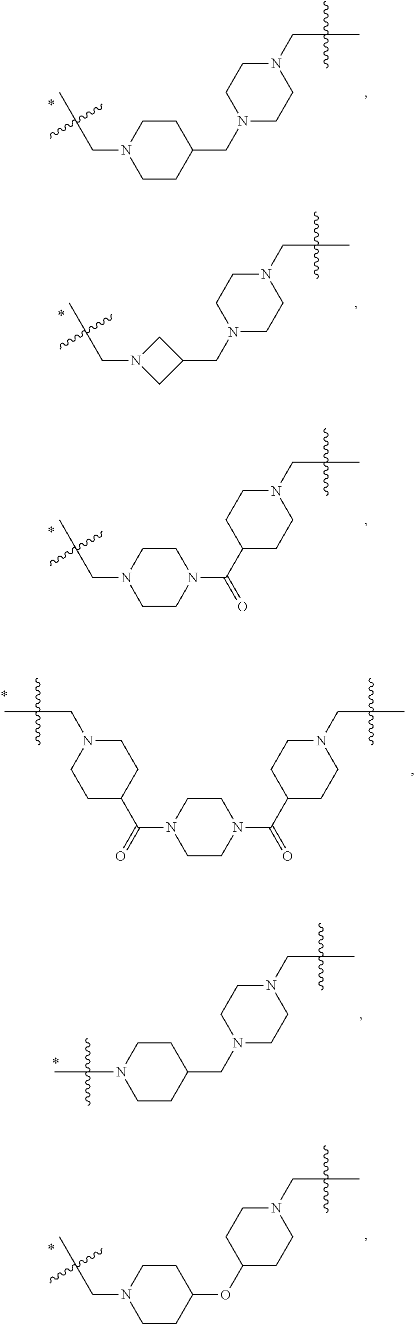 [SMILES: *CN1CCC(CN2CCN(C*)CC2)CC1 ; *CN1CCN(CC2CN(C*)C2)CC1 ; *CN1CCC(C(=O)N2CCN(C*)CC2)CC1 ; *CN1CCC(C(=O)N2CCN(C(=O)C3CCN(C*)CC3)CC2)CC1 ; *CN1CCN(CC2CCN(*)CC2)CC1 ; *CN1CCC(OC2CCN(C*)CC2)CC1]

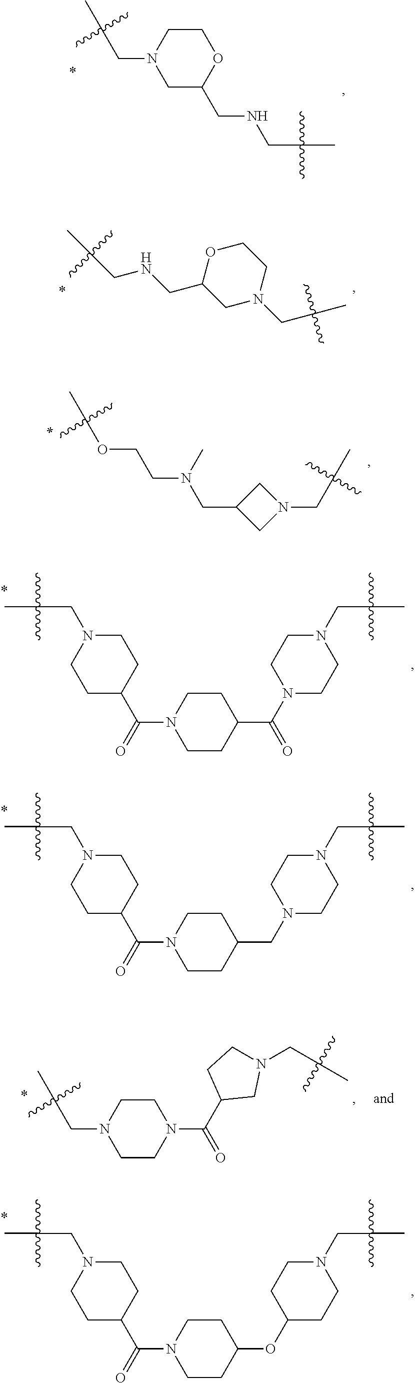

wherein the * indicates the point of attachment to Hs, wherein the remaining variables are as described above for Formula Ia, IIa, Ib, or IIb or any one of the fourth or sixth to twenty-sixth embodiments.

Additional compounds are further disclosed in the Exemplification and are included in the present disclosure. Pharmaceutically acceptable salts thereof as well as the neutral forms are included.

### 4. Uses, Formulation and Administration

Compounds and compositions described herein are generally useful as anticancer therapies. In one aspect, the disclosed compounds and compositions behave as chaperone-mediated protein degraders (CHAMPs) in which one portion of the compounds is responsible for binding KRAS(G12D) and the other portion is responsible for binding to HSP90 or other chaperone proteins or protein components of chaperone complexes (e.g., members of the HSP70 family). Their mechanisms of action include, but are not limited to, degrading KRAS(G12D) and thereby impeding down-stream signals that may result in inhibition of cancer cell growth and/or induction of cancer cell death or other KRAS or KRAS(G12D) functions. In one aspect, the disclosed compounds effectuate the degradation of KRAS(G12D).

In one aspect, the disclosed compounds and compositions include chaperone or chaperone complex binders that have a range of different binding affinities. In different embodiments, it is desirable to use a high-affinity binder, a moderate-affinity binder or a low-affinity binder. Since a HSP90-binding moiety that interacts with the N-terminal ATP-binding pocket of HSP90 may inhibit HSP90 activity and induce the degradation of HSP90 client proteins (Schopf et al., Nat Rev Mol Cell Biol, 2017, 18:345-360), some CHAMP molecules may not only induce the degradation of the specified target protein or proteins (which may or may not be HSP90 client proteins), but also simultaneously induce the degradation of HSP90 client proteins. EGFR and ERBB2 (HER2) are two such HSP90 client proteins (Xu et al., J Biol Chem, 2001, 276:3702-3708). Such combinations of degradation activities may increase the biological activity of CHAMP molecules over that of other TPD technologies directed towards the same target(s) and may evade mechanisms of resistance to KRAS(G12D) inhibitors and degraders such as that mediated by the EGFR pathway.

In one aspect, the disclosed compounds and compositions behave as tumor-targeted CHAMPs in which one portion of the compounds is responsible for binding KRAS(G12D) and the other portion is responsible for binding to HSP90 or other chaperone proteins or protein components of chaperone complexes (e.g., members of the HSP70 family). In one aspect, the disclosed compounds and compositions have prolonged pharmacokinetic exposures in cancer cells and tumors relative to normal cells, tissues and organs (Kamal et al., Nature, 2003, 425:407-410; Vilenchik et al., Chem Biol, 2004, 11:787-797). In one aspect, the disclosed compounds have increased therapeutic indexes relative to other KRAS(G12D) degraders and inhibitors.

Thus, provided herein are methods of treating conditions which are responsive to the degradation or inhibition of KRAS(G12D) comprising administering to a subject in need thereof, a therapeutically effective amount of one or more compounds or compositions described herein. Also provided is the use of one or more compounds or compositions described herein in the manufacture of a medicament for treating conditions which are responsive to the degradation or inhibition of KRAS(G12D). Further provided is the use of a compound or composition described herein for treating conditions which are responsive to the degradation or inhibition of KRAS(G12D).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Chemical Synthesis

The representative examples that follow are intended to help illustrate the present disclosure, and are not intended to, nor should they be construed to, limit the scope of the invention. General starting materials were obtained from commercial sources or prepared in other examples, unless otherwise noted.

### Preparation of Intermediates

The following schemes and synthetic procedures illustrate the synthesis of intermediates used in the synthesis of claimed compounds. Other intermediates can be prepared in a similar manner.

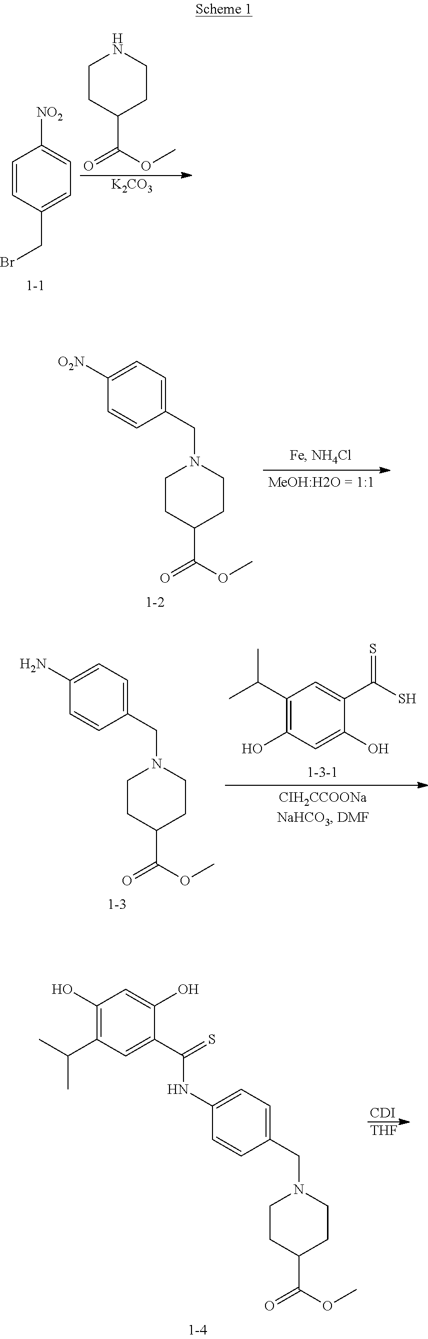

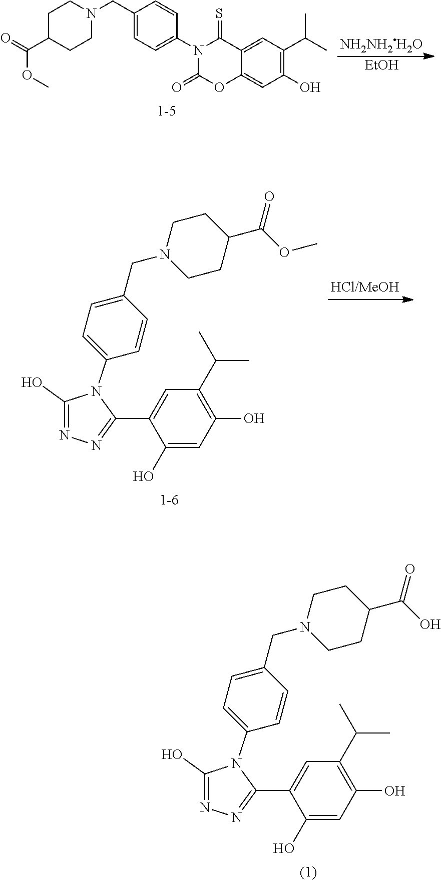

### Step 1: Methyl 1-(4-nitrobenzyl)piperidine-4-carboxylate (1-2)

To the mixture of 1-(bromomethyl)-4-nitrobenzene (40 g, 185.16 mmol) and methyl piperidine-4-carboxylate (26.51 g, 185.16 mmol) in DMF (300 mL) was added K₂CO₃ (51.18 g, 370.22 mmol). The mixture was stirred at r.t. overnight. The mixture was poured into 500 mL of H2O. It was extracted with ethyl acetate (500 mL*2). Combined organic phase was washed with H₂O and brine. It was dried (Na₂SO₄), filtered and concentrated to give desired 1-2 as yellow solid (49 g).

### Step 2: methyl 1-(4-aminobenzyl)piperidine-4-carboxylate (1-3)

To the mixture of methyl 1-(4-nitrobenzyl)piperidine-4-carboxylate (5.0 g, 17.97 mmol) and NH₄Cl (4.81 g, 89.83 mmol) in MeOH/H₂O (25 mL/25 mL) was added Fe powder (5.02 g 89.83 mmol). The mixture was stirred at reflux overnight. After cooled to r.t., the mixture was filtered and the filtrate was concentrated. It was extracted with ethyl acetate (50 mL*2). Combined organic phase was washed with H2O and brine. It was dried (Na₂SO₄), filtered and concentrated to give 1-3 as brown oil (3.8 g)

### Step 3: Methyl 1-(4-(2,4-dihydroxy-5-isopropylphenylthioamido)benzyl)piperidine-4-carboxylate (1-4)

To a solution of compound 3a (20.69 g, 90.61 mmol), ClCH₂COONa (10.55 g, 90.61 mmol) and NaHCO₃ (15.22 g, 181.22 mmol) in DMF (100 mL) was stirred at 40° C. for 3 hours. Compound 1-3 (15.0 g, 60.41 mmol) in DMF (50 mL) was added to the mixture. After the resulting mixture was heated at 80° C. overnight. The reaction mixture was poured into water and solid precipitated was collected by filtration. The mother liquid was extracted with ethyl acetate (200 mL*2). Combined organic phase was washed with H2O and brine. It was dried (Na₂SO₄), filtered and concentrated. It was combined with the solid filtered and purified by SGC to give 1-4 (22.1 g) as a yellow solid.

### Step 4: methyl 1-(4-(7-hydroxy-6-isopropyl-2-oxo-4-thioxo-2H-benzo[e][1,3]oxazin-3(4H)-yl)benzyl)piperidine-4-carboxylate (1-5)

The solution of compound 1-4 (8.0 g, 18.08 mmol) and CDI (11.72 g, 72.3 mmol) in THF (80 mL) was stirred for 2 hours at room temperature. The reaction solution was poured into brine (1 L) and extracted with EtOAc (200 mL*3). The combine organic layers was washed with brine, dried over Na₂SO₄ and concentrated to give the 1-5 (crude) which was used for further reaction without purification.

### Step 5: Methyl-1-(4-(3-(2,4-dihydroxy-5-isopropylphenyl)-5-hydroxy-4H-1,2,4-triazol-4-yl)benzyl)piperidine-4-carboxylate (1-6)

To a solution of compound 1-5 (8.0 g, 17.07 mmol) in EtOH (80 mL) was added NH₂NH₂H2O (1.11 g, 22.2 mmol). Then the resulting mixture was stirred at room temperature overnight. The precipitated solid was filtered and dried to give 1-6 (4 g) as a white solid.

### Step 6: 1-(4-(3-(2,4-dihydroxy-5-isopropylphenyl)-5-hydroxy-4H-1,2,4-triazol-4-yl)benzyl)piperidine-4-carboxylic acid; Intermediate (1)

To a solution of compound 1-6 (4.0 g 8.57 mmol) in MeOH/THF (1:1, 30 mL) was added the solution (15 mL) of LiOH H₂O (1.8 g, 42.87 mmol). Then the resulting mixture was stirred at room temperature for 3 hours. The pH was adjusted to 5-6 with 2N HCl. The precipitated solid was filtered and dried to give intermediate (1) (2.5 g) as a white solid.

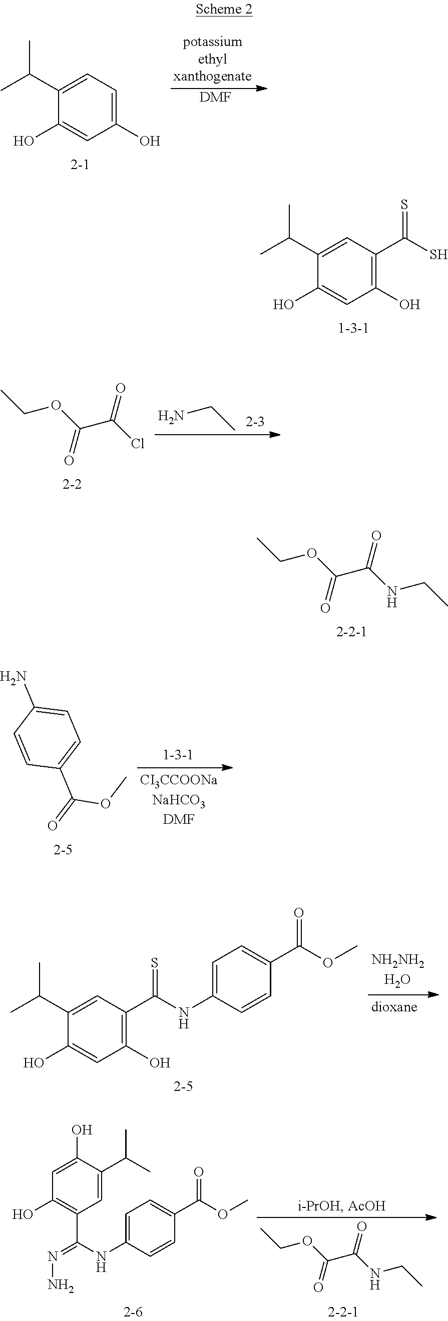

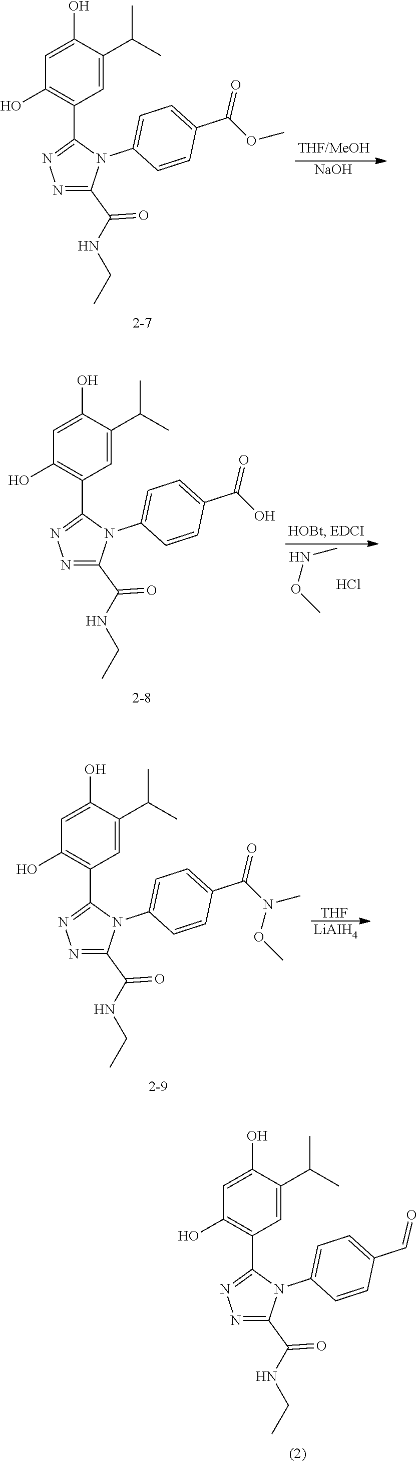

### Preparation of Compound 1-3-1

To a solution of 4-isopropylbenzene-1,3-diol (100 g, 658 mmol) in DMF (500 mL) was added compound potassium O-ethyl carbonodithioate (137 g, 855 mmol). The resulting mixture was stirred at 100° C. for 14 h under argon. It was poured into ice water (2 L), adjusted the pH to 2 with 2 N HCl solution and yellow solid formed. The solid was washed with water and brine, dried to give compound 1-3-1 as yellow solid (120 g, 80% yield)

### Preparation of Compound 2-2-1

At 0° C., triethylamine (7.78 g, 76.9 mmol) and ethylamine hydrochloride (2.98 g, 36.6 mmol) were dissolved in dichloromethane (100 mL). Oxalyl chloride monoethyl ester (5.0 g, 36.6 mmol) was slowly added dropwise to the reaction system at 0° C. for 2 hours. After the reaction, filter, the reaction solution was diluted with methylene chloride, and then washed successively with saturated sodium bicarbonate solution, Washed with brine. The organic phase was dried over anhydrous sodium sulfate, filter, concentrate to give compound 2-2-1 (5 g).

### Step 1: Compound 2-5

To a solution of compound 1-3-1 (10 g, 44 mmol), ClCH₂COONa (7 g, 60 mmol) and NaHCO₃ (10.1 g, 120 mmol) in DMF (80 mL) was stirred at 40° C. for 3 hours. Compound 2-4 (6.0 g, 40 mmol) was added to the reaction. After the resulting mixture was heated at 80° C. overnight. The reaction mixture was poured into ice-water solid precipitated was collected by filtration to give compound 2-5 (7.0 g, 50% yield) as a yellow solid. MS-ESI (m/z) 346.2 [M+H]⁺.

### Step 2: Compound 2-6

The solution of compound 2-5 (7 g, 20 mmol) and NH₂NH₂ H₂O (2 g, 40 mmol) in dioxane (70 mL) was stirred for 6 hours or overnight at 30° C. The reaction solution was poured into H₂O and extracted with EtOAc (200 mL*3). The combine organic layers was washed with brine, dried over Na₂SO₄ and concentrated to give the Compound 2-6 (7 g, crude) which was used for further reaction without purification.

MS-ESI (m/z) 344.1 [M+H]⁺.

### Step 3: Compound 2-7

To a solution of compound 2-6 (7 g, 20 mmol) in i-PrOH (100 mL) was added compound 2-2-1 (4.35 g, 30 mmol) and AcOH (0.5 mL). Then the resulting mixture was stirred at reflux overnight. It was concentrated and purified by silica gel chromatography (DCM:MeOH=20:1) to give compound 2-7 (6 g) as yellow solid.

MS-ESI (m/z) 425.2 [M+H]⁺.

### Step 4: Compound 2-8

The solution of compound 2-7 (7.3 g, 17.2 mmol) in THF/MeOH (1:1, 100 mL) was added NaOH solution (3.4 g, 50 mL). The mixture was stirred at room temperature overnight. It was concentrated to remove THF and MeOH. 1 N HCl was added to adjust the pH to 3. The solid precipitated was collected by filtration and dried to give compound 2-8 (4.4 g) as yellow solid. MS-ESI (m/z) 411.3 [M+H]⁺.

### Step 5: Compound 2-9

The solution of compound 2-8 (4.43 g, 10.79 mmol) in DMF (36 mL) was added N,O-dimethylhydroxylamine HCl salt (1.16 g, 11.87 mmol) and DIEA (4.19 g, 32.38 mmol). HOBt (1.75 g, 12.95 mmol) and EDCI (2.48 g, 12.95 mmol) was added. The mixture was stirred at r.t. overnight. The reaction solution was poured into H₂O and extracted with EtOAc (200 mL*3). The combine organic layers was washed with brine, dried over Na₂SO₄ and concentrated then purified by silica gel chromatography (DCM:MeOH=20:1) to give compound 2-9 (1.8 g) as yellow solid. MS-ESI (m/z) 454.2 [M+H]⁺.

### Step 6: Intermediate (2)

The solution of compound 2-9 (5.1 g, 12.8 mmol) in THF (50 mL) was added LiAlH₄ (2.5 M in THF, 13.8 mL) at 0° C. and stirred at room temperature for 1 hour. The reaction solution was quenched with H₂O (1 g) 10% NaOH (1 mL) and H₂O (3 mL). Brine was added and extracted with THF (40 mL*3). The combined organic phase was dried (Na₂SO₄) and concentrated to give intermediate (2) (1.7 g) as a white solid. MS-ESI (m/z) 395.2 [M+H]⁺.

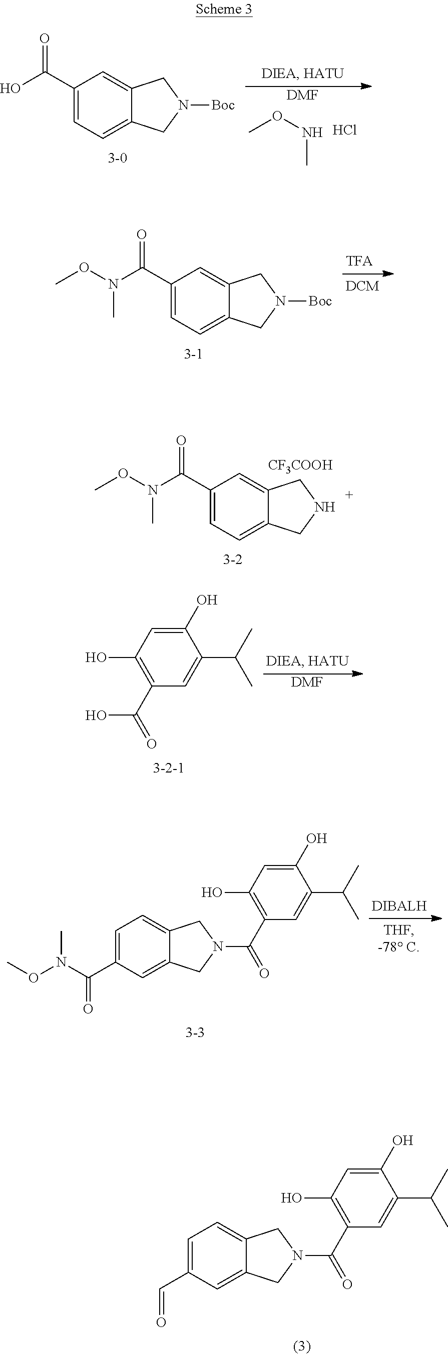

### Step 1: Compound 3-1

The solution of compound 3-0 (93 g, 353 mmol) in DMF (1 L) was added N,O-dimethylhydroxylamine HCl salt (37.9 g, 388.5 mmol), DIEA (137 g, 1.06 mol) and HATU (147.7 g, 388.5 mmol). The mixture was stirred at r.t. overnight. The reaction solution was poured into H₂O and extracted with EtOAc (1 LX3). The combine organic layers was washed with brine, dried over Na₂SO₄ and concentrated then purified by SGC (PE:EA=3:1) to give compound 3-1 (70 g) as yellow solid

MS-ESI (m/z) 307.1 [M+H]⁺.

### Step 2: Compound 3-2

To a solution of compound 3-1 (70 g, 228.5 mmol) in DCM (560 mL) was added TFA (140 mL) slowly. It was stirred at r.t. overnight. It was concentrated to give compound 3-2 (70 g) as yellow solid.

MS-ESI (m/z) 207.1 [M+H]⁺.

### Step 3: Compound 3-3

The solution of compound 3-2 (52.8 g, 165 mmol) in DMF (400 mL) was added compound 3-2-1 (35.6 g, 181.3 mmol), DIEA (198.2 g, 1.53 mol) and HATU (70 g, 181 mmol). The mixture was stirred at r.t. overnight. The reaction solution was poured into H₂O and extracted with EtOAc (1 L*3). The combine organic layers was washed with brine, dried over Na₂SO₄ and concentrated then purified by SGC (PE:EA=2:1) to give compound 3-3 (23 g) as yellow solid. MS-ESI (m/z) 384.9 [M+H]⁺.

### Step 4: Intermediate (3)

The solution of compound 3-3 (23 g, 50.8 mmol) in THF (400 mL) was added DIBAL-H (1.5 M in THF, 135.6 mL) at −78° C. and stirred at this temperature for 4 hours. The reaction solution was quenched with HCl (1N, aq) carefully and extracted with ethyl acetate (400 mL*3). The combined organic phase was dried (Na₂SO₄) and concentrated then purified by SGC (PE:EA=2:1) to give intermediate (3) (13 g) as white solid MS-ESI (m/z) 326.0 [M+H]⁺.

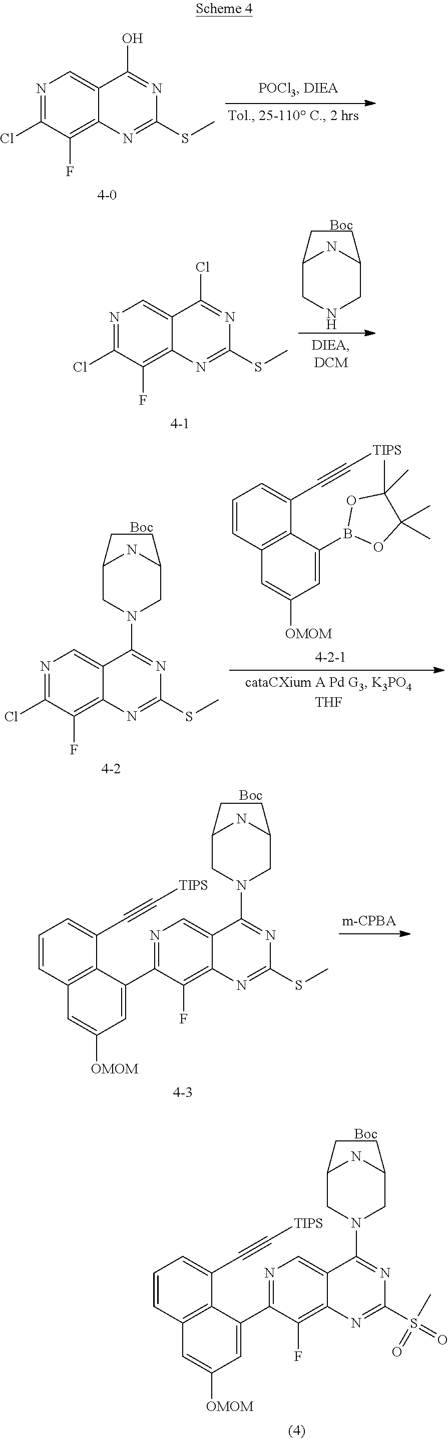

### Step 1: 4,7-dichloro-8-fluoro-2-(methylthio)pyrido[4,3-d]pyrimidine; 4-1

To a solution of 7-chloro-8-fluoro-2-methylsulfanyl-pyrido[4,3-d]pyrimidin-4-ol (SM 5 67.0 g, 273 mmol, 1 eq) in Tol. (350 mL) was added POCl₃ (209 g, 1.36 mol, 127 mL, 5 eq) at 25° C., then DIEA (77.6 g, 600 mmol, 105 mL, 2.2 eq) was added into the mixture blow 40° C. and stirred at 110° C. for 2 hrs. LCMS showed desired MS was detected. The residue was poured into Sat·NaHCO₃ slowly (keep pH=8) and extracted with EtOAc (500 mL*2), washed with sat·NaCl (400 mL), dried over Na₂SO₄, filtered and concentrated under reduced pressure to give compound (67.3 g, 255 mmol, 93.5% yield) as a brown solid. LCMS: m/z 264.0 [M+H]⁺.

### Step 2: tert-butyl 3-(7-chloro-8-fluoro-2-(methylthio)pyrido[4,3-d]pyrimidin-4-yl)-3,8-diazabicyclo[3.2.1]octane-8-carboxylate; 4-2

To a solution of compound 4-1 (67.3 g, 255 mmol, 1 eq) in DCM (600 mL) was added a solution of tert-butyl 3,8-diazabicyclo[3.2.1]octane-8-carboxylate (48.7 g, 229 mmol, 0.9 eq) in DCM (100 mL) at −40° C. Then DIEA (98.9 g, 765 mmol, 133 mL, 3 eq) was added at −40° C. and stirred for 0.5 hr. TLC (SiO₂, Petroleum ether:ethyl acetate=5:1, R1 R_{f}=0.46, P1 R_{f}=0.22) showed a new spot was formed. The mixture was quenched by H₂O (1000 mL) and extracted with DCM (600 mL*2), washed with sat·NaCl (600 mL), dried over Na₂SO₄, filtered and concentrated under reduced pressure to give residue. The solid was stirred in EtOAc (100 mL) and filtered to give compound 4-2 (92.2 g, 209 mmol, 82.2% yield) as a yellow solid. ¹H NMR (400 MHz, CDCl₃) δ 8.72 (s, 1H), 4.57-4.33 (m, 4H), 3.76-3.54 (m, 2H), 2.62 (s, 3H), 2.01-1.89 (m, 2H), 1.69 (d, J=7.4 Hz, 2H), 1.52 (s, 9H). LCMS: m/z 441.0 [M+H]⁺.

### Step 3: tert-butyl 3-(8-fluoro-7-(3-(methoxymethoxy)-8-((triisopropylsilyl)ethynyl)naphthalen-1-yl)-2-(methylthio)pyrido[4,3-d]pyrimidin-4-yl)-3,8-diazabicyclo[3.2.1]octane-8-carboxylate; 4-3

To a solution of compound 4-2 (3.77 g, 5.92 mmol, 1.3 eq) in THF (20 mL) was added K₃PO₄ (1.5 M, 9.10 mL, 3 eq) and [2-(2-aminophenyl)phenyl]palladium(1+) bis(1-adamantyl)-butyl-phosphanemethanesulfonate (497 mg, 683 umol, 0.15 eq), then stirred at 65° C. for 10 hrs. LCMS showed desired MS was detected. The reaction mixture was concentrated to give a residue. The residue was purified by column chromatography (SiO₂, Petroleum ether:Ethyl acetate=10:1 to 0:1, SiO₂, Petroleum ether:Ethyl acetate=3:1, P1 R^{f}-0.3) to give compound 4-3 (4.2 g, crude) as a brown oil. LCMS: m/z 772.3 [M+H]⁺.

### Step 4: tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate; Intermediate (4)

To a solution of compound 4-3 (11.5 g, 14.5 mmol, 1.00 eq) in DCM (100 mL) was added m-CPBA (6.50 g, 32.0 mmol, 85% purity, 2.20 eq). The mixture was stirred at 0° C. for 1 hr. TLC (PE:EA=3:1 R1 R_{f}=0.3 P1 R_{f}=0.2) indicated Reactant 1 was consumed completely and the reaction was clean according to TLC. The reaction mixture was quenched by addition aqueous Na₂SO₃ at 0° C. The reaction mixture was diluted with H₂O 200 mLand extracted with DCM 200 mL (100 mL*2). The combined organic layers were washed with aqueous NaCl 200 mL (100 mL*2), dried over Na₂SO₄, filtered and concentrated under reduced pressure to give a residue. The crude product was purified by flash silica gel column chromatography (PE:EA=5: 1-3:1) to afford intermediate (4) as brown oil.

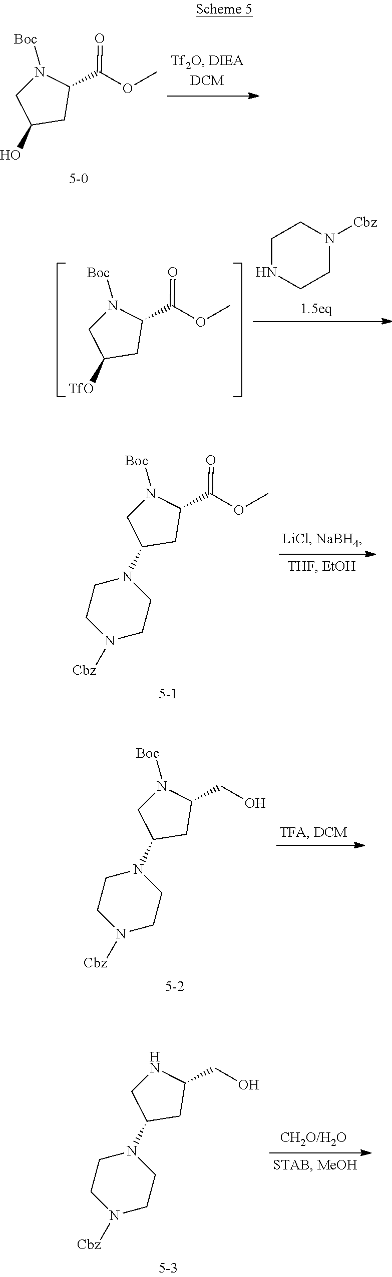

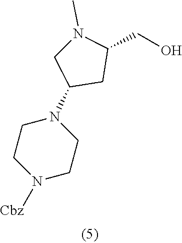 [SMILES: CN1C[C@@H](N2CCN(C(=O)OCc3ccccc3)CC2)C[C@H]1CO]

Scheme 5 illustrate the synthesis of a linker intermediate (5). Following are the details of synthetic procedure

### Step 1: 1-tert-butyl 2-methyl (2S,4S)-4-{4-[(benzyloxy)carbon yl]piperazin-1-yl}pyrrolidine-1,2-dicarboxylate; 5-1

To a stirred solution of 1-tert-butyl 2-methyl (2S,4R)-4-hydroxypyrrolidine-1,2-dicarboxylate (30 g, 122.312 mmol, 1 equiv) and DIEA (79.04 g, 611.560 mmol, 5 equiv) in DCM (600 mL) was added Tf₂O (51.76 g, 183.462 mmol, 1.50 equiv) dropwise at −78° C. under nitrogen atmosphere. The mixture was stirred 30 min at −78° C., then benzyl piperazine-1-carboxylate (40.41 g, 183.468 mmol, 1.5 equiv) was added at −40° C. The mixture was stirred 3 h at room temperature. The reaction was quenched by the addition of Water (500 mL) at room temperature. The resulting mixture was extracted with CH₂Cl₂ (2×300 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (2:1) to afford compound 5-1 (26 g, 47.50%) as a colorless oil.

### Step 2: benzyl 4-[(3S,5S)-1-(tert-butoxycarbonyl)-5-(hydroxymethyl)pyrrolidin-3-yl]piperazine-1-carboxylate; 5-2

To a stirred solution of compound 5-1 (26 g, 58.096 mmol, 1 equiv) and LiCl (4.93 g, 116.192 mmol, 2 equiv) in THF (250 mL, 3085.692 mmol) and EtOH (250 mL, 4303.328 mmol) was added NaBH4 (4.40 g, 116.192 mmol, 2 equiv) in portions at room temperature under nitrogen atmosphere. The mixture was stirred 3 h at room temperature. The reaction was quenched with Water (500 mL) at room temperature. The resulting mixture was extracted with CH2Cl2 (3×400 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (1:1) to afford compound 5-2 (14.4 g, 59.08%) as a colorless oil.

### Step 3: benzyl 4-[(3S,5S)-5-(hydroxymethyl)pyrrolidin-3-yl]piperazine-1-carboxylate; 5-3

To a stirred solution of compound 5-2 (14.4 g, 34.325 mmol, 1 equiv) in DCM (150 mL) was added TFA (80 mL, 1077.046 mmol, 31.38 equiv) at room temperature under nitrogen atmosphere. The mixture was stirred room temperature. The resulting mixture was concentrated under vacuum. The crude product was used in the next step directly without further purification.

### Step 4: benzyl 4-[(3S,5S)-5-(hydroxymethyl)-1-methylpyrrolidin-3-yl]piperazine-1-carboxylate; Intermediate (5)

To a stirred solution of compound 5-3 (12 g, 37.570 mmol, 1 equiv) and HCHO in water (60 mL, 37%) in MeOH (250 mL, 6174.708 mmol) was added STAB (31.85 g, 150.280 mmol, 4 equiv) in portions at room temperature under nitrogen atmosphere. The mixture was stirred 3 h at room temperature. The reaction was quenched with Water (300 mL) at room temperature. The resulting mixture was extracted with CH2Cl2 (3×200 mL). The combined organic layers were washed with brine (1×200 mL), dried over anhydrous Na₂SO₄. After filtration, the filtrate was concentrated under reduced pressure. The residue was purified by silica gel column chromatography, eluted with CH2Cl2/MeOH (10:1) to afford intermediate 5 (5.5 g, 93.91%) as a colorless oil. 1H NMR (400 MHz, DMSO-d6) δ 7.43-7.24 (m, 5H), 5.07 (s, 2H), 4.42 (s, 1H), 3.46 (dd, J=10.8, 4.4 Hz, 1H), 3.36-3.32 (m, 6H), 2.96 (dd, J=10.1, 3.5 Hz, 1H), 2.89-2.86 (m, 1H), 2.34 (m, 4H), 2.29-2.14 (m, 4H), 1.97-1.92 (m, 1H), 1.52-1.40 (m, 1H); LCMS (ES, m/z):334 [M+H]+

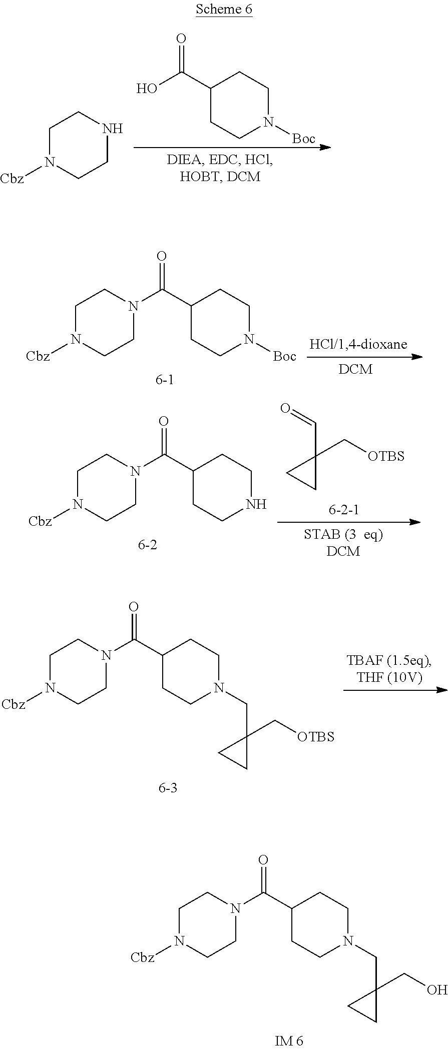

### Step 1: benzyl 4-[1-(tert-butoxycarbonyl) piperidine-4-carbonyl]piperazine-1-carboxylate; 6-1

To a stirred mixture of benzyl piperazine-1-carboxylate (10 g, 45.398 mmol, 1 equiv) and 1-(tert-butoxycarbonyl) piperidine-4-carboxylic acid (10.41 g, 45.398 mmol, 1 equiv) in DCM (100 mL) was added DIEA (17.60 g, 136.194 mmol, 3 equiv), EDC.HCl (11.31 g, 59.017 mmol, 1.3 equiv) and HOBT (7.97 g, 59.017 mmol, 1.3 equiv) in portions at room temperature under nitrogen atmosphere. The resulting mixture was stirred for 2 h at room temperature under nitrogen atmosphere. The reaction was quenched with Water at room temperature. The resulting mixture was extracted with CH₂Cl₂ (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EA (1:1) to afford compound 6-1 (8.2 g, 41.86%) as a yellow solid. LCMS (ES, m/z):432 [M+H]⁺

### Step 2: Compound 6-2

To a stirred solution of compound 6-1 (8.2 g, 19.002 mmol, 1 equiv) in DCM (80 mL) was added HCl(gas) in 1,4-dioxane (40.00 mL, 1316.511 mmol, 69.28 equiv) dropwise at 0° C. under nitrogen atmosphere. The resulting mixture was stirred for 2 h at 0° C. under nitrogen atmosphere. The resulting mixture was concentrated under reduced pressure. The crude product was used in the next step directly without further purification. LCMS (ES, m/z):332 [M+H]⁺

### Step 2: Compound 6-3

To a stirred mixture of compound 6-2 (10.5 g, 31.682 mmol, 1 equiv) and 1-1[(tert-butyldimethylsilyl)oxy]methylIcyclopropane-1-carbaldehyde (compound 6-2-1, 8.15 g, 38.018 mmol, 1.2 equiv) in DCM (100 mL) was added STAB (20.14 g, 95.046 mmol, 3 equiv) in portions at 0° C. under nitrogen atmosphere. The resulting mixture was stirred for 3 h at room temperature under nitrogen atmosphere. The reaction was quenched with sat. NaHCO₃ (aq.) at 0° C. The resulting mixture was extracted with CH₂Cl₂ (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH (10:1) to afford compound 6-3 (4 g) as a yellow oil. LCMS (ES, m/z):530 [M+H]⁺

### Step 3: Benzyl 4-(1-{[1-(hydroxymethyl) cyclopropyl]methyl}piperidine-4-carbonyl) piperazine-1-carboxylate; Intermediate (6)

A solution of compound 6-3 (4 g, 7.550 mmol, 1 equiv) in DCM (40 mL) was treated with TBAF (2.96 g, 11.325 mmol, 1.5 equiv) in THF (11 mL) for 2 h at room temperature under nitrogen atmosphere. The reaction was quenched with water at room temperature. The resulting mixture was extracted with CH₂Cl₂ (1×30 mL). The combined organic layers were washed with water (3×20 mL), dried over anhydrous Na₂SO₄. After filtration, the filtrate was concentrated under reduced pressure. The crude product intermediate (6) (1.6 g), which was used in the next step directly without further purification. LCMS (ES, m/z):416 [M+H]⁺

### Preparation of Compounds

The compounds claimed herein were prepared following the procedures which were exemplified by following examples.

### Example 1

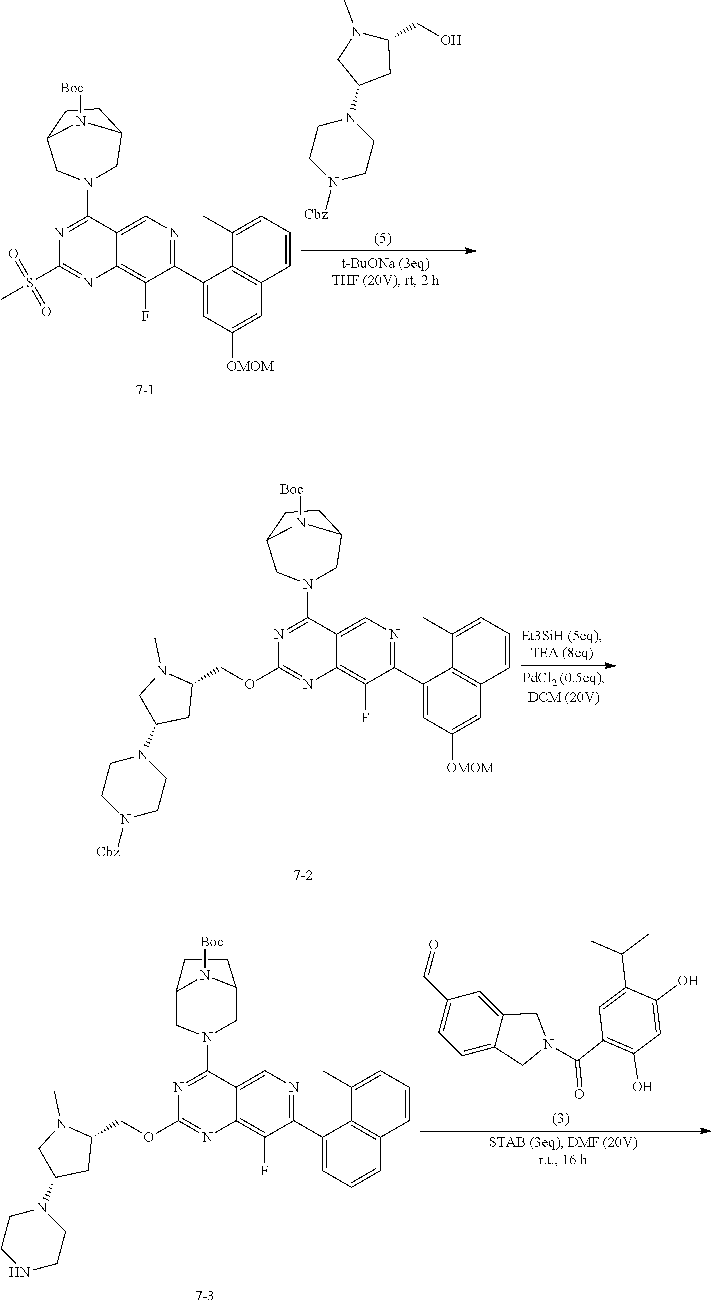

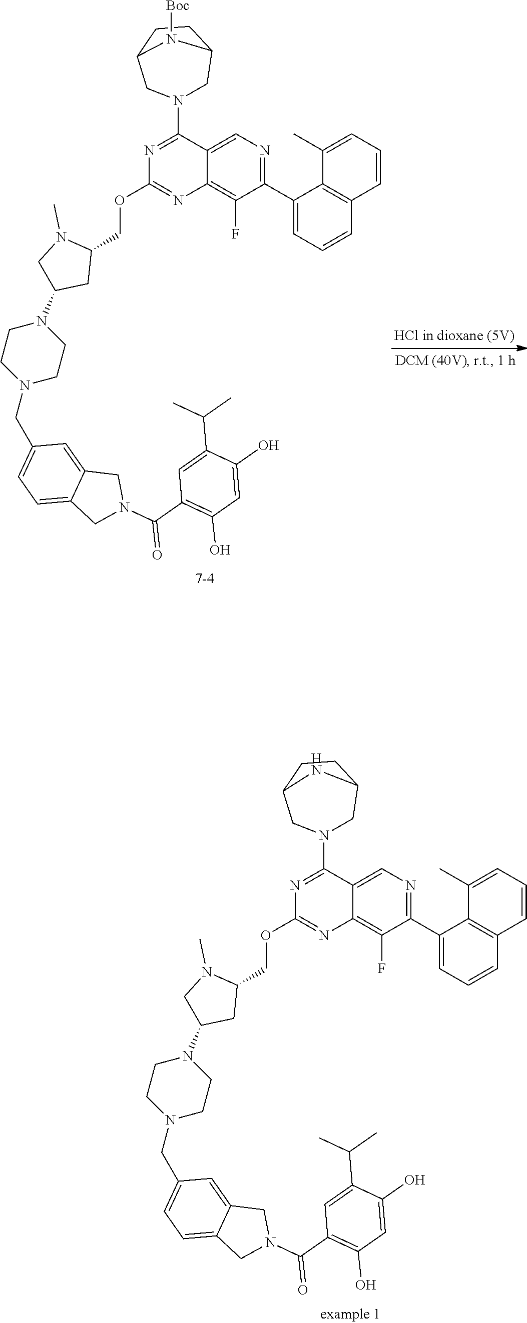 [SMILES: Cc1cccc2cccc(-c3ncc4c(N5CC6CCC(C5)N6C(=O)OC(C)(C)C)nc(OC[C@@H]5C[C@H](N6CCN(Cc7ccc8c(c7)CN(C(=O)c7cc(C(C)C)c(O)cc7O)C8)CC6)CN5C)nc4c3F)c12 ; Cc1cccc2cccc(-c3ncc4c(N5CC6CCC(C5)N6)nc(OC[C@@H]5C[C@H](N6CCN(Cc7ccc8c(c7)CN(C(=O)c7cc(C(C)C)c(O)cc7O)C8)CC6)CN5C)nc4c3F)c12]

### Step 1: Compound 7-2

To a stirred solution of tert-butyl 3-(8-fluoro-7-(3-(methoxymethoxy)-8-methylnaphthalen-1-yl)-2-(methylsulfonyl)pyrido[4,3-d]pyrimidin-4-yl)-3,8-diazabicyclo[3.2.1]octane-8-carboxylate (compound 7-1, 1 g, 1.174 mmol, 1 equiv) and benzyl 4-[(3S,5S)-5-(hydroxymethyl)-1-methylpyrrolidin-3-yl]piperazine-1-carboxylate (0.71 g, 1.761 mmol, 1.5 equiv) in THF (20 mL, 246.855 mmol) was added t-BuONa (0.34 g, 3.522 mmol, 3 equiv) at 0° C. under nitrogen atmosphere. The mixture was stirred 2 h at room temperature. After the reaction completed, it was quenched by aq. NH₄C₁. The resulting mixture was extracted with CH₂Cl₂ (3×10 mL). The resulting mixture was concentrated under reduced pressure. The residue was purified by silica gel column chromatography, eluted with CH₂Cl₂/MeOH (10:1) to afford compound 7-2 (1 g, 72.61%) as a yellow solid.

### Step 2: Compound 7-3

To a stirred solution of compound 7-3 and Et₃SiH (148.63 mg, 1.280 mmol, 5 equiv) in DCM (6 mL) was added TEA (206.95 mg, 2.048 mmol, 8 equiv) and PdCl₂ (9.07 mg, 0.051 mmol, 0.2 equiv) at room temperature under nitrogen atmosphere. The mixture was stirred 1 h at room temperature. After the reaction completed, the resulting mixture was filtered, the filter cake was washed with DCM (2×10 mL). The filtrate was concentrated under reduced pressure. The crude product was used in the next step directly without further purification.

### Step 3: Compound 7-4

To a stirred solution of compound 7-3 (60 mg, 0.086 mmol, 1 equiv) and 2-(2,4-dihydroxy-5-isopropylbenzoyl)-1,3-dihydroisoindole-5-car baldehyde (42.02 mg, 0.129 mmol, 1.5 equiv) in DMF (1.5 mL) was added STAB (54.74 mg, 0.258 mmol, 3 equiv) at room temperature under nitrogen atmosphere. The mixture was stirred 16 h at room temperature. The product was precipitated by the addition of brine. The crude product mixture was used in the next step directly without further purification.

### Step 4: Example 1

To a stirred solution of compound 7-4 (70 mg, 0.070 mmol, 1 equiv) in DCM (1.5 mL) was added HCl (gas) in 1,4-dioxane (0.3 mL, 9.874 mmol, 141.93 equiv) at room temperature under nitrogen atmosphere. The mixture was stirred 1 h at room temperature. The reaction was detected by LC-MS. After the reaction completed, the crude product was purified by Prep-HPLC with the following conditions to afford compound of example 1 (9.65 mg, 15.02%) as a white solid. LCMS-PH—RNS-2021-1228-0 (ES, m/z):906 [M+H]⁺; ¹H-NMR-PH—RNS-2021-1228-0 (400 MHz, DMSO-d₆, ppm) δ 10.07 (br, 1H), 9.60 (s, 1H), 9.10 (s, 1H), 8.07 (dd, J=8.2 Hz, 1H), 7.91 (d, J=8.2 Hz, 1H), 7.63-7.55 (m, 1H), 7.50-7.41 (m, 2H), 7.35-7.12 (m, 4H), 7.04 (s, 1H), 6.39 (s, 1H), 4.75 (s, 4H), 4.55-4.25 (m, 4H), 3.67-3.54 (m, 4H), 3.44-3.39 (m, 2H), 3.32-3.24 (m, 4H), 3.13-3.05 (m, 1H), 2.99 (d, J=10.2 Hz, 1H), 2.88 (s, 1H), 2.62 (s, 1H), 2.49-2.43 (m, 3H), 2.42-2.25 (m, 5H), 2.06 (s, 1H), 1.95 (s, 3H), 1.64 (s, 5H), 1.13 (d, J=6.8 Hz, 6H).

### Example 2

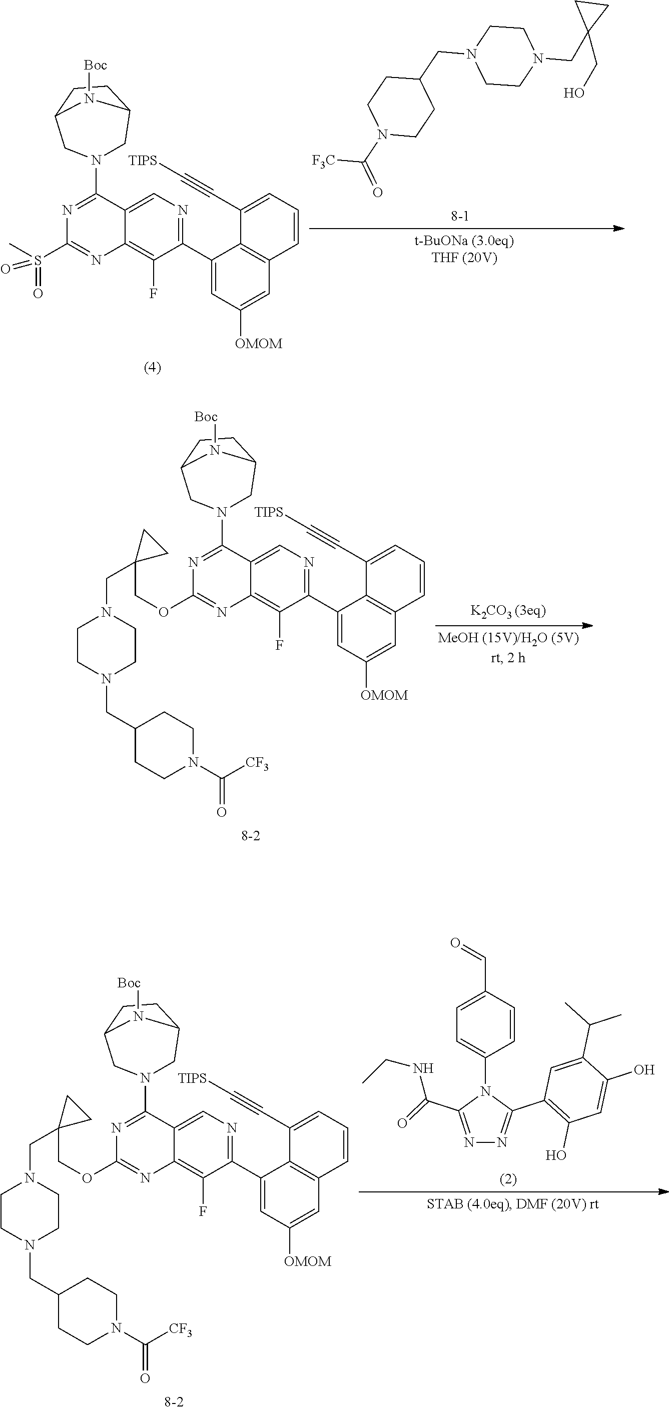

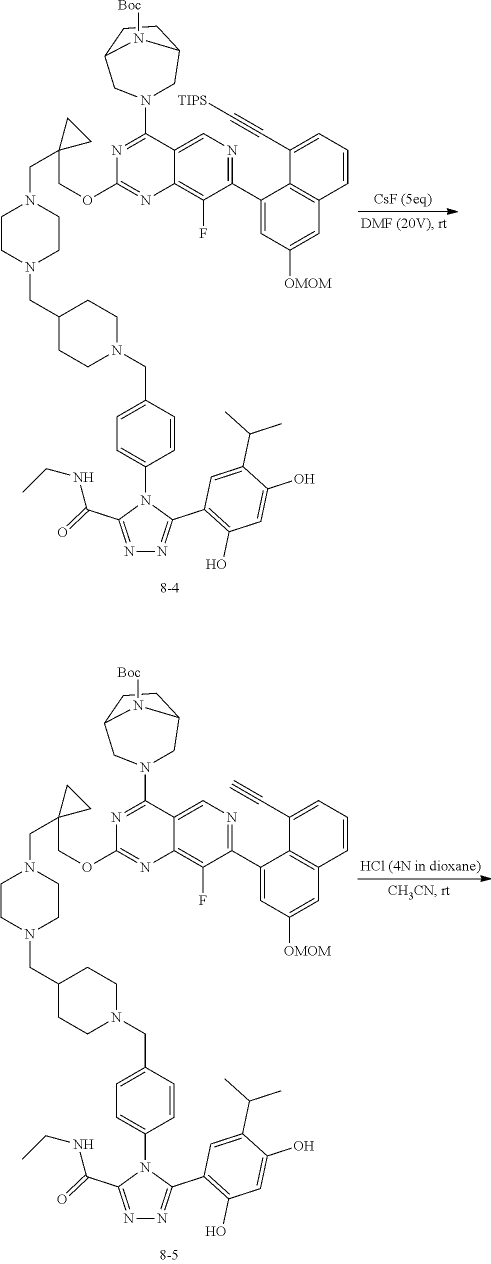

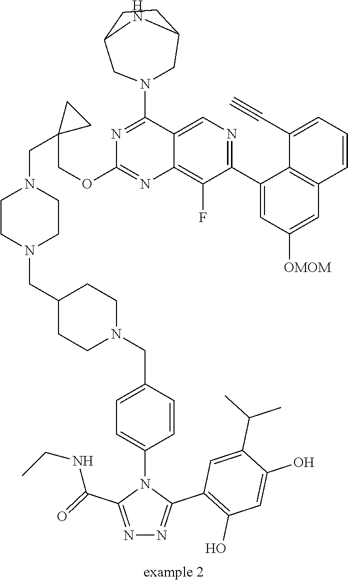

### Step 1: Compound 8-2; tert-butyl 3-{8-fluoro-7-[3-(methoxymethoxy)-8-[2-(triisopropylsilyl)ethynyl]naphthalen-1-yl]-2-[({1-[(4-{[1-(2,2,2-trifluoroacetyl)pi peridin-4-yl]methyl}piperazin-1-yl)methyl]cyclopropyl}methoxy)methyl]pyrido[4,3-d]pyrimidin-4-yl}-3,8-diazabicyclo[3.2.1]octane-8-carboxylate

A solution of tert-butyl 3-{8-fluoro-2-methanesulfonyl-7-[3-(methoxymethoxy)-8-[2-(triisopropylsilyl) ethynyl]naphthalen-1-yl]pyrido[4,3-d]pyrimidin-4-yl}-3,8-diazabicyclo[3.2.1]octane-8-carboxylate (IM 4, 500 mg, 0.622 mmol, 1 equiv) and 2,2,2-trifluoro-1-{4-[(4-{[1-(hydroxymethyl)cyclopropyl]methyl}piperazin-1-yl)methyl]piperidin-1-yl}ethanone (compound 8-1, 338.99 mg, 0.933 mmol, 1.5 equiv) and t-BuONa (179.28 mg, 1.866 mmol, 3 equiv) in THF was stirred for 2 h at room temperature under nitrogen atmosphere. Desired product could be detected by LCMS. The resulting mixture was washed with 20 mL of water. The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford compound 8-2 as a yellow solid. LCMS-PH—RNS-2021-1606-1:(ES, m/z):1087 [M+H]⁺

### Step 2: Compound 8-3; tert-butyl 3-{8-fluoro-7-[3-(methoxy methoxy)-8-[2-(triisopropylsilyl)ethynyl]naphthalen-1-yl]-2-[(1-{[4-(piperidin-4-ylmethyl) piperazin-1-yl]methyl}cyclopropyl)methoxy]pyrido[4,3-d]pyrimidin-4-yl}-3,8-diazabicyclo[3.2.1]octane-8-carboxylate

A solution of Intermediate 8-2 (400 mg, 0.147 mmol, 1 equiv, 40%) and K₂CO₃ (61.01 mg, 0.441 mmol, 3 equiv) in MeOH and water was stirred for 2 h at room temperature under air atmosphere. The desired product could be detected by LCMS. The resulting mixture was extracted with CH₂Cl₂ (3×20 mL). The combined organic layers were dried over anhydrous Na₂SO₄. After filtration, the filtrate was concentrated under reduced pressure. to afford compound 8-3 (366 mg, 250.90%) as a yellow solid. The crude product was used in the next step directly without further purification. LCMS-PH—RNS-2021-1606-2:(ES, m/z):991 [M+H]⁺

### Step 3: Compound 8-4; tert-butyl 3-[2-({1-[(4-{[1-({4-[3-(2,4-dihydroxy-5-isopropylphenyl)-5-(ethylcarbamoyl)-1,2,4-triazol-4-yl]phenyl}methyl)piperidin-4-yl]methyl}piperazin-1-yl)methyl]cyclopropyl}methoxy)-8-fluoro-7-[3-(methoxymethoxy)-8-[2-(triisopropylsilyl)ethynyl]naphthalen-1-yl]pyrido[4,3-d]pyrimidin-4-yl]-3,8-diazabicyclo[3.2.1]octane-8-carboxylate

To a stirred solution of compound 8-3 (59.68 mg, 0.152 mmol, 1.5 equiv) in DMF was added STAB (85.51 mg, 0.404 mmol, 4 equiv) in portions at room temperature under air atmosphere. The resulting mixture was stirred for additional overnight at room temperature. The residue was purified by reverse flash chromatography with the following conditions: column, C18; mobile phase, MeCN in Water (0.1% NH₃·H₂O), 10% to 100% gradient in 10 min; detector, UV 254 nm. This resulted in compound 8-4 (106 mg, 76.72%) as a yellow solid. LCMS-PH—RNS-2021-1606-3:(ES, m/z):1369 [M+H]⁺

### Step 4: Compound 8-5; tert-butyl 3-[2-({1-[(4-{[1-({4-[3-(2,4-dihydroxy-5-isopropylphenyl)-5-(ethylcarbamoyl)-1,2,4-triazol-4-yl]phenyl}methyl)piperidin-4-yl]methyl}piperazin-1-yl)methyl]cyclopropyl}methoxy)-7-[8-ethynyl-3-(methoxymethoxy)naphthalen-1-yl]-8-fluoropyrido[4,3-d]pyrimidin-4-yl]-3,8-diazabicyclo[3.2.1]octane-8-carboxylate

A mixture of compound 8-4 (106 mg, 0.077 mmol, 1 equiv) and CsF (58.77 mg, 0.385 mmol, 5 equiv) in DMF was stirred for 40 min at room temperature under air atmosphere. The resulting mixture was extracted with EtOAc (4×10 mL). The combined organic layers were dried over anhydrous Na₂SO₄. After filtration, the filtrate was concentrated under reduced pressure. The crude product was used in the next step directly without further purification. LCMS-PH—RNS-2021-1606-4:(ES, m/z):1213 [M+H]⁺

### Step 5: Example 2; 4-(4-{[4-({4-[(1-{[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]methyl}cyclopropyl)methyl]piperazin-1-yl}methyl)piperidin-1-yl]methyl}phenyl)-5-(2,4-dihydroxy-5-isopropylphenyl)-N-ethyl-1,2,4-triazole-3-carboxamide

A solution of compound 8-5 (80 mg, 0.066 mmol, 1 equiv) and HCl(gas) in 1,4-dioxane (0.99 mL, 3.960 mmol, 60 equiv) in MeCN was stirred for 1.5 h at room temperature under air atmosphere. Desired product could be detected by LCMS. The mixture was allowed to cool down to 0° C. The mixture was basified to pH 8 with NH₃·H₂O. The resulting mixture was extracted with CH₂Cl₂ (3×15 mL). The combined organic layers were dried over anhydrous Na₂SO₄. After filtration, the filtrate was concentrated under reduced pressure. The residue was purified by reverse flash chromatography with the following conditions: column, C₁₈; mobile phase, MeCN in Water (10 mmol/L NH₄HCO₃), 10% to 100% gradient in 10 min; detector, UV 254 nm. The crude product was purified by Prep-HPLC to afford example 2 (4.08 mg, 5.25%) as a yellow solid. LCMS-PH—RNS-2021-1606-0:(ES, m/z):1069 [M+H]⁺; ¹H NMR-PH—RNS-2021-1606-0: (400 MHz, DMSO-d₆) δ 10.62 (s, 1H), 10.15 (s, 1H), 9.77 (s, 1H), 9.02 (s, 1H), 8.96 (t, J=5.8 Hz, 1H), 7.88 (dd, J=8.0, 1.8 Hz, 1H), 7.49-7.39 (m, 2H), 7.38-7.32 (m, 3H), 7.28 (d, J=8.2 Hz, 2H), 7.11 (d, J=2.6 Hz, 1H), 6.57 (s, 1H), 6.34 (s, 1H), 4.50 (d, J=12.0 Hz, 1H), 4.33-4.21 (m, 3H), 3.65 (d, J=12.2 Hz, 2H), 3.59-3.54 (m, 4H), 3.52 (s, 1H), 3.46 (s, 2H), 3.16 (p, J=7.0 Hz, 2H), 2.90 (p, J=6.8 Hz, 1H), 2.83-2.63 (m, 2H), 2.42-2.16 (m, 7H), 2.06 (d, J=7.0 Hz, 2H), 1.91 (t, J=11.2 Hz, 2H), 1.64 (d, J=2.6 Hz, 6H), 1.45 (s, 2H), 1.23 (s, 2H), 1.14-0.98 (m, 4H), 0.80 (d, J=6.8 Hz, 6H), 0.63 (s, 2H), 0.40 (s, 2H).

### Example 3

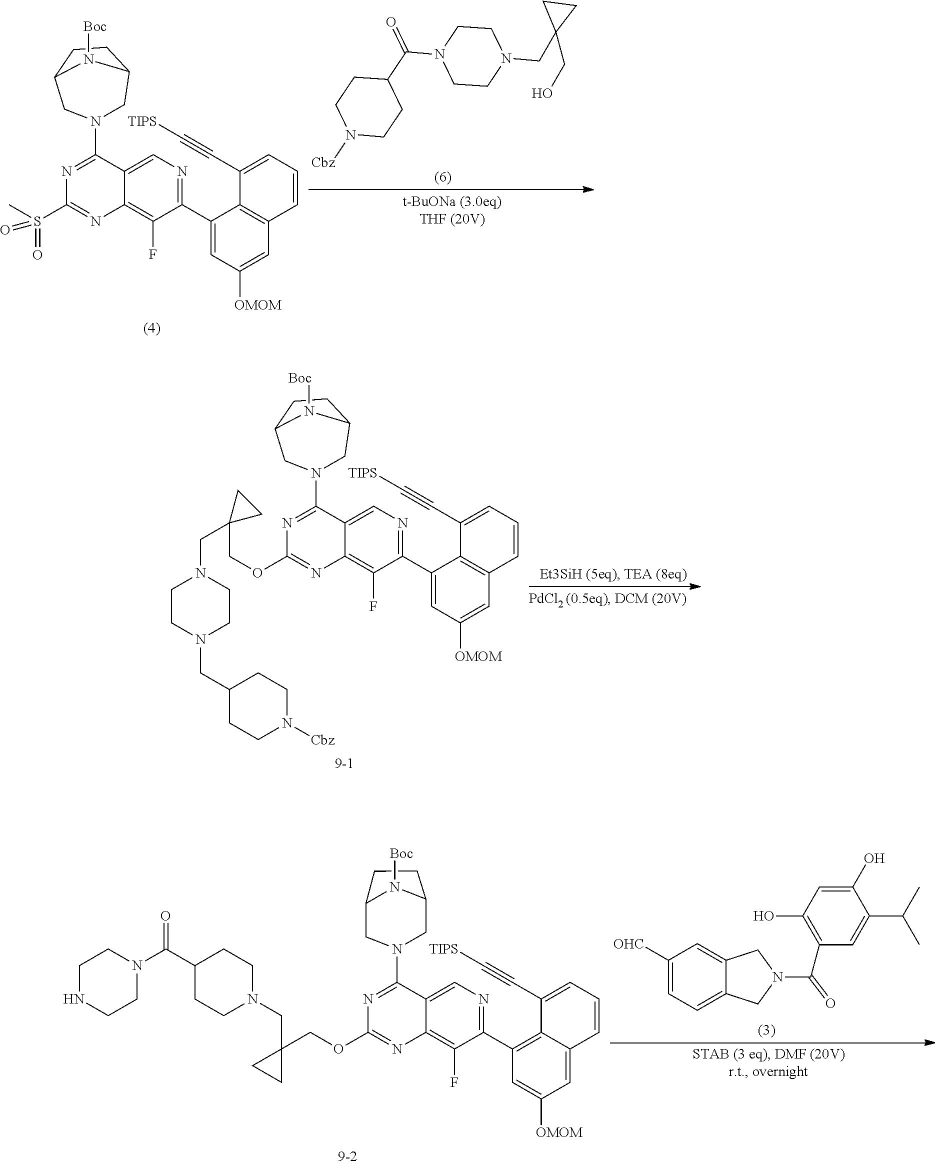

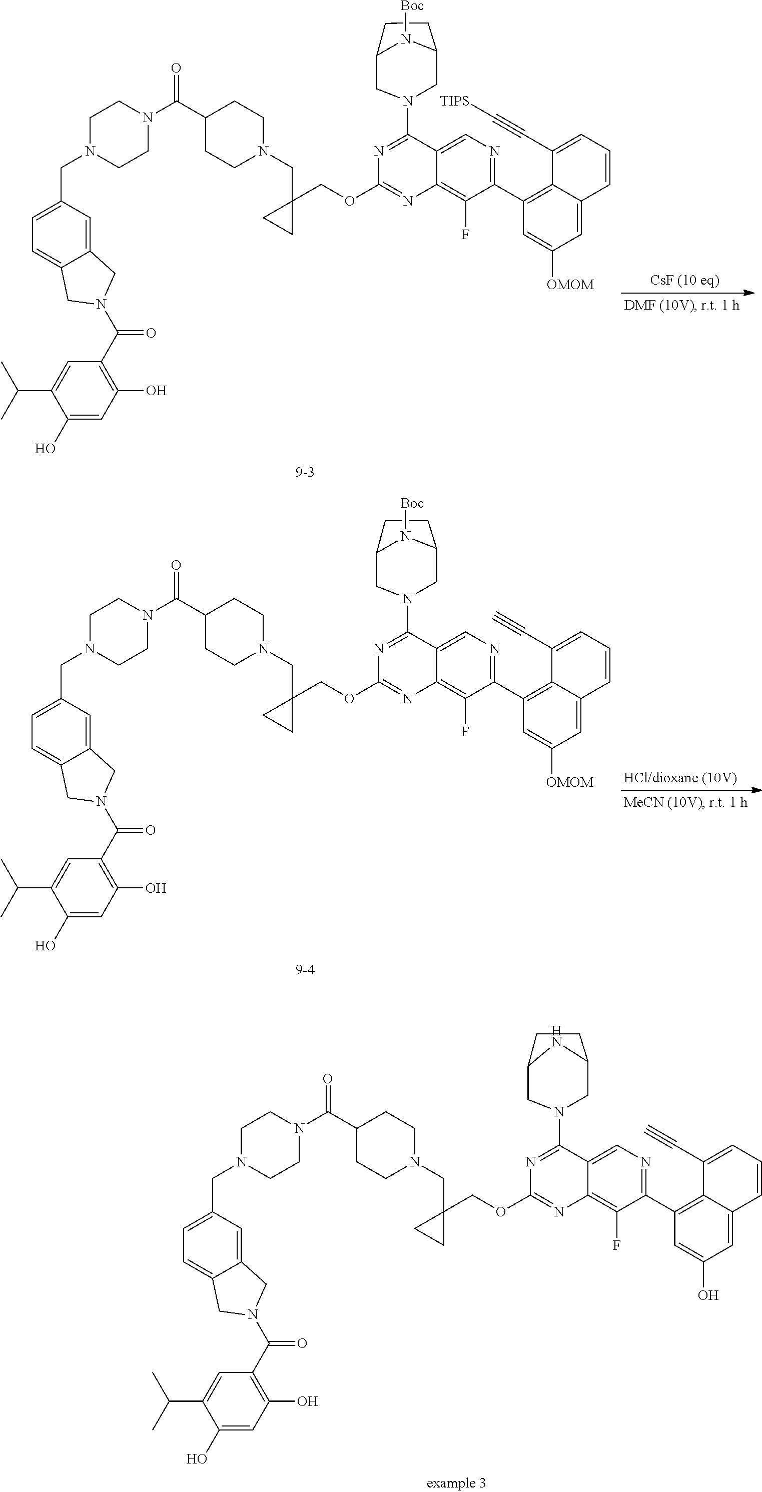

### Step 1: Compound 9-1; tert-butyl 3-(2-((1-((4-((1-((benzyloxy)carbonyl)piperidin-4-yl)methyl)piperazin-1-yl)methyl)cyclopropyl)methoxy)-8-fluoro-7-(3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate

A solution of tert-butyl 3-{8-fluoro-2-methanesulfonyl-7-[3-(methoxymethoxy)-8-[2-(triisopropylsilyl) ethynyl]naphthalen-1-yl]pyrido[4,3-d]pyrimidin-4-yl}-3,8-diazabicyclo[3.2.1]octane-8-carboxylate (500 mg, 0.622 mmol, 1 equiv) and benzyl 4-(1-{[1-(hydroxymethyl) cyclopropyl]methyl}piperidine-4-carbonyl) piperazine-1-carboxylate (350 mg, 0.933 mmol, 1.5 equiv) and t-BuONa (179.28 mg, 1.866 mmol, 3 equiv) in THF was stirred for 2 h at room temperature under nitrogen atmosphere. Desired product could be detected by LCMS. The resulting mixture was washed with 20 mL of water. The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford compound 9-1 as a yellow solid.

### Step 2: Compound 9-2; tert-butyl 3-{8-fluoro-7-[3-(methoxymethoxy)-8-[2-(triisopropylsilyl)ethynyl]naphthalen-1-yl]-2-[(1-{[4-(piperazine-1-carbonyl) piperidin-1-yl]methyl}cyclopropyl)methoxy]pyrido[4,3-d]pyrimidin-4-yl}-3,8-diazabicyclo[3.2.1]octane-8-carboxylate

To a stirred solution of compound 9-1 and Et₃SiH (120 mg, 1.10 mmol, 5 equiv) in DCM (6 mL) was added TEA (180 mg, 1.80 mmol, 8 equiv) and PdCl₂ (9.07 mg, 0.051 mmol, 0.2 equiv) at room temperature under nitrogen atmosphere. The mixture was stirred 1 h at room temperature. After the reaction completed, the resulting mixture was filtered, the filter cake was washed with DCM (2×10 mL). The filtrate was concentrated under reduced pressure. The crude product was used in the next step directly without further purification.

### Step 3: Compound 9-3; tert-butyl 3-{2-[(1-{[4-(4-{[2-(2,4-dihydroxy-5-isopropylbenzoyl)-1,3-dihydroisoindol-5-yl]methyl}piperazine-1-carbonyl) piperidin-1-yl]methyl}cyclopropyl)methoxy]-8-fluoro-7-[3-(methoxymethoxy)-8-[2-(triisopropylsilyl)ethynyl]naphthalen-1-yl]pyrido[4,3-d]pyrimidin-4-yl}-3,8-diazabicyclo[3.2.1]octane-8-carboxylate

To a stirred solution of compound 9-3 and 2-(2,4-dihydroxy-5-isopropylbenzo yl)-1,3-dihydroisoindole-5-carbaldehyde (42.72 mg, 0.131 mmol, 1.2 equiv) in D MF was added STAB (69.57 mg, 0.327 mmol, 3 equiv) in portions at room temperature. The resulting mixture was stirred for overnight at room temperature. The reaction was quenched with water at room temperature. The resulting mixture was extracted with EtOAc (3×10 mL). The combined organic layers were dried over anhydrous Na₂SO₄. After filtration, the filtrate was concentrated under reduced pressure. The residue was purified by reverse flash chromatography with the following conditions: column, C₁₈ silica gel; mobile phase, MeCN in Water (10 mmol/L NH4HCO3), 10% to 50% gradient in 10 min; detector, UV 254 nm. This results in compound 9-3 (90 mg, 62.57%) as a yellow solid. LCMS-PH—RNS-2022-1013-4 (ES, m/z): 1315 [M+H]⁺

### Step 4: Compound 9-4; tert-butyl 3-{2-[(1-{[4-(4-{[2-(2,4-dihydroxy-5-isopropylbenzoyl)-1,3-dihydroisoindol-5-yl]methyl}piperazine-1-carbonyl) piperidin-1-yl]methyl}cyclopropyl)methoxy]-8-fluoro-7-[3-(methoxymethoxy)-8-[2-(triisopropylsilyl)ethynyl]naphthalen-1-yl]pyrido[4,3-d]pyrimidin-4-yl}-3,8-diazabicyclo[3.2.1]octane-8-carboxylate

To a stirred solution of compound 9-3 (110 mg, 0.084 mmol, 1 equiv) in D MF was added CsF (127.09 mg, 0.840 mmol, 10 equiv) in portions at room temp erature. The resulting mixture was stirred for 2 h at room temperature. The reaction was quenched with water at room temperature. The resulting mixture was extra cted with EtOAc (3×10 mL). The combined organic layers were dried over anhydrous Na₂SO₄. After filtration, the filtrate was concentrated under reduced pressure. The crude product was used in the next step directly without further purification. LCMS-PH—RNS-2022-1013-5 (ES, m/z): 1158 [M+H]⁺

### Step 5: Example 3; 4-{5-[(4-{1-[(1-{[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]methyl}cyclopropyl)methyl]piperidine-4-carbonyl}piperazin-1-yl)methyl]-1,3-dihydroisoindole-2-carbonyl}-6-isopropylbenzene-1,3-diol

To a stirred solution of compound 9-4 (90 mg, 0.068 mmol, 1 equiv) in DMF was added HCl (gas) in 1,4-dioxane (1 mL, 32.913 mmol, 480.79 equiv) in portions at room temperature. The resulting mixture was stirred for 1 h at room temperature. The reaction was quenched with ammonia water at room temperature. The resulting mixture was extracted with EtOAc (3×10 mL). The combined organic layers were dried over anhydrous Na₂SO₄. After filtration, the filtrate was concentrated under reduced pressure. The crude product was purified by Prep-HPLC to afford the compound of example 3 (20.59 mg, 27.05%) as a yellow solid. LCMS-PH—RNS-2022-1013-0 (ES, m/z): 1014 [M+H]⁺; ¹H NMR-PH—RNS-2022-1013-C NMR (400 MHz, DMSO-d₆) δ 10.23-10.08 (m, 2H), 9.61 (s, 1H), 9.01 (s, 1H), 7.88 (dd, J=8.2, 1.6 Hz, 1H), 7.49-7.37 (i, 2H), 7.33 (d, J9=2.6 Hz, 1H), 7.22 (s, 3H), 7.12 (d, J 82.4 Hz, 1H), 7.04 (s, 1H), 6.39 (s, 1H), 4.76 (s, 4H), 4.49 (d, J=11.8 Hz, 1H), 4.25 (q, J=11.4, 9.0 Hz, 3H), 3.64 (d, J=12.2 Hz, 1H), 3.60 (s, 1H), 3.54 (s, 2H), 3.48 (d, J=13.4 Hz, 7H), 3.49-3.37 (s, 1H), 3.09 (p, J=7.0 Hz, 1H), 2.96 (t, J=12.2 Hz, 2H), 2.39-2.16 (m, 6H), 1.88 (s, 2H), 1.65 (s, 4H), 1.52 (s, 4H), 1.13 (d), J=6.8 Hz, 6H), 0.63 (s, 2H), 0.39 (s, 2H).

The following compounds in Table 1 were prepared according to the methods described above using the appropriate starting materials.

TABLE 1

| | Structure |
|---|---|
| | LCMS: m/z |
| Cmp | [M + H]⁺ |
| 4 | 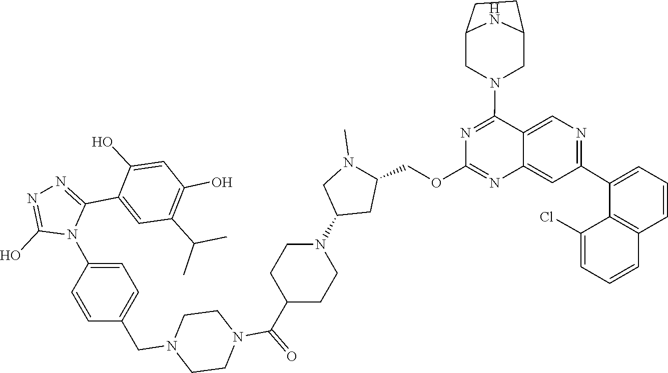 [SMILES: CC(C)c1cc(-c2nnc(O)n2-c2ccc(CN3CCN(C(=O)C4CCN([C@H]5C[C@@H](COc6nc(N7CC8CCC(C7)N8)c7cnc(-c8cccc9cccc(Cl)c89)cc7n6)N(C)C5)CC4)CC3)cc2)c(O)cc1O] |
| | 1033.2 |
| 5 | 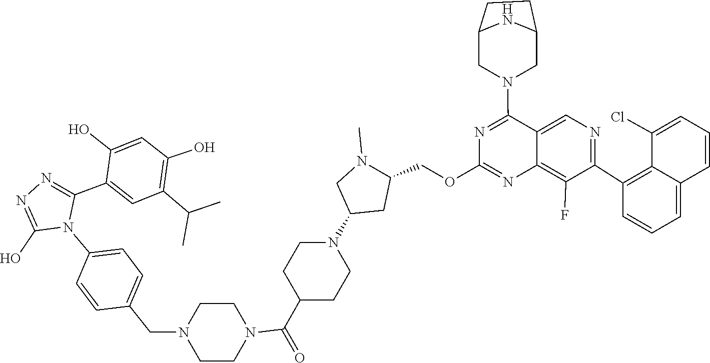 [SMILES: CC(C)c1cc(-c2nnc(O)n2-c2ccc(CN3CCN(C(=O)C4CCN([C@H]5C[C@@H](COc6nc(N7CC8CCC(C7)N8)c7cnc(-c8cccc9cccc(Cl)c89)c(F)c7n6)N(C)C5)CC4)CC3)cc2)c(O)cc1O] |
| | 1051.1 |
| 6 | 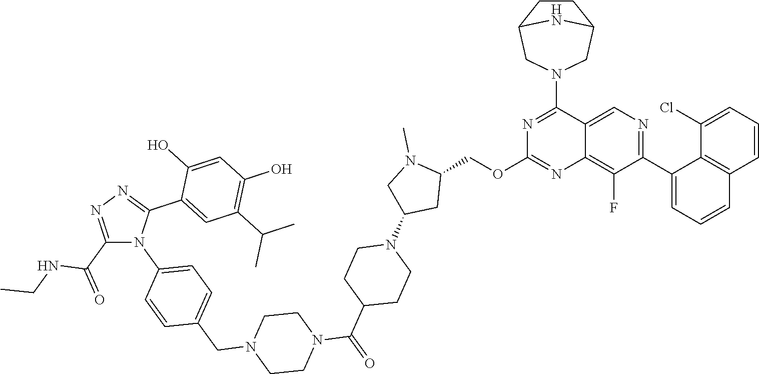 [SMILES: CCNC(=O)c1nnc(-c2cc(C(C)C)c(O)cc2O)n1-c1ccc(CN2CCN(C(=O)C3CCN([C@H]4C[C@@H](COc5nc(N6CC7CCC(C6)N7)c6cnc(-c7cccc8cccc(Cl)c78)c(F)c6n5)N(C)C4)CC3)CC2)cc1] |
| | 1106.1 |
| 7 | 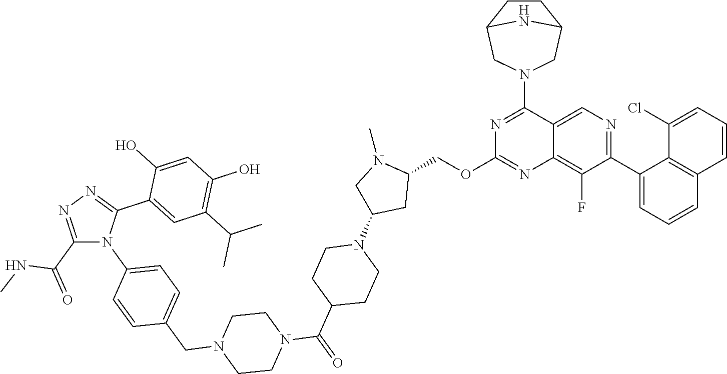 [SMILES: CNC(=O)c1nnc(-c2cc(C(C)C)c(O)cc2O)n1-c1ccc(CN2CCN(C(=O)C3CCN([C@H]4C[C@@H](COc5nc(N6CC7CCC(C6)N7)c6cnc(-c7cccc8cccc(Cl)c78)c(F)c6n5)N(C)C4)CC3)CC2)cc1] |
| | 1092.1 |
| 8 | 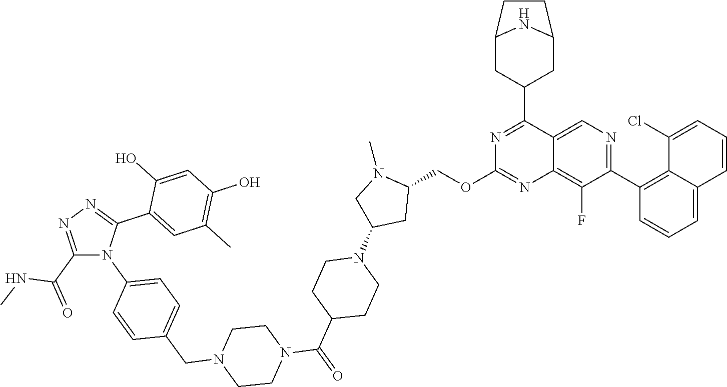 [SMILES: CNC(=O)c1nnc(-c2cc(C)c(O)cc2O)n1-c1ccc(CN2CCN(C(=O)C3CCN([C@H]4C[C@@H](COc5nc(C6CC7CCC(C6)N7)c6cnc(-c7cccc8cccc(Cl)c78)c(F)c6n5)N(C)C4)CC3)CC2)cc1] |
| | 1023.1 |
| 9 | 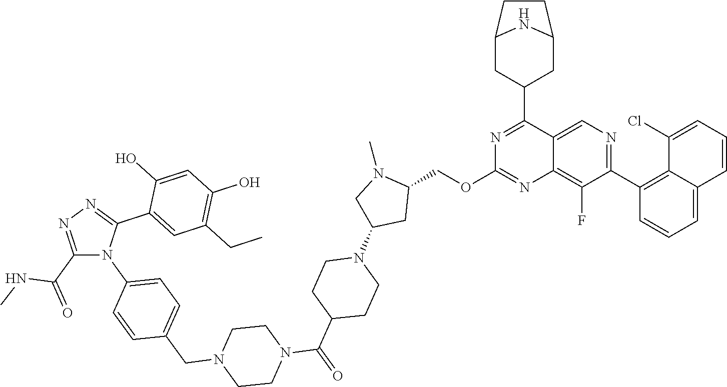 [SMILES: CCc1cc(-c2nnc(C(=O)NC)n2-c2ccc(CN3CCN(C(=O)C4CCN([C@H]5C[C@@H](COc6nc(C7CC8CCC(C7)N8)c7cnc(-c8cccc9cccc(Cl)c89)c(F)c7n6)N(C)C5)CC4)CC3)cc2)c(O)cc1O] |
| | 1037.1 |
| 10 | 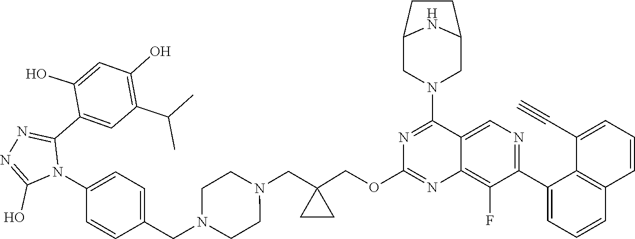 [SMILES: C#Cc1cccc2cccc(-c3ncc4c(N5CC6CCC(C5)N6)nc(OCC5(CN6CCN(Cc7ccc(-n8c(O)nnc8-c8cc(C(C)C)c(O)cc8O)cc7)CC6)CC5)nc4c3F)c12] |
| | 901.1 |
| 11 | 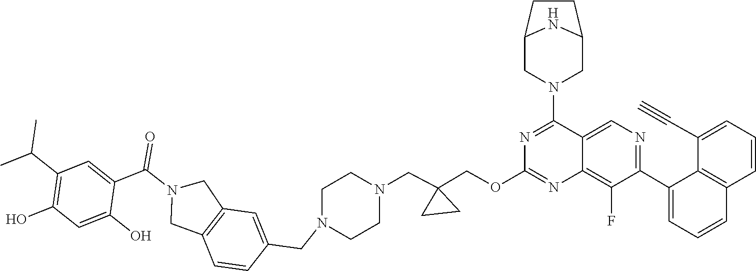 [SMILES: C#Cc1cccc2cccc(-c3ncc4c(N5CC6CCC(C5)N6)nc(OCC5(CN6CCN(Cc7ccc8c(c7)CN(C(=O)c7cc(C(C)C)c(O)cc7O)C8)CC6)CC5)nc4c3F)c12] |
| | 887.1 |
| 12 | 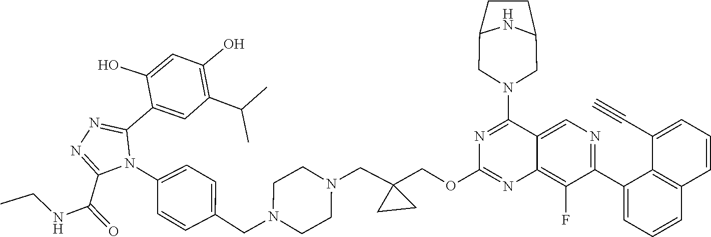 [SMILES: C#Cc1cccc2cccc(-c3ncc4c(N5CC6CCC(C5)N6)nc(OCC5(CN6CCN(Cc7ccc(-n8c(C(=O)NCC)nnc8-c8cc(C(C)C)c(O)cc8O)cc7)CC6)CC5)nc4c3F)c12] |
| | 956.1 |
| 13 | 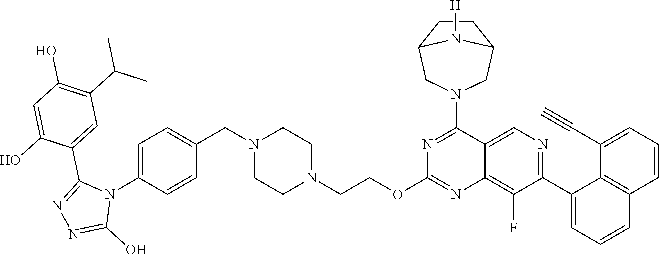 [SMILES: C#Cc1cccc2cccc(-c3ncc4c(N5CC6CCC(C5)N6)nc(OCCN5CCN(Cc6ccc(-n7c(O)nnc7-c7cc(C(C)C)c(O)cc7O)cc6)CC5)nc4c3F)c12] |
| | 861.5 |
| 14 | 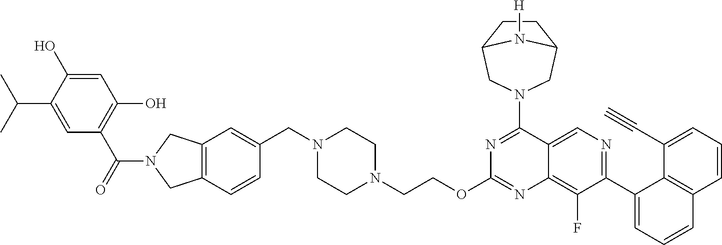 [SMILES: C#Cc1cccc2cccc(-c3ncc4c(N5CC6CCC(C5)N6)nc(OCCN5CCN(Cc6ccc7c(c6)CN(C(=O)c6cc(C(C)C)c(O)cc6O)C7)CC5)nc4c3F)c12] |
| | 847.2 |
| 15 | 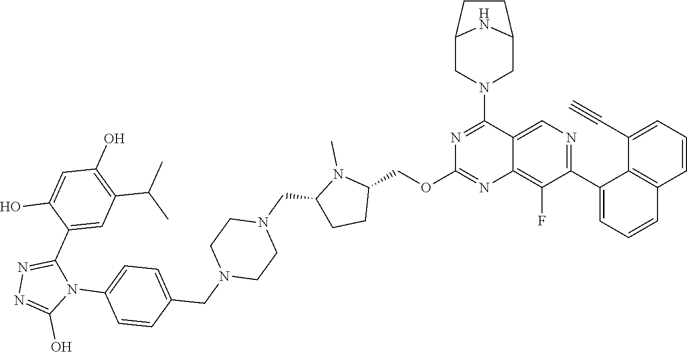 [SMILES: C#Cc1cccc2cccc(-c3ncc4c(N5CC6CCC(C5)N6)nc(OC[C@@H]5CC[C@H](CN6CCN(Cc7ccc(-n8c(O)nnc8-c8cc(C(C)C)c(O)cc8O)cc7)CC6)N5C)nc4c3F)c12] |
| | 944.4 |
| 16 | 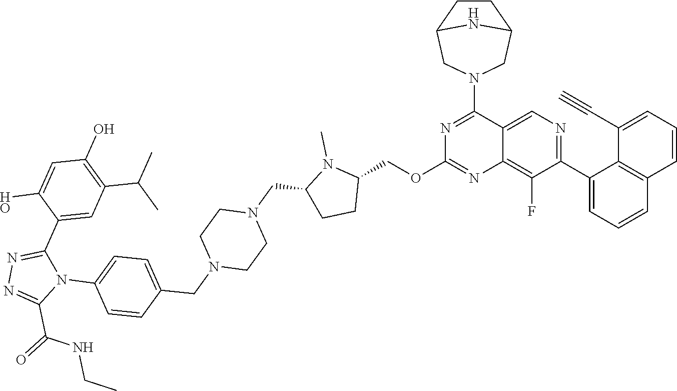 [SMILES: C#Cc1cccc2cccc(-c3ncc4c(N5CC6CCC(C5)N6)nc(OC[C@@H]5CC[C@H](CN6CCN(Cc7ccc(-n8c(C(=O)NCC)nnc8-c8cc(C(C)C)c(O)cc8O)cc7)CC6)N5C)nc4c3F)c12] |
| | 999.2 |
| 17 | 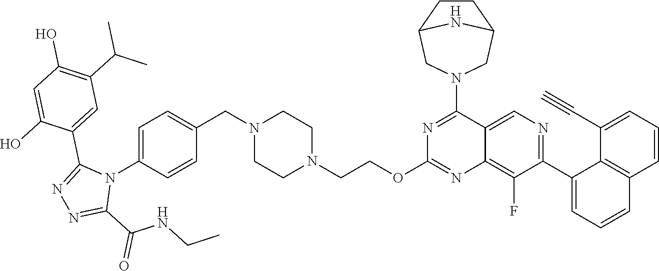 [SMILES: C#Cc1cccc2cccc(-c3ncc4c(N5CC6CCC(C5)N6)nc(OCCN5CCN(Cc6ccc(-n7c(C(=O)NCC)nnc7-c7cc(C(C)C)c(O)cc7O)cc6)CC5)nc4c3F)c12] |
| | 917.2 |
| 18 | 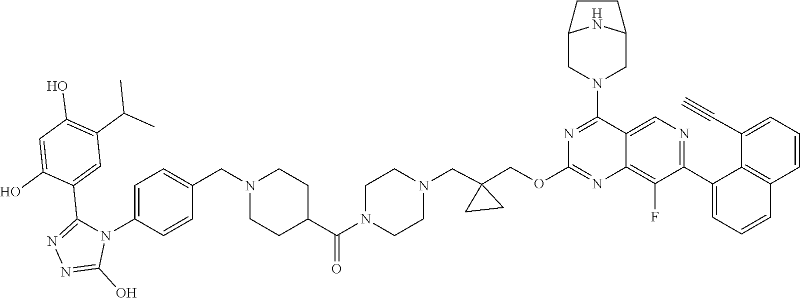 [SMILES: C#Cc1cccc2cccc(-c3ncc4c(N5CC6CCC(C5)N6)nc(OCC5(CN6CCN(C(=O)C7CCN(Cc8ccc(-n9c(O)nnc9-c9cc(C(C)C)c(O)cc9O)cc8)CC7)CC6)CC5)nc4c3F)c12] |
| | 1012.2 |
| 19 | 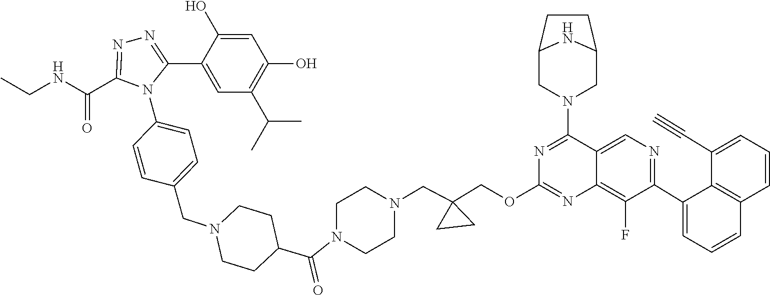 [SMILES: C#Cc1cccc2cccc(-c3ncc4c(N5CC6CCC(C5)N6)nc(OCC5(CN6CCN(C(=O)C7CCN(Cc8ccc(-n9c(C(=O)NCC)nnc9-c9cc(C(C)C)c(O)cc9O)cc8)CC7)CC6)CC5)nc4c3F)c12] |
| | 1067.3 |
| 20 | 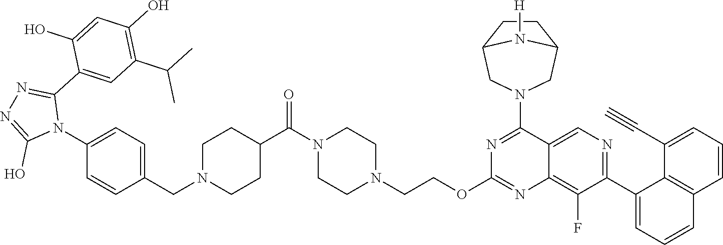 [SMILES: C#Cc1cccc2cccc(-c3ncc4c(N5CC6CCC(C5)N6)nc(OCCN5CCN(C(=O)C6CCN(Cc7ccc(-n8c(O)nnc8-c8cc(C(C)C)c(O)cc8O)cc7)CC6)CC5)nc4c3F)c12] |
| | 972.2 |
| 21 | 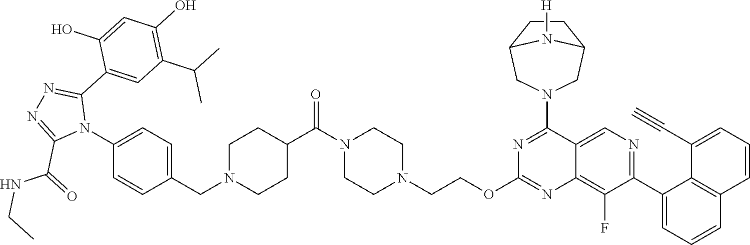 [SMILES: C#Cc1cccc2cccc(-c3ncc4c(N5CC6CCC(C5)N6)nc(OCCN5CCN(C(=O)C6CCN(Cc7ccc(-n8c(C(=O)NCC)nnc8-c8cc(C(C)C)c(O)cc8O)cc7)CC6)CC5)nc4c3F)c12] |
| | 1027.2 |
| 22 | 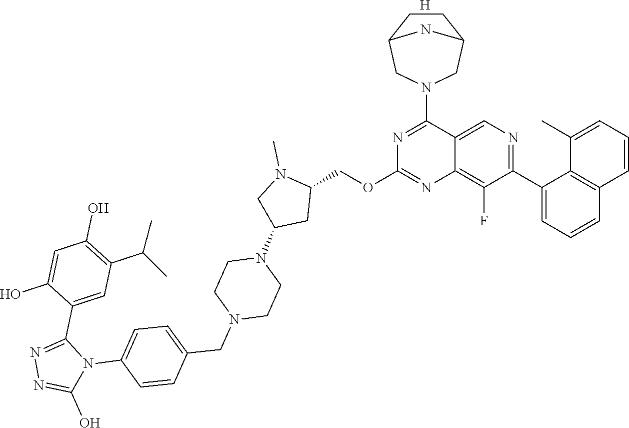 |
| | 920.2 |
| 23 | 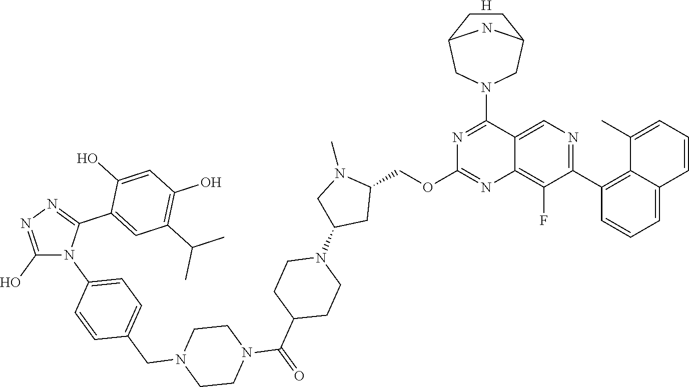 |
| | 1031.2 |
| 24 | 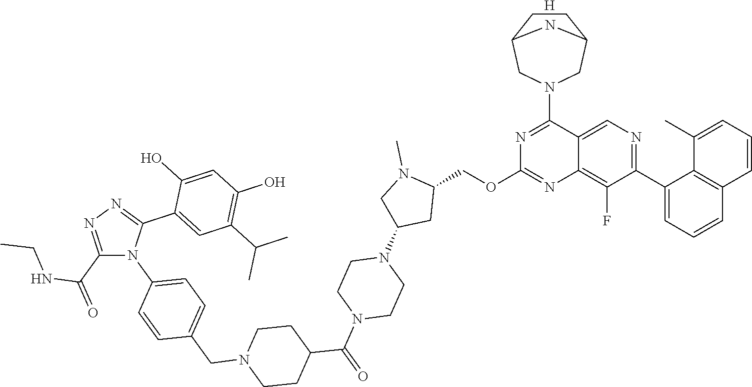 |
| | 1086.2 |
| 25 | 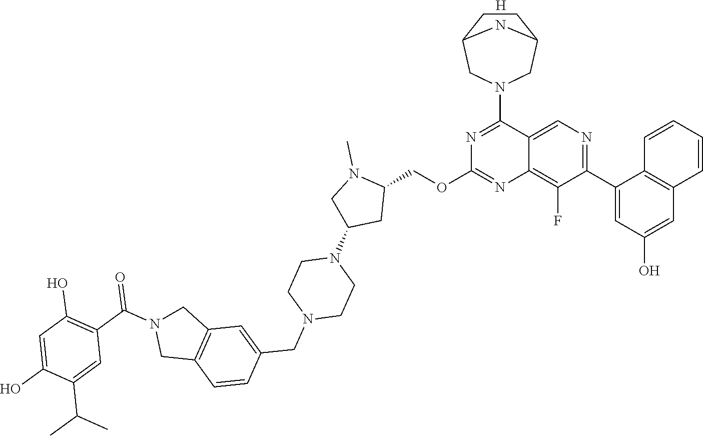 |
| | 908.2 |
| 26 | 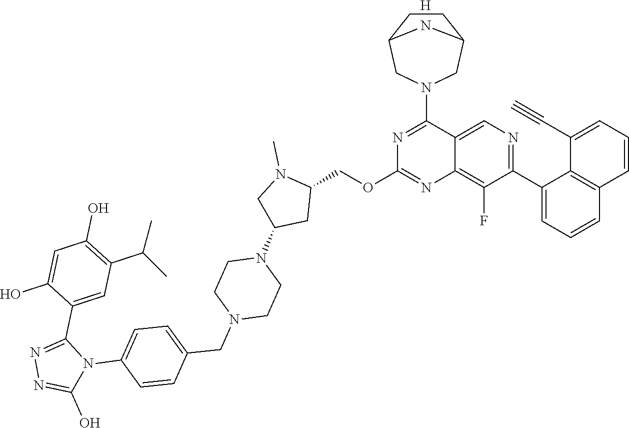 |
| | 930.2 |
| 27 | 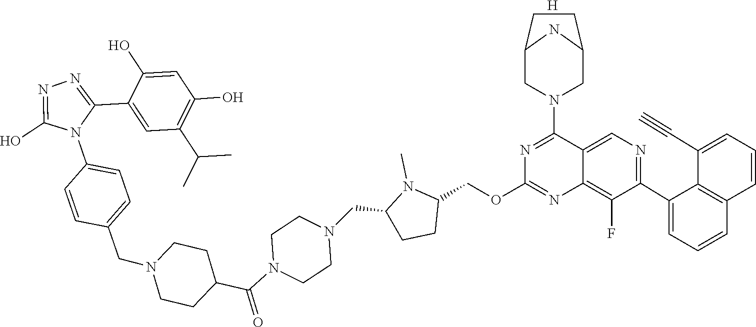 |
| | 1055.3 |
| 28 | 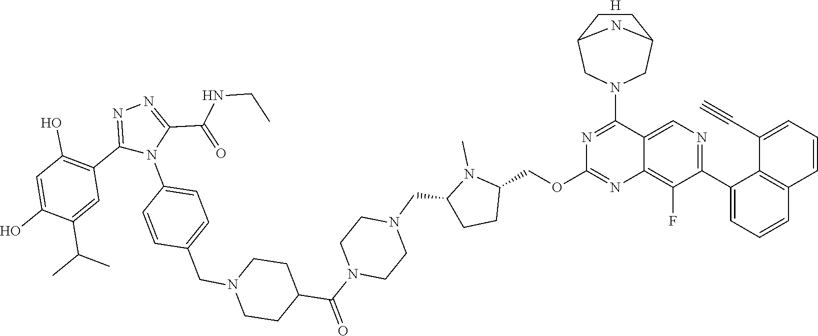 |
| | 1110.3 |
| 29 | 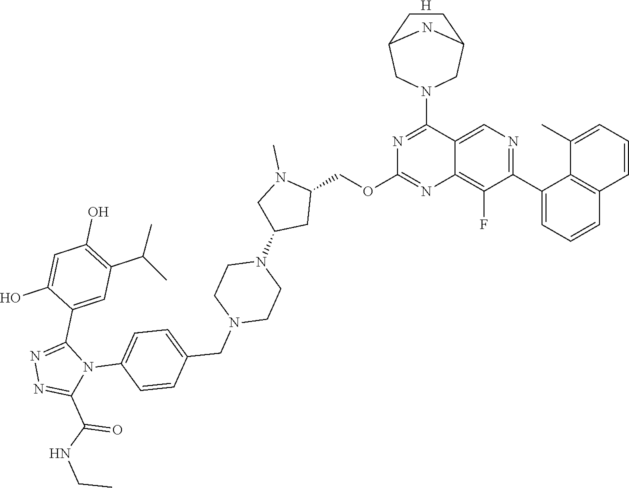 |
| | 975.2 |
| 30 | 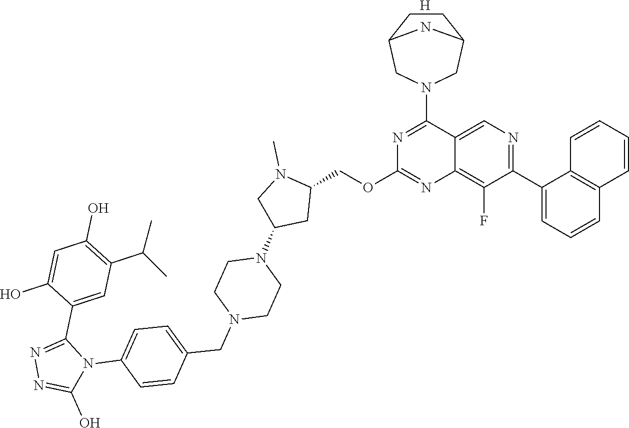 |
| | 906.1 |
| 31 | 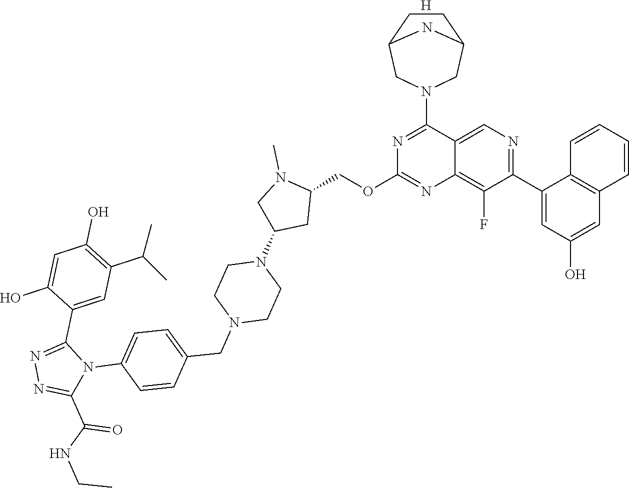 |
| | 977.2 |
| 32 | 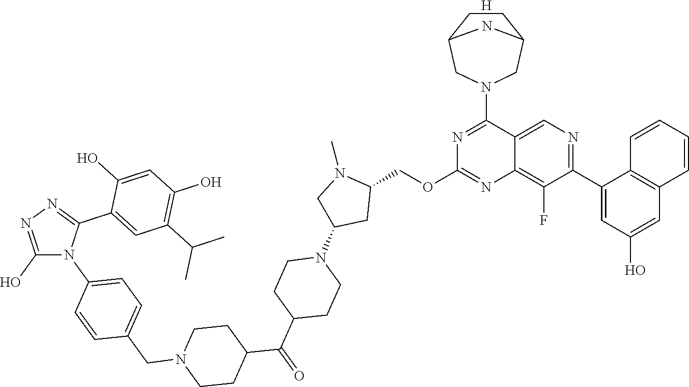 |
| | 1033.2 |
| 33 | 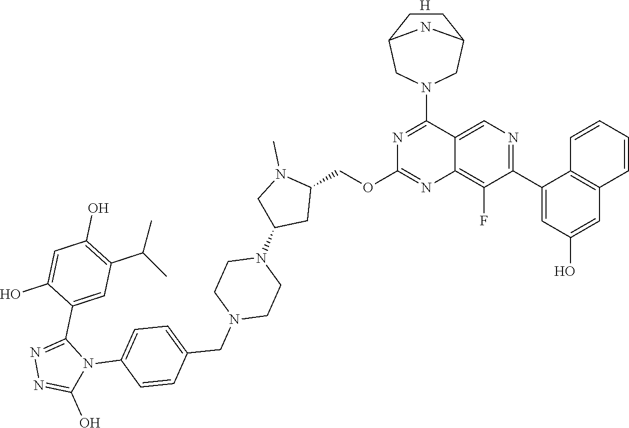 |
| | 922.1 |
| 34 | 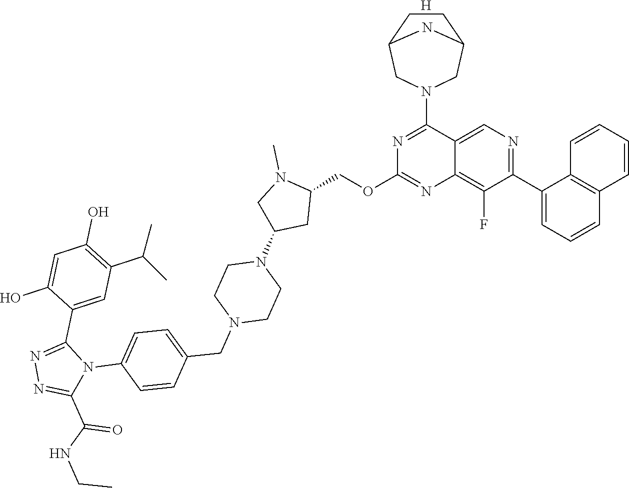 |
| | 961.2 |
| 35 | 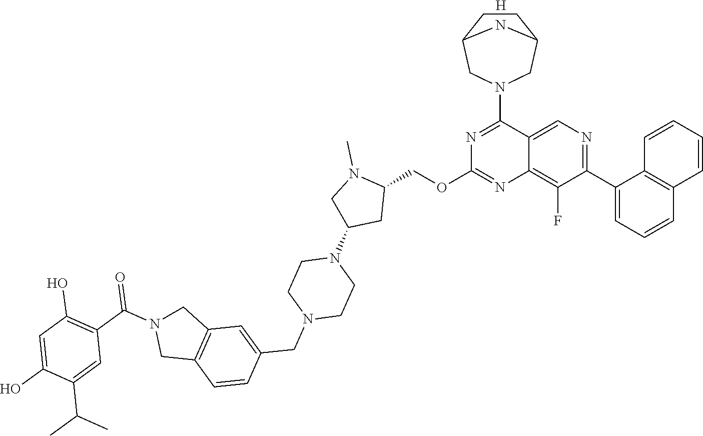 |
| | 892.1 |
| 36 | 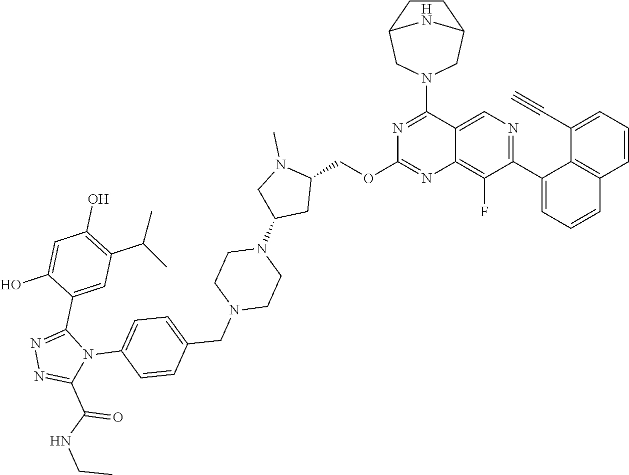 [SMILES: C#Cc1cccc2cccc(-c3ncc4c(N5CC6CCC(C5)N6)nc(OC[C@@H]5C[C@H](N6CCN(Cc7ccc(-n8c(C(=O)NCC)nnc8-c8cc(C(C)C)c(O)cc8O)cc7)CC6)CN5C)nc4c3F)c12] |
| | 985.2 |
| 37 | 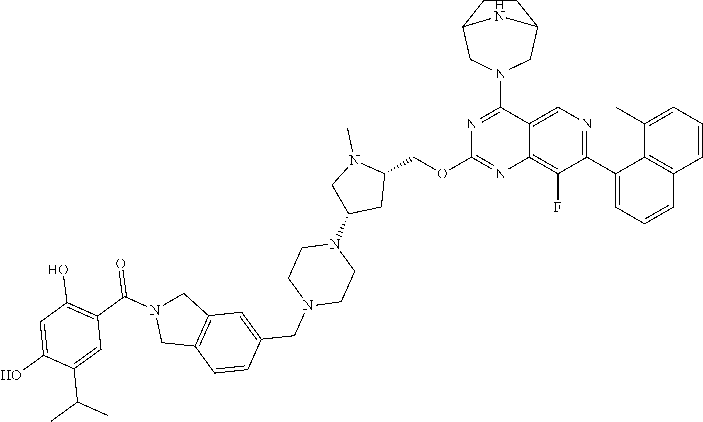 [SMILES: Cc1cccc2cccc(-c3ncc4c(N5CC6CCC(C5)N6)nc(OC[C@@H]5C[C@H](N6CCN(Cc7ccc8c(c7)CN(C(=O)c7cc(C(C)C)c(O)cc7O)C8)CC6)CN5C)nc4c3F)c12] |
| | 916.2 |
| 38 | 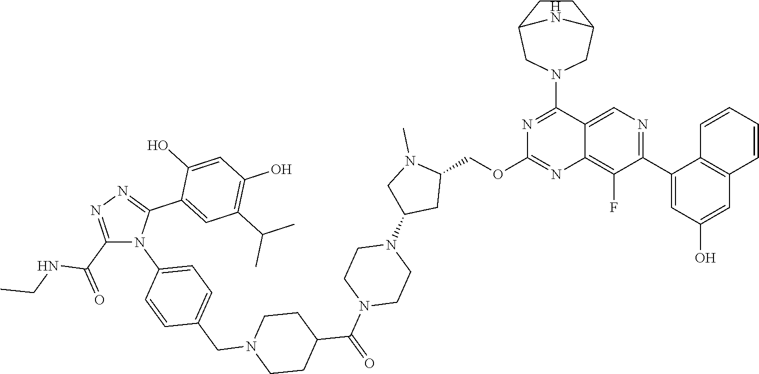 [SMILES: CCNC(=O)c1nnc(-c2cc(C(C)C)c(O)cc2O)n1-c1ccc(CN2CCC(C(=O)N3CCN([C@H]4C[C@@H](COc5nc(N6CC7CCC(C6)N7)c6cnc(-c7cc(O)cc8ccccc78)c(F)c6n5)N(C)C4)CC3)CC2)cc1] |
| | 1088.3 |
| 39 | 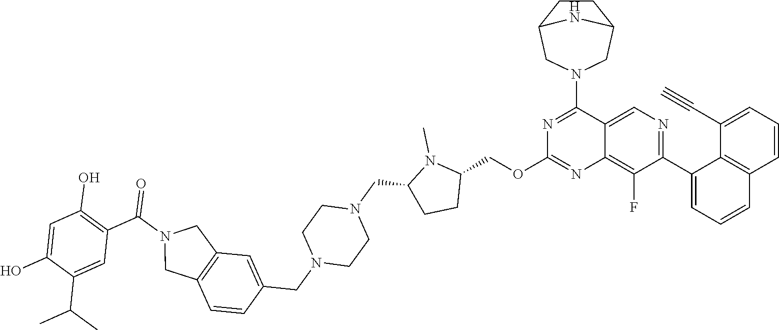 [SMILES: C#Cc1cccc2cccc(-c3ncc4c(N5CC6CCC(C5)N6)nc(OC[C@@H]5CC[C@H](CN6CCN(Cc7ccc8c(c7)CN(C(=O)c7cc(C(C)C)c(O)cc7O)C8)CC6)N5C)nc4c3F)c12] |
| | 930.4 |
| 40 | 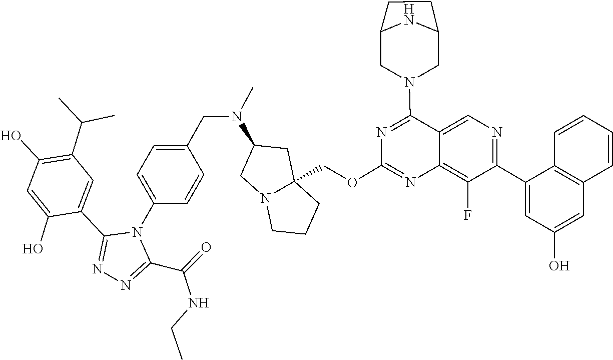 [SMILES: CCNC(=O)c1nnc(-c2cc(C(C)C)c(O)cc2O)n1-c1ccc(CN(C)[C@@H]2CN3CCC[C@]3(COc3nc(N4CC5CCC(C4)N5)c4cnc(-c5cc(O)cc6ccccc56)c(F)c4n3)C2)cc1] |
| | 948.1 |
| 41 | 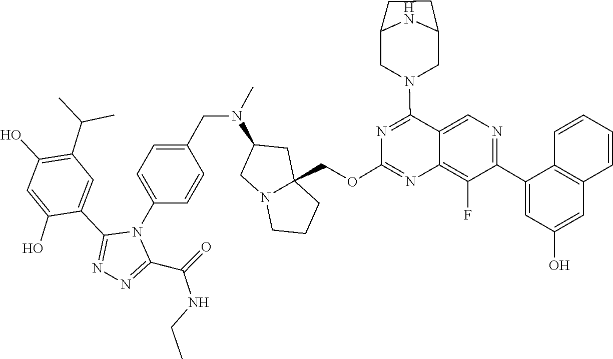 [SMILES: CCNC(=O)c1nnc(-c2cc(C(C)C)c(O)cc2O)n1-c1ccc(CN(C)[C@@H]2CN3CCC[C@@]3(COc3nc(N4CC5CCC(C4)N5)c4cnc(-c5cc(O)cc6ccccc56)c(F)c4n3)C2)cc1] |
| | 948.1 |
| 42 | 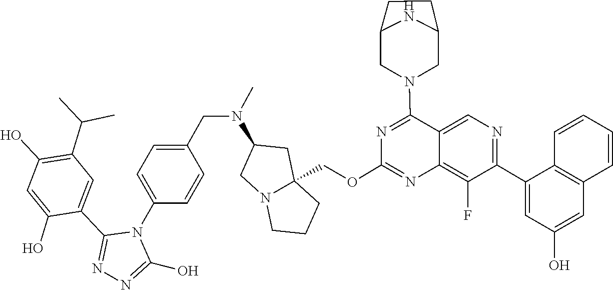 [SMILES: CC(C)c1cc(-c2nnc(O)n2-c2ccc(CN(C)[C@@H]3CN4CCC[C@]4(COc4nc(N5CC6CCC(C5)N6)c5cnc(-c6cc(O)cc7ccccc67)c(F)c5n4)C3)cc2)c(O)cc1O] |
| | 893.0 |
| 43 | 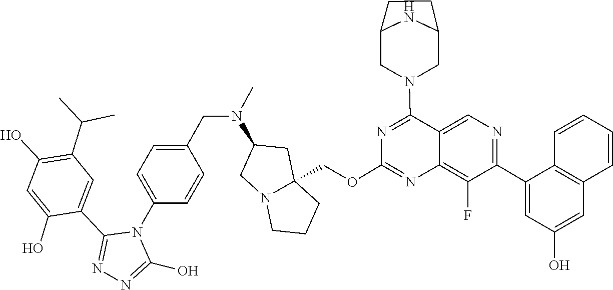 [SMILES: CC(C)c1cc(-c2nnc(O)n2-c2ccc(CN(C)[C@@H]3CN4CCC[C@]4(COc4nc(N5CC6CCC(C5)N6)c5cnc(-c6cc(O)cc7ccccc67)c(F)c5n4)C3)cc2)c(O)cc1O] |
| | 893.0 |
| 44 | 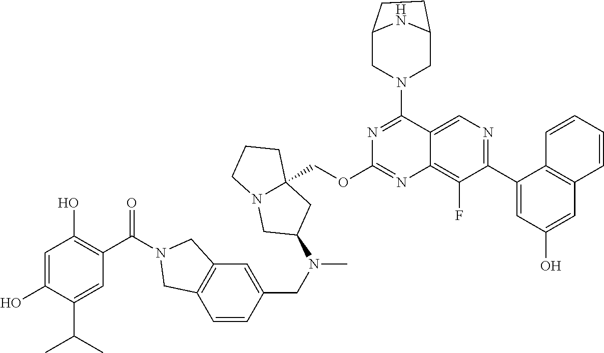 [SMILES: CC(C)c1cc(C(=O)N2Cc3ccc(CN(C)[C@H]4CN5CCC[C@@]5(COc5nc(N6CC7CCC(C6)N7)c6cnc(-c7cc(O)cc8ccccc78)c(F)c6n5)C4)cc3C2)c(O)cc1O] |
| | 879.1 |
| 45 | 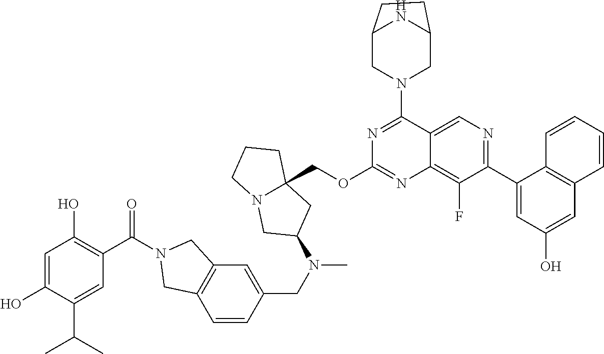 [SMILES: CC(C)c1cc(C(=O)N2Cc3ccc(CN(C)[C@H]4CN5CCC[C@]5(COc5nc(N6CC7CCC(C6)N7)c6cnc(-c7cc(O)cc8ccccc78)c(F)c6n5)C4)cc3C2)c(O)cc1O] |
| | 879.2 |
| 46 | 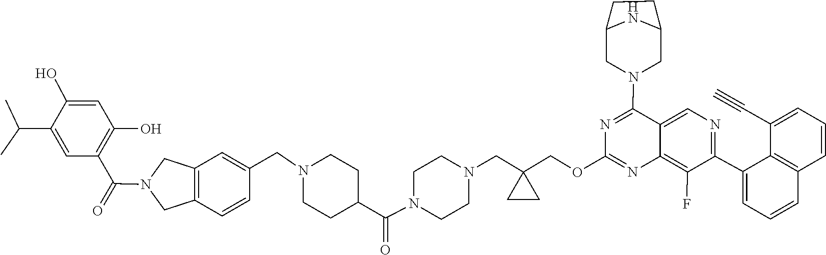 [SMILES: C#Cc1cccc2cccc(-c3ncc4c(N5CC6CCC(C5)N6)nc(OCC5(CN6CCN(C(=O)C7CCN(Cc8ccc9c(c8)CN(C(=O)c8cc(C(C)C)c(O)cc8O)C9)CC7)CC6)CC5)nc4c3F)c12] |
| | 998.2 |
| 47 | 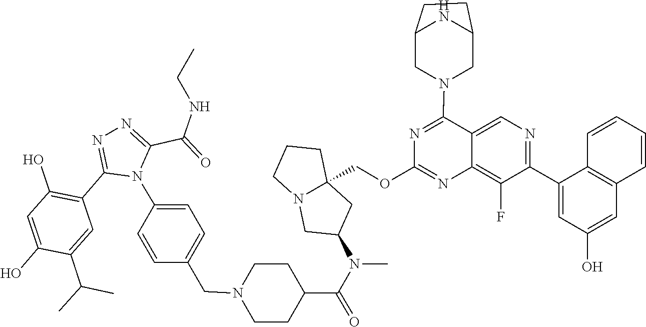 [SMILES: CCNC(=O)c1nnc(-c2cc(C(C)C)c(O)cc2O)n1-c1ccc(CN2CCC(C(=O)N(C)[C@H]3CN4CCC[C@@]4(COc4nc(N5CC6CCC(C5)N6)c5cnc(-c6cc(O)cc7ccccc67)c(F)c5n4)C3)CC2)cc1] |
| | 1059.3 |
| 48 | 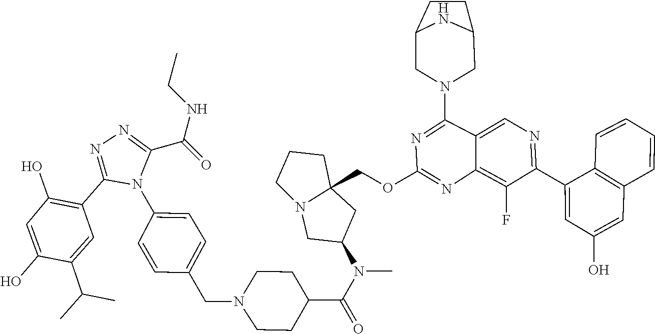 [SMILES: CCNC(=O)c1nnc(-c2cc(C(C)C)c(O)cc2O)n1-c1ccc(CN2CCC(C(=O)N(C)[C@H]3CN4CCC[C@]4(COc4nc(N5CC6CCC(C5)N6)c5cnc(-c6cc(O)cc7ccccc67)c(F)c5n4)C3)CC2)cc1] |
| | 1059.3 |
| 49 | 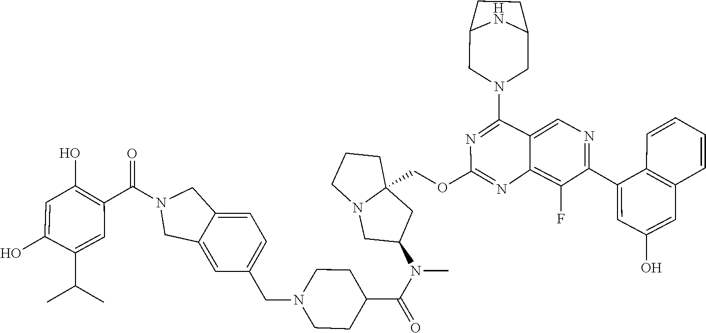 [SMILES: CC(C)c1cc(C(=O)N2Cc3ccc(CN4CCC(C(=O)N(C)[C@H]5CN6CCC[C@@]6(COc6nc(N7CC8CCC(C7)N8)c7cnc(-c8cc(O)cc9ccccc89)c(F)c7n6)C5)CC4)cc3C2)c(O)cc1O] |
| | 990.2 |
| 50 | 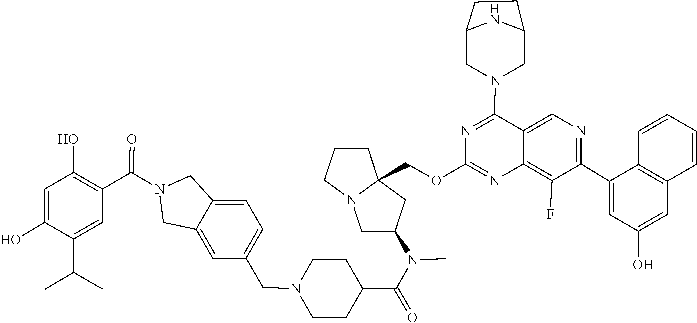 [SMILES: CC(C)c1cc(C(=O)N2Cc3ccc(CN4CCC(C(=O)N(C)[C@H]5CN6CCC[C@]6(COc6nc(N7CC8CCC(C7)N8)c7cnc(-c8cc(O)cc9ccccc89)c(F)c7n6)C5)CC4)cc3C2)c(O)cc1O] |
| | 990.2 |
| 51 | 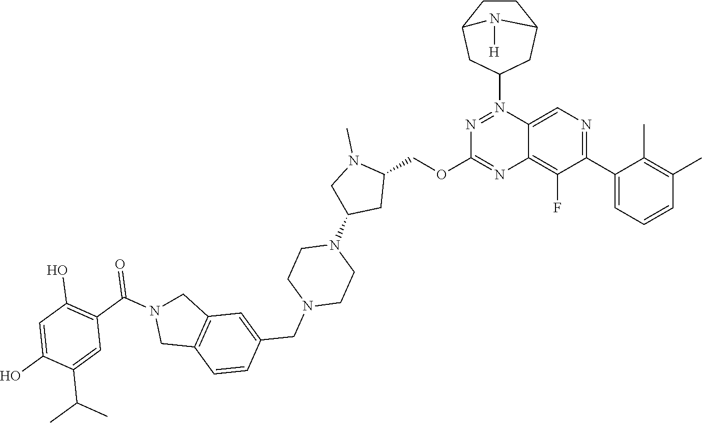 |
| | 870.3 |
| 52 | 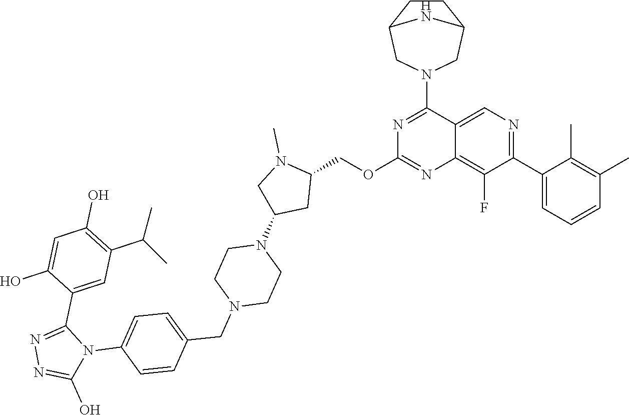 [SMILES: Cc1cccc(-c2ncc3c(N4CC5CCC(C4)N5)nc(OC[C@@H]4C[C@H](N5CCN(Cc6ccc(-n7c(O)nnc7-c7cc(C(C)C)c(O)cc7O)cc6)CC5)CN4C)nc3c2F)c1C] |
| | 884.6 |
| 53 | 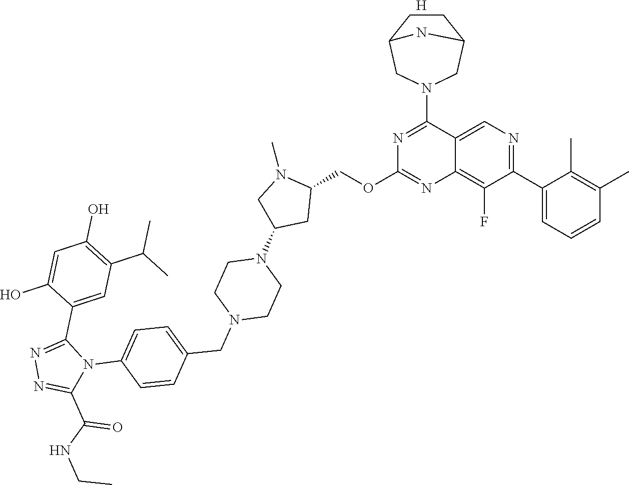 |
| | 939.1 |
| 54 | 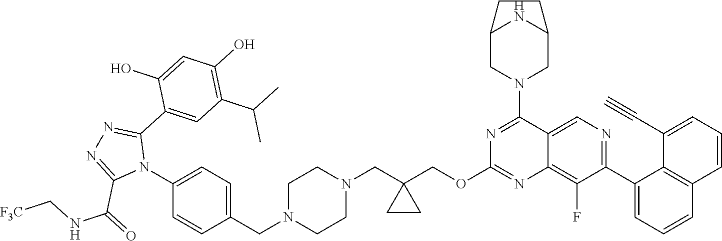 [SMILES: C#Cc1cccc2cccc(-c3ncc4c(N5CC6CCC(C5)N6)nc(OCC5(CN6CCN(Cc7ccc(-n8c(C(=O)NCC(F)(F)F)nnc8-c8cc(C(C)C)c(O)cc8O)cc7)CC6)CC5)nc4c3F)c12] |
| | 1010.1 |
| 55 | 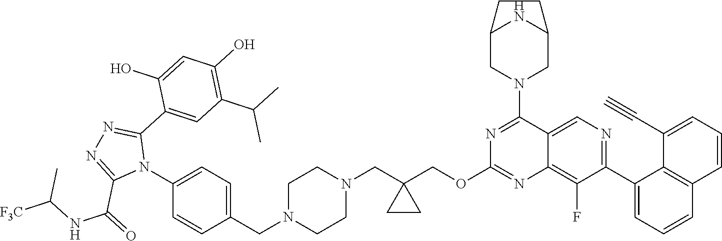 [SMILES: C#Cc1cccc2cccc(-c3ncc4c(N5CC6CCC(C5)N6)nc(OCC5(CN6CCN(Cc7ccc(-n8c(C(=O)NC(C)C(F)(F)F)nnc8-c8cc(C(C)C)c(O)cc8O)cc7)CC6)CC5)nc4c3F)c12] |
| | 1024.1 |
| 56 | 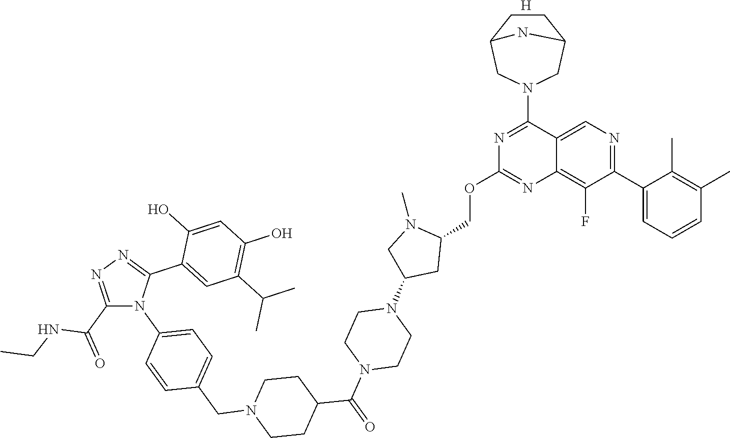 |
| | 1245.3 |
| 57 | 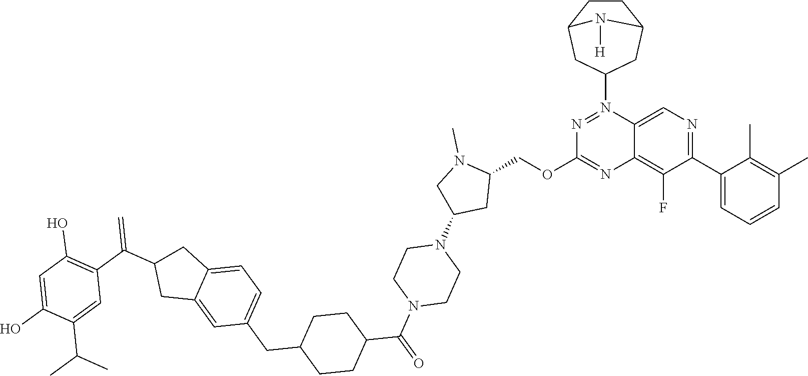 |
| | 981.2 |
| 58 | 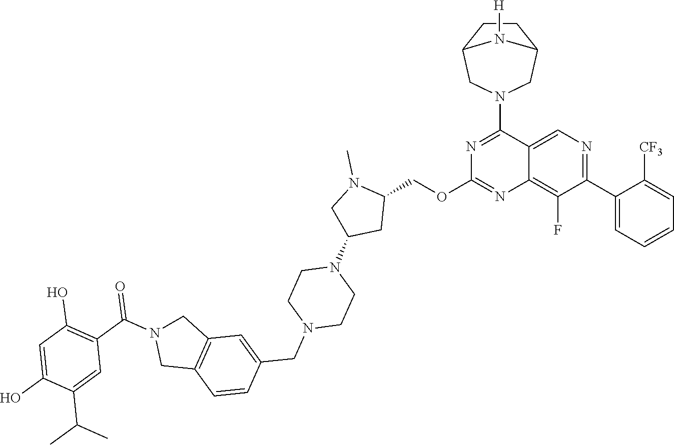 [SMILES: CC(C)c1cc(C(=O)N2Cc3ccc(CN4CCN([C@H]5C[C@@H](COc6nc(N7CC8CCC(C7)N8)c7cnc(-c8ccccc8C(F)(F)F)c(F)c7n6)N(C)C5)CC4)cc3C2)c(O)cc1O] |
| | 910.1 |
| 59 | 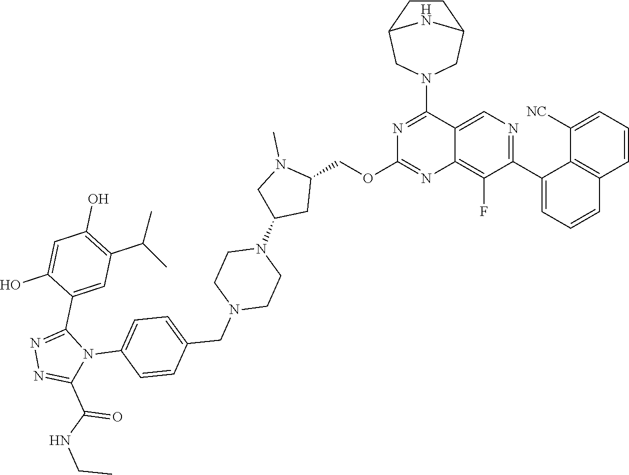 [SMILES: CCNC(=O)c1nnc(-c2cc(C(C)C)c(O)cc2O)n1-c1ccc(CN2CCN([C@H]3C[C@@H](COc4nc(N5CC6CCC(C5)N6)c5cnc(-c6cccc7cccc(C#N)c67)c(F)c5n4)N(C)C3)CC2)cc1] |
| | 986.1 |
| 60 | 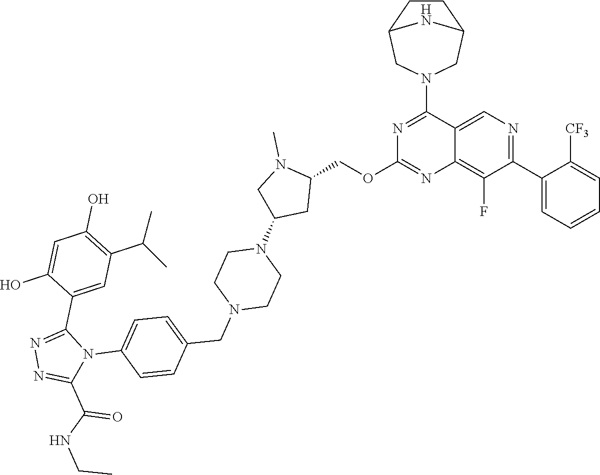 [SMILES: CCNC(=O)c1nnc(-c2cc(C(C)C)c(O)cc2O)n1-c1ccc(CN2CCN([C@H]3C[C@@H](COc4nc(N5CC6CCC(C5)N6)c5cnc(-c6ccccc6C(F)(F)F)c(F)c5n4)N(C)C3)CC2)cc1] |
| | 979.1 |
| 61 | 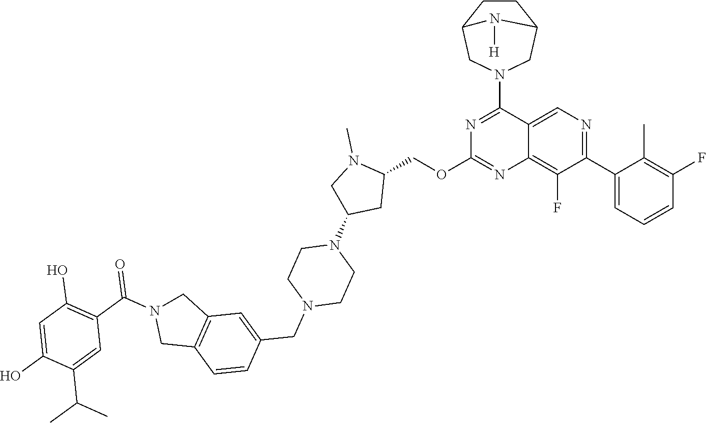 [SMILES: Cc1c(F)cccc1-c1ncc2c(N3CC4CCC(C3)N4)nc(OC[C@@H]3C[C@H](N4CCN(Cc5ccc6c(c5)CN(C(=O)c5cc(C(C)C)c(O)cc5O)C6)CC4)CN3C)nc2c1F] |
| | 874.1 |
| 62 | 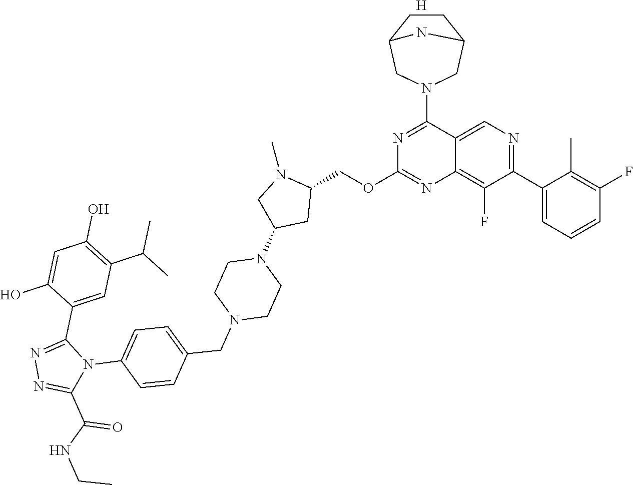 |
| | 943.1 |
| 63 | 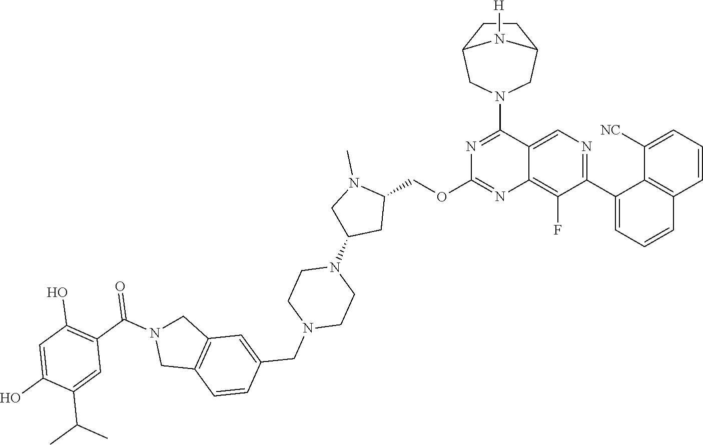 [SMILES: CC(C)c1cc(C(=O)N2Cc3ccc(CN4CCN([C@H]5C[C@@H](COc6nc(N7CC8CCC(C7)N8)c7cnc(-c8cccc9cccc(C#N)c89)c(F)c7n6)N(C)C5)CC4)cc3C2)c(O)cc1O] |
| | 917.1 |
| 64 | 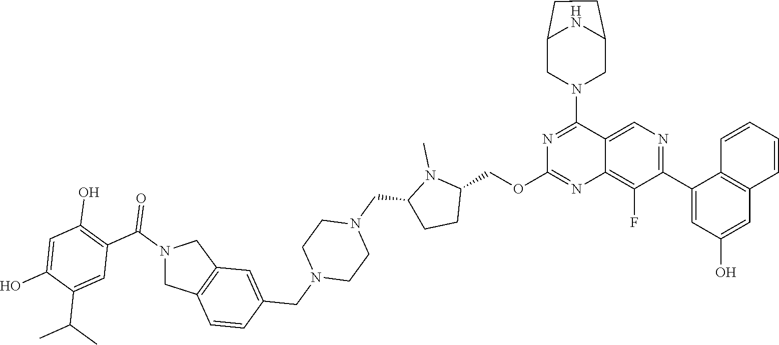 [SMILES: CC(C)c1cc(C(=O)N2Cc3ccc(CN4CCN(C[C@H]5CC[C@@H](COc6nc(N7CC8CCC(C7)N8)c7cnc(-c8cc(O)cc9ccccc89)c(F)c7n6)N5C)CC4)cc3C2)c(O)cc1O] |
| | 922.1 |
| 65 | 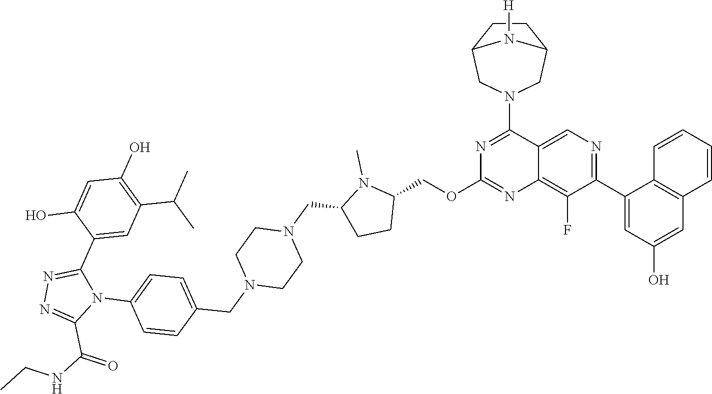 [SMILES: CCNC(=O)c1nnc(-c2cc(C(C)C)c(O)cc2O)n1-c1ccc(CN2CCN(C[C@H]3CC[C@@H](COc4nc(N5CC6CCC(C5)N6)c5cnc(-c6cc(O)cc7ccccc67)c(F)c5n4)N3C)CC2)cc1] |
| | 991.7 |
| 66 | 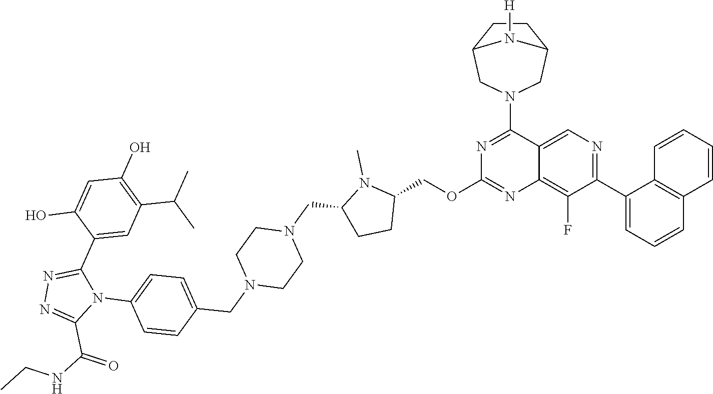 [SMILES: CCNC(=O)c1nnc(-c2cc(C(C)C)c(O)cc2O)n1-c1ccc(CN2CCN(C[C@H]3CC[C@@H](COc4nc(N5CC6CCC(C5)N6)c5cnc(-c6cccc7ccccc67)c(F)c5n4)N3C)CC2)cc1] |
| | 975.1 |
| 67 | 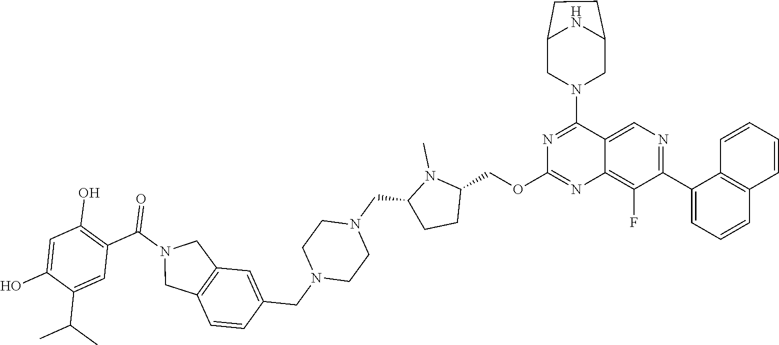 [SMILES: CC(C)c1cc(C(=O)N2Cc3ccc(CN4CCN(C[C@H]5CC[C@@H](COc6nc(N7CC8CCC(C7)N8)c7cnc(-c8cccc9ccccc89)c(F)c7n6)N5C)CC4)cc3C2)c(O)cc1O] |
| | 906.3 |
| 68 | 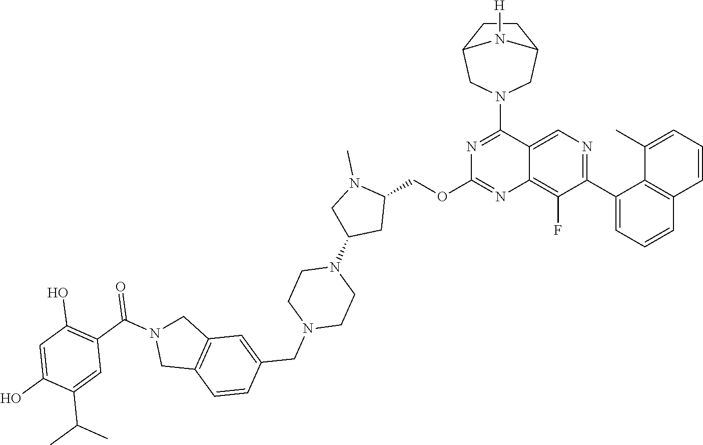 [SMILES: Cc1cccc2cccc(-c3ncc4c(N5CC6CCC(C5)N6)nc(OC[C@@H]5C[C@H](N6CCN(Cc7ccc8c(c7)CN(C(=O)c7cc(C(C)C)c(O)cc7O)C8)CC6)CN5C)nc4c3F)c12] |
| | 906.5 |
| 69 | 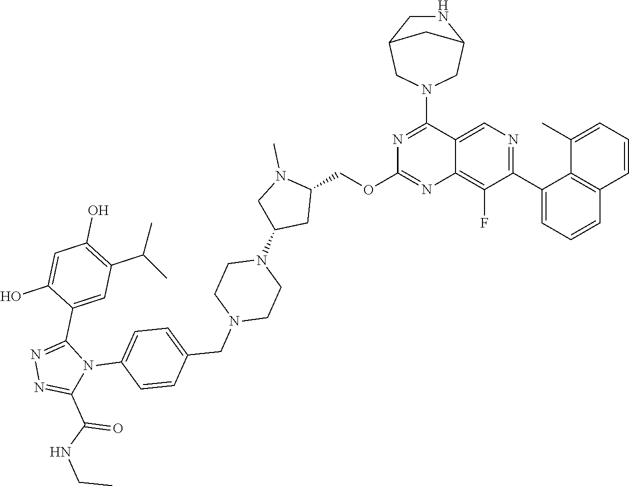 [SMILES: CCNC(=O)c1nnc(-c2cc(C(C)C)c(O)cc2O)n1-c1ccc(CN2CCN([C@H]3C[C@@H](COc4nc(N5CC6CNC(C6)C5)c5cnc(-c6cccc7cccc(C)c67)c(F)c5n4)N(C)C3)CC2)cc1] |
| | 975.1 |
| 70 | 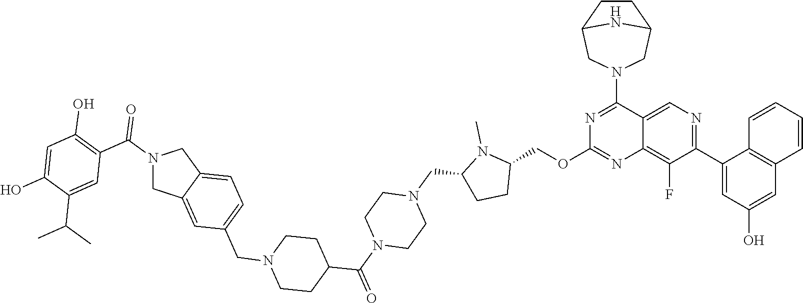 [SMILES: CC(C)c1cc(C(=O)N2Cc3ccc(CN4CCC(C(=O)N5CCN(C[C@H]6CC[C@@H](COc7nc(N8CC9CCC(C8)N9)c8cnc(-c9cc(O)cc%10ccccc9%10)c(F)c8n7)N6C)CC5)CC4)cc3C2)c(O)cc1O] |
| | 1033.1 |
| 71 | 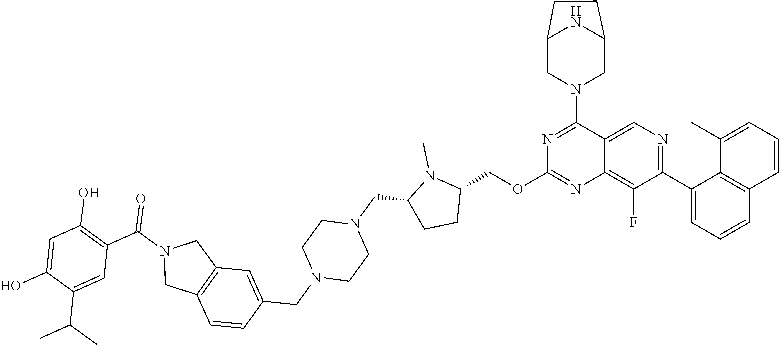 [SMILES: Cc1cccc2cccc(-c3ncc4c(N5CC6CCC(C5)N6)nc(OC[C@@H]5CC[C@H](CN6CCN(Cc7ccc8c(c7)CN(C(=O)c7cc(C(C)C)c(O)cc7O)C8)CC6)N5C)nc4c3F)c12] |
| | 920.1 |
| 72 | 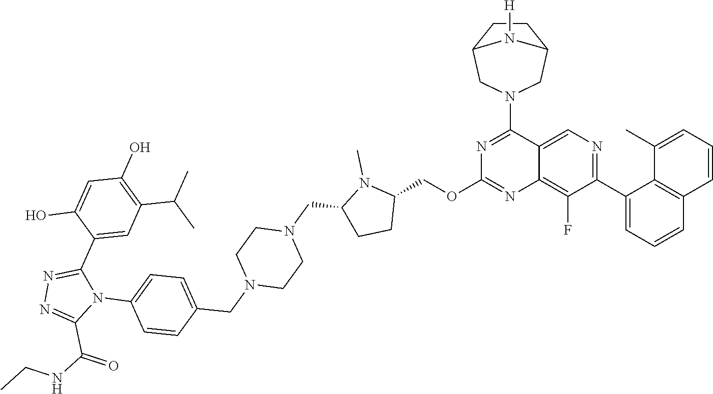 [SMILES: CCNC(=O)c1nnc(-c2cc(C(C)C)c(O)cc2O)n1-c1ccc(CN2CCN(C[C@H]3CC[C@@H](COc4nc(N5CC6CCC(C5)N6)c5cnc(-c6cccc7cccc(C)c67)c(F)c5n4)N3C)CC2)cc1] |
| | 1164.4 |
| 73 | 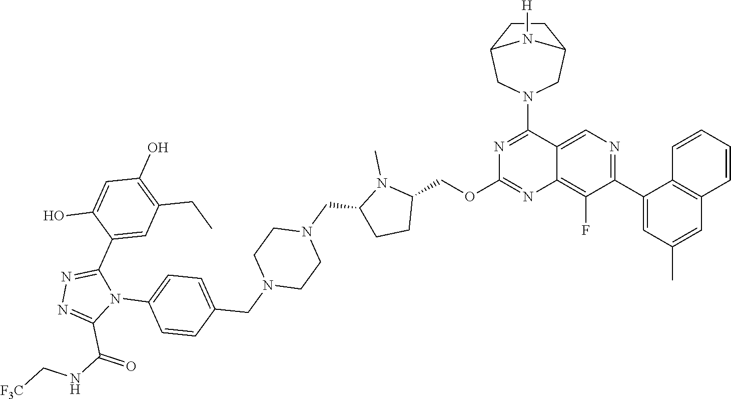 [SMILES: CCc1cc(-c2nnc(C(=O)NCC(F)(F)F)n2-c2ccc(CN3CCN(C[C@H]4CC[C@@H](COc5nc(N6CC7CCC(C6)N7)c6cnc(-c7cc(C)cc8ccccc78)c(F)c6n5)N4C)CC3)cc2)c(O)cc1O] |
| | 1031.1 |
| 74 | 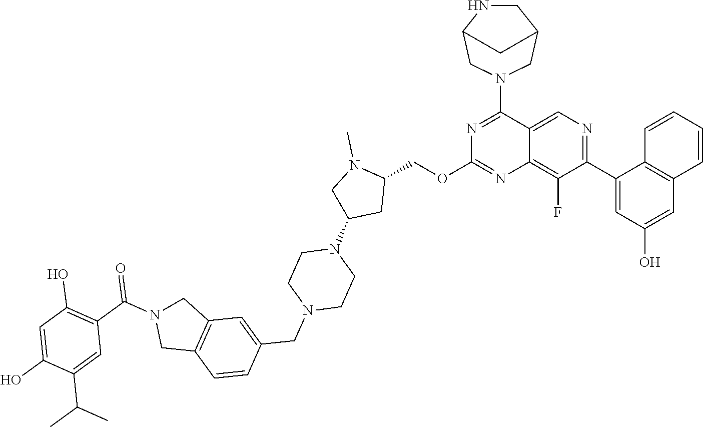 [SMILES: CC(C)c1cc(C(=O)N2Cc3ccc(CN4CCN([C@H]5C[C@@H](COc6nc(N7CC8CNC(C8)C7)c7cnc(-c8cc(O)cc9ccccc89)c(F)c7n6)N(C)C5)CC4)cc3C2)c(O)cc1O] |
| | 908.1 |
| 75 | 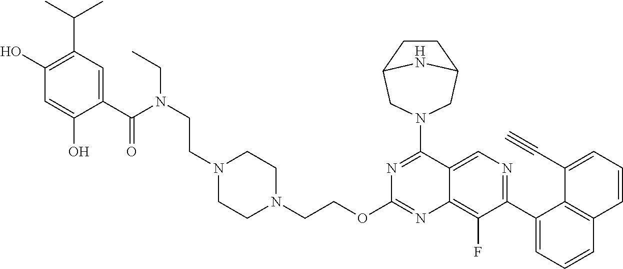 [SMILES: C#Cc1cccc2cccc(-c3ncc4c(N5CC6CCC(C5)N6)nc(OCCN5CCN(CCN(CC)C(=O)c6cc(C(C)C)c(O)cc6O)CC5)nc4c3F)c12] |
| | 787.0 |
| 76 | 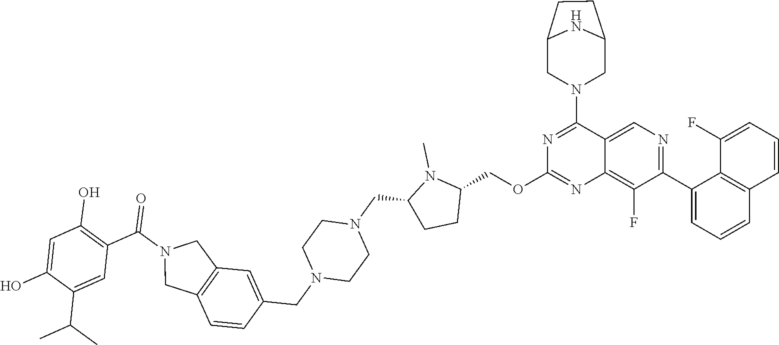 [SMILES: CC(C)c1cc(C(=O)N2Cc3ccc(CN4CCN(C[C@H]5CC[C@@H](COc6nc(N7CC8CCC(C7)N8)c7cnc(-c8cccc9cccc(F)c89)c(F)c7n6)N5C)CC4)cc3C2)c(O)cc1O] |
| | 910.1 |
| 77 | 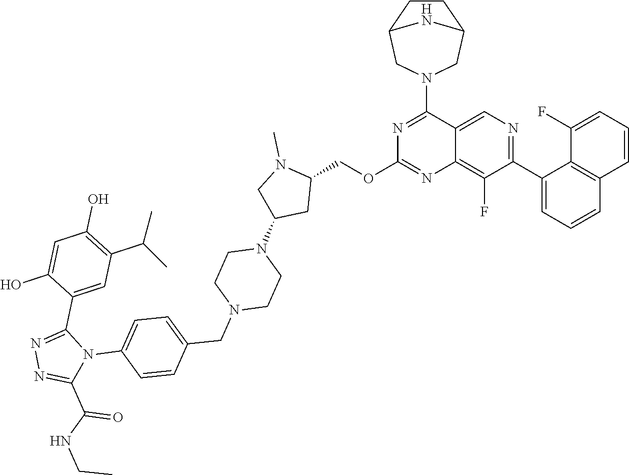 [SMILES: CCNC(=O)c1nnc(-c2cc(C(C)C)c(O)cc2O)n1-c1ccc(CN2CCN([C@H]3C[C@@H](COc4nc(N5CC6CCC(C5)N6)c5cnc(-c6cccc7cccc(F)c67)c(F)c5n4)N(C)C3)CC2)cc1] |
| | 979.1 |
| 78 | 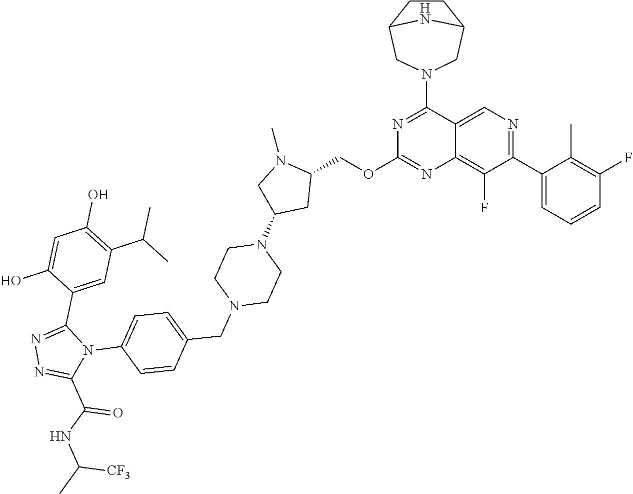 [SMILES: Cc1c(F)cccc1-c1ncc2c(N3CC4CCC(C3)N4)nc(OC[C@@H]3C[C@H](N4CCN(Cc5ccc(-n6c(C(=O)NC(C)C(F)(F)F)nnc6-c6cc(C(C)C)c(O)cc6O)cc5)CC4)CN3C)nc2c1F] |
| | 1011.1 |
| 79 | 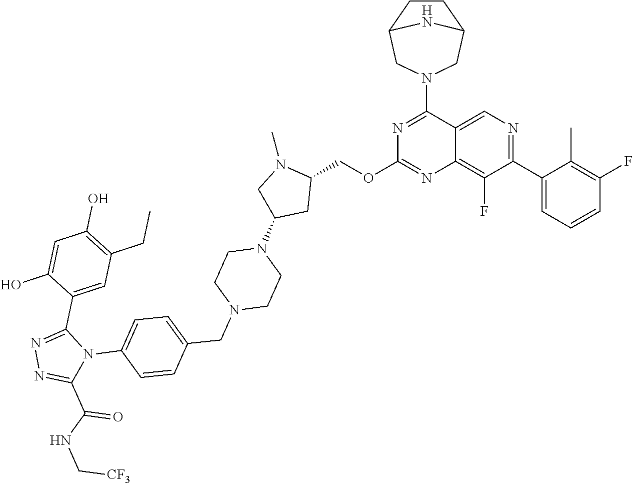 [SMILES: CCc1cc(-c2nnc(C(=O)NCC(F)(F)F)n2-c2ccc(CN3CCN([C@H]4C[C@@H](COc5nc(N6CC7CCC(C6)N7)c6cnc(-c7cccc(F)c7C)c(F)c6n5)N(C)C4)CC3)cc2)c(O)cc1O] |
| | 983.0 |
| 80 | 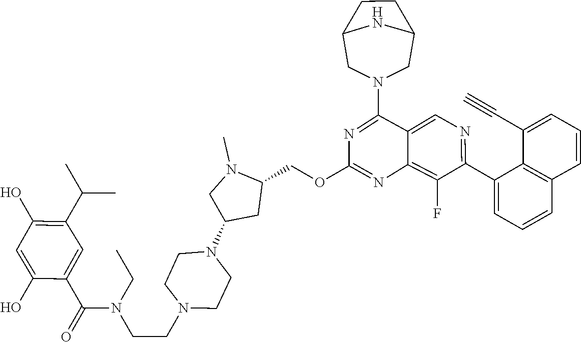 [SMILES: C#Cc1cccc2cccc(-c3ncc4c(N5CC6CCC(C5)N6)nc(OC[C@@H]5C[C@H](N6CCN(CCN(CC)C(=O)c7cc(C(C)C)c(O)cc7O)CC6)CN5C)nc4c3F)c12] |
| | 856.2 |
| 81 | 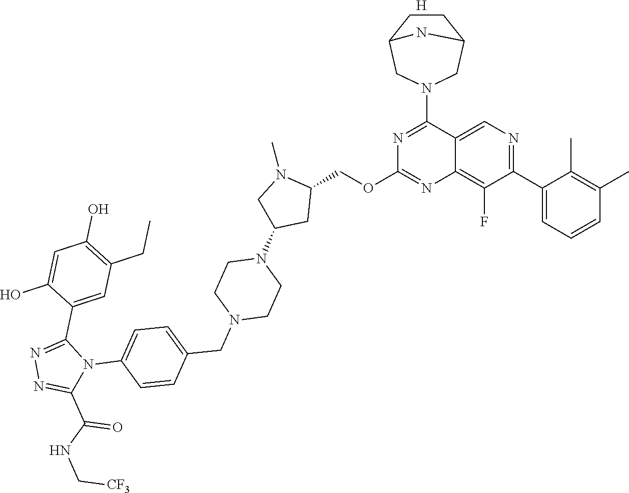 |
| | 979.1 |
| 82 | 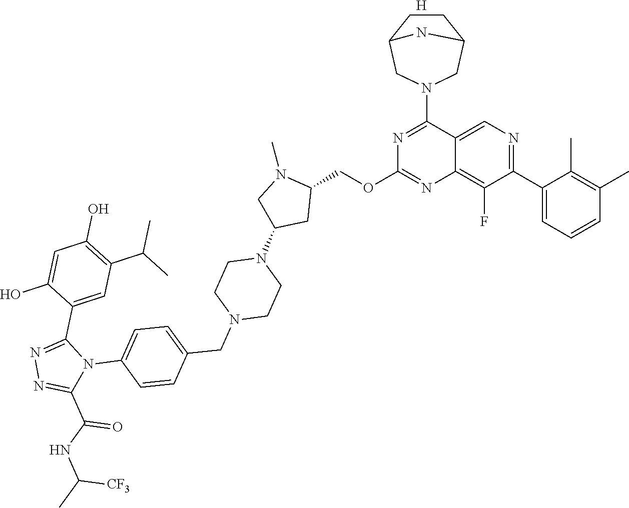 |
| | 1007.0 |
| 83 | 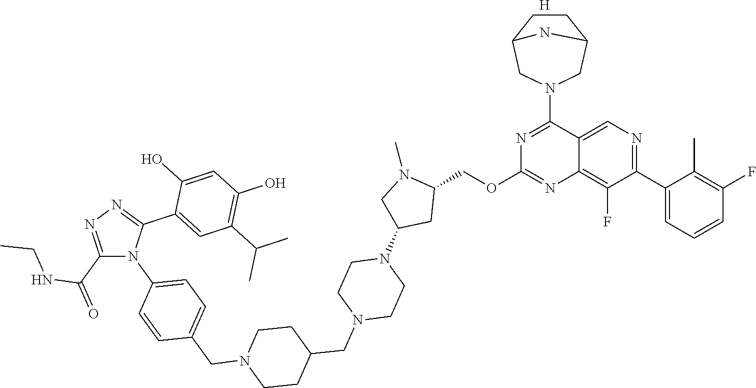 |
| | 1054.2 |
| 84 | 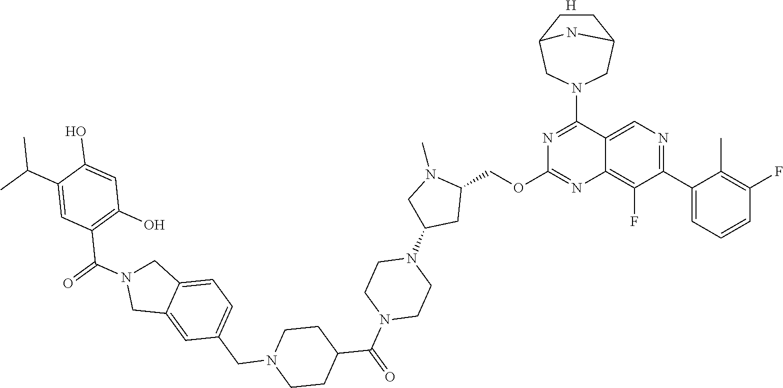 |
| | 985.1 |
| 85 | 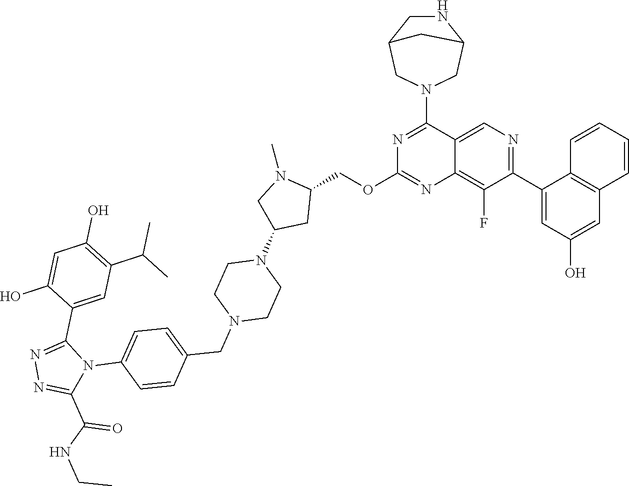 [SMILES: CCNC(=O)c1nnc(-c2cc(C(C)C)c(O)cc2O)n1-c1ccc(CN2CCN([C@H]3C[C@@H](COc4nc(N5CC6CNC(C6)C5)c5cnc(-c6cc(O)cc7ccccc67)c(F)c5n4)N(C)C3)CC2)cc1] |
| | 977.1 |
| 86 | 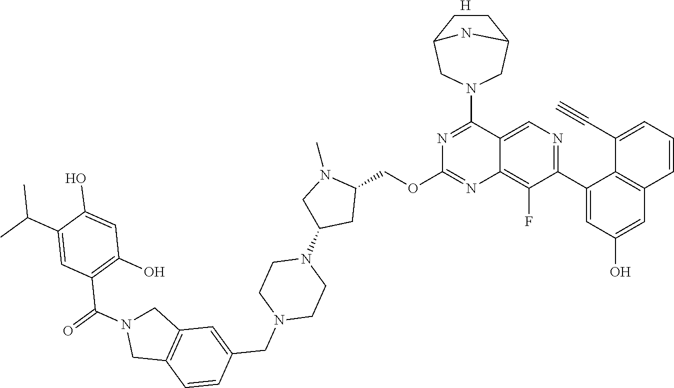 |
| | 932.1 |
| 87 | 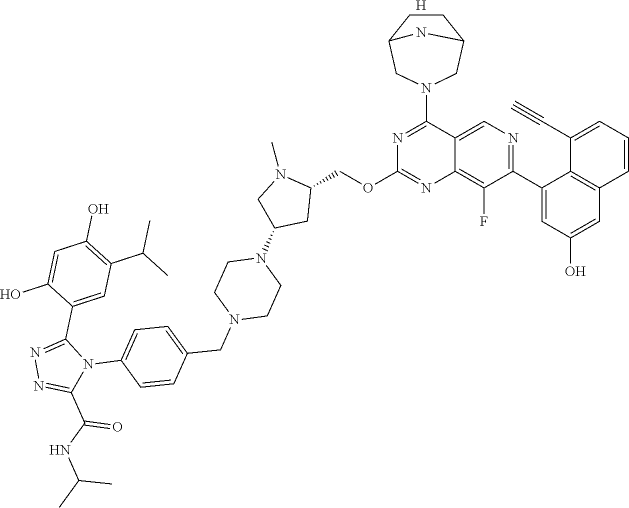 |
| | 1015.0 |
| 88 | 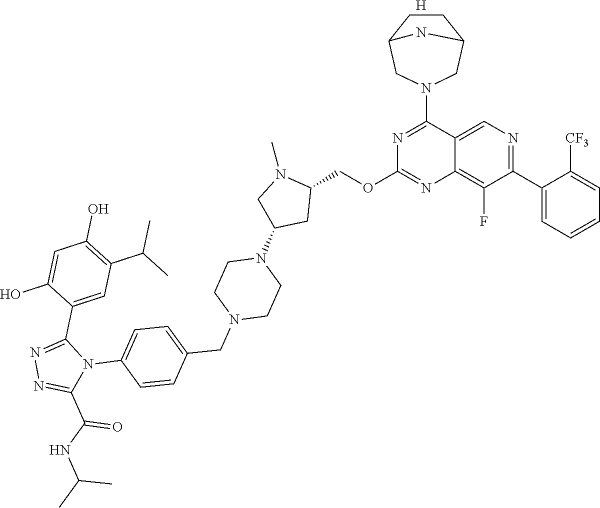 |
| | 993.1 |
| 89 | 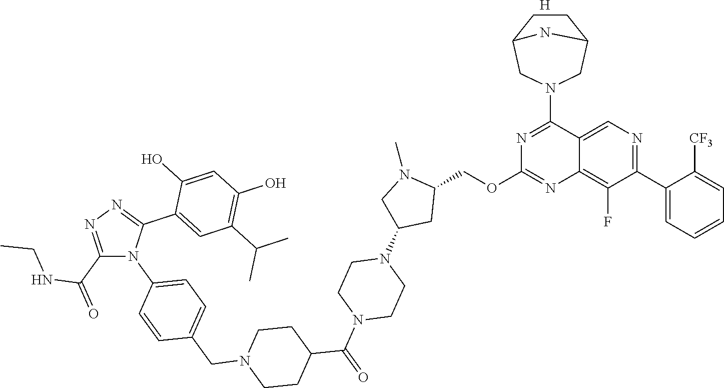 |
| | 1090.1 |
| 90 | 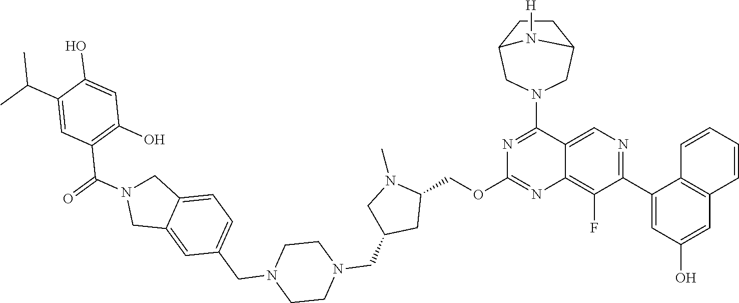 [SMILES: CC(C)c1cc(C(=O)N2Cc3ccc(CN4CCN(C[C@H]5C[C@@H](COc6nc(N7CC8CCC(C7)N8)c7cnc(-c8cc(O)cc9ccccc89)c(F)c7n6)N(C)C5)CC4)cc3C2)c(O)cc1O] |
| | 922.1 |
| 91 | 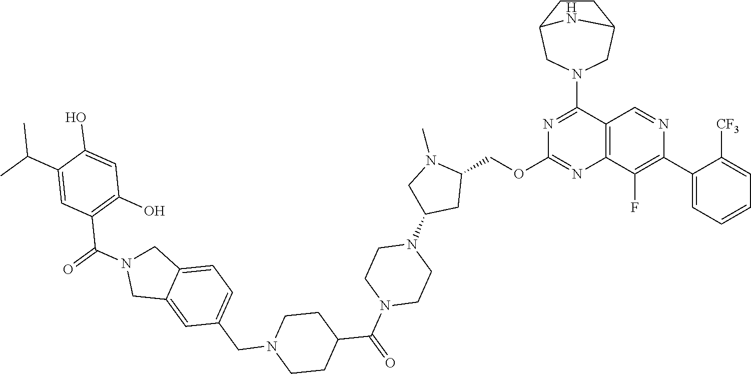 [SMILES: CC(C)c1cc(C(=O)N2Cc3ccc(CN4CCC(C(=O)N5CCN([C@H]6C[C@@H](COc7nc(N8CC9CCC(C8)N9)c8cnc(-c9ccccc9C(F)(F)F)c(F)c8n7)N(C)C6)CC5)CC4)cc3C2)c(O)cc1O] |
| | 1021.1 |
| 92 | 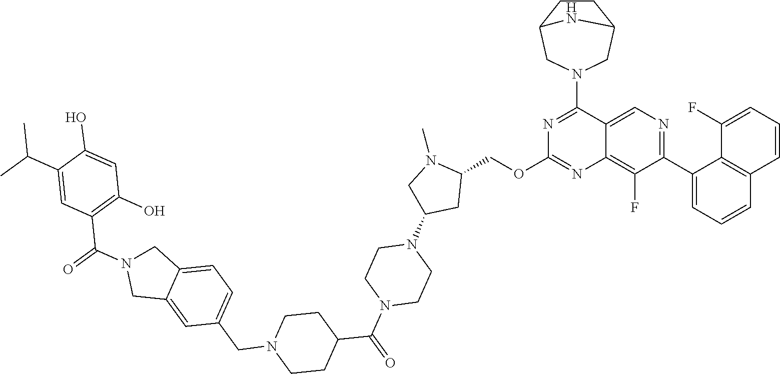 [SMILES: CC(C)c1cc(C(=O)N2Cc3ccc(CN4CCC(C(=O)N5CCN([C@H]6C[C@@H](COc7nc(N8CC9CCC(C8)N9)c8cnc(-c9cccc%10cccc(F)c9%10)c(F)c8n7)N(C)C6)CC5)CC4)cc3C2)c(O)cc1O] |
| | 1021.1 |
| 93 | 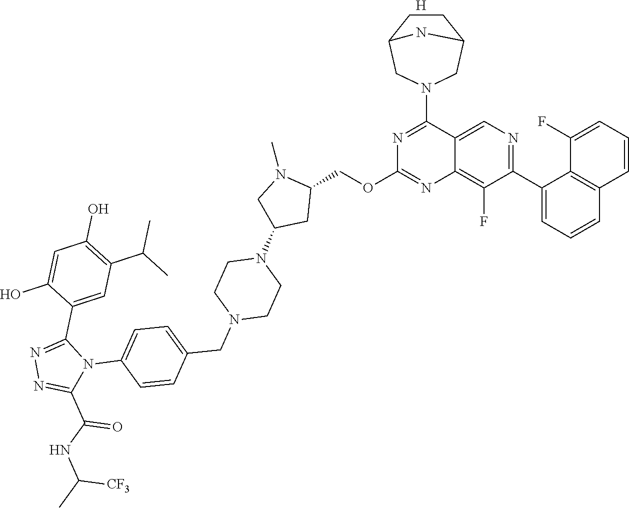 |
| | 1047.2 |
| 94 | 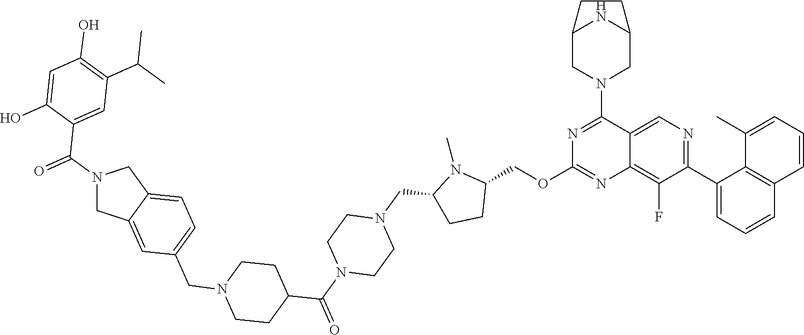 [SMILES: Cc1cccc2cccc(-c3ncc4c(N5CC6CCC(C5)N6)nc(OC[C@@H]5CC[C@H](CN6CCN(C(=O)C7CCN(Cc8ccc9c(c8)CN(C(=O)c8cc(C(C)C)c(O)cc8O)C9)CC7)CC6)N5C)nc4c3F)c12] |
| | 1031.2 |
| 95 | 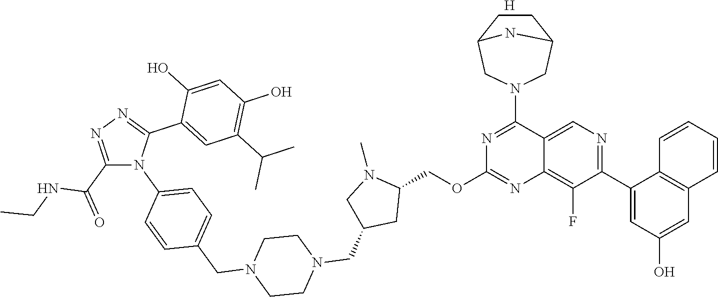 |
| | 991.1 |
| 96 | 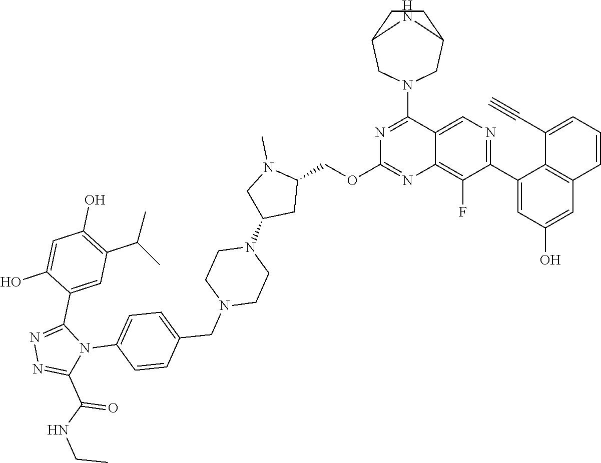 [SMILES: C#Cc1cccc2cc(O)cc(-c3ncc4c(N5CC6CCC(C5)N6)nc(OC[C@@H]5C[C@H](N6CCN(Cc7ccc(-n8c(C(=O)NCC)nnc8-c8cc(C(C)C)c(O)cc8O)cc7)CC6)CN5C)nc4c3F)c12] |
| | 1001.1 |
| 97 | 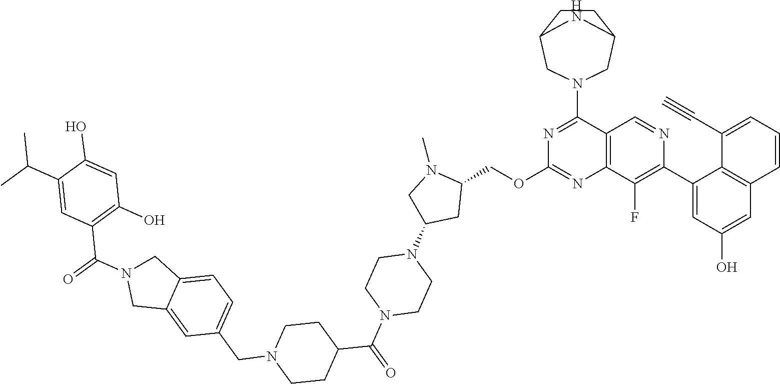 [SMILES: C#Cc1cccc2cc(O)cc(-c3ncc4c(N5CC6CCC(C5)N6)nc(OC[C@@H]5C[C@H](N6CCN(C(=O)C7CCN(Cc8ccc9c(c8)CN(C(=O)c8cc(C(C)C)c(O)cc8O)C9)CC7)CC6)CN5C)nc4c3F)c12] |
| | 1043.1 |
| 98 | 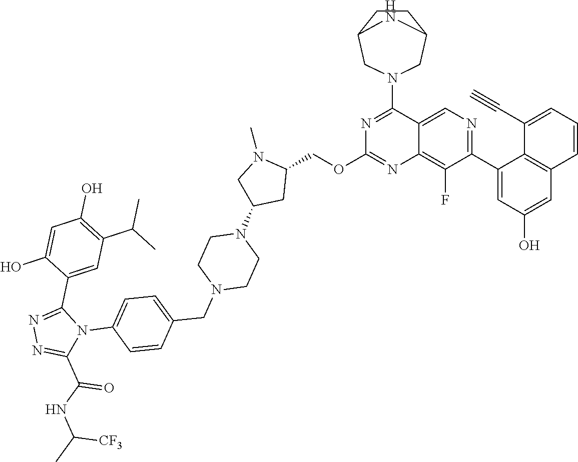 [SMILES: C#Cc1cccc2cc(O)cc(-c3ncc4c(N5CC6CCC(C5)N6)nc(OC[C@@H]5C[C@H](N6CCN(Cc7ccc(-n8c(C(=O)NC(C)C(F)(F)F)nnc8-c8cc(C(C)C)c(O)cc8O)cc7)CC6)CN5C)nc4c3F)c12] |
| | 1069.0 |
| 99 | 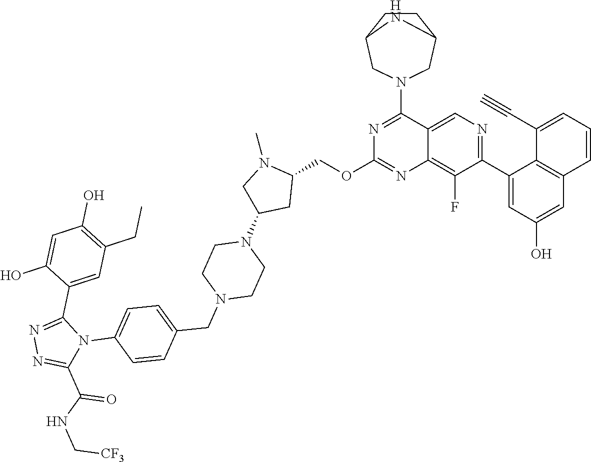 [SMILES: C#Cc1cccc2cc(O)cc(-c3ncc4c(N5CC6CCC(C5)N6)nc(OC[C@@H]5C[C@H](N6CCN(Cc7ccc(-n8c(C(=O)NCC(F)(F)F)nnc8-c8cc(CC)c(O)cc8O)cc7)CC6)CN5C)nc4c3F)c12] |
| | 1041.0 |
| 100 | 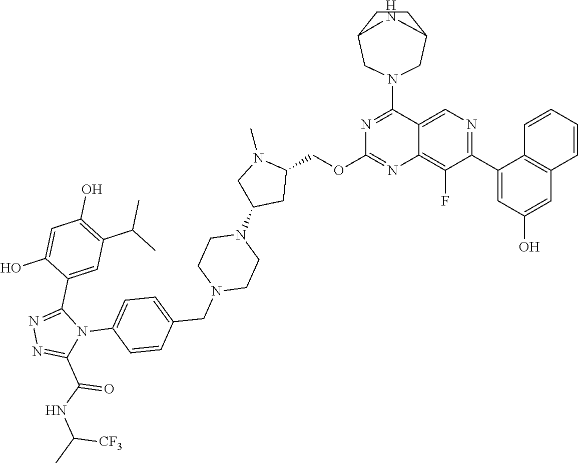 [SMILES: CC(C)c1cc(-c2nnc(C(=O)NC(C)C(F)(F)F)n2-c2ccc(CN3CCN([C@H]4C[C@@H](COc5nc(N6CC7CCC(C6)N7)c6cnc(-c7cc(O)cc8ccccc78)c(F)c6n5)N(C)C4)CC3)cc2)c(O)cc1O] |
| | 1045.1 |
| 101 | 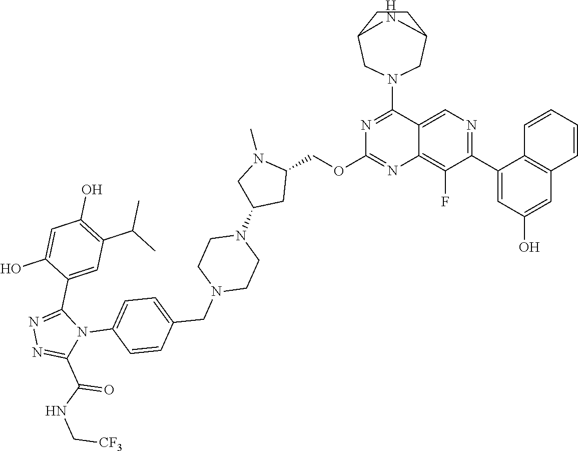 [SMILES: CC(C)c1cc(-c2nnc(C(=O)NCC(F)(F)F)n2-c2ccc(CN3CCN([C@H]4C[C@@H](COc5nc(N6CC7CCC(C6)N7)c6cnc(-c7cc(O)cc8ccccc78)c(F)c6n5)N(C)C4)CC3)cc2)c(O)cc1O] |
| | 1031.1 |
| 102 | 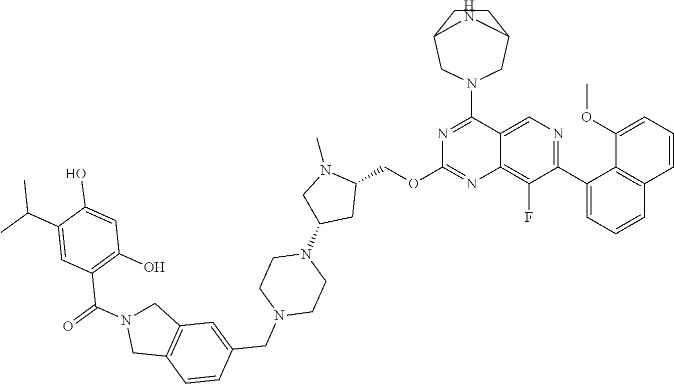 [SMILES: COc1cccc2cccc(-c3ncc4c(N5CC6CCC(C5)N6)nc(OC[C@@H]5C[C@H](N6CCN(Cc7ccc8c(c7)CN(C(=O)c7cc(C(C)C)c(O)cc7O)C8)CC6)CN5C)nc4c3F)c12] |
| | 922.1 |
| 103 | 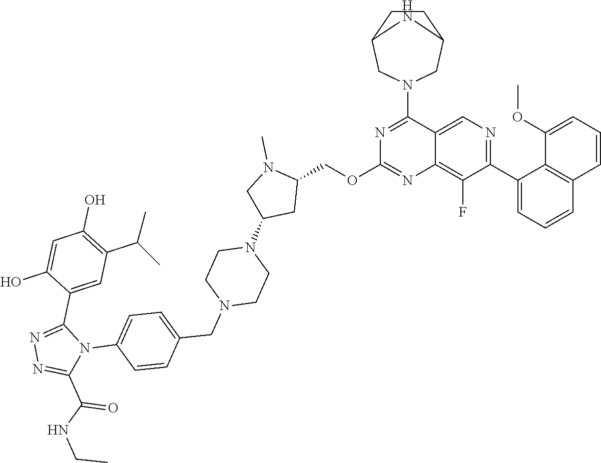 [SMILES: CCNC(=O)c1nnc(-c2cc(C(C)C)c(O)cc2O)n1-c1ccc(CN2CCN([C@H]3C[C@@H](COc4nc(N5CC6CCC(C5)N6)c5cnc(-c6cccc7cccc(OC)c67)c(F)c5n4)N(C)C3)CC2)cc1] |
| | 991.1 |
| 104 | 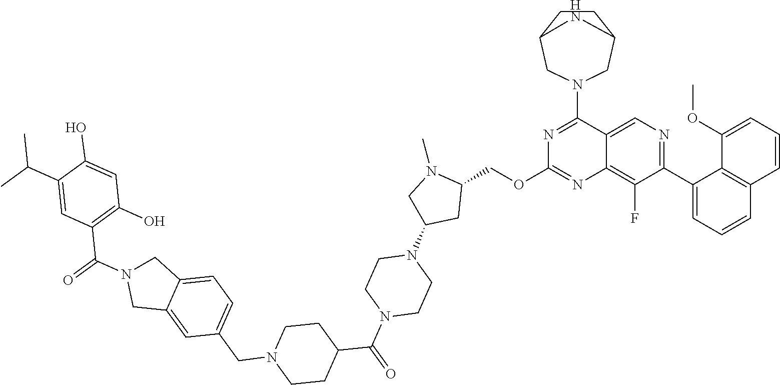 [SMILES: COc1cccc2cccc(-c3ncc4c(N5CC6CCC(C5)N6)nc(OC[C@@H]5C[C@H](N6CCN(C(=O)C7CCN(Cc8ccc9c(c8)CN(C(=O)c8cc(C(C)C)c(O)cc8O)C9)CC7)CC6)CN5C)nc4c3F)c12] |
| | 1033.1 |
| 105 | 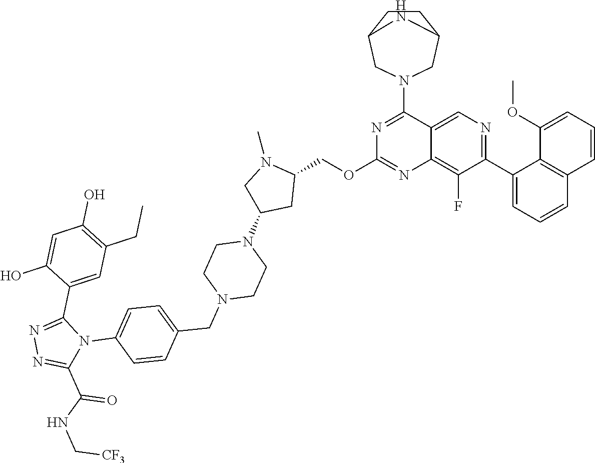 [SMILES: CCc1cc(-c2nnc(C(=O)NCC(F)(F)F)n2-c2ccc(CN3CCN([C@H]4C[C@@H](COc5nc(N6CC7CCC(C6)N7)c6cnc(-c7cccc8cccc(OC)c78)c(F)c6n5)N(C)C4)CC3)cc2)c(O)cc1O] |
| | 1031.1 |
| 106 | 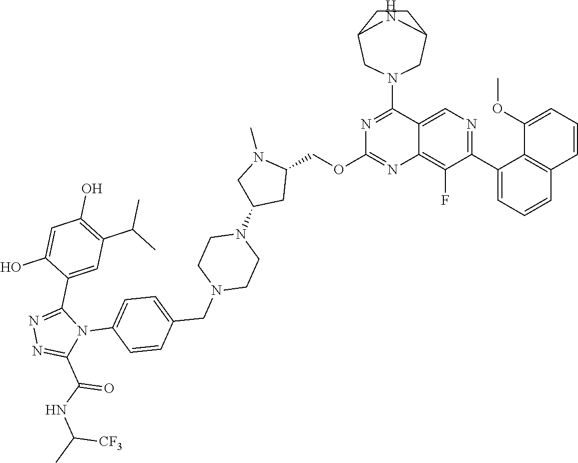 [SMILES: COc1cccc2cccc(-c3ncc4c(N5CC6CCC(C5)N6)nc(OC[C@@H]5C[C@H](N6CCN(Cc7ccc(-n8c(C(=O)NC(C)C(F)(F)F)nnc8-c8cc(C(C)C)c(O)cc8O)cc7)CC6)CN5C)nc4c3F)c12] |
| | 1059.2 |
| 107 | 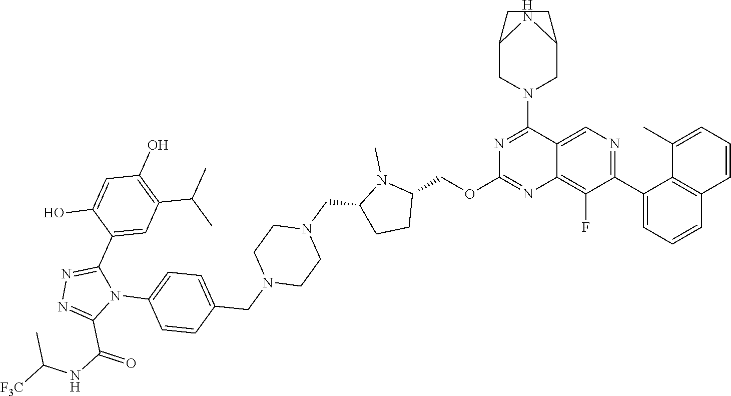 [SMILES: Cc1cccc2cccc(-c3ncc4c(N5CC6CCC(C5)N6)nc(OC[C@@H]5CC[C@H](CN6CCN(Cc7ccc(-n8c(C(=O)NC(C)C(F)(F)F)nnc8-c8cc(C(C)C)c(O)cc8O)cc7)CC6)N5C)nc4c3F)c12] |
| | 1057.2 |
| 108 | 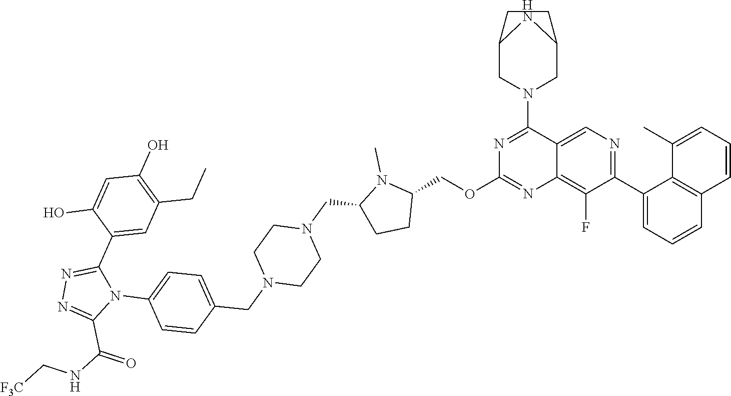 [SMILES: CCc1cc(-c2nnc(C(=O)NCC(F)(F)F)n2-c2ccc(CN3CCN(C[C@H]4CC[C@@H](COc5nc(N6CC7CCC(C6)N7)c6cnc(-c7cccc8cccc(C)c78)c(F)c6n5)N4C)CC3)cc2)c(O)cc1O] |
| | 1029.1 |
| 109 | 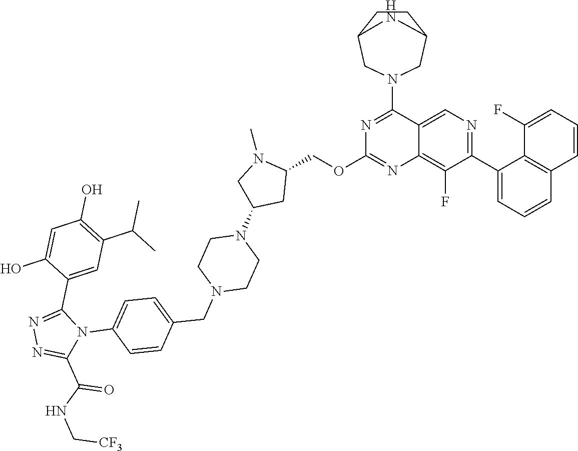 [SMILES: CC(C)c1cc(-c2nnc(C(=O)NCC(F)(F)F)n2-c2ccc(CN3CCN([C@H]4C[C@@H](COc5nc(N6CC7CCC(C6)N7)c6cnc(-c7cccc8cccc(F)c78)c(F)c6n5)N(C)C4)CC3)cc2)c(O)cc1O] |
| | 1020.0 |
| 110 | 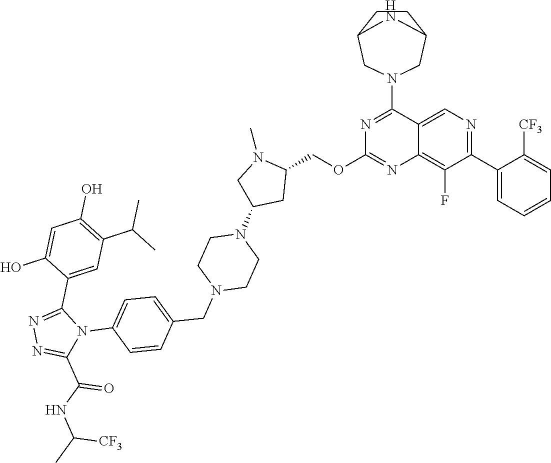 [SMILES: CC(C)c1cc(-c2nnc(C(=O)NC(C)C(F)(F)F)n2-c2ccc(CN3CCN([C@H]4C[C@@H](COc5nc(N6CC7CCC(C6)N7)c6cnc(-c7ccccc7C(F)(F)F)c(F)c6n5)N(C)C4)CC3)cc2)c(O)cc1O] |
| | 1047.2 |
| 111 | 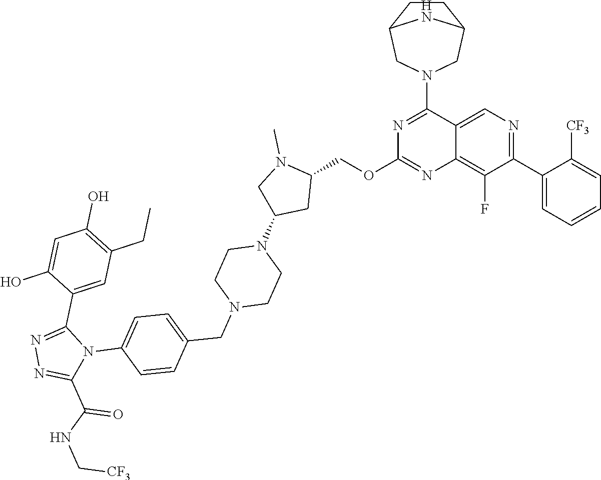 [SMILES: CCc1cc(-c2nnc(C(=O)NCC(F)(F)F)n2-c2ccc(CN3CCN([C@H]4C[C@@H](COc5nc(N6CC7CCC(C6)N7)c6cnc(-c7ccccc7C(F)(F)F)c(F)c6n5)N(C)C4)CC3)cc2)c(O)cc1O] |
| | 1019.0 |
| 112 | 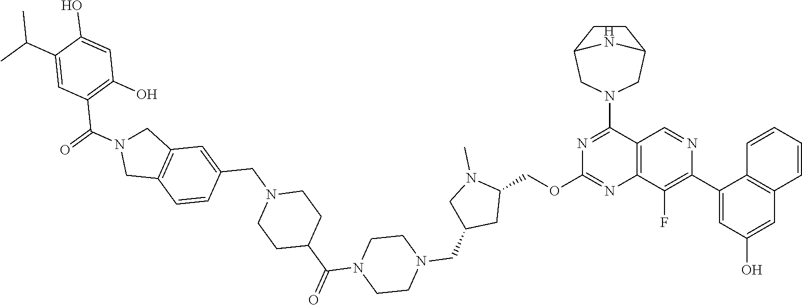 [SMILES: CC(C)c1cc(C(=O)N2Cc3ccc(CN4CCC(C(=O)N5CCN(C[C@H]6C[C@@H](COc7nc(N8CC9CCC(C8)N9)c8cnc(-c9cc(O)cc%10ccccc9%10)c(F)c8n7)N(C)C6)CC5)CC4)cc3C2)c(O)cc1O] |
| | 1033.2 |
| 113 | 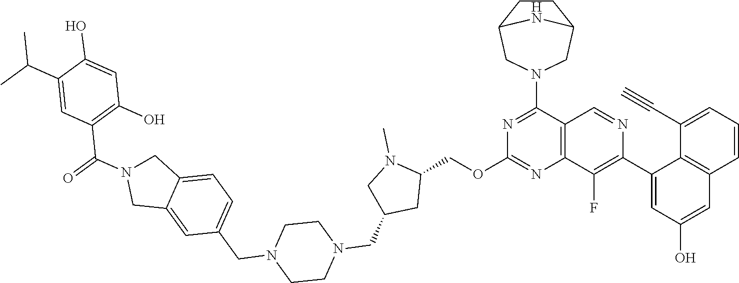 [SMILES: C#Cc1cccc2cc(O)cc(-c3ncc4c(N5CC6CCC(C5)N6)nc(OC[C@@H]5C[C@H](CN6CCN(Cc7ccc8c(c7)CN(C(=O)c7cc(C(C)C)c(O)cc7O)C8)CC6)CN5C)nc4c3F)c12] |
| | 946.1 |
| 114 | 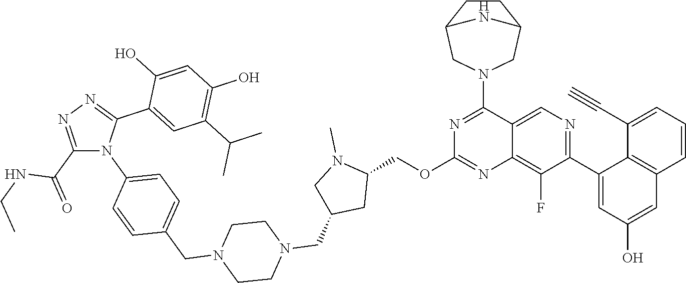 [SMILES: C#Cc1cccc2cc(O)cc(-c3ncc4c(N5CC6CCC(C5)N6)nc(OC[C@@H]5C[C@H](CN6CCN(Cc7ccc(-n8c(C(=O)NCC)nnc8-c8cc(C(C)C)c(O)cc8O)cc7)CC6)CN5C)nc4c3F)c12] |
| | 1015.1 |
| 115 | 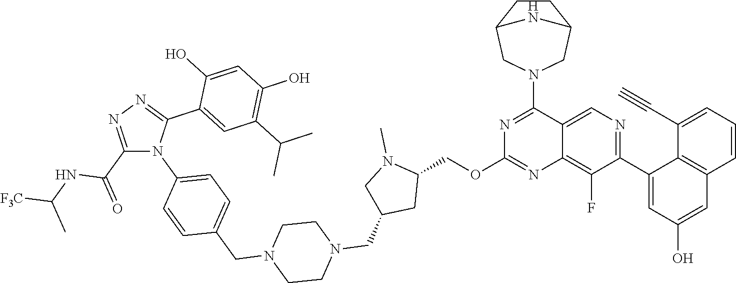 [SMILES: C#Cc1cccc2cc(O)cc(-c3ncc4c(N5CC6CCC(C5)N6)nc(OC[C@@H]5C[C@H](CN6CCN(Cc7ccc(-n8c(C(=O)NC(C)C(F)(F)F)nnc8-c8cc(C(C)C)c(O)cc8O)cc7)CC6)CN5C)nc4c3F)c12] |
| | 1083.1 |
| 116 | 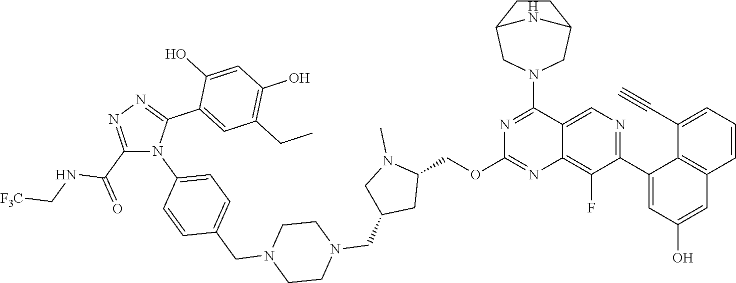 [SMILES: C#Cc1cccc2cc(O)cc(-c3ncc4c(N5CC6CCC(C5)N6)nc(OC[C@@H]5C[C@H](CN6CCN(Cc7ccc(-n8c(C(=O)NCC(F)(F)F)nnc8-c8cc(CC)c(O)cc8O)cc7)CC6)CN5C)nc4c3F)c12] |
| | 1055.1 |
| 117 | 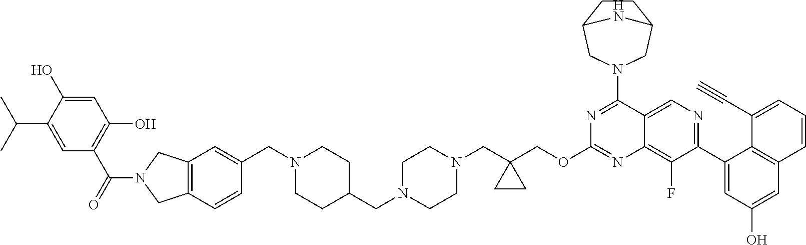 [SMILES: C#Cc1cccc2cc(O)cc(-c3ncc4c(N5CC6CCC(C5)N6)nc(OCC5(CN6CCN(CC7CCN(Cc8ccc9c(c8)CN(C(=O)c8cc(C(C)C)c(O)cc8O)C9)CC7)CC6)CC5)nc4c3F)c12] |
| | 1000.0 |
| 118 | 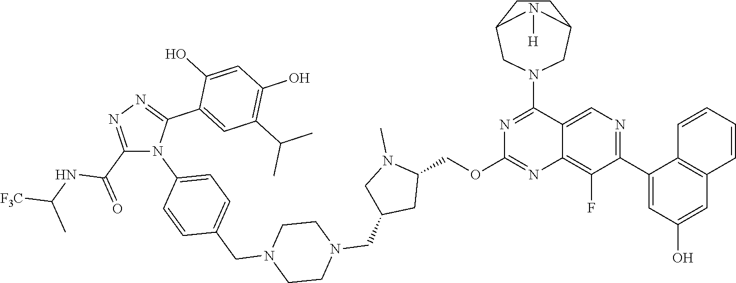 [SMILES: CC(C)c1cc(-c2nnc(C(=O)NC(C)C(F)(F)F)n2-c2ccc(CN3CCN(C[C@H]4C[C@@H](COc5nc(N6CC7CCC(C6)N7)c6cnc(-c7cc(O)cc8ccccc78)c(F)c6n5)N(C)C4)CC3)cc2)c(O)cc1O] |
| | 1059.1 |
| 119 | 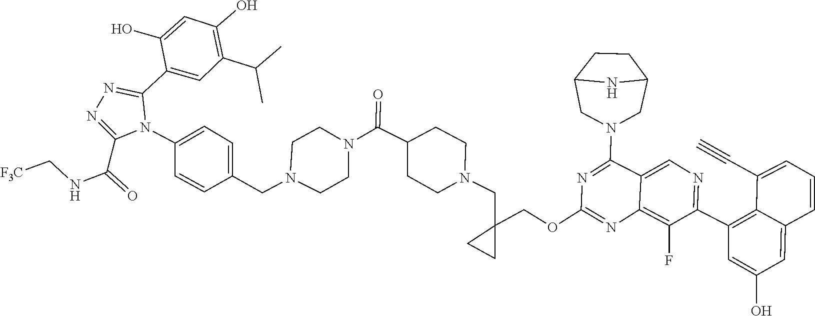 [SMILES: C#Cc1cccc2cc(O)cc(-c3ncc4c(N5CC6CCC(C5)N6)nc(OCC5(CN6CCC(C(=O)N7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1137.5 |
| 120 | 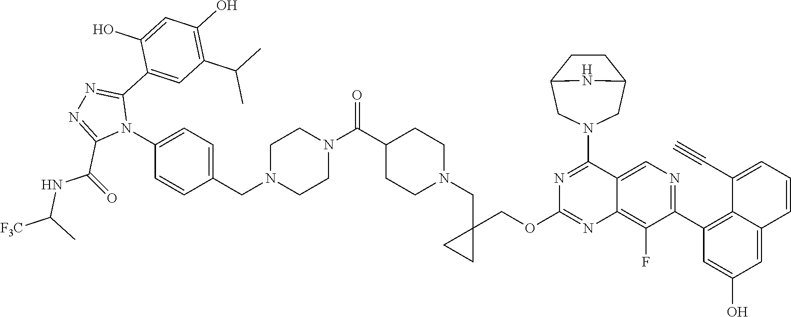 [SMILES: C#Cc1cccc2cc(O)cc(-c3ncc4c(N5CC6CCC(C5)N6)nc(OCC5(CN6CCC(C(=O)N7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1151.5 |
| 121 | 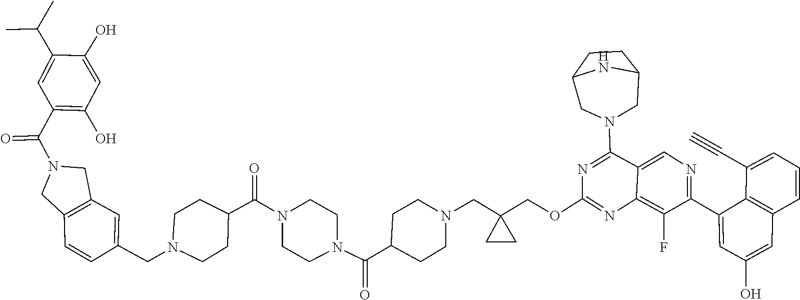 [SMILES: C#Cc1cccc2cc(O)cc(-c3ncc4c(N5CC6CCC(C5)N6)nc(OCC5(CN6CCC(C(=O)N7CCN(C(=O)C8CCN(Cc9ccc%10c(c9)CN(C(=O)c9cc(C(C)C)c(O)cc9O)C%10)CC8)CC7)CC6)CC5)nc4c3F)c12] |
| | 1125.2 |
| 122 | 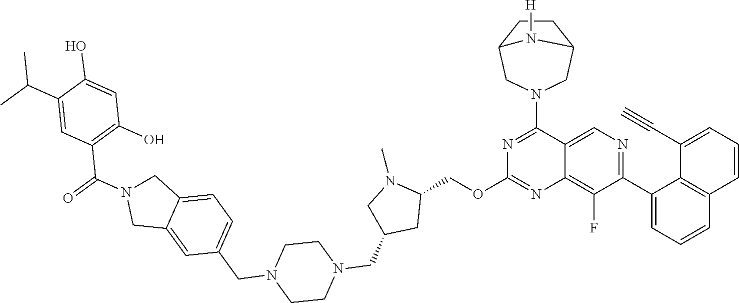 [SMILES: C#Cc1cccc2cccc(-c3ncc4c(N5CC6CCC(C5)N6)nc(OC[C@@H]5C[C@H](CN6CCN(Cc7ccc8c(c7)CN(C(=O)c7cc(C(C)C)c(O)cc7O)C8)CC6)CN5C)nc4c3F)c12] |
| | 930.1 |
| 123 | 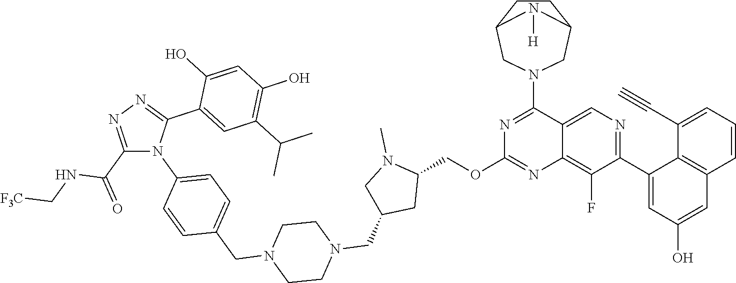 [SMILES: C#Cc1cccc2cc(O)cc(-c3ncc4c(N5CC6CCC(C5)N6)nc(OC[C@@H]5C[C@H](CN6CCN(Cc7ccc(-n8c(C(=O)NCC(F)(F)F)nnc8-c8cc(C(C)C)c(O)cc8O)cc7)CC6)CN5C)nc4c3F)c12] |
| | 1053.1 |
| 124 | 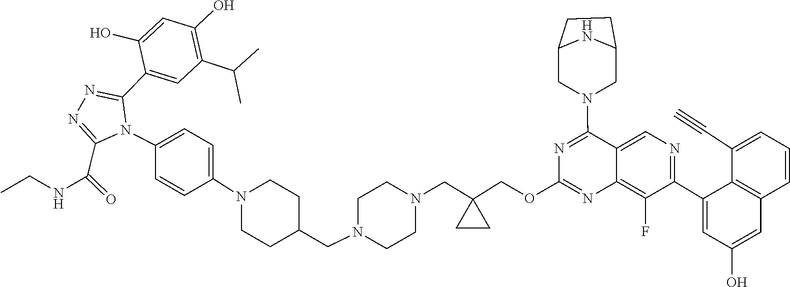 [SMILES: C#Cc1cccc2cc(O)cc(-c3ncc4c(N5CC6CCC(C5)N6)nc(OCC5(CN6CCN(CC7CCN(c8ccc(-n9c(C(=O)NCC)nnc9-c9cc(C(C)C)c(O)cc9O)cc8)CC7)CC6)CC5)nc4c3F)c12] |
| | 1055.1 |
| 125 | 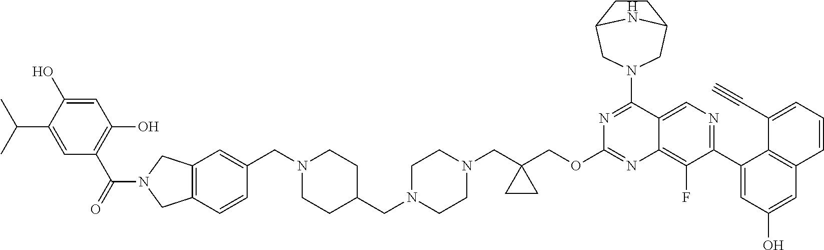 [SMILES: C#Cc1cccc2cc(O)cc(-c3ncc4c(N5CC6CCC(C5)N6)nc(OCC5(CN6CCN(CC7CCN(Cc8ccc9c(c8)CN(C(=O)c8cc(C(C)C)c(O)cc8O)C9)CC7)CC6)CC5)nc4c3F)c12] |
| | 1001.1 |
| 126 | 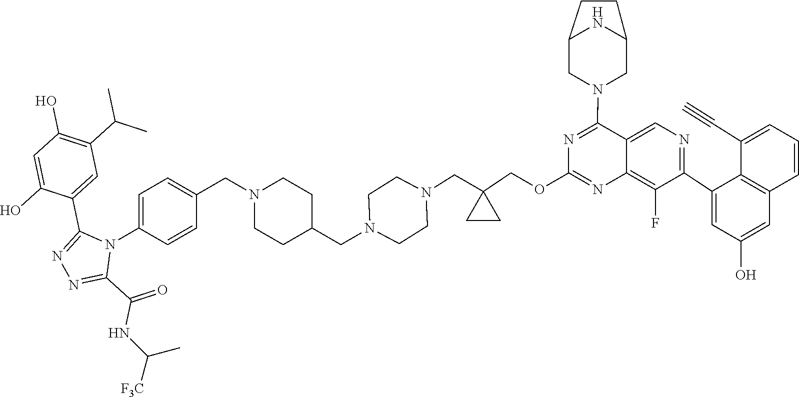 [SMILES: C#Cc1cccc2cc(O)cc(-c3ncc4c(N5CC6CCC(C5)N6)nc(OCC5(CN6CCN(CC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1137.0 |
| 127 | 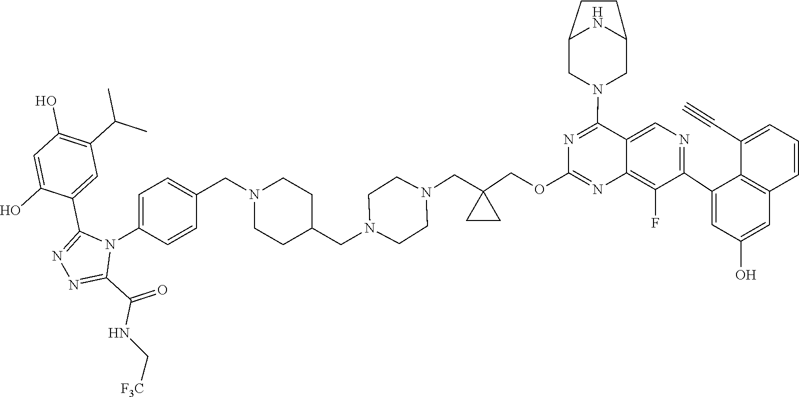 [SMILES: C#Cc1cccc2cc(O)cc(-c3ncc4c(N5CC6CCC(C5)N6)nc(OCC5(CN6CCN(CC7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1123.3 |
| 128 | 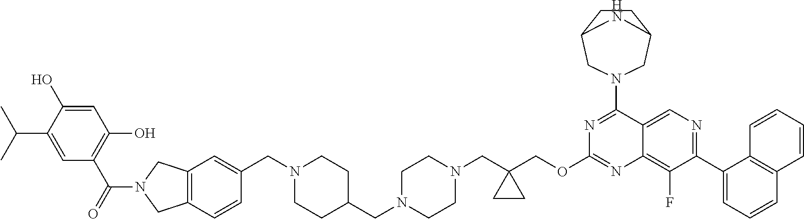 [SMILES: CC(C)c1cc(C(=O)N2Cc3ccc(CN4CCC(CN5CCN(CC6(COc7nc(N8CC9CCC(C8)N9)c8cnc(-c9cccc%10ccccc9%10)c(F)c8n7)CC6)CC5)CC4)cc3C2)c(O)cc1O] |
| | 960.2 |
| 129 | 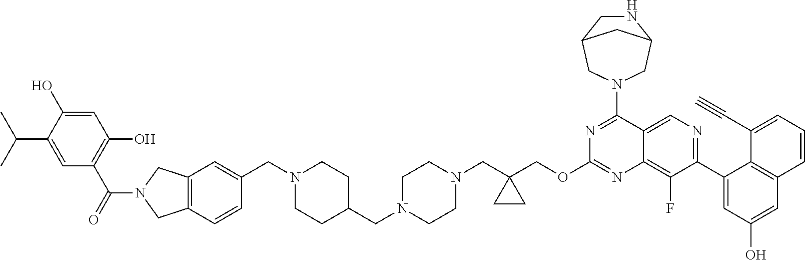 [SMILES: C#Cc1cccc2cc(O)cc(-c3ncc4c(N5CC6CNC(C6)C5)nc(OCC5(CN6CCN(CC7CCN(Cc8ccc9c(c8)CN(C(=O)c8cc(C(C)C)c(O)cc8O)C9)CC7)CC6)CC5)nc4c3F)c12] |
| | 1000.1 |
| 130 | 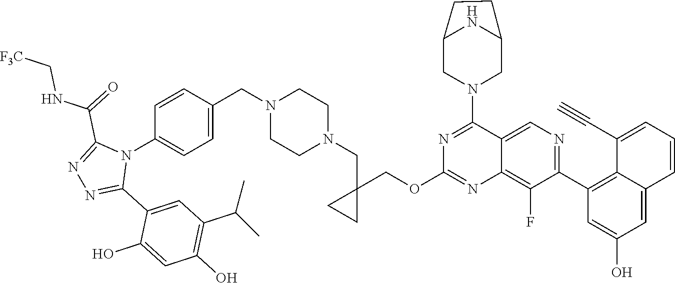 [SMILES: C#Cc1cccc2cc(O)cc(-c3ncc4c(N5CC6CCC(C5)N6)nc(OCC5(CN6CCN(Cc7ccc(-n8c(C(=O)NCC(F)(F)F)nnc8-c8cc(C(C)C)c(O)cc8O)cc7)CC6)CC5)nc4c3F)c12] |
| | 1026.1 |
| 131 | 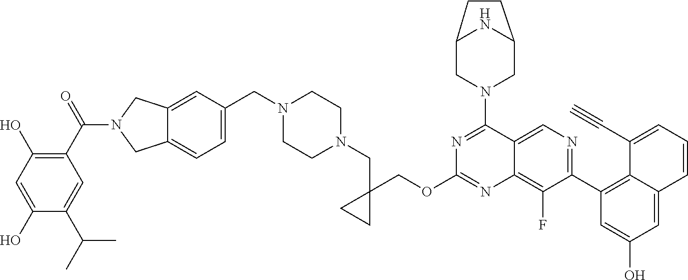 [SMILES: C#Cc1cccc2cc(O)cc(-c3ncc4c(N5CC6CCC(C5)N6)nc(OCC5(CN6CCN(Cc7ccc8c(c7)CN(C(=O)c7cc(C(C)C)c(O)cc7O)C8)CC6)CC5)nc4c3F)c12] |
| | 903.1 |
| 132 | 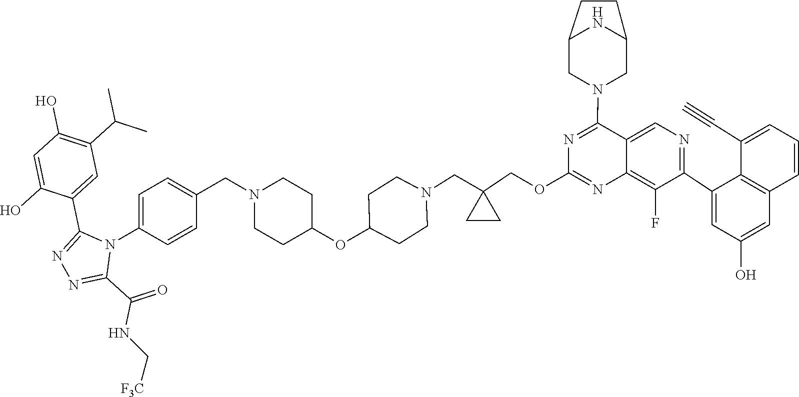 [SMILES: C#Cc1cccc2cc(O)cc(-c3ncc4c(N5CC6CCC(C5)N6)nc(OCC5(CN6CCC(OC7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1124.0 |
| 133 | 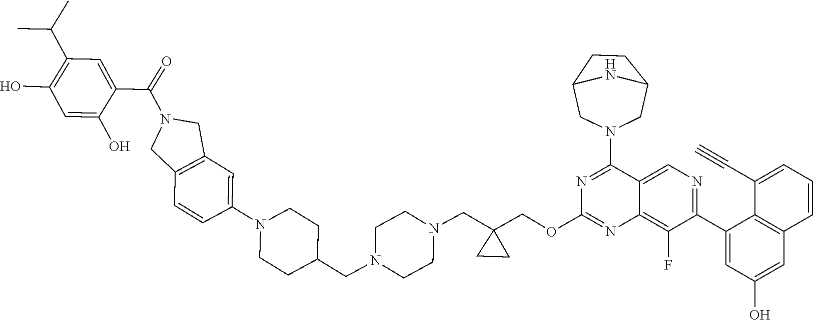 [SMILES: C#Cc1cccc2cc(O)cc(-c3ncc4c(N5CC6CCC(C5)N6)nc(OCC5(CN6CCN(CC7CCN(c8ccc9c(c8)CN(C(=O)c8cc(C(C)C)c(O)cc8O)C9)CC7)CC6)CC5)nc4c3F)c12] |
| | 986.0 |
| 134 | 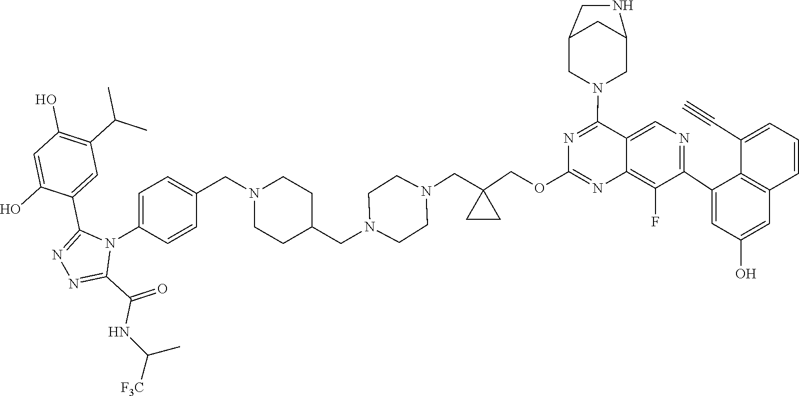 [SMILES: C#Cc1cccc2cc(O)cc(-c3ncc4c(N5CC6CNC(C6)C5)nc(OCC5(CN6CCN(CC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1137.0 |
| 135 | 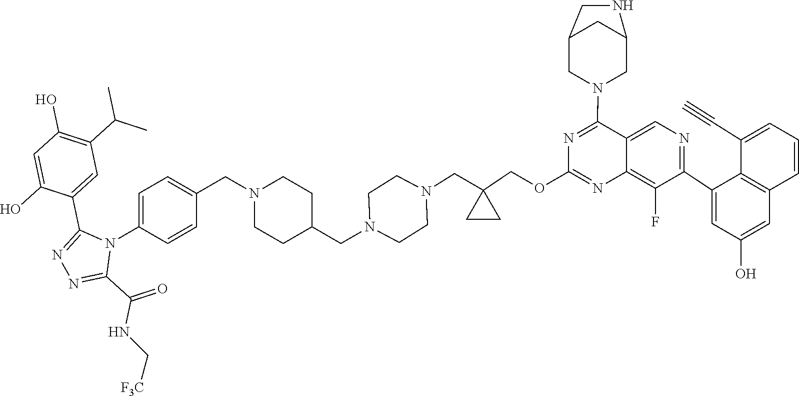 [SMILES: C#Cc1cccc2cc(O)cc(-c3ncc4c(N5CC6CNC(C6)C5)nc(OCC5(CN6CCN(CC7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1123.1 |
| 136 | 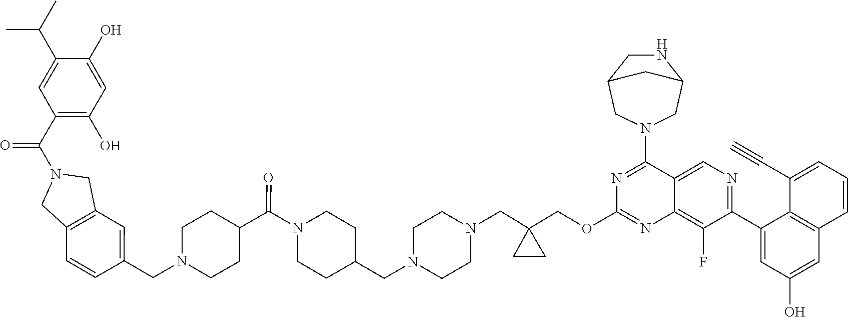 [SMILES: C#Cc1cccc2cc(O)cc(-c3ncc4c(N5CC6CNC(C6)C5)nc(OCC5(CN6CCN(CC7CCN(C(=O)C8CCN(Cc9ccc%10c(c9)CN(C(=O)c9cc(C(C)C)c(O)cc9O)C%10)CC8)CC7)CC6)CC5)nc4c3F)c12] |
| | 1111.1 |
| 137 | 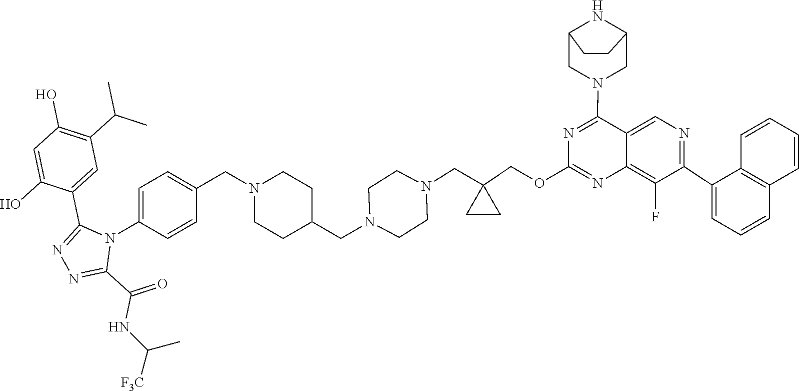 [SMILES: CC(C)c1cc(-c2nnc(C(=O)NC(C)C(F)(F)F)n2-c2ccc(CN3CCC(CN4CCN(CC5(COc6nc(N7CC8CCC(C7)N8)c7cnc(-c8cccc9ccccc89)c(F)c7n6)CC5)CC4)CC3)cc2)c(O)cc1O] |
| | 1097.1 |
| 138 | 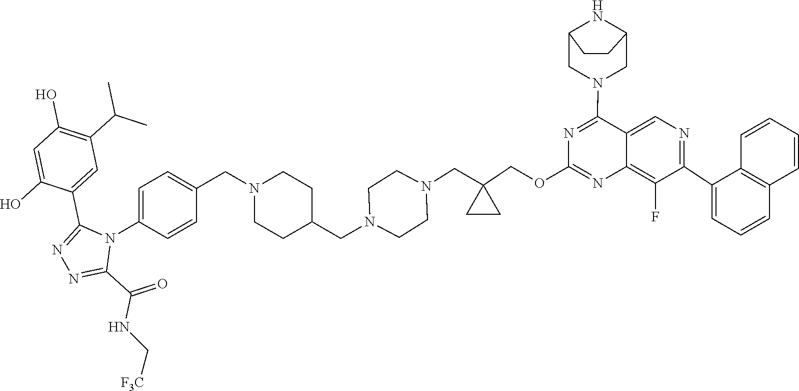 [SMILES: CC(C)c1cc(-c2nnc(C(=O)NCC(F)(F)F)n2-c2ccc(CN3CCC(CN4CCN(CC5(COc6nc(N7CC8CCC(C7)N8)c7cnc(-c8cccc9ccccc89)c(F)c7n6)CC5)CC4)CC3)cc2)c(O)cc1O] |
| | 1083.2 |
| 139 | 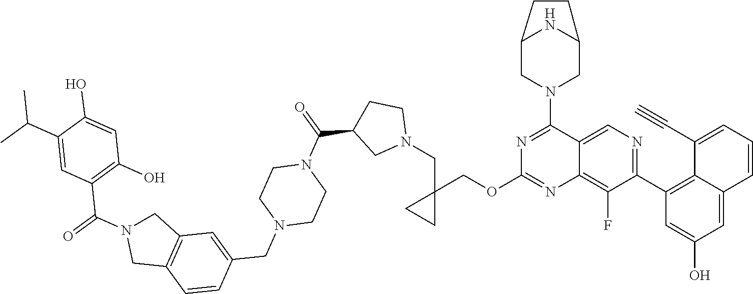 [SMILES: C#Cc1cccc2cc(O)cc(-c3ncc4c(N5CC6CCC(C5)N6)nc(OCC5(CN6CC[C@H](C(=O)N7CCN(Cc8ccc9c(c8)CN(C(=O)c8cc(C(C)C)c(O)cc8O)C9)CC7)C6)CC5)nc4c3F)c12] |
| | 1000.2 |
| 140 | 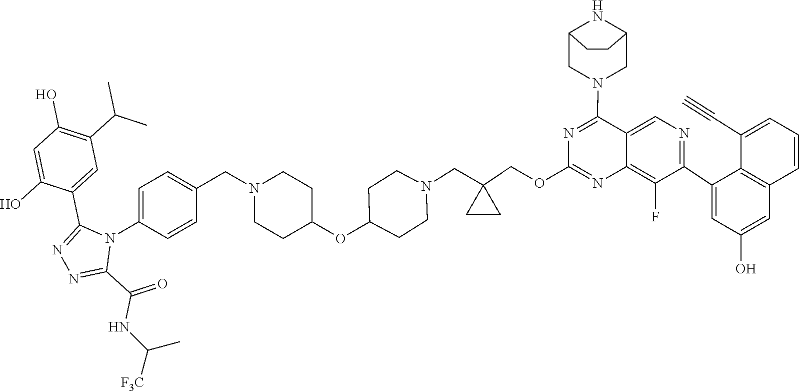 [SMILES: C#Cc1cccc2cc(O)cc(-c3ncc4c(N5CC6CCC(C5)N6)nc(OCC5(CN6CCC(OC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1138.3 |
| 141 | 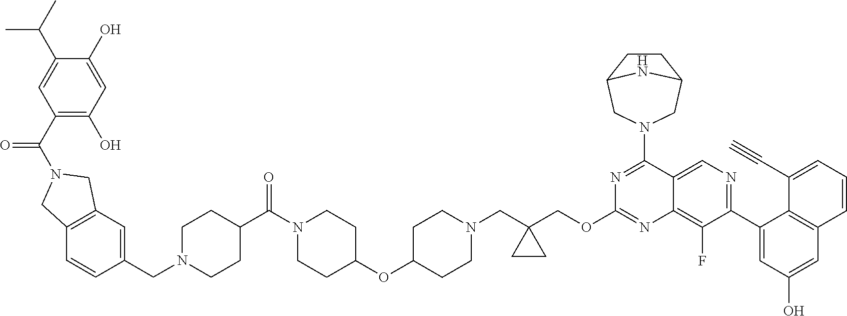 [SMILES: C#Cc1cccc2cc(O)cc(-c3ncc4c(N5CC6CCC(C5)N6)nc(OCC5(CN6CCC(OC7CCN(C(=O)C8CCN(Cc9ccc%10c(c9)CN(C(=O)c9cc(C(C)C)c(O)cc9O)C%10)CC8)CC7)CC6)CC5)nc4c3F)c12] |
| | 1112.3 |
| 142 | 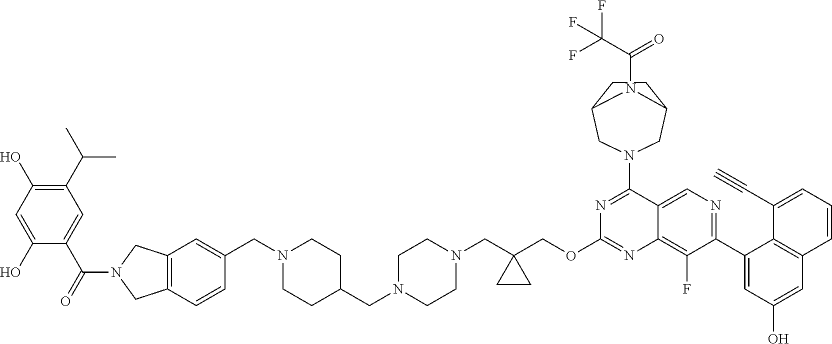 [SMILES: C#Cc1cccc2cc(O)cc(-c3ncc4c(N5CC6CCC(C5)N6C(=O)C(F)(F)F)nc(OCC5(CN6CCN(CC7CCN(Cc8ccc9c(c8)CN(C(=O)c8cc(C(C)C)c(O)cc8O)C9)CC7)CC6)CC5)nc4c3F)c12] |
| | 1096.0 |
| 143 | 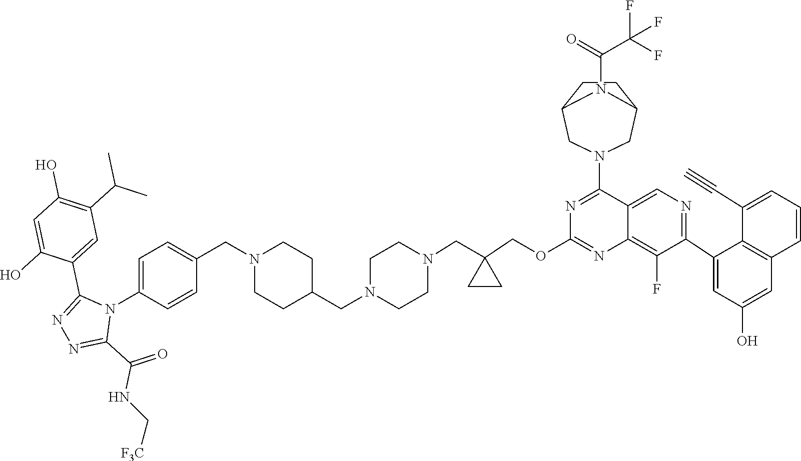 [SMILES: C#Cc1cccc2cc(O)cc(-c3ncc4c(N5CC6CCC(C5)N6C(=O)C(F)(F)F)nc(OCC5(CN6CCN(CC7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1219.0 |
| 144 | 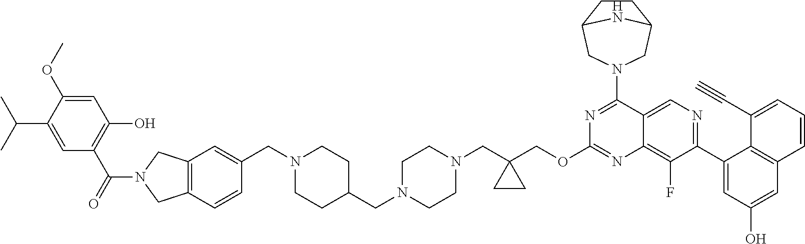 [SMILES: C#Cc1cccc2cc(O)cc(-c3ncc4c(N5CC6CCC(C5)N6)nc(OCC5(CN6CCN(CC7CCN(Cc8ccc9c(c8)CN(C(=O)c8cc(C(C)C)c(OC)cc8O)C9)CC7)CC6)CC5)nc4c3F)c12] |
| | 1014.0 |
| 145 | 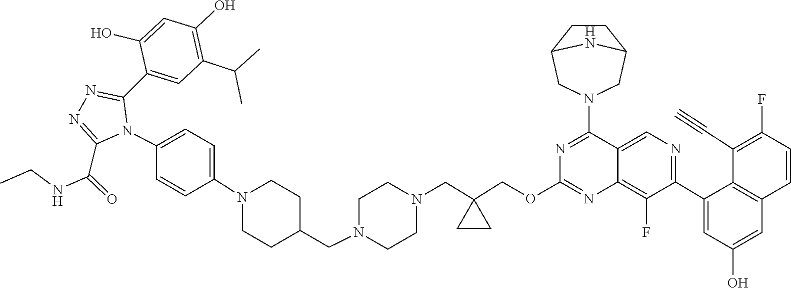 [SMILES: C#Cc1c(F)ccc2cc(O)cc(-c3ncc4c(N5CC6CCC(C5)N6)nc(OCC5(CN6CCN(CC7CCN(c8ccc(-n9c(C(=O)NCC)nnc9-c9cc(C(C)C)c(O)cc9O)cc8)CC7)CC6)CC5)nc4c3F)c12] |
| | 1072.9 |
| 146 | 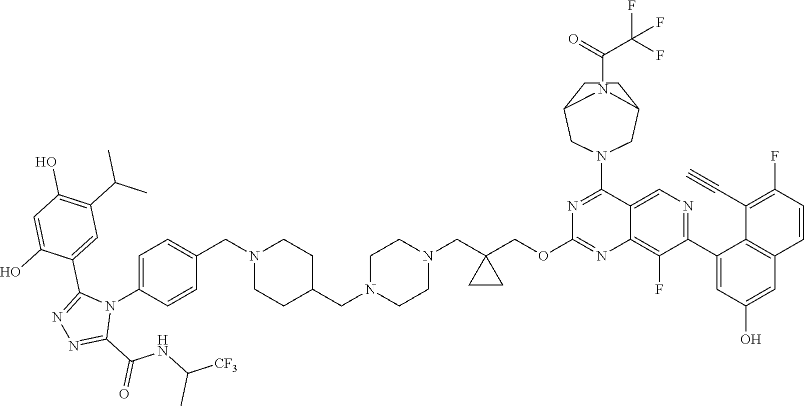 [SMILES: C#Cc1c(F)ccc2cc(O)cc(-c3ncc4c(N5CC6CCC(C5)N6C(=O)C(F)(F)F)nc(OCC5(CN6CCN(CC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1232.9 |
| 147 | 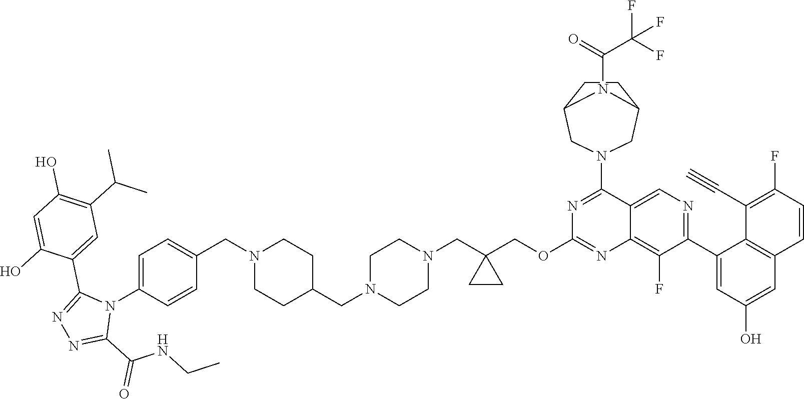 [SMILES: C#Cc1c(F)ccc2cc(O)cc(-c3ncc4c(N5CC6CCC(C5)N6C(=O)C(F)(F)F)nc(OCC5(CN6CCN(CC7CCN(Cc8ccc(-n9c(C(=O)NCC)nnc9-c9cc(C(C)C)c(O)cc9O)cc8)CC7)CC6)CC5)nc4c3F)c12] |
| | 1165.0 |
| 148 | 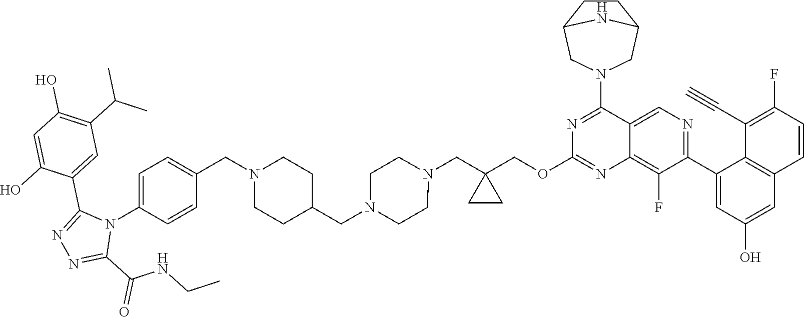 [SMILES: C#Cc1c(F)ccc2cc(O)cc(-c3ncc4c(N5CC6CCC(C5)N6)nc(OCC5(CN6CCN(CC7CCN(Cc8ccc(-n9c(C(=O)NCC)nnc9-c9cc(C(C)C)c(O)cc9O)cc8)CC7)CC6)CC5)nc4c3F)c12] |
| | 1087.3 |
| 149 | 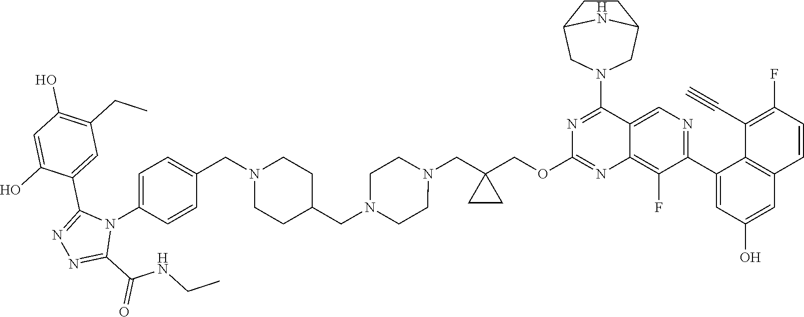 [SMILES: C#Cc1c(F)ccc2cc(O)cc(-c3ncc4c(N5CC6CCC(C5)N6)nc(OCC5(CN6CCN(CC7CCN(Cc8ccc(-n9c(C(=O)NCC)nnc9-c9cc(CC)c(O)cc9O)cc8)CC7)CC6)CC5)nc4c3F)c12] |
| | 1073.3 |
| 150 | 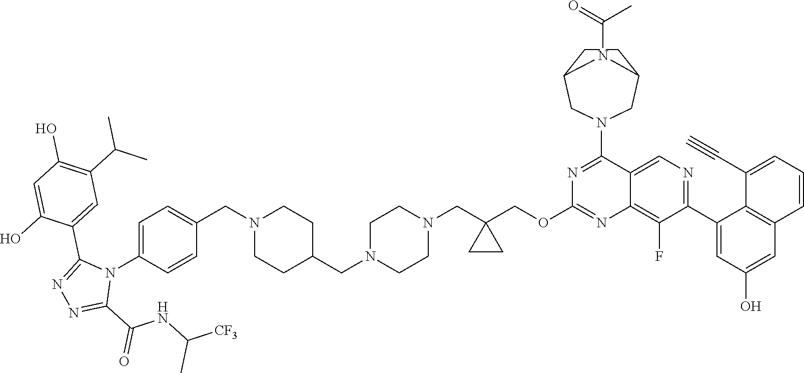 [SMILES: C#Cc1cccc2cc(O)cc(-c3ncc4c(N5CC6CCC(C5)N6C(C)=O)nc(OCC5(CN6CCN(CC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1179.3 |
| 151 | 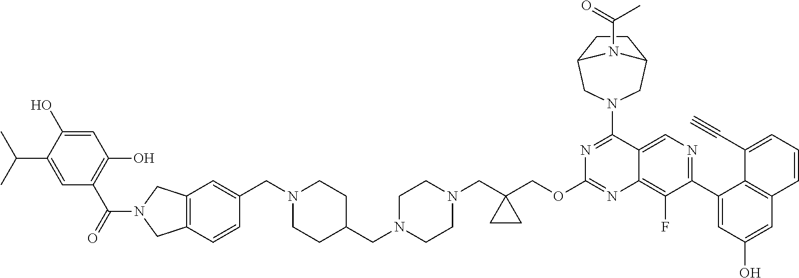 [SMILES: C#Cc1cccc2cc(O)cc(-c3ncc4c(N5CC6CCC(C5)N6C(C)=O)nc(OCC5(CN6CCN(CC7CCN(Cc8ccc9c(c8)CN(C(=O)c8cc(C(C)C)c(O)cc8O)C9)CC7)CC6)CC5)nc4c3F)c12] |
| | 1041.9 |
| 152 | 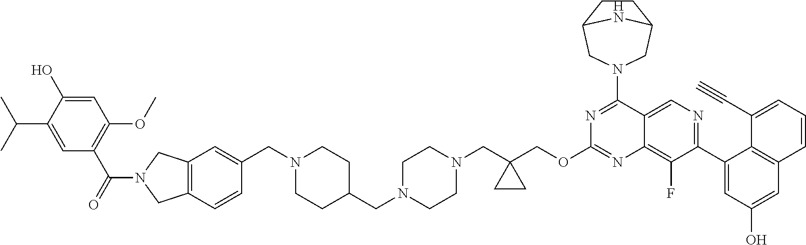 [SMILES: C#Cc1cccc2cc(O)cc(-c3ncc4c(N5CC6CCC(C5)N6)nc(OCC5(CN6CCN(CC7CCN(Cc8ccc9c(c8)CN(C(=O)c8cc(C(C)C)c(O)cc8OC)C9)CC7)CC6)CC5)nc4c3F)c12] |
| | 1014.1 |
| 153 | 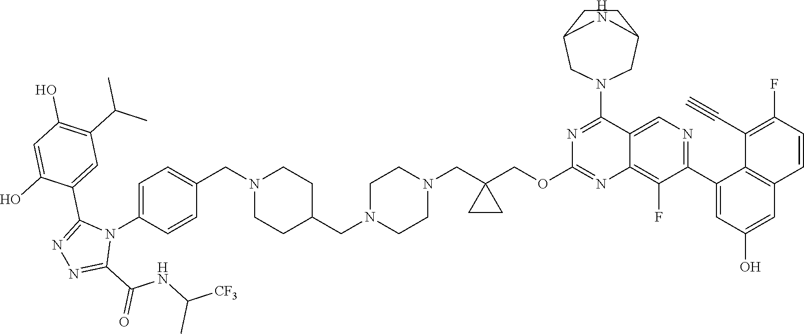 [SMILES: C#Cc1c(F)ccc2cc(O)cc(-c3ncc4c(N5CC6CCC(C5)N6)nc(OCC5(CN6CCN(CC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1154.9 |
| 154 | 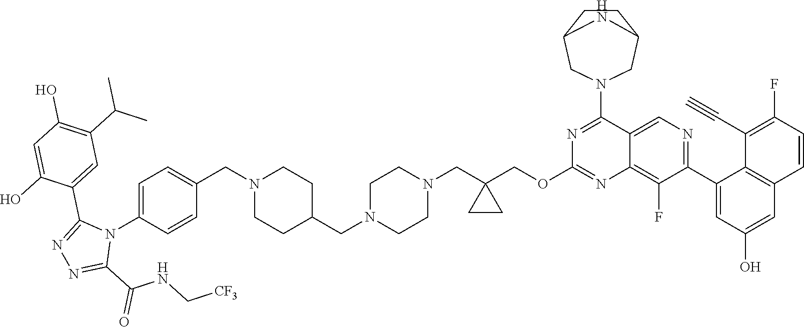 [SMILES: C#Cc1c(F)ccc2cc(O)cc(-c3ncc4c(N5CC6CCC(C5)N6)nc(OCC5(CN6CCN(CC7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1140.9 |
| 155 | 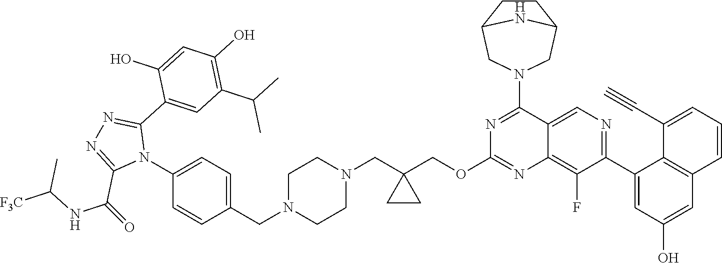 [SMILES: C#Cc1cccc2cc(O)cc(-c3ncc4c(N5CC6CCC(C5)N6)nc(OCC5(CN6CCN(Cc7ccc(-n8c(C(=O)NC(C)C(F)(F)F)nnc8-c8cc(C(C)C)c(O)cc8O)cc7)CC6)CC5)nc4c3F)c12] |
| | 1040.2 |
| 156 | 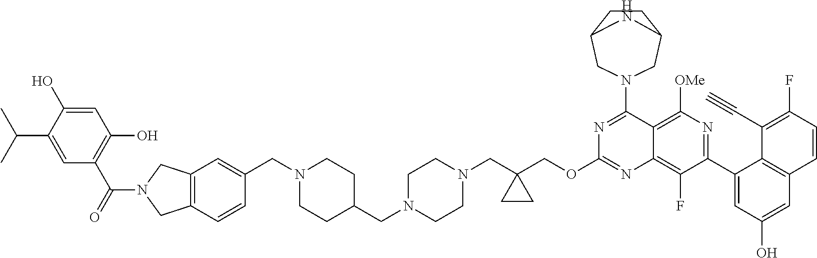 [SMILES: C#Cc1c(F)ccc2cc(O)cc(-c3nc(OC)c4c(N5CC6CCC(C5)N6)nc(OCC5(CN6CCN(CC7CCN(Cc8ccc9c(c8)CN(C(=O)c8cc(C(C)C)c(O)cc8O)C9)CC7)CC6)CC5)nc4c3F)c12] |
| | 1048.0 |
| 157 | 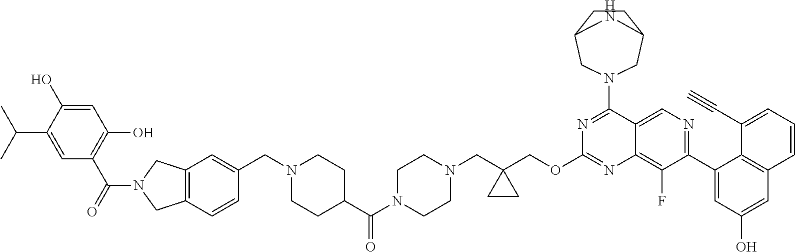 [SMILES: C#Cc1cccc2cc(O)cc(-c3ncc4c(N5CC6CCC(C5)N6)nc(OCC5(CN6CCN(C(=O)C7CCN(Cc8ccc9c(c8)CN(C(=O)c8cc(C(C)C)c(O)cc8O)C9)CC7)CC6)CC5)nc4c3F)c12] |
| | 1013.9 |
| 158 | 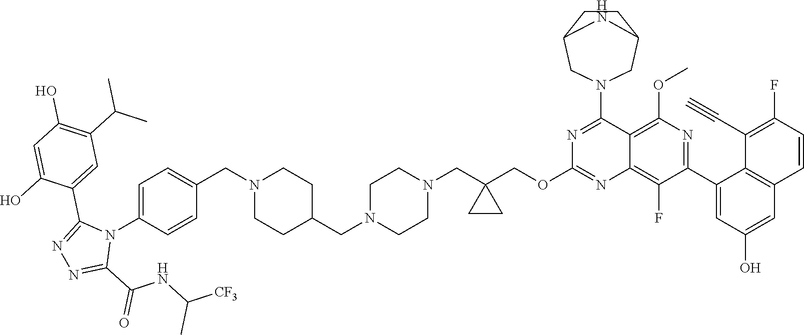 [SMILES: C#Cc1c(F)ccc2cc(O)cc(-c3nc(OC)c4c(N5CC6CCC(C5)N6)nc(OCC5(CN6CCN(CC7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1185.9 |
| 159 | 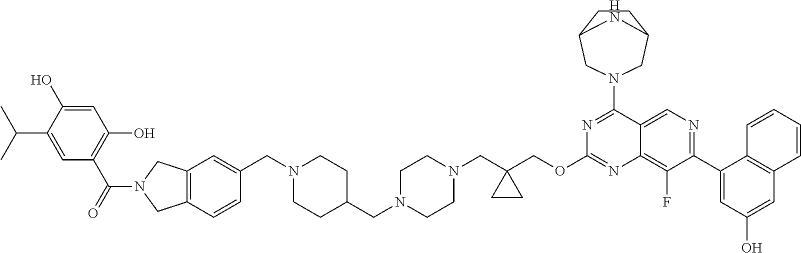 [SMILES: CC(C)c1cc(C(=O)N2Cc3ccc(CN4CCC(CN5CCN(CC6(COc7nc(N8CC9CCC(C8)N9)c8cnc(-c9cc(O)cc%10ccccc9%10)c(F)c8n7)CC6)CC5)CC4)cc3C2)c(O)cc1O] |
| | 976.1 |
| 160 | 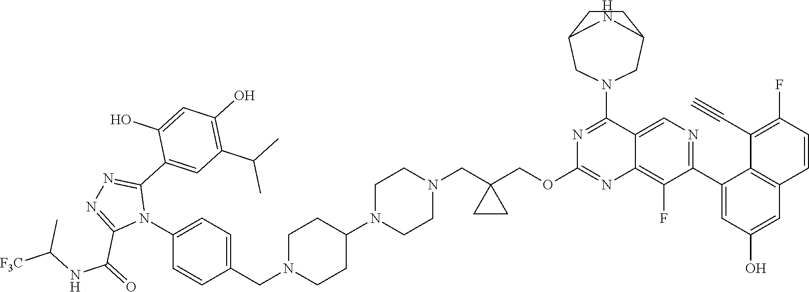 [SMILES: C#Cc1c(F)ccc2cc(O)cc(-c3ncc4c(N5CC6CCC(C5)N6)nc(OCC5(CN6CCN(C7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1140.9 |
| 161 | 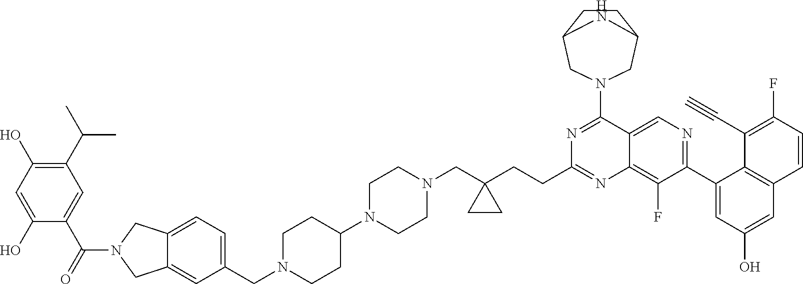 [SMILES: C#Cc1c(F)ccc2cc(O)cc(-c3ncc4c(N5CC6CCC(C5)N6)nc(CCC5(CN6CCN(C7CCN(Cc8ccc9c(c8)CN(C(=O)c8cc(C(C)C)c(O)cc8O)C9)CC7)CC6)CC5)nc4c3F)c12] |
| | 1004.6 |
| 162 | 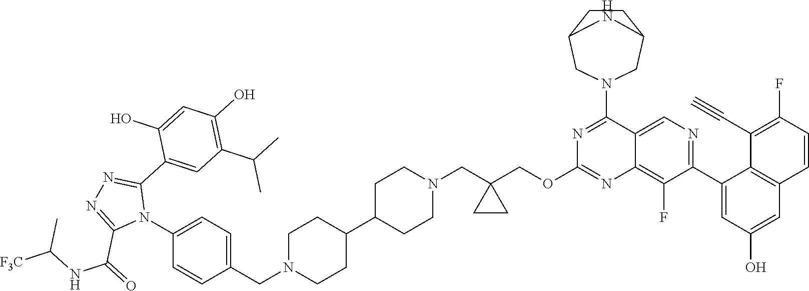 [SMILES: C#Cc1c(F)ccc2cc(O)cc(-c3ncc4c(N5CC6CCC(C5)N6)nc(OCC5(CN6CCC(C7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1140.0 |
| 163 | 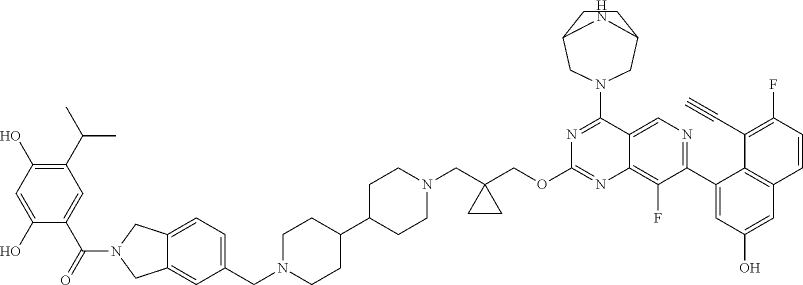 [SMILES: C#Cc1c(F)ccc2cc(O)cc(-c3ncc4c(N5CC6CCC(C5)N6)nc(OCC5(CN6CCC(C7CCN(Cc8ccc9c(c8)CN(C(=O)c8cc(C(C)C)c(O)cc8O)C9)CC7)CC6)CC5)nc4c3F)c12] |
| | 1003.0 |
| 164 | 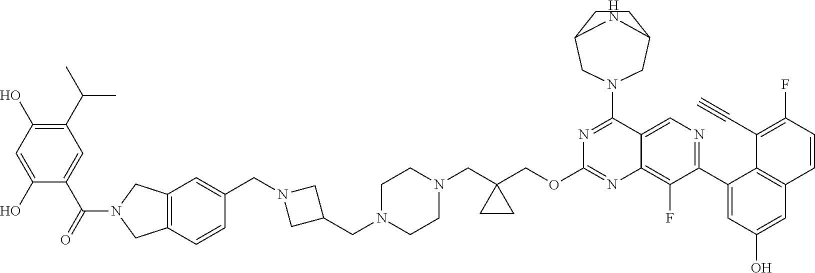 [SMILES: C#Cc1c(F)ccc2cc(O)cc(-c3ncc4c(N5CC6CCC(C5)N6)nc(OCC5(CN6CCN(CC7CN(Cc8ccc9c(c8)CN(C(=O)c8cc(C(C)C)c(O)cc8O)C9)C7)CC6)CC5)nc4c3F)c12] |
| | 990.2 |
| 165 | 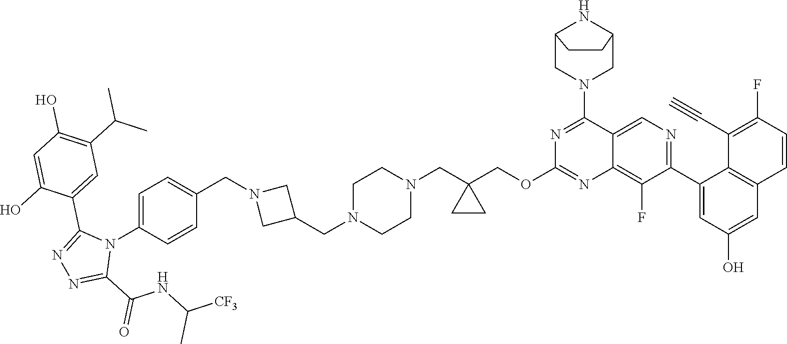 [SMILES: C#Cc1c(F)ccc2cc(O)cc(-c3ncc4c(N5CC6CCC(C5)N6)nc(OCC5(CN6CCN(CC7CN(Cc8ccc(-n9c(C(=O)NC(C)C(F)(F)F)nnc9-c9cc(C(C)C)c(O)cc9O)cc8)C7)CC6)CC5)nc4c3F)c12] |
| | 1126.9 |
| 166 | 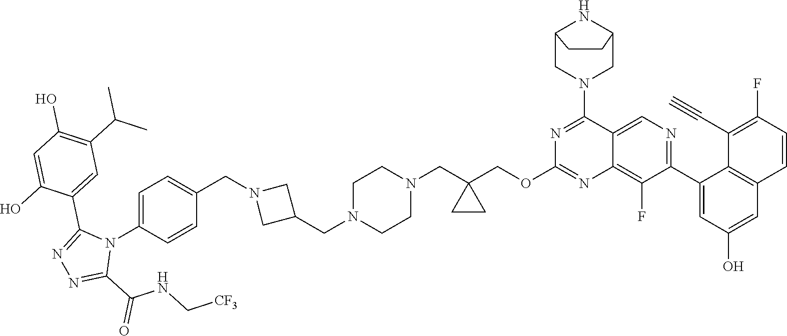 [SMILES: C#Cc1c(F)ccc2cc(O)cc(-c3ncc4c(N5CC6CCC(C5)N6)nc(OCC5(CN6CCN(CC7CN(Cc8ccc(-n9c(C(=O)NCC(F)(F)F)nnc9-c9cc(C(C)C)c(O)cc9O)cc8)C7)CC6)CC5)nc4c3F)c12] |
| | 1113.0 |
| 167 | 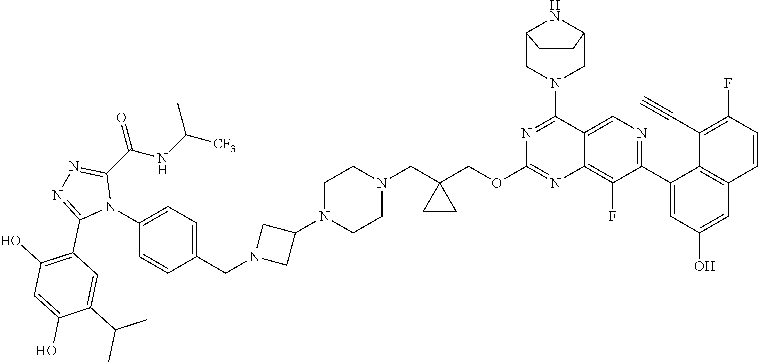 [SMILES: C#Cc1c(F)ccc2cc(O)cc(-c3ncc4c(N5CC6CCC(C5)N6)nc(OCC5(CN6CCN(C7CN(Cc8ccc(-n9c(C(=O)NC(C)C(F)(F)F)nnc9-c9cc(C(C)C)c(O)cc9O)cc8)C7)CC6)CC5)nc4c3F)c12] |
| | 1112.9 |
| 168 | 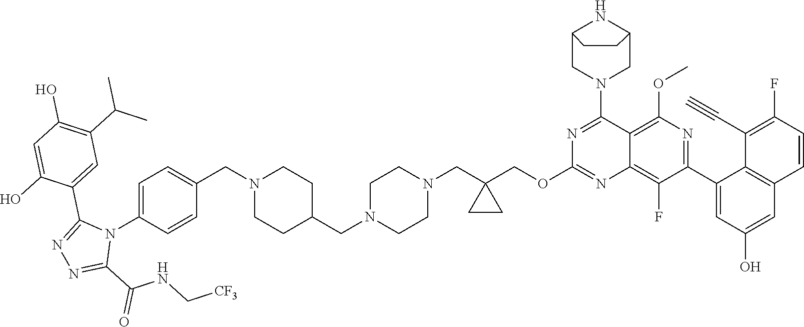 [SMILES: C#Cc1c(F)ccc2cc(O)cc(-c3nc(OC)c4c(N5CC6CCC(C5)N6)nc(OCC5(CN6CCN(CC7CCN(Cc8ccc(-n9c(C(=O)NCC(F)(F)F)nnc9-c9cc(C(C)C)c(O)cc9O)cc8)CC7)CC6)CC5)nc4c3F)c12] |
| | 1171.0 |
| 169 | 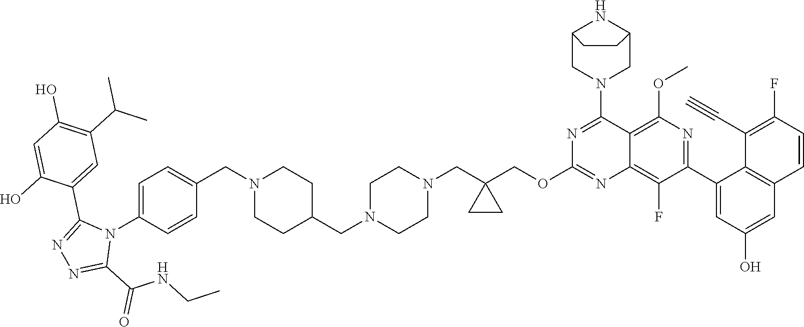 [SMILES: C#Cc1c(F)ccc2cc(O)cc(-c3nc(OC)c4c(N5CC6CCC(C5)N6)nc(OCC5(CN6CCN(CC7CCN(Cc8ccc(-n9c(C(=O)NCC)nnc9-c9cc(C(C)C)c(O)cc9O)cc8)CC7)CC6)CC5)nc4c3F)c12] |
| | 1117.2 |
| 170 | 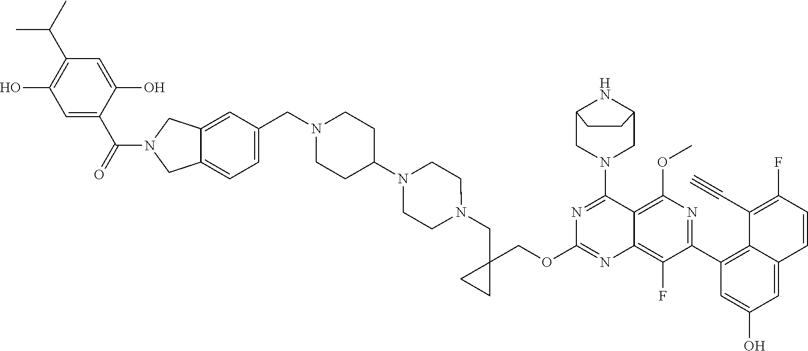 [SMILES: C#Cc1c(F)ccc2cc(O)cc(-c3nc(OC)c4c(N5CC6CCC(C5)N6)nc(OCC5(CN6CCN(C7CCN(Cc8ccc9c(c8)CN(C(=O)c8cc(O)c(C(C)C)cc8O)C9)CC7)CC6)CC5)nc4c3F)c12] |
| | 1033.9 |
| 172 | 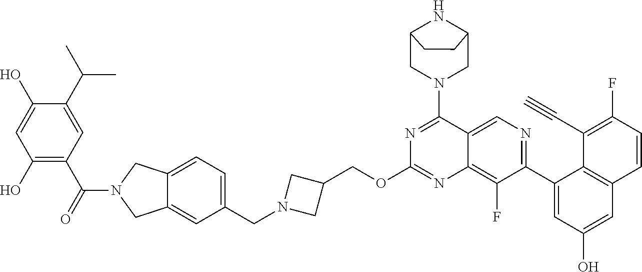 [SMILES: C#Cc1c(F)ccc2cc(O)cc(-c3ncc4c(N5CC6CCC(C5)N6)nc(OCC5CN(Cc6ccc7c(c6)CN(C(=O)c6cc(C(C)C)c(O)cc6O)C7)C5)nc4c3F)c12] |
| | 838.1 |
| 173 | 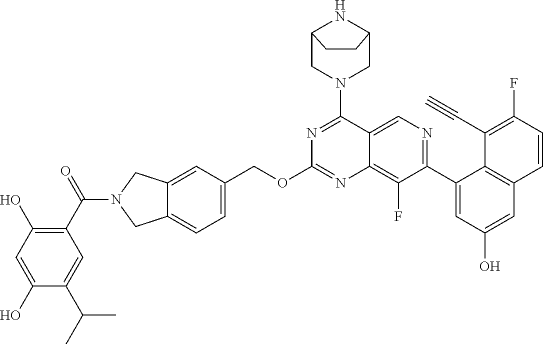 [SMILES: C#Cc1c(F)ccc2cc(O)cc(-c3ncc4c(N5CC6CCC(C5)N6)nc(OCc5ccc6c(c5)CN(C(=O)c5cc(C(C)C)c(O)cc5O)C6)nc4c3F)c12] |
| | 769.1 |
| 174 | 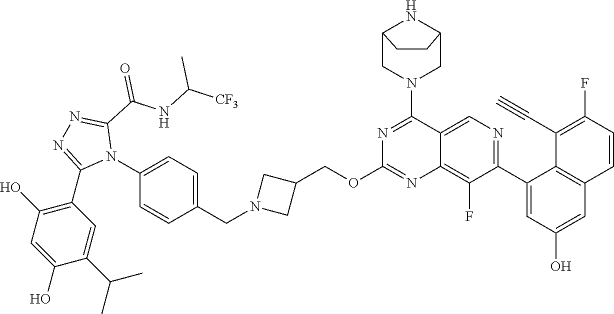 [SMILES: C#Cc1c(F)ccc2cc(O)cc(-c3ncc4c(N5CC6CCC(C5)N6)nc(OCC5CN(Cc6ccc(-n7c(C(=O)NC(C)C(F)(F)F)nnc7-c7cc(C(C)C)c(O)cc7O)cc6)C5)nc4c3F)c12] |
| | 975.0 |
| 175 | 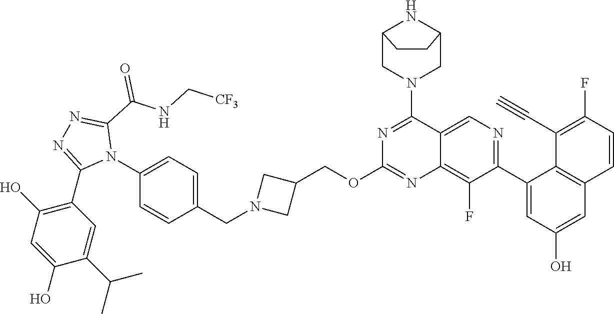 [SMILES: C#Cc1c(F)ccc2cc(O)cc(-c3ncc4c(N5CC6CCC(C5)N6)nc(OCC5CN(Cc6ccc(-n7c(C(=O)NCC(F)(F)F)nnc7-c7cc(C(C)C)c(O)cc7O)cc6)C5)nc4c3F)c12] |
| | 961.1 |
| 176 | 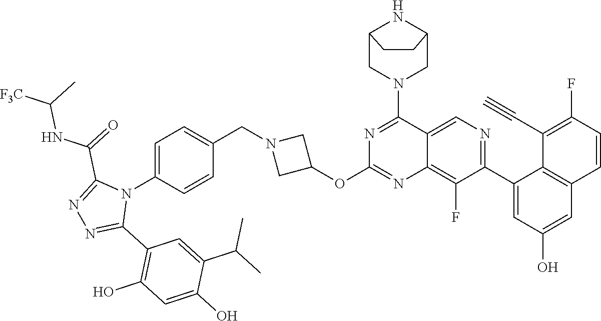 [SMILES: C#Cc1c(F)ccc2cc(O)cc(-c3ncc4c(N5CC6CCC(C5)N6)nc(OC5CN(Cc6ccc(-n7c(C(=O)NC(C)C(F)(F)F)nnc7-c7cc(C(C)C)c(O)cc7O)cc6)C5)nc4c3F)c12] |
| | 961.1 |
| 177 | 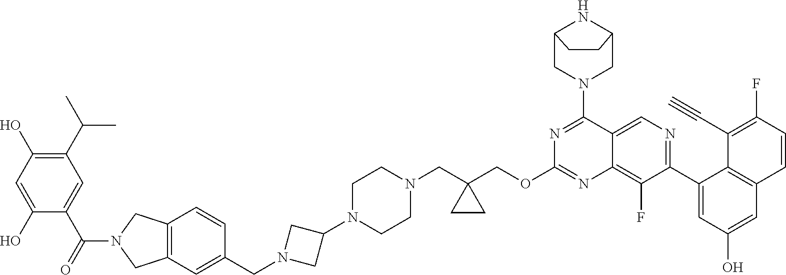 [SMILES: C#Cc1c(F)ccc2cc(O)cc(-c3ncc4c(N5CC6CCC(C5)N6)nc(OCC5(CN6CCN(C7CN(Cc8ccc9c(c8)CN(C(=O)c8cc(C(C)C)c(O)cc8O)C9)C7)CC6)CC5)nc4c3F)c12] |
| | 976.1 |
| 178 | 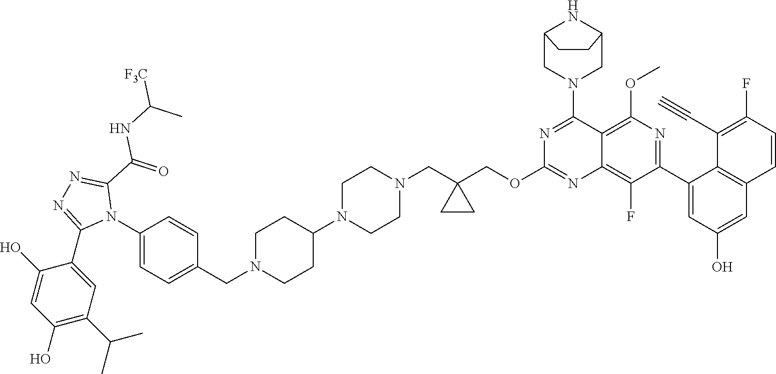 [SMILES: C#Cc1c(F)ccc2cc(O)cc(-c3nc(OC)c4c(N5CC6CCC(C5)N6)nc(OCC5(CN6CCN(C7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1171.2 |
| 179 | 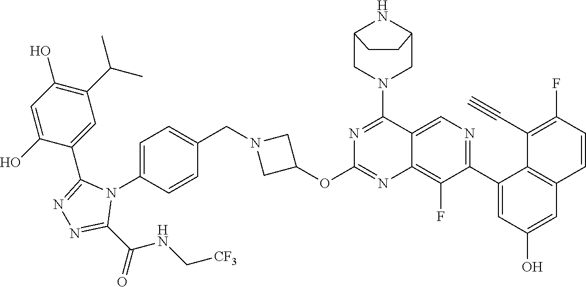 [SMILES: C#Cc1c(F)ccc2cc(O)cc(-c3ncc4c(N5CC6CCC(C5)N6)nc(OC5CN(Cc6ccc(-n7c(C(=O)NCC(F)(F)F)nnc7-c7cc(C(C)C)c(O)cc7O)cc6)C5)nc4c3F)c12] |
| | 947.0 |
| 180 | 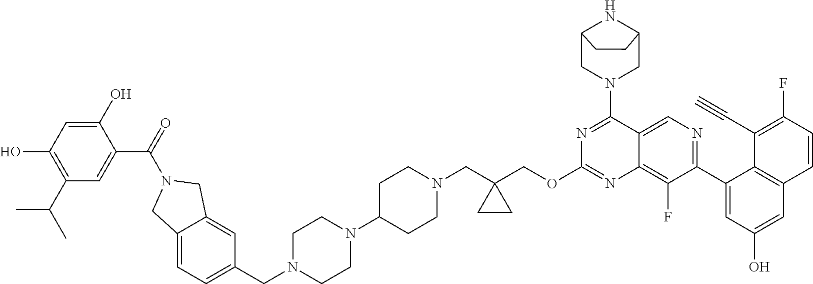 [SMILES: C#Cc1c(F)ccc2cc(O)cc(-c3ncc4c(N5CC6CCC(C5)N6)nc(OCC5(CN6CCC(N7CCN(Cc8ccc9c(c8)CN(C(=O)c8cc(C(C)C)c(O)cc8O)C9)CC7)CC6)CC5)nc4c3F)c12] |
| | 1004.2 |
| 181 | 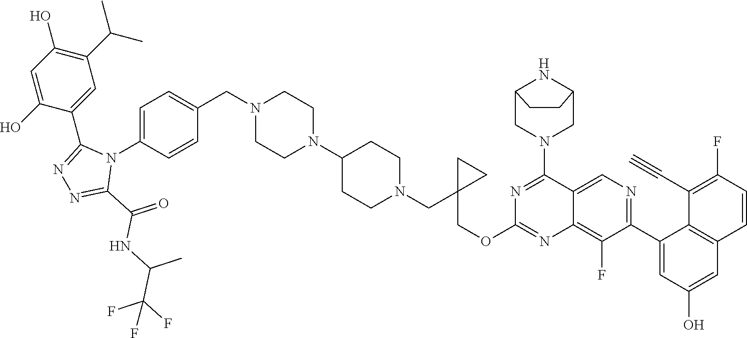 [SMILES: C#Cc1c(F)ccc2cc(O)cc(-c3ncc4c(N5CC6CCC(C5)N6)nc(OCC5(CN6CCC(N7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1141.2 |
| 182 | 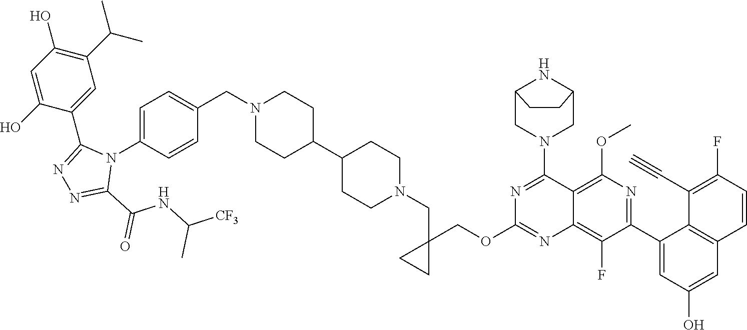 [SMILES: C#Cc1c(F)ccc2cc(O)cc(-c3nc(OC)c4c(N5CC6CCC(C5)N6)nc(OCC5(CN6CCC(C7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1170.7 |
| 183 | 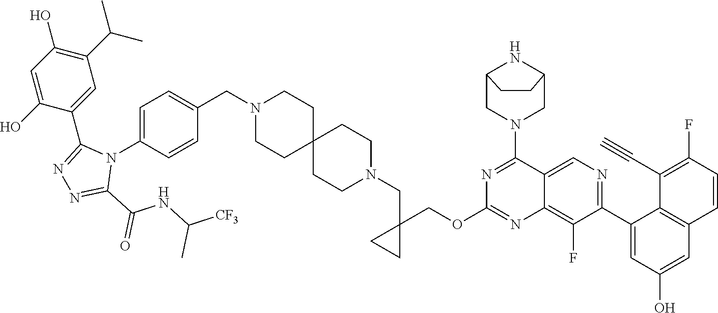 [SMILES: C#Cc1c(F)ccc2cc(O)cc(-c3ncc4c(N5CC6CCC(C5)N6)nc(OCC5(CN6CCC7(CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1126.5 |
| 184 | 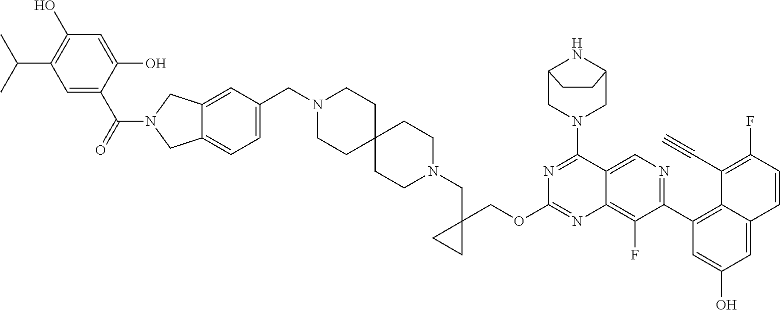 [SMILES: C#Cc1c(F)ccc2cc(O)cc(-c3ncc4c(N5CC6CCC(C5)N6)nc(OCC5(CN6CCC7(CCN(Cc8ccc9c(c8)CN(C(=O)c8cc(C(C)C)c(O)cc8O)C9)CC7)CC6)CC5)nc4c3F)c12] |
| | 989.2 |
| 185 | 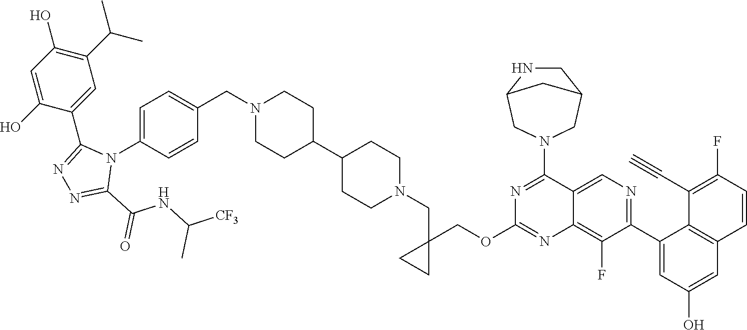 [SMILES: C#Cc1c(F)ccc2cc(O)cc(-c3ncc4c(N5CC6CNC(C6)C5)nc(OCC5(CN6CCC(C7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1140.7 |
| 186 | 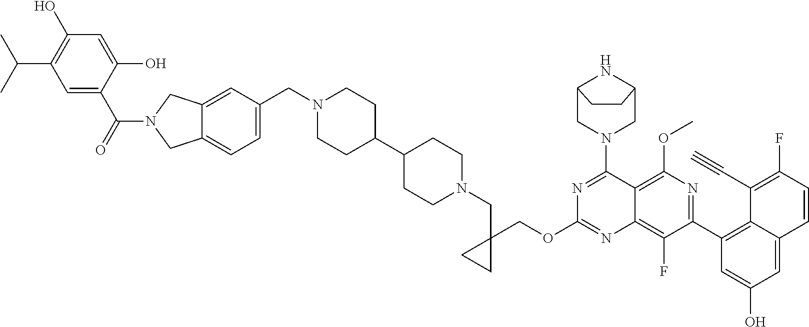 [SMILES: C#Cc1c(F)ccc2cc(O)cc(-c3nc(OC)c4c(N5CC6CCC(C5)N6)nc(OCC5(CN6CCC(C7CCN(Cc8ccc9c(c8)CN(C(=O)c8cc(C(C)C)c(O)cc8O)C9)CC7)CC6)CC5)nc4c3F)c12] |
| | 1033.7 |
| 187 | 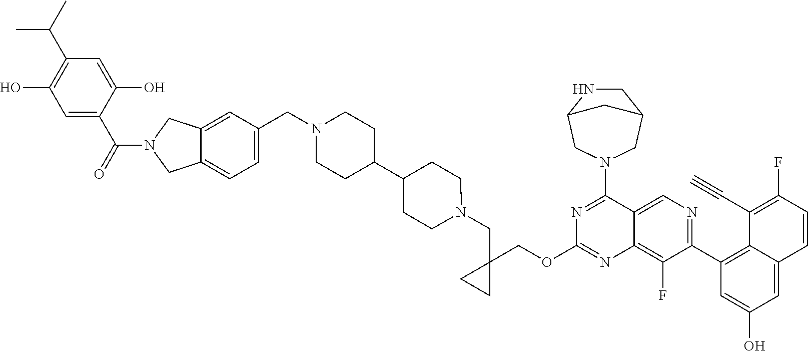 [SMILES: C#Cc1c(F)ccc2cc(O)cc(-c3ncc4c(N5CC6CNC(C6)C5)nc(OCC5(CN6CCC(C7CCN(Cc8ccc9c(c8)CN(C(=O)c8cc(O)c(C(C)C)cc8O)C9)CC7)CC6)CC5)nc4c3F)c12] |
| | 1003.3 |
| 188 | 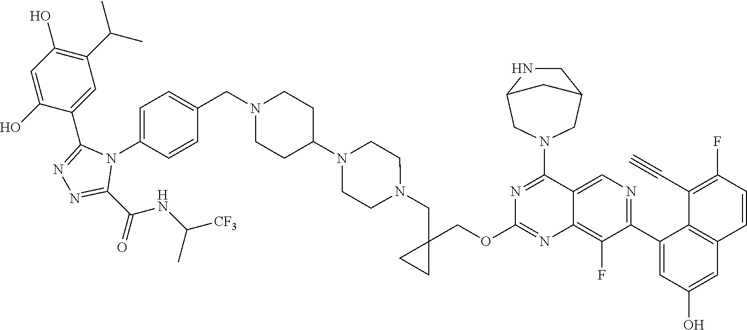 [SMILES: C#Cc1c(F)ccc2cc(O)cc(-c3ncc4c(N5CC6CNC(C6)C5)nc(OCC5(CN6CCN(C7CCN(Cc8ccc(-n9c(C(=O)NC(C)C(F)(F)F)nnc9-c9cc(C(C)C)c(O)cc9O)cc8)CC7)CC6)CC5)nc4c3F)c12] |
| | 1141.3 |
| 189 | 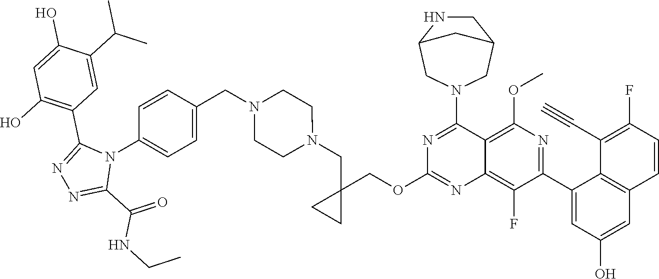 [SMILES: C#Cc1c(F)ccc2cc(O)cc(-c3nc(OC)c4c(N5CC6CNC(C6)C5)nc(OCC5(CN6CCN(Cc7ccc(-n8c(C(=O)NCC)nnc8-c8cc(C(C)C)c(O)cc8O)cc7)CC6)CC5)nc4c3F)c12] |
| | 1020.2 |
| 190 | 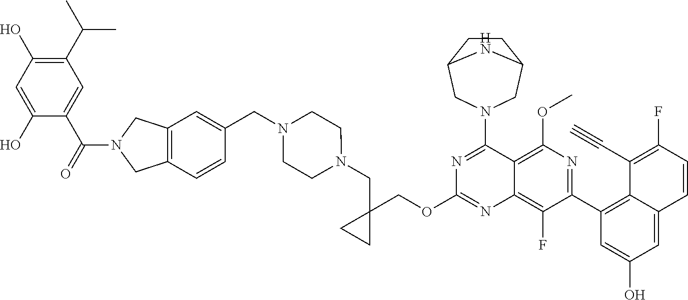 [SMILES: C#Cc1c(F)ccc2cc(O)cc(-c3nc(OC)c4c(N5CC6CCC(C5)N6)nc(OCC5(CN6CCN(Cc7ccc8c(c7)CN(C(=O)c7cc(C(C)C)c(O)cc7O)C8)CC6)CC5)nc4c3F)c12] |
| | 951.3 |
| 191 | 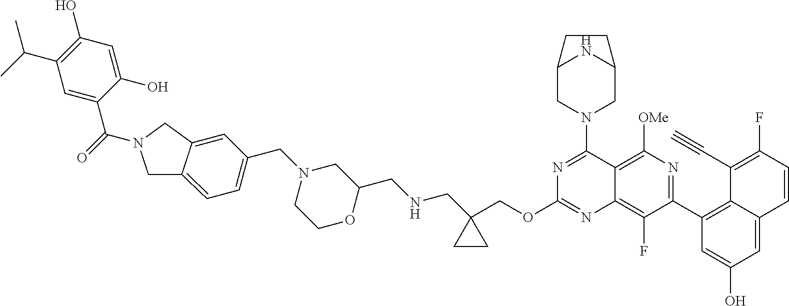 [SMILES: C#Cc1c(F)ccc2cc(O)cc(-c3nc(OC)c4c(N5CC6CCC(C5)N6)nc(OCC5(CNCC6CN(Cc7ccc8c(c7)CN(C(=O)c7cc(C(C)C)c(O)cc7O)C8)CCO6)CC5)nc4c3F)c12] |
| | 981.1 |
| 192 | 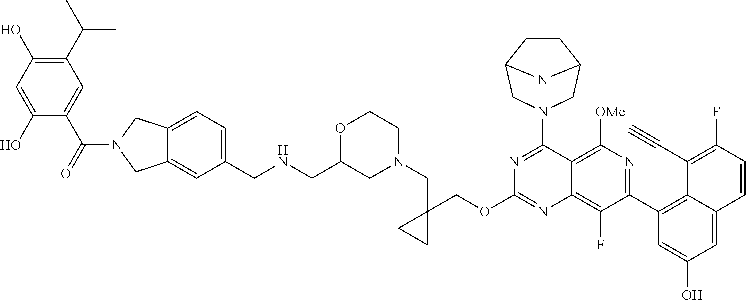 [SMILES: C#Cc1c(F)ccc2cc(O)cc(-c3nc(OC)c4c(N5CC6CCC(C5)N6)nc(OCC5(CN6CCOC(CNCc7ccc8c(c7)CN(C(=O)c7cc(C(C)C)c(O)cc7O)C8)C6)CC5)nc4c3F)c12] |
| | 981.1 |
| 193 | 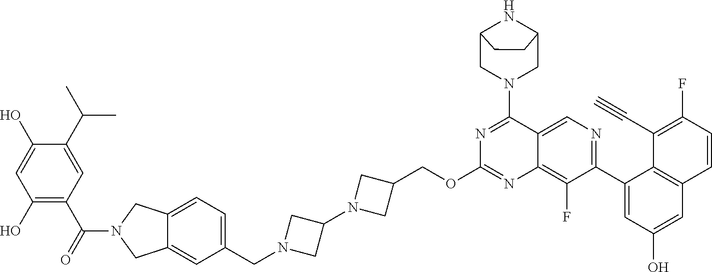 [SMILES: C#Cc1c(F)ccc2cc(O)cc(-c3ncc4c(N5CC6CCC(C5)N6)nc(OCC5CN(C6CN(Cc7ccc8c(c7)CN(C(=O)c7cc(C(C)C)c(O)cc7O)C8)C6)C5)nc4c3F)c12] |
| | 893.2 |
| 194 | 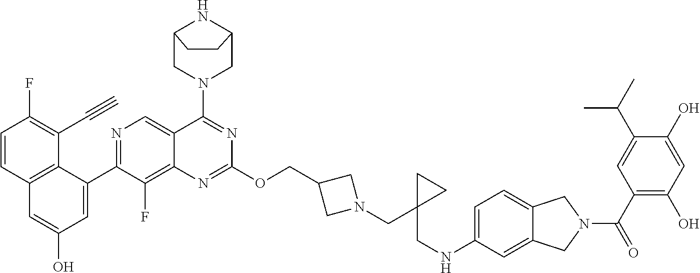 [SMILES: C#Cc1c(F)ccc2cc(O)cc(-c3ncc4c(N5CC6CCC(C5)N6)nc(OCC5CN(CC6(CNc7ccc8c(c7)CN(C(=O)c7cc(C(C)C)c(O)cc7O)C8)CC6)C5)nc4c3F)c12] |
| | 907.2 |
| 195 | 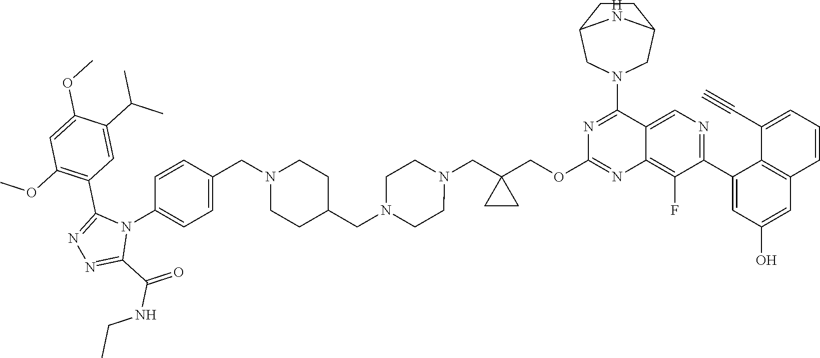 [SMILES: C#Cc1cccc2cc(O)cc(-c3ncc4c(N5CC6CCC(C5)N6)nc(OCC5(CN6CCN(CC7CCN(Cc8ccc(-n9c(C(=O)NCC)nnc9-c9cc(C(C)C)c(OC)cc9OC)cc8)CC7)CC6)CC5)nc4c3F)c12] |
| | 1097.7 |
| 196 | 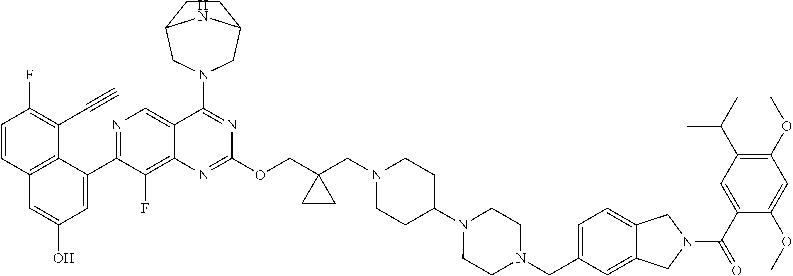 [SMILES: C#Cc1c(F)ccc2cc(O)cc(-c3ncc4c(N5CC6CCC(C5)N6)nc(OCC5(CN6CCC(N7CCN(Cc8ccc9c(c8)CN(C(=O)c8cc(C(C)C)c(OC)cc8OC)C9)CC7)CC6)CC5)nc4c3F)c12] |
| | 1032.2 |
| 197 | 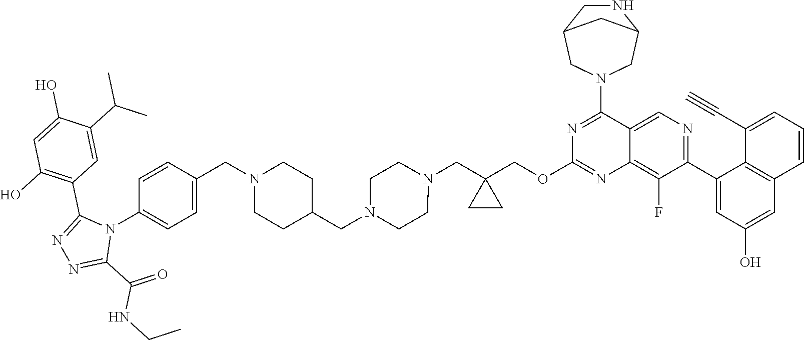 [SMILES: C#Cc1cccc2cc(O)cc(-c3ncc4c(N5CC6CNC(C6)C5)nc(OCC5(CN6CCN(CC7CCN(Cc8ccc(-n9c(C(=O)NCC)nnc9-c9cc(C(C)C)c(O)cc9O)cc8)CC7)CC6)CC5)nc4c3F)c12] |
| | 1069.3 |
| 198 | 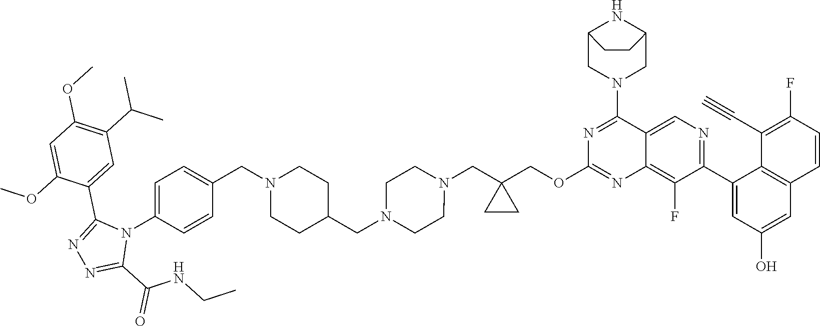 [SMILES: C#Cc1c(F)ccc2cc(O)cc(-c3ncc4c(N5CC6CCC(C5)N6)nc(OCC5(CN6CCN(CC7CCN(Cc8ccc(-n9c(C(=O)NCC)nnc9-c9cc(C(C)C)c(OC)cc9OC)cc8)CC7)CC6)CC5)nc4c3F)c12] |
| | 1115.7 |
| 199 | 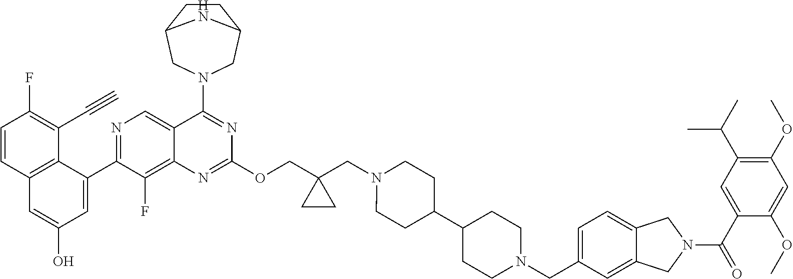 [SMILES: C#Cc1c(F)ccc2cc(O)cc(-c3ncc4c(N5CC6CCC(C5)N6)nc(OCC5(CN6CCC(C7CCN(Cc8ccc9c(c8)CN(C(=O)c8cc(C(C)C)c(OC)cc8OC)C9)CC7)CC6)CC5)nc4c3F)c12] |
| | 1031.3 |
| 200 | 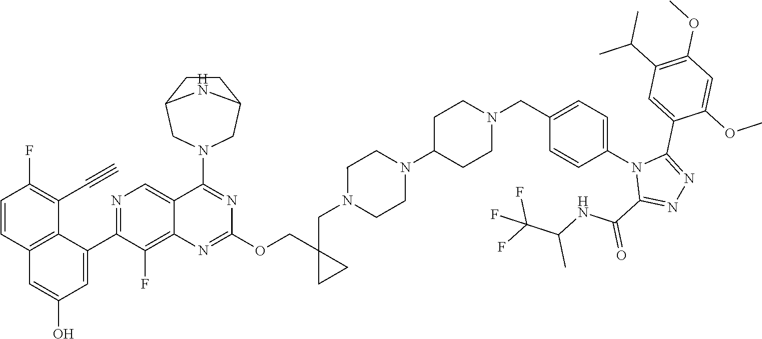 [SMILES: C#Cc1c(F)ccc2cc(O)cc(-c3ncc4c(N5CC6CCC(C5)N6)nc(OCC5(CN6CCN(C7CCN(Cc8ccc(-n9c(C(=O)NC(C)C(F)(F)F)nnc9-c9cc(C(C)C)c(OC)cc9OC)cc8)CC7)CC6)CC5)nc4c3F)c12] |
| | 1069.3 |
| 201 | 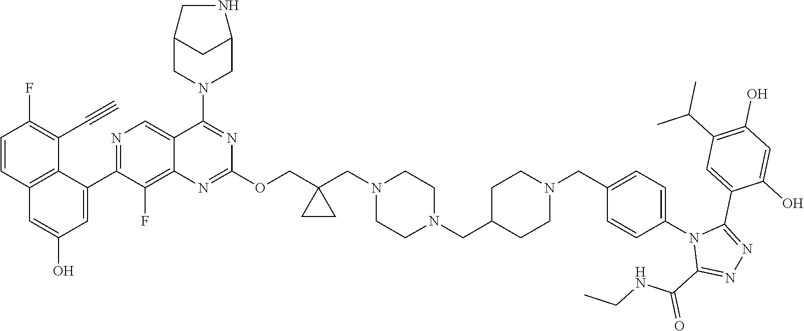 [SMILES: C#Cc1c(F)ccc2cc(O)cc(-c3ncc4c(N5CC6CNC(C6)C5)nc(OCC5(CN6CCN(CC7CCN(Cc8ccc(-n9c(C(=O)NCC)nnc9-c9cc(C(C)C)c(O)cc9O)cc8)CC7)CC6)CC5)nc4c3F)c12] |
| | 1087.7 |
| 202 | 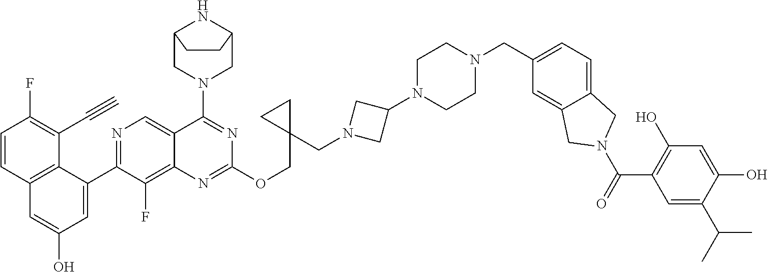 [SMILES: C#Cc1c(F)ccc2cc(O)cc(-c3ncc4c(N5CC6CCC(C5)N6)nc(OCC5(CN6CC(N7CCN(Cc8ccc9c(c8)CN(C(=O)c8cc(C(C)C)c(O)cc8O)C9)CC7)C6)CC5)nc4c3F)c12] |
| | 976.2 |
| 203 | 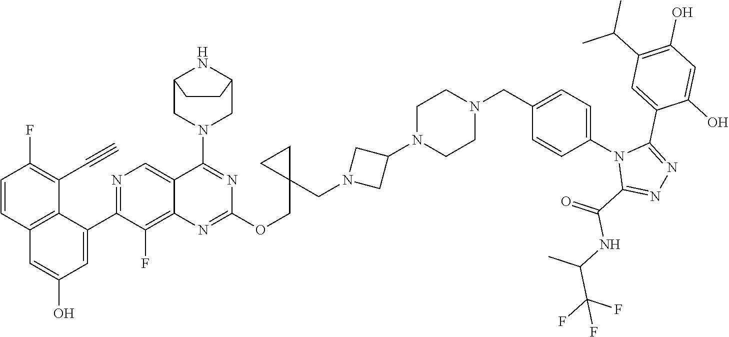 [SMILES: C#Cc1c(F)ccc2cc(O)cc(-c3ncc4c(N5CC6CCC(C5)N6)nc(OCC5(CN6CC(N7CCN(Cc8ccc(-n9c(C(=O)NC(C)C(F)(F)F)nnc9-c9cc(C(C)C)c(O)cc9O)cc8)CC7)C6)CC5)nc4c3F)c12] |
| | 1113.2 |
| 204 | 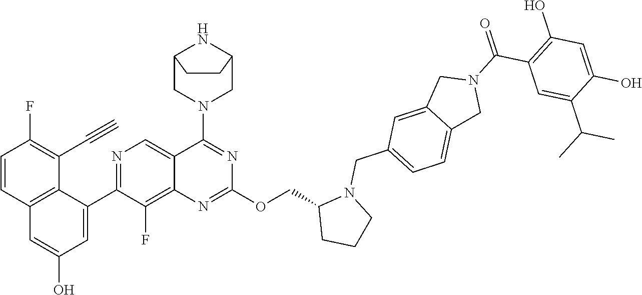 [SMILES: C#Cc1c(F)ccc2cc(O)cc(-c3ncc4c(N5CC6CCC(C5)N6)nc(OC[C@H]5CCCN5Cc5ccc6c(c5)CN(C(=O)c5cc(C(C)C)c(O)cc5O)C6)nc4c3F)c12] |
| | 852.4 |
| 205 | 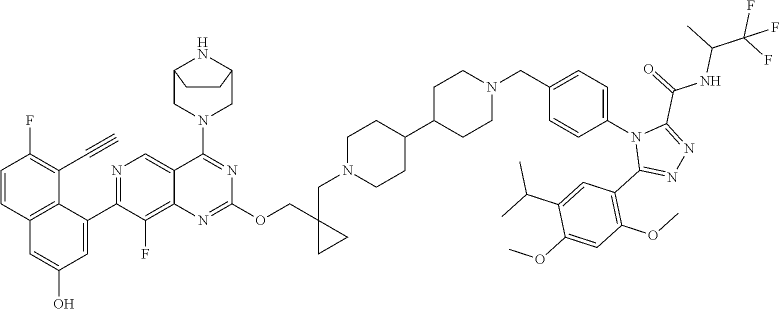 [SMILES: C#Cc1c(F)ccc2cc(O)cc(-c3ncc4c(N5CC6CCC(C5)N6)nc(OCC5(CN6CCC(C7CCN(Cc8ccc(-n9c(C(=O)NC(C)C(F)(F)F)nnc9-c9cc(C(C)C)c(OC)cc9OC)cc8)CC7)CC6)CC5)nc4c3F)c12] |
| | 1168.3 |
| 206 | 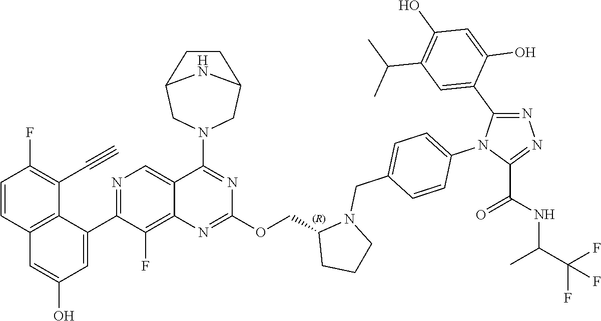 |
| | 989.2 |
| 207 | 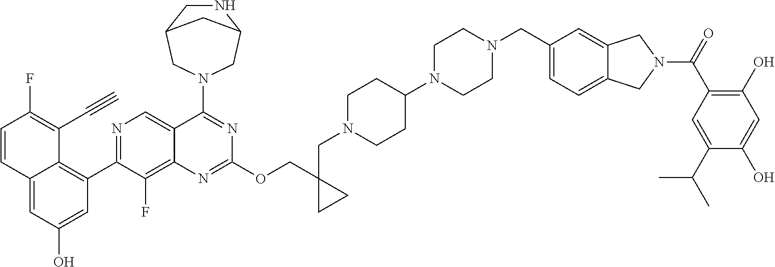 [SMILES: C#Cc1c(F)ccc2cc(O)cc(-c3ncc4c(N5CC6CNC(C6)C5)nc(OCC5(CN6CCC(N7CCN(Cc8ccc9c(c8)CN(C(=O)c8cc(C(C)C)c(O)cc8O)C9)CC7)CC6)CC5)nc4c3F)c12] |
| | 1004.3 |
| 208 | 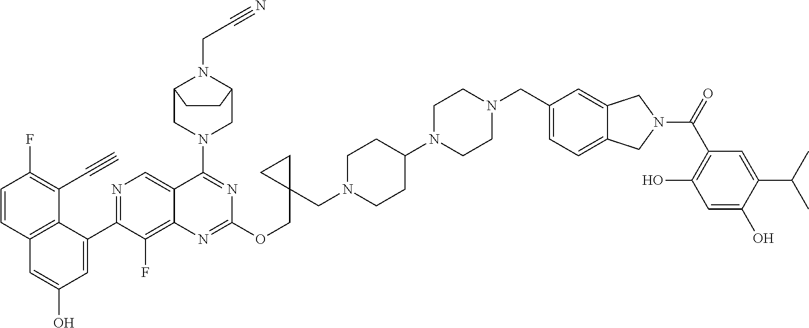 [SMILES: C#Cc1c(F)ccc2cc(O)cc(-c3ncc4c(N5CC6CCC(C5)N6CC#N)nc(OCC5(CN6CCC(N7CCN(Cc8ccc9c(c8)CN(C(=O)c8cc(C(C)C)c(O)cc8O)C9)CC7)CC6)CC5)nc4c3F)c12] |
| | 1043.3 |
| 209 | 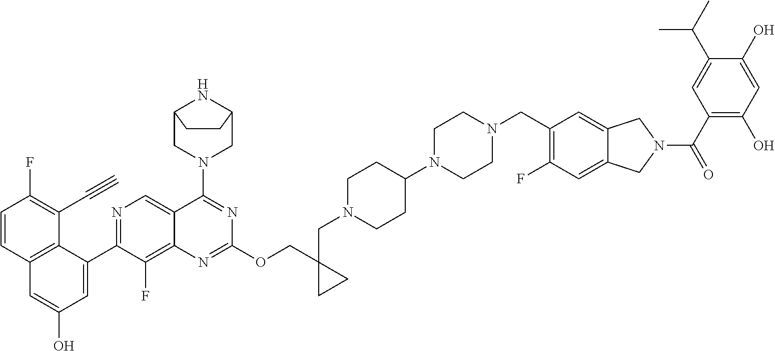 [SMILES: C#Cc1c(F)ccc2cc(O)cc(-c3ncc4c(N5CC6CCC(C5)N6)nc(OCC5(CN6CCC(N7CCN(Cc8cc9c(cc8F)CN(C(=O)c8cc(C(C)C)c(O)cc8O)C9)CC7)CC6)CC5)nc4c3F)c12] |
| | 1022.4 |
| 210 | 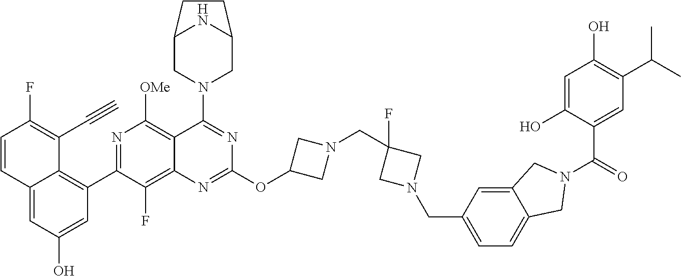 [SMILES: C#Cc1c(F)ccc2cc(O)cc(-c3nc(OC)c4c(N5CC6CCC(C5)N6)nc(OC5CN(CC6(F)CN(Cc7ccc8c(c7)CN(C(=O)c7cc(C(C)C)c(O)cc7O)C8)C6)C5)nc4c3F)c12] |
| | 941.6 |
| 211 | 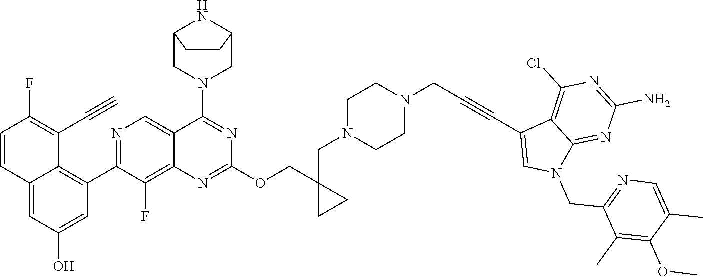 [SMILES: C#Cc1c(F)ccc2cc(O)cc(-c3ncc4c(N5CC6CCC(C5)N6)nc(OCC5(CN6CCN(CC#Cc7cn(Cc8ncc(C)c(OC)c8C)c8nc(N)nc(Cl)c78)CC6)CC5)nc4c3F)c12] |
| | 965.2 |
| 213 | 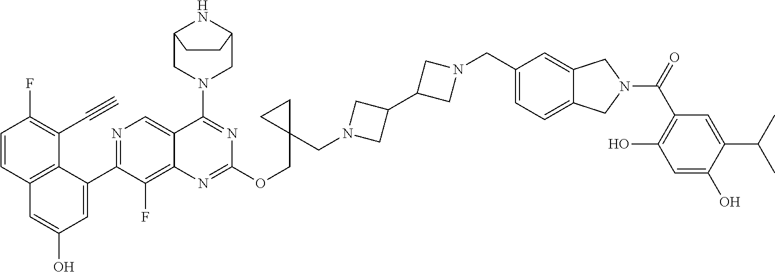 [SMILES: C#Cc1c(F)ccc2cc(O)cc(-c3ncc4c(N5CC6CCC(C5)N6)nc(OCC5(CN6CC(C7CN(Cc8ccc9c(c8)CN(C(=O)c8cc(C(C)C)c(O)cc8O)C9)C7)C6)CC5)nc4c3F)c12] |
| | 947.7 |
| 214 | 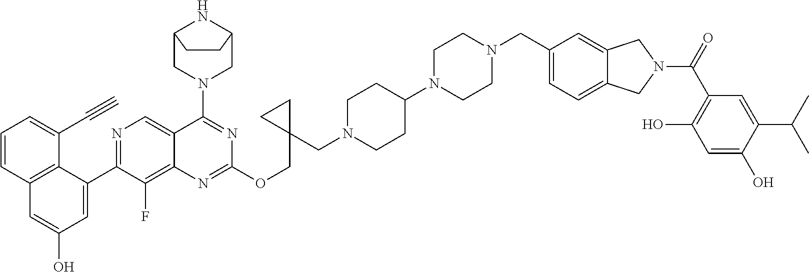 [SMILES: C#Cc1cccc2cc(O)cc(-c3ncc4c(N5CC6CCC(C5)N6)nc(OCC5(CN6CCC(N7CCN(Cc8ccc9c(c8)CN(C(=O)c8cc(C(C)C)c(O)cc8O)C9)CC7)CC6)CC5)nc4c3F)c12] |
| | 986.1 |
| 215 | 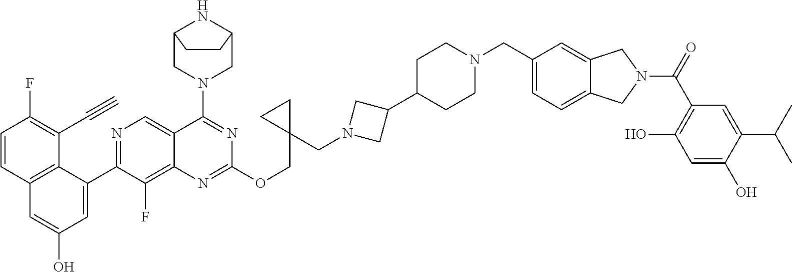 [SMILES: C#Cc1c(F)ccc2cc(O)cc(-c3ncc4c(N5CC6CCC(C5)N6)nc(OCC5(CN6CC(C7CCN(Cc8ccc9c(c8)CN(C(=O)c8cc(C(C)C)c(O)cc8O)C9)CC7)C6)CC5)nc4c3F)c12] |
| | 975.4 |
| 216 | 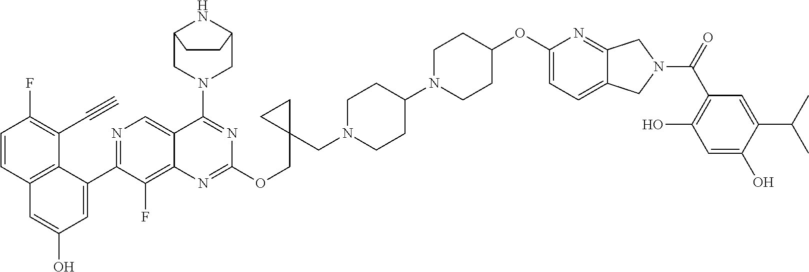 [SMILES: C#Cc1c(F)ccc2cc(O)cc(-c3ncc4c(N5CC6CCC(C5)N6)nc(OCC5(CN6CCC(N7CCC(Oc8ccc9c(n8)CN(C(=O)c8cc(C(C)C)c(O)cc8O)C9)CC7)CC6)CC5)nc4c3F)c12] |
| | 1006.3 |
| 218 | 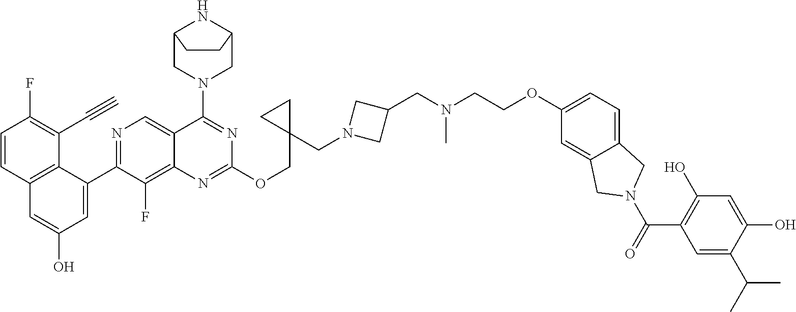 [SMILES: C#Cc1c(F)ccc2cc(O)cc(-c3ncc4c(N5CC6CCC(C5)N6)nc(OCC5(CN6CC(CN(C)CCOc7ccc8c(c7)CN(C(=O)c7cc(C(C)C)c(O)cc7O)C8)C6)CC5)nc4c3F)c12] |
| | 965.4 |
| | ¹H NMR |
| Comp | (DMSO-d₆, 400 MHz) |
| 4 | 11.91 (s, 1H), 9.59 (s, 1H), 9.39 (s, 1H), 9.22 (s, 1H), 8.14 (dd, J = 8.3, 1.4 Hz, 1H), 8.06 (dd, J = 8.2, 1.4 |
| | Hz, 1H), 7.70-7.51 (m, 4H), 7.49 (s, 1H), 7.37 (d, J = 5.5 Hz, 1H), 7.30 (d, J = 8.4 Hz, 2H), 7.17-7.10 |
| | (m, 2H), 6.76 (s, 1H), 6.27 (s, 1H), 5.09-5.03 (m, 1H), 4.56-4.29 (m, 4H), 3.83 (s, 2H), 3.70 (t, J = 6.7 Hz, |
| | 3H), 3.45 (s, 4H), 3.06 (s, 2H), 2.96 (p, J = 6.9 Hz, 3H), 2.73 (d, J = 7.3 Hz, 1H), 2.67 (p, J = 1.9 Hz, 5H), 2.32 (s, 5H), 2.26 |
| | (s, 2H), 2.15 (s, 3H), 1.77 (s, 4H), 1.59 (s, 5H), 1.39 (s, 3H), 0.94 (d, J = 6.9 Hz, 6H). |
| 5 | 11.92 (s, 1H), 9.59 (s, 1H), 9.40 (s, 1H), 9.08 (s, 1H), 8.23-8.16 (m, 1H), 8.09 (dd, J = 8.3, 1.3 Hz, 1H), 7.71 (t, J = 7.6 Hz, 1H), 7.68- |
| | 7.60 (m, 2H), 7.56 (t, J = 7.7 Hz, 1H), 7.30 (d, J = 8.1 Hz, 2H), 7.13 (d, J = 8.1 Hz, 2H), 6.77 (s, 1H), 6.26 (s, 1H), 4.51-4.41 (m, 3H), |
| | 4.39-4.21 (m, 1H), 3.60 (s, 2H), 3.53 (s, 2H), 3.44 (s, 6H), 2.97 (t, J = 7.0 Hz, 3H), 2.67 (p, J = 1.9 Hz, 2H), 2.58 (s, 2H), 2.40-2.22 |
| | (m, 8H), 2.13-1.91 (m, 3H), 1.71-1.41 (m, 9H), 1.24 (s, 1H), 0.94 (d, J = 6.9 Hz, 6H). |
| 6 | 10.54 (s, 1H), 9.74 (s, 1H), 9.08 (s, 1H), 8.94 (t, J = 5.9 Hz, 1H), 8.19 (dd, J = 8.4, 1.4 Hz, 1H), 8.09 (dd, J = 8.3, 1.3 Hz, 1H), 7.71 |
| | (dd, J = 8.2, 7.1 Hz, 1H), 7.68-7.59 (m, 2H), 7.59-7.52 (m, 1H), 7.38 (d, J = 8.2 Hz, 2H), 7.30 (d, J = 8.0 Hz, 2H), 6.59 (s, 1H), 6.34 (s, |
| | 1H), 4.52-4.38 (m, 3H), 4.36-4.21 (m, 1H), 3.67-3.57 (m, 2H), 3.54-3.46 (m, 4H), 3.44 (s, 4H), 3.21-3.10 (m, 2H), 3.05-2.89 |
| | (m, 3H), 2.86-2.79 (m, 2H), 2.67 (p, J = 1.9 Hz, 1H), 2.37 (s, 2H), 2.34-2.29 (m, 6H), 2.11-2.02 (m, 3H), 1.74-1.42 (m, 10H), |
| | 1.03 (t, J = 7.2 Hz, 3H), 0.81 (d, J = 6.9 Hz, 6H). |
| 7 | 10.55 (s, 1H), 9.75 (s, 1H), 9.08 (s, 1H), 8.88 (d, J = 4.8 Hz, 1H), 8.19 (dd, J = 8.3, 1.4 Hz, 1H), 8.09 (dd, J = 8.3, 1.4 |
| | Hz, 1H), 7.71 (dd, J = 8.2, 7.1 Hz, 1H), 7.68-7.60 (m, 2H), 7.58-7.50 (m, 1H), 7.38 (d, |
| | J = 8.2 Hz, 2H), 7.30 (d, J = 8.0 Hz, 2H), 6.59 (s, 1H), 6.34 (s, 1H), 4.59-4.25 (m, 4H), 3.60 (t, J = 9.9 Hz, 2H), 3.54 |
| | (s, 2H), 3.51 (s, 2H), 3.45 (s, 3H), 2.98 (d, J = 1.0 Hz, 1H), 2.95-2.89 (m, 2H), 2.86-2.74 (m, 2H), 2.71-2.66 |
| | (m, 4H), 2.63-2.57 (m, 2H), 2.54-2.43 (m, 2H), 2.37 (s, 6H), 2.12-1.93 (m, 3H), 1.72-1.51 (m, 10H), 1.24 (s, 1H), 0.91-0.79 (m, 6H). |
| 8 | 11.87 (s, 1H), 9.56 (s, 1H), 9.31 (s, 1H), 9.08 (s, 1H), 8.19 (dd, J = 8.3, 1.4 Hz, 1H), 8.09 (dd, J = 8.3, 1.4 Hz, 1H), 7.72 (t, J = 7.6 |
| | Hz, 1H), 7.70-7.48 (m, 3H), 7.26 (d, J = 8.2 Hz, 2H), 7.11 (d, J = 8.1 Hz, 2H), 6.89 (s, 1H), 6.22 (s, 1H), 4.55-4.29 (m, 4H), |
| | 3.59 (d, J = 14.6 Hz, 4H), 3.43 (s, 7H), 2.82 (s, 2H), 2.59 (s, 2H), 2.51 (d, J = 1.8 Hz, 1H), 2.31-2.22 (m, 9H), 2.03 (s, 3H), |
| | 1.94 (s, 3H), 1.71-1.40 (m, 9H), 1.24 (s, 1H), 0.88-0.79 (m, 1H) |
| 9 | 11.87 (s, 1H), 9.52 (s, 1H), 9.33 (s, 1H), 9.08 (s, 1H), 8.19 (d, J = 8.2 Hz, 1H), 8.09 (d, J = 8.1 Hz, 1H), 7.71 (t, J = 7.6 Hz, 1H), |
| | 7.69-7.60 (m, 2H), 7.56 (t, J = 7.8 Hz, 1H), 7.28 (d, J = 8.0 Hz, 2H), 7.12 (d, J = 8.0 Hz, 2H), 6.81 (s, 1H), 6.24 (s, 1H), 4.52-4.39 (m, |
| | 3H), 4.37-4.31 (m, 1H), 3.70-3.57 (m, 2H), 3.54 (s, 2H), 3.49-3.42 (m, 6H), 3.04-2.96 (m, 1H), 2.89-2.80 (m, 1H), 2.84-2.78 (m, 2H), |
| | 2.71-2.64 (m, 1H), 2.59 (s, 2H), 2.39-2.28 (m, 5H), 2.26 (s, 5H), 2.15-2.08 (m, 3H), 1.63 (s, 5H), 1.61-1.51 (m, 4H), 1.24 (s, 1H), |
| | 0.99-0.91 (m, 3H). |
| 10 | 11.90 (s, 1H), 9.60 (d, J = 7.2 Hz, 1H), 9.41 (s, 1H), 9.03 (s, 1H), 8.23-8.05 (m, 2H), 7.79-7.65 (m, 2H), 7.62-7.50 (m, 2H), 7.26 (d, |
| | J = 8.2 Hz, 2H), 7.15-6.98 (m, 2H), 6.75 (s, 1H), 6.26 (s, 1H), 4.57-4.31 (m, 1H), 4.26 (d, J = 5.2 Hz, 3H), 3.73-3.61 (m, 2H), 3.53 |
| | (d, J = 8.2 Hz, 3H), 3.42-3.27 (m, 2H), 2.94 (p, J = 6.9 Hz, 1H), 2.46-2.06 (m, 11H), 1.66 (s, 4H), 0.92 (d, J = 6.9 Hz, 6H), 0.63 (d, J = |
| | 4.6 Hz, 2H), 0.40 (d, J = 4.9 Hz, 2H). |
| 11 | 10.06 (s, 1H), 9.61 (s, 1H), 9.04 (s, 1H), 8.22-8.08 (m, 2H), 7.79-7.64 (m, 2H), 7.64-7.53 (m, 2H), 7.25-7.13 (m, 3H), 7.04 (s, 1H), |
| | 6.39 (s, 1H), 4.75 (s, 4H), 4.62-4.45 (m, 1H), 4.36-4.09 (m, 3H), 3.82-3.51 (m, 5H), 3.41 (s, 3H), 3.09 (p, J = 6.9 Hz, 1H), 2.46-2.12 |
| | (m, 10H), 1.70 (s, 4H), 1.13 (d, J = 6.9 Hz, 6H), 0.62 (s, J = 4.8 Hz, 2H), 0.40 (d, J = 4.8 Hz, 2H). |
| 12 | 10.62 (s, 1H), 9.68 (d, J = 52.8 Hz, 1H), 9.04 (s, 1H), 8.94 (t, J = 5.9 Hz, 1H), 8.24-8.03 (m, 2H), 7.87-7.64 (m, 2H), 7.63-7.50 (m, |
| | 2H), 7.46-7.22 (m, 4H), 6.56 (s, 1H), 6.34 (s, 1H), 4.48 (s, 1H), 4.38-4.18 (m, 3H), 3.81-3.62 (m, 2H), 3.58-3.47 (m, 3H), 3.45 (s, 2H), |
| | 3.15 (p, J = 6.9 Hz, 3H), 2.87 (p, J = 6.8 Hz, 1H), 2.42-2.18 (m, 9H), 1.67 (s, 4H), 1.24 (s, 1H), 1.03 (t, J = 7.2 Hz, 3H), 0.78 (d, J = 6.9 |
| | Hz, 6H), 0.63 (s, 2H), 0.40 (s, 2H). |
| 13 | 11.87 (s, 1H), 9.56 (s, 1H), 9.39 (s, 1H), 9.04 (s, 1H), 8.22-8.08 (m, 2H), 7.77-7.65 (m, 2H), 7.61-7.52 (m, 2H), 7.28 (d, J = 7.9 Hz, 2H), |
| | 7.12 (d, J = 7.8 Hz, 2H), 6.73 (d, J = 2.8 Hz, 1H), 6.26 (d, J = 1.9 Hz, 1H), 4.51-4.40 (m, 3H), 4.38-4.29 (m, 1H), 3.64 (dd, J = 8.5, |
| | 4.5 Hz, 2H), 3.60-3.52 (m, 3H), 3.42 (s, 2H), 2.95 (t, J = 6.8 Hz, 2H), 2.70 (p, J = 5.8 Hz, 1H), 2.48 (s, 2H), 2.36 (s, 5H), 1.66 (s, 4H), |
| | 0.91 (d, J = 6.8 Hz, 6H). |
| 14 | 10.07 (s, 1H), 9.61 (s, 1H), 9.04 (s, 1H), 8.22-8.03 (m, 2H), 7.78-7.69 (m, 2H), 7.61-7.52 (m, 2H), 7.35-7.12 (m, 3H), 7.04 (s, 1H), |
| | 6.39 (s, 1H), 4.75 (s, 4H), 4.50-4.40 (m, 3H), 4.38-4.28 (m, 1H), 3.66 (s, 1H), 3.65-3.56 (m, 1H), 3.56-3.49 (m, 3H), 3.43 (s, 2H), |
| | 3.32 (s, 3H), 3.09 (p, J = 6.9 Hz, 1H), 2.69 (t, J = 5.8 Hz, 2H), 2.50-2.43 (m, 2H), 2.35 (s, 4H), 1.65 (s, 4H), 1.13 (d, J = 6.9 Hz, 6H). |
| 15 | 11.92 (s, 1H), 9.59 (s, 1H), 9.41 (s, 1H), 9.06 (s, 1H), 8.14 (td, J = 8.4, 1.4 Hz, 2H), 7.75-7.65 (m, 2H), 7.62-7.53 (m, 2H), 7.27 (d, J = 8.0 |
| | Hz, 2H), 7.15-7.09 (m, 2H), 6.75 (s, 1H), 6.26 (s, 1H), 4.62-4.51 (m, 1H), 4.41-4.32 (m, 2H), 4.21-4.12 (m, 1H), 3.83-3.66 (m, 4H), 3.41 |
| | (s, 2H), 2.96 (p, J = 6.9 Hz, 1H), 2.76 (s, 1H), 2.67 (p, J = 1.8 Hz, 1H), 2.54 (s, 1H), 2.49-2.22 (m, 11H), 2.19 (s, 1H), 1.92-1.80 |
| | (m, 2H), 1.77 (s, 3H), 1.58 (d, J = 8.6 Hz, 1H), 1.48 (s, 1H), 0.93 (d, J = 6.9 Hz, 6H). |
| 16 | 10.60 (s, 1H), 9.75 (s, 1H), 9.04 (s, 1H), 8.94 (t, J = 5.9 Hz, 1H), 8.28-8.01 (m, 2H), 7.78-7.65 (m, 2H), 7.62-7.52 (m, 2H), 7.45- |
| | 7.33 (m, 2H), 7.29 (d, J = 8.2 Hz, 2H), 6.57 (s, 1H), 6.34 (s, 1H), 4.51-4.42 (m, 1H), 4.41-4.27 (m, 2H), 4.17-4.08 (m, 1H), 3.69-3.59 |
| | (m, 2H), 3.60-3.50 (m, 3H), 3.47 (s, 2H), 3.22-3.10 (m, 2H), 2.89 (p, J = 6.9 Hz, 1H), 2.78-2.71 (m, 1H), 2.55 (d, J = 6.4 Hz, 1H), |
| | 2.45-2.18 (m, 12H), 1.95-1.76 (m, 2H), 1.65 (s, 4H), 1.60-1.47 (m, 2H), 1.03 (t, J = 7.2 Hz, 3H), 0.80 (d, J = 6.9 Hz, 6H). |
| 17 | 10.62 (s, 1H), 9.77 (s, 1H), 9.04 (s, 1H), 8.94 (t, J = 5.9 Hz, 1H), 8.13-8.26 (m, 2H), 7.80-7.65 (m, 2H), 7.62-7.52 (m, 2H), 7.46-7.34 |
| | (m, 2H), 7.29 (d, J = 8.3 Hz, 2H), 6.57 (s, 1H), 6.34 (s, 1H), 4.46 (q, J = 7.4 Hz, 3H), 4.36-4.28 (m, 1H), 3.65 (d, J = 5.0 Hz, 2H), 3.59- |
| | 3.50 (m, 3H) 3.48 (s, 2H), 3.21-3.10 (m, 2H), 2.88 (p, J = 6.9 Hz, 1H), 2.71 (t, J = 5.7 Hz, 2H), 2.49-2.39 (m, 4H), 1.65 (s, 4H), 1.03 (t, |
| | J = 7.2 Hz, 3H), 0.79 (d, J = 6.9 Hz, 6H). |
| 18 | 11.91 (s, 1H), 9.58 (s, 1H), 9.41 (s, 1H), 9.03 (s, 1H), 8.13 (ddd, J = 8.6, 7.4, 1.4 Hz, 2H), 7.78-7.65 (m, 2H), 7.63-7.47 (m, 2H), |
| | 7.38-7.22 (m, 2H), 7.20-7.07 (m, 2H), 6.75 (s, 1H), 6.26 (s, 1H), 4.56-4.37 (m, 1H), 4.30 (d, J = 7.7 Hz, 3H), 3.65 (s, 2H), 3.61- |
| | 3.52 (m, 3H), 3.40 (d, J = 8.4 Hz, 7H), 3.07-2.86 (m, 1H), 2.81-2.74 (m, 2H), 2.53 (s, 1H), 2.41 (s, 2H), 2.42-2.32 (m, 4H), 1.95 (s, 2H), |
| | 1.66 (s, 4H), 1.52 (s, 4H), 0.93 (d, J = 6.9 Hz, 6H), 0.65 (d, J = 4.9 Hz, 2H), 0.42 (d, J = 5.0 Hz, 2H). |
| 19 | 10.62 (s, 1H), 9.75 (s, 1H), 9.04 (s, 1H), 8.94 (s, 1H), 8.14 (s, 2H), 7.82-7.65 (m, 2H), 7.62-7.51 (m, 2H), 7.37 (d, J = 8.4 Hz, 2H), 7.34- |
| | 7.21 (m, 2H), 6.57 (s, 1H), 6.34 (s, 1H), 4.90-4.32 (m, 1H), 4.30 (s, 3H), 3.65 (d, J = 3.4 Hz, 2H), 3.54 (t, J = 5.4 Hz, 3H), 3.51-3.38 (m, |
| | 7H), 3.16 (dd, J = 7.3, 6.0 Hz, 2H), 2.90 (q, J = 6.8 Hz, 1H), 2.82 (m, 2H), 2.73-2.52 (m, 1H), 2.37 (d, J = 32.1 Hz, 6H), 2.03-1.92 |
| | (m, 2H), 1.66 (s, 4H), 1.61-1.54 (m, 4H), 1.03 (t, J = 7.2 Hz, 3H), 0.80 (d, J = 6.9 Hz, 6H), 0.65 (d, J = 5.3 Hz, 2H), 0.42 (s, 2H). |
| 20 | 11.91 (s, 1H), 9.59 (s, 1H), 9.42 (s, 1H), 9.04 (s, 1H), 8.14 (td, J = 8.4, 1.4 Hz, 2H), 7.77-7.47 (m, 4H), 7.32-7.05 (m, 4H), 6.76 (s, 1H), |
| | 6.26 (s, 1H), 4.50-4.23 (m, 4H), 3.71-3.51 (m, 5H), 3.49-3.38 (m, 6H), 2.96 (p, J = 6.9 Hz, 1H), 2.86-2.68 (m, 4H), 2.50 (m, 1H), 2.41 |
| | (s, 4H), 2.02-1.88 (m, 2H), 1.73-1.59 (m, 4H), 1.54 (s, 4H), 0.93 (d, J = 6.9 Hz, 6H). |
| 21 | 10.63 (s, 1H), 9.76 (s, 1H), 9.05 (s, 1H), 8.94 (t, J = 5.9 Hz, 1H), 8.14 (td, J = 8.2, 1.4 Hz, 2H), 7.75-7.65 (m, 2H), |
| | 7.62-7.48 (m, 2H), 7.37 (d, J = 8.3 Hz, 2H), 7.30 (d, J = 8.3 Hz, 2H), 6.58 (s, 1H), 6.35 (s, 1H), |
| | 4.53-4.43 (m, 3H), 4.38-4.29 (m, 1H), 3.68-3.66 (m, 1H), 3.65-3.61 (m, 1H), 3.59-3.51 (m, 3H), 3.48-3.40 (m, 6H) |
| | , 3.32 (s, 3H), 3.22-3.11 (m, 2H), 2.90 (p, J = 6.9 Hz, 1H), 2.85-2.78 (m, 2H), 2.73 (t, J = 5.8 Hz, 2H), |
| | 2.59-2.56 (m, 1H), 2.43 (d, J = 5.7 Hz, 2H), 2.09-1.94 (m, 2H), 1.66 (s, 4H), 1.63-1.48 (m, 4H), 1.03 (t, J = 7.2 |
| | Hz, 3H), 0.80 (d, J = 6.9 Hz, 6H). |
| 22 | 9.59 (s, 1H), 9.44 (s, 1H), 9.09 (s, 1H), 8.07 (d, J = 8.2 Hz, 1H), 7.91 (d, J = 8.2 Hz, 1H), 7.59 (t, J = 7.6 Hz, 1H), 7.45 (d, |
| | J = 7.3 Hz, 2H), 7.32 (d, J = 7.1 Hz, 1H), 7.27 (d, J = 7.9 Hz, 2H), 7.11 (d, J = 7.9 Hz, 2H), 6.76 (s, 1H), 6.25 (s, 1H), |
| | 4.51-4.31 (m, 4H), 3.67-3.44 (m, 4H), 3.40 (s, 3H), 3.02-2.89 (m, 2H), 2.88 (s, 1H), 2.66-2.52 (m, 2H), 2.31-2.21 (m, 13H), |
| | 2.06 (s, 2H), 1.95 (s, 3H), 1.61 (s, 6H), 0.93 (d, J = 6.7 Hz, 6H). |
| 23 | 11.90 (s, 1H), 9.58 (s, 1H), 9.41 (s, 1H), 9.10 (s, 1H), 8.07 (dd, J = 8.4, 1.4 Hz, 1H), 7.91 (d, J = 8.3 Hz, 1H), 7.59 (dd, J = 8.2, |
| | 7.0 Hz, 1H), 7.46 (dd, J = 8.1, 6.8 Hz, 2H), 7.32 (d, J = 7.1 Hz, 1H), 7.27 (d, J = 8.1 Hz, 2H), 7.15-7.09 (m, 2H), |
| | 6.75 (s, 1H), 6.26 (s, 1H), 4.50-4.35 (m, 4H), 3.67-3.52 (m, 4H), 3.39 (d, J = 5.1 Hz, 6H), 3.04-2.90 (m, 3H), 2.79-2.71 (m, 2H), 2.60 |
| | (d, J = 9.2 Hz, 3H), 2.37 (s, 2H), 2.34-2.29 (m, 6H), 2.11-2.06 (m, 1H), 1.95 (s, 5H), 1.62 (s, 5H), 1.53 (s, |
| | 4H), 0.93 (d, J = 6.9 Hz, 6H). |
| 24 | 10.62 (s, 1H), 9.75 (s, 1H), 9.10 (s, 1H), 8.94 (t, J = 5.9 Hz, 1H), 8.07 (dd, J = 8.2, 1.4 Hz, 1H), 7.91 (d, J = 8.0 Hz, 1H), 7.59 |
| | (t, J = 7.6 Hz, 1H), 7.46 (t, J = 7.6 Hz, 2H), 7.40-7.26 (m, 5H), 6.57 (s, 1H), 6.34 (s, 1H), 4.53-4.29 (m, 4H), 3.67-3.57 (m, |
| | 4H), 3.45 (d, J = 5.4 Hz, 6H), 3.20-3.11 (m, 1H), 3.06-2.95 (m, 2H), 2.92-2.88 (m, 1H), 2.81 (d, J = 10.7 Hz, 2H), 2.62-2.58 (m, |
| | 2H), 2.39 (s, 2H), 2.33 (s, 2H), 2.30 (s, 6H), 2.12-2.08 (m, 1H), 1.96 (s, 5H), 1.68-1.60 (m, 5H), 1.56 (s, 4H), 1.03 (t, J = 7.2 Hz, 3H), |
| | 0.80 (d, J = 6.9 Hz, 6H). |
| 25 | 10.07 (s, 1H), 9.96 (s, 1H), 9.60 (s, 1H), 9.16 (s, 1H), 7.80 (d, J = 8.2 Hz, 1H), 7.55 (d, J = 8.4 Hz, 1H), 7.43 (ddd, J = 8.2, |
| | 6.8, 1.2 Hz, 1H), 7.29-7.26 (m, 2H), 7.25-7.12 (m, 3H), 7.04 (s, 1H), 6.39 (s, 1H), 4.76 (s, 4H), 4.48-4.42 (m, 3H), |
| | 4.36-4.33 (m 1H), 3.60 (dd, J = 12.2, 6.4 Hz, 2H), 3.54 (s, 2H), 3.42 (s, 2H), 3.28-3.22 (m, 4H), 3.09 (p, J = 6.9 Hz, 1H), 3.01-2.95 |
| | (m, 1H), 2.88 (s, 1H), 2.63-2.58 (m, 1H), 2.42-2.32 (m, 5H), 2.34-2.32 (m, 4H), 2.08-2.02 (m, 1H), 1.73-1.53 (m, 5H), 1.13 |
| | (d, J = 6.9 Hz, 6H). |
| 26 | 11.90 (s, 1H), 9.58 (s, 1H), 9.43 (s, 1H), 9.04 (s, 1H), 8.19-8.09 (m, 2H), 7.75-7.65 (m, 2H), 7.62-7.52 (m, 2H), 7.31-7.24 (m, 2H), |
| | 7.15-7.07 (m, 2H), 6.76 (s, 1H), 6.25 (s, 1H), 4.50-4.37 (m, 2H), 4.35-4.26 (m, 2H), 3.66 (d, J = 4.8 Hz, 2H), 3.56-3.51 (m, 3H), 3.04-2.91 |
| | (m, 2H),2.90-2.82 (m, 2H), 2.74-2.65 (m, 1H), 2.61 (s, 1H), 2.43-2.20 (m, 12H), 2.12-2.01 (m, 1H), 1.64 (s, 5H), 0.93 (d, J = 6.9 Hz, 6H). |
| 27 | 11.91 (s, 1H), 9.59 (s, 1H), 9.42 (s, 1H), 9.04 (s, 1H), 8.18-8.10 (m, 2H), 7.76-7.64 (m, 2H), 7.62-7.52 (m, 2H), 7.32-7.25 (m, 2H), |
| | 7.17-7.09 (m, 2H), 6.75 (s, 1H), 6.26 (s, 1H), 4.51-4.44 (m, 1H), 4.41-4.27 (m, 2H), 4.18-4.08 (m, 1H), 3.67 (d, J = 1.3 Hz, 1H), 3.65- |
| | 3.60 (m, 1H), 3.59-3.51 (m, 3H), 3.47-3.36 (m, 6H), 3.33-3.31 (m, 1H), 2.96 (p, J = 6.9 Hz, 1H), 2.79-2.73 (m, 3H), 2.57- |
| | 2.51 (m, 1H), 2.44-2.27 (m, 8H), 2.24-2.15 (m, 1H), 1.99-1.91 (m, 2H), 1.89-1.81 (m, 2H), 1.65 (s, 4H), 1.52 (d, J = 9.4 Hz, |
| | 6H), 0.93 (d, J = 6.9 Hz, 6H). |
| 28 | 10.62 (s, 1H), 9.76 (s, 1H), 9.04 (s, 1H), 8.95 (t, J = 5.9 Hz, 1H), 8.22-8.09 (m, 2H), 7.76-7.65 (m, 2H), 7.62-7.53 (m, 2H), |
| | 7.37 (d, J = 8.0 Hz, 2H), 7.29 (d, J = 8.3 Hz, 2H), 6.57 (s, 1H), 6.34 (s, 1H), 4.51-4.43 (m, 1H), 4.41-4.28 (m, 2H), |
| | 4.21-4.12 (m, 1H), 3.69-3.60 (m, 2H), 3.59-3.52 (m, 3H), 3.51-3.39 (m, 7H), 3.20-3.11 (m, 2H), 2.90 (p, J = 6.9 Hz, |
| | 1H), 2.84-2.77 (m, 2H), 2.75 (d, J = 5.6 Hz, 1H), 2.59-2.53 (m, 1H), 2.44-2.35 (m, 6H), 2.34-2.29 (m, 2H), 2.26-2.16 (m, |
| | 1H), 2.04-1.93 (m, 2H), 1.96-1.83 (m, 2H), 1.66 (s, 4H), 1.56 (s, 6H), 1.03 (t, J = 7.1 Hz, 3H), 0.80 (d, J = 6.9 Hz, 6H). |
| 29 | 10.60 (s, 1H), 9.75 (s, 1H), 9.10 (s, 1H), 8.95 (t, J = 5.9 Hz, 1H), 8.07 (dd, J = 8.3, 1.5 Hz, 1H), 7.91 (d, J = 8.1 Hz, 1H), 7.59 (t, J = |
| | 7.6 Hz, 1H), 7.50-7.41 (m, 2H), 7.38-7.26 (m, 5H), 6.57 (s, 1H), 6.33 (s, 1H), 4.54-4.25 (m, 4H), 3.68-3.49 (m, 4H), 3.47 (s, 2H), 3.20-3.07 |
| | (m 3H), 3.04-3.01 (m, 1H), 2.92-2.85 (m, 2H), 2.63 (s, 1H), 2.39 (s, 6H), 2.30 (d, J = 1.6 Hz, 6H), 2.07 (s, 1H), 1.95 |
| | (s, 3H), 1.66-1.58 (m, 5H), 1.03 (t, J = 7.2 Hz, 3H), 0.80 (d, J = 6.8 Hz, 6H). |
| 30 | 11.91 (s, 1H), 9.67-9.45 (m, 2H), 9.17 (s, 1H), 8.18-7.94 (m, 2H), 7.80-7.62 (m, 3H), 7.60-7.51 (m, 2H), 7.35-7.20 (m, 2H), |
| | 7.17-7.00 (m, 2H), 6.76 (s, 1H), 6.25 (s, 1H), 4.51-4.39 (m, 3H), 4.37-4.29 (m, 1H), 3.60 (m, 2H), 3.53 (s, 2H), 3.40 |
| | (s, 2H), 3.04-2.92 (m, 2H), 2.89-2.71 (m, 1H), 2.70-2.56 (m, 1H), 2.51-2.30 (m, 12H), 2.15-2.06 (m, 1H), |
| | 1.62 (s, 5H), 0.93 (d, J = 6.9 Hz, 6H). |
| 31 | 10.60 (s, 1H), 9.96 (s, 1H), 9.76 (s, 1H), 9.16 (s, 1H), 8.94 (t, J = 5.9 Hz, 1H), 7.87-7.74 (m, 1H), 7.60-7.52 (m, 1H), |
| | 7.43 (ddd, J = 8.2, 6.8, 1.2 Hz, 1H), 7.40-7.33 (m, 2H), 7.32-7.25 (m, 3H), 7.25-7.19 (m, 2H), 6.57 (s, 1H), 6.33 (s, 1H), |
| | 4.55-4.29 (m, 4H), 3.71-3.57 (m, 2H), 3.53 (s, 2H), 3.47 (s, 2H), 3.21-3.09 (m, 2H), 3.04-2.98 (m, 1H), 2.96-2.81 |
| | (m, 2H), 2.70-2.58 (m, 1H), 2.46-2.32 (m, 7H), 2.31 (s, 5H), 2.15-2.01 (m, 1H), 1.71-1.51 (m, 5H), 1.03 (t, J = 7.2 |
| | Hz, 3H), 0.80 (d, J = 6.9 Hz, 6H). |
| 32 | 11.90 (s, 1H), 9.97 (s, 1H), 9.59 (s, 1H), 9.41 (s, 1H), 9.16 (s, 1H), 7.79 (d, J = 8.2 Hz, 1H), 7.56 (d, J = 8.4 Hz, 1H), |
| | 7.43 (ddd, J = 8.2, 6.8, 1.2 Hz, 1H), 7.27 (dd, J = 8.5, 3.5 Hz, 3H), 7.23 (dd, J = 6.5, 1.9 Hz, 2H), 7.17-7.07 |
| | (m, 2H), 6.75 (s, 1H), 6.26 (s, 1H), 4.48-4.31 (m, 4H), 3.64-3.58 (m, 2H), 3.53 (s, 2H), 3.40 (s, 6H), 3.06-2.88 |
| | (m, 3H), 2.79-3.74 (m, 2H), 2.62-2.58 (m, 1H), 2.31 (d, J = 9.7 Hz, 8H), 2.15-2.05 (m, 1H), 1.94 (t, J = 8.9 |
| | Hz, 2H), 1.63 (s, 5H), 1.59-1.44 (m, 4H), 0.93 (d, J = 6.9 Hz, 6H). |
| 33 | 11.90 (s, 1H), 9.95 (s, 1H), 9.58 (s, 1H), 9.43 (s, 1H), 9.16 (s, 1H), 7.87-7.72 (m, 1H), 7.56 (d, J = 8.4 Hz, 1H), 7.43 (ddd, |
| | J = 8.2, 6.8, 1.2 Hz, 1H), 7.28 (d, J = 2.2 Hz, 2H), 7.26 (d, J = 3.2 Hz, 1H), 7.24 (t, J = 1.5 Hz, 1H), 7.22 (d, J = 2.4 Hz, |
| | 1H), 7.14-7.09 (m, 2H), 6.76 (s, 1H), 6.25 (s, 1H), 4.49-4.40 (m, 3H), 4.38-3.30 (m, 1H), 3.68-3.57 (m, 2H), 3.53 (s, |
| | 2H), 3.41 (s, 2H), 3.05-2.92 (m, 2H), 2.88 (dd, J = 7.6, 3.9 Hz, 1H), 2.71-2.57 (m, 1H), 2.46-2.32 (m, 6H), 2.30 (s, 6H), |
| | 2.15-2.05 (m, 1H), 1.62 (s, 5H), 0.93 (d, J = 6.9 Hz, 6H). |
| 34 | 10.60 (s, 1H), 9.74 (s, 1H), 9.18 (s, 1H), 8.94 (t, J = 5.9 Hz, 1H), 8.13-8.01 (m, 2H), 7.73 (d, J = 8.4 Hz, 1H), 7.70-7.62 |
| | (m, 2H), 7.57 (ddd, J = 8.2, 6.8, 1.3 Hz, 1H), 7.50 (ddd, J = 8.3, 6.8, 1.4 Hz, 1H), 7.39-7.32 (m, 2H), 7.31-7.24 (m, |
| | 2H), 6.57 (s, 1H), 6.33 (s, 1H), 4.51-4.41 (m, 3H), 4.40-4.30 (m, 1H), 3.70-3.60 (m, 2H), 3.54 (s, 2H), 3.47 (s, 2H), 3.16 (dd, |
| | J = 7.3, 5.9 Hz, 2H), 3.10-3.01 (m, 1H), 2.89 (p, J = 6.9 Hz, 2H), 2.70-2.59 (m, 1H), 2.39-2.35 (m, 10H), 2.31 (s, 3H), 2.12-2.04 |
| | (m, 1H), 1.66-1.56 (m, 5H), 1.03 (t, J = 7.2 Hz, 3H), 0.80 (d, J = 6.9 Hz, 6H). |
| 35 | 10.07 (s, 1H), 9.60 (s, 1H), 9.17 (s, 1H), 8.12-8.07 (m, 2H), 7.73 (d, J = 8.4 Hz, 1H), 7.69-7.62 (m, 2H), 7.58 (ddd, |
| | J = 8.1, 6.7, 1.3 Hz, 1H), 7.50 (ddd, J = 8.3, 6.8, 1.4 Hz, 1H), 7.25-7.19 (m 3H), 7.04 (s, 1H), 6.39 (s, |
| | 1H), 4.76 (s, 4H), 4.48-4.39 (m, 3H), 4.38-4.30 (m, 1H), 3.69-3.59 (m, 2H), 3.52 (s, 2H), 3.41 (s, 2H), 3.09 (d, J = 6.9 Hz, |
| | 1H), 3.05-2.90 (m, 1H), 2.88 (d, J = 7.4 Hz, 1H), 2.62 (dd, J = 9.7, 5.5 Hz, 1H), 2.39-2.32 (m, 9H), 2.29 (s, 3H), 2.15- |
| | 1.99 (m, 1H), 1.62 (s, 5H), 1.13 (d, J = 6.9 Hz, 6H). |
| 36 | 10.60 (s, 1H), 9.75 (s, 1H), 9.04 (s, 1H), 8.93 (t, J = 5.9 Hz, 1H), 8.22-8.08 (m, 2H), 7.75-7.64 (m, 2H), 7.62-7.52 (m, |
| | 2H), 7.39-7.33 (m, 2H), 7.32-7.25 (m, 2H), 6.57 (s, 1H), 6.34 (s, 1H), 4.52-4.38 (m, 2H), 4.36-4.25 (m, 2H), 3.69- |
| | 3.59 (m, 2H), 3.56-3.51 (m, 2H), 3.47 (s, 2H), 3.23-3.21 (m, 1H), 3.20-3.10 (m, 2H), 3.05-2.97 (m, 1H), 2.89 (dd, J = 9.0, 4.8 Hz, |
| | 2H), 2.63 (s, 1H), 2.39 (s, 6H), 2.33-2.31 (m, 2H), 2.30 (s, 3H), 2.29 (s, 1H), 2.14-2.03 (m, 1H), 1.70-1.52 (m, 5H), 1.03 |
| | (t, J = 7.2 Hz, 3H), 0.80 (d, J = 6.9 Hz, 6H). |
| 37 | 10.09 (s, 1H), 9.60 (s, 1H), 9.04 (s, 1H), 8.22-8.09 (m, 2H), 7.75-7.65 (m, 2H), 7.62-7.52 (m, 2H), 7.39-7.12 (m, 3H), |
| | 7.04 (s, 1H), 6.39 (s, 1H), 4.76 (s, 4H), 4.50-4.36 (m, 2H), 4.35-4.26 (m, 2H), 3.67-3.58 (m, 2H), 3.52 (s, 2H), 3.41 (s, |
| | 2H), 3.13-3.05 (m, 1H), 3.02-2.95 (m, 1H), 2.87 (s, 1H), 2.61 (s, 1H), 2.43-2.36 (m, 5H), 2.34-2.17 (s, 6H), 2.11-2.02 (m, 1H), |
| | 1.76-1.47 (m, 6H), 1.13 (d, J = 6.9 Hz, 6H). |
| 38 | 10.62 (s, 1H), 9.97 (s, 1H), 9.75 (s, 1H), 9.16 (s, 1H), 8.95 (t, J = 5.9 Hz, 1H), 7.80 (d, J = 8.3 Hz, 1H), 7.56 (d, |
| | J = 8.4 Hz, 1H), 7.49-7.41 (m, 1H), 7.41-7.34 (m, 2H), 7.33-7.27 (m, 3H), 7.27-7.19 (m, 2H), 6.57 (s, 1H), 6.34 (s, 1H), 4.53- |
| | 4.41 (m, 3H), 4.38-4.35 (m, 1H), 3.65-3.55 (m, 2H), 3.53 (s, 2H), 3.46 (s, 6H), 3.21-3.10 (m, 2H), 3.02-2.92 (m, 2H), 2.90 |
| | (p, J = 6.8 Hz, 1H), 2.84-2.80 (m, 2H), 2.67 (p, J = 1.9 Hz, 1H), 2.39 (s, 2H), 2.36-2.21 (m, 7H), 2.12-2.05 (m, 1H), 1.98 (s, 2H), |
| | 1.69-1.50 (m, 9H), 1.03 (t, J = 7.2 Hz, 3H), 0.80 (d, J = 6.9 Hz, 6H). |
| 39 | 10.06 (s, 1H), 9.60 (s, 1H), 9.03 (d, J = 1.3 Hz, 1H), 8.14 (d, J = 8.4, 2H), 7.75-7.65 (m, 2H), 7.61-7.52 (m, 2H), 7.39-7.11 |
| | (m, 3H), 7.04 (s, 1H), 6.39 (s, 1H), 4.76 (s, 4H), 4.46 (d, J = 12.0 Hz, 1H), 4.40-4.27 (m, 2H), 4.12 (d, J = 7.6 Hz, 1H), |
| | 3.66 (d, J = 3.3 Hz, 1H), 3.62 (d, J = 12.0 Hz, 1H), 3.55 (d, J = 12.8 Hz, 3H), 3.47-3.35 (m, 3H), 3.13-3.05 (m, 1H), 2.73 (s, |
| | 1H), 2.46-2.28 (m, 12H), 2.17 (d, J = 6.7 Hz, 1H), 1.96-1.79 (m, 2H), 1.65 (s, 4H), 1.56 (t, J = 7.8 Hz, 1H), 1.47 |
| | (s, 1H), 1.13 (d, J = 6.9 Hz, 6H). |
| 40 | 10.54 (s, 1H), 10.08-9.62 (m, 2H), 9.16 (s, 1H), 8.94 (t, J = 5.9 Hz, 1H), 7.80 (d, J = 8.4 Hz, 1H), 7.55 (d, J = 8.3 Hz, 1H), 7.43 |
| | (ddd, J = 8.2, 6.7, 1.2 Hz, 1H), 7.35 (d, J = 8.4 Hz, 2H), 7.28 (q, J = 2.2 Hz, 2H), 7.27-7.20 (m, 3H), 6.59 (s, 1H), 6.33 (s, |
| | 1H), 4.56-4.41 (m, 2H), 4.17-4.00 (m, 2H), 3.64-3.57 (m, 2H), 3.53 (d, J = 9.5 Hz, 3H), 3.51-3.42 (m, 1H), 3.28 (s, 1H), 3.21- |
| | 3.09 (m, 3H), 2.95-2.83 (m, 2H), 2.69-2.60 (m, 1H), 2.46 (d, J = 9.0 Hz, 1H), 2.39-2.30 (m, 1H), 2.04 (s, 3H), 1.97-1.85 |
| | (m, 2H), 1.84-1.68 (m, 2H), 1.63 (s, 4H), 1.59-1.49 (m, 1H), 1.03 (t, J = 7.2 Hz, 3H), 0.81 (d, J = 6.9 Hz, 6H). |
| 41 | 10.55 (s, 1H), 9.96 (s, 1H), 9.73 (s, 1H), 9.16 (s, 1H), 8.93 (t, J = 5.9 Hz, 1H), 7.79 (d, J = 8.4 Hz, 1H), 7.58-7.51 (m, 1H), 7.43 (ddd, |
| | J = 8.2, 6.7, 1.3 Hz, 1H), 7.39-7.33 (m, 2H), 7.31-7.23 (m, 3H), 7.21 (q, J = 3.7, 2.9 Hz, 2H), 6.57 (s, 1H), 6.33 (s, 1H), 4.45 (t, J = 10.0 |
| | Hz, 2H), 4.13-4.09 (m, 2H), 3.65-3.44 (m, 6H), 3.14 (td, J = 7.3, 5.9 Hz, 2H), 3.06 (s, 2H), 2.93 (dt, J = 9.6, 4.9 Hz, 1H), 2.90- |
| | 2.83 (m, 1H), 2.83-2.76 (m, 1H), 2.58 (d, J = 6.4 Hz, 1H), 2.18-2.10 (m, 1H), 2.05 (s, 4H), 1.83-1.68 (m, 3H), 1.63 (s, 5H), |
| | 1.03 (t, J = 7.2 Hz, 3H), 0.79 (dd, J = 6.9, 4.0 Hz, 6H). |
| 42 | 11.89 (s, 1H), 9.96 (s, 1H), 9.68-9.31 (m, 2H), 9.16 (s, 1H), 7.80 (d, J = 8.3 Hz, 1H), 7.55 (d, J = 8.3 Hz, 1H), 7.43 (ddd, |
| | J = 8.1, 6.7, 1.2 Hz, 1H), 7.30-7.26 (m, 2H), 7.26-7.20 (m, 3H), 7.13-7.08 (m, 2H), 6.77 (s, 1H), 6.25 (s, 1H), 4.51-4.40 |
| | (m, 2H), 4.14-4.09 (m, 1H), 4.08-4.02 (m, 1H), 3.66-3.59 (m, 2H), 3.54 (s, 2H), 3.49-3.39 (m, 2H), 3.36 (s, 1H), 3.28-3.23 |
| | (m, 1H), 3.14-3.01 (m, 1H), 3.00-2.85 (m, 1H), 2.70-2.60 (m, 1H), 2.45 (t, J = 9.3 Hz, 1H), 2.37-2.27 (m, 1H), 1.98 |
| | (s, 3H), 1.95-1.80 (m, 2H), 1.80-1.67 (m, 2H), 1.63 (s, 4H), 1.56-1.49 (m, 1H), 0.94 (d, J = 6.9 Hz, 6H). |
| 43 | 11.89 (s, 1H), 9.96 (s, 1H), 9.65-9.25 (m, 2H), 9.16 (s, 1H), 7.80 (d, J = 8.3 Hz, 1H), 7.55 (d, J = 8.2 Hz, 1H), 7.43 (ddd, J = |
| | 8.1, 6.7, 1.2 Hz, 1H), 7.31-7.19 (m, 5H), 7.13-7.06 (m, 2H), 6.76 (s, 1H), 6.24 (s, 1H), 4.45 (t, J = 9.6 Hz, 2H), 4.22- |
| | 4.05 (m, 2H), 3.64-3.56 (m, 2H), 3.52-3.46 (m, 3H), 3.42-3.36 (m, 1H), 3.08-2.98 (m, 2H), 2.97-2.87 (m, 2H), 2.82-2.73 |
| | (m, 1H), 2.61-2.54 (m, 1H), 2.14-2.06 (m, 1H), 2.00 (s, 4H), 1.80-1.67 (m, 3H), 1.62 (s, 5H), 0.93 (dd, J = 6.9, 1.5 Hz, 6H). |
| 44 | 10.31-9.90 (m, 2H), 9.61 (s, 1H), 9.17 (s, 1H), 7.80 (d, J = 8.3 Hz, 1H), 7.55 (d, J = 8.2 Hz, 1H), 7.43 (ddd, J = 8.2, 6.8, 1.3 Hz, 1H), |
| | 7.31-7.15 (m, 6H), 7.05 (s, 1H), 6.39 (s, 1H), 4.75 (s, 4H), 4.51-4.41 (m, 2H), 4.17-3.99 (m, 2H), 3.69-3.53 (m, 4H), 3.51-3.34 |
| | (m, 3H), 3.25-3.24 (m, 1H), 3.09 (p, J = 6.4, 5.8 Hz, 2H), 2.90 (s, 1H), 2.70-2.61 (m, 1H), 2.30 (d, J = 5.5 Hz, 1H), |
| | 1.99 (s, 3H), 1.95-1.70 (m, 4H), 1.66 (s, 4H), 1.59-1.49 (m, 1H), 1.13 (d, J = 6.9 Hz, 6H). |
| 45 | 10.31-9.90 (m, 2H), 9.60 (s, 1H), 9.16 (s, 1H), 7.80 (d, J = 8.3 Hz, 1H), 7.55 (d, J = 8.5 Hz, 1H), 7.43 (ddd, J = 8.2. |
| | 6.8, 1.2 Hz, 1H), 7.31-7.16 (m, 6H), 7.04 (s, 1H), 6.39 (s, 1H), 4.75 (s, 4H), 4.51-4.37 (m, 2H), 4.23-4.06 (m, 2H), 3.66-3.49 |
| | (m, 5H), 3.47-3.40 (m, 1H), 3.13-2.98 (m, 3H), 2.97-2.90 (m, 1H), 2.86-2.73 (m, 1H), 2.57 (d, J = 8.8 Hz, 1H), 2.05-2.15 |
| | (m, 1H), 2.00 (s, 4H), 1.84-1.69 (m, 3H), 1.64 (s, 5H), 1.23 (s, 1H), 1.13 (d, J = 6.9 Hz, 6H). |
| 46 | 10.06 (s, 1H), 9.59 (s, 1H), 9.03 (s, 1H), 8.13 (ddd, J = 8.4, 7.0, 1.4 Hz, 2H), 7.75-7.65 (m, 2H), 7.60-7.52 (m, 2H), 7.36-7.12 |
| | (m, 3H), 7.05 (s, 1H), 6.39 (s, 1H), 4.76 (s, 4H), 4.51-4.41 (m, 1H), 4.30 (d, J = 8.3 Hz, 3H), 3.65 (s, 2H), 3.59- |
| | 3.47 (m, 3H), 3.43 (s, 6H), 3.09 (p, J = 6.9 Hz, 1H), 2.81-2.72 (m, 2H), 2.39 (s, 2H), 2.35-2.28 (m, 5H), 1.99-1.89 (m, 2H), |
| | 1.66 (s, 4H), 1.53 (s, 4H), 1.13 (d, J = 6.9 Hz, 6H), 0.64 (s, 2H), 0.41 (s, 2H). |
| 47 | 10.81-10.55 (m, 1H), 9.95 (s, 1H), 9.75 (s, 1H), 9.17 (d, J = 5.9 Hz, 1H), 8.94 (t, J = 5.9 Hz, 1H), 7.89-7.74 (m, 1H), 7.55 |
| | (d, J = 8.4 Hz, 1H), 7.51-7.34 (m, 2H), 7.29 (d, J = 8.6 Hz, 2H), 7.25-7.14 (m, 4H), 6.64-6.51 (m, 1H), 6.34 (s, 1H), 5.20- |
| | 4.61 (m, 1H), 4.46 (s, 2H), 4.26-3.95 (m, 2H), 3.72-3.54 (m, 4H), 3.47 (s, 1H), 3.22 (s, 2H), 3.20-3.09 (m, 3H), 3.06 |
| | (s, 1H), 2.98-2.81 (m, 5H), 2.77-2.66 (m, 2H), 2.62 (s, 1H), 2.09 (s, 1H), 2.06-1.91 (m, 4H), 1.86-1.76 (m, 3H), 1.69-1.65 |
| | (m, 4H), 1.61-1.52 (m, 4H), 1.03 (t, J = 7.2 Hz, 3H), 0.84-0.68 (m, 6H). |
| 48 | 10.61 (s, 1H), 10.05-9.97 (m, 1H), 9.77 (s, 1H), 9.24 (s, 1H), 8.97 (d, J = 6.4 Hz, 1H), 7.82 (d, J = 8.3 Hz, 1H), 7.54 (d, |
| | (d, J = 8.4 Hz, 1H), 7.50-7.19 (m, 8H), 6.61-6.53 (m, 1H), 6.35 (s, 1H), 5.15-4.90 (m, 1H), 4.65 (s, 2H), 4.31-4.04 (m, |
| | 3H), 3.84 (s, 2H), 3.49 (s, 2H), 3.16 (p, J = 6.9 Hz, 3H), 3.01-2.85 (m, 6H), 2.81-2.73 (m, 2H), 2.15-1.95 (m, 4H), 1.91 |
| | (s, 5H), 1.60 (s, 4H), 1.25 (d, J = 9.5 Hz, 5H), 1.04 (t, J = 7.2 Hz, 3H), 0.90-0.75 (m, 6H). |
| 49 | 10.13-9.91 (s, 2H), 9.61 (s, 1H), 9.16 (s, 1H), 7.80 (d, J = 8.3 Hz, 1H), 7.55 (d, J = 8.5 Hz, 1H), 7.48-7.40 (m, 1H), 7.33-7.15 |
| | (m, 6H), 7.04 (s, 1H), 6.39 (s, 1H), 4.76 (s, 4H), 4.50-4.42 (m, 2H), 4.16 (s, 2H), 3.69-3.46 (m, 4H), 3.42 (s, 2H), |
| | 3.31-3.21 (m, 2H), 3.11-3.03 (m, 2H), 2.99-2.70 (m, 6H), 2.67 (p, J = 1.9 Hz, 1H), 2.07-1.75 (m, 7H), 1.65 (s, 6H), 1.56 |
| | (s, 4H), 1.13 (d, J = 6.9 Hz, 6H). |
| 50 | 10.13-9.91 (m, 2H), 9.61 (s, 1H), 9.17 (d, J = 2.9 Hz, 1H), 7.80 (d, J = 8.5 Hz, 1H), 7.57 (d, J = 8.0 Hz, 1H), 7.48-7.35 (m, 1H), |
| | 7.30-7.15 (m, 5H), 7.04 (s, 1H), 6.97 (d, J = 7.6 Hz, 1H), 6.40 (d, J = 3.6 Hz, 1H), 5.17-4.56 (m, 5H), 4.55-4.35 (m, 2H), |
| | 4.28-3.95 (m, 2H), 3.71-3.51 (m, 4H), 3.43 (s, 1H), 3.31-3.21 (m, 2H), 3.11-3.03 (m, 2H), 2.90 (s, 3H), 2.78 (s, 1H), |
| | 2.76-2.54 (m, 4H), 2.13-1.88 (m, 3H), 1.86-1.80 (m, 4H), 1.66 (s, 4H), 1.54 (s, 5H), 1.13 (d, J = 6.9 Hz, 6H). |
| 51 | 10.07 (s, 1H), 9.60 (s, 1H), 9.09 (s, 1H), 7.37-7.10 (m, 6H), 7.04 (s, 1H), 6.39 (s, 1H), 4.76 (s, 4H), 4.48-4.23 (m, 4H), |
| | 3.62-3.50 (m, 4H), 3.42 (s, 2H), 3.12-3.06 (m, 1H), 3.00-2.97 (m, 1H), 2.90-2.87 (m, 1H), 2.62-2.59 |
| | (m, 1H), 2.48-2.42 (m, 2H), 2.32 (s, 4H), 2.29 (s, 9H), 2.07-2.03 (m, 4H), 1.61-1.58 (m, 5H), 1.13 (d, J = 6.9 Hz, 6H). |
| 52 | 11.91 (s, 1H), 9.58 (s, 1H), 9.44 (s, 1H), 9.09 (s, 1H), 7.32-7.25 (m, 3H), 7.25-7.15 (m, 2H), 7.15-7.08 (m, 2H), 6.67 |
| | (s, 1H), 6.25 (s, 1H), 4.43-4.39 (m, 3H), 4.35-4.31 (m, 1H), 3.63-3.47 (m, 4H), 3.41 (s, 2H), 3.03-2.89 (m, 2H), 2.89- |
| | 2.60 (m, 1H), 2.65-2.57 (m, 1H), 2.40-2.32 (m, 6H), 2.31 (s, 9H), 2.11-2.00 (m, 4H), 1.60 (s, 5H), 0.93 (d, J = 6.9 Hz, 6H). |
| 53 | 10.60 (s, 1H), 9.74 (s, 1H), 9.09 (s, 1H), 8.93 (t, J = 5.9 Hz, 1H), 7.36 (d, J = 8.4 Hz, 2H), 7.31-7.25 (m, 3H), 7.25-7.15 |
| | (m, 2H), 6.57 (s, 1H), 6.33 (s, 1H), 4.44-4.41 (m, 3H), 4.34-4.31 (m, 1H), 3.63-3.52 (m, 4H), 3.47 (s, 2H), 3.22-3.10 (m, 2H), |
| | 3.01-2.99 (m, 1H), 2.88 (m, 2H), 2.66-2.62 (m, 1H), 2.39 (s, 6H), 2.31 (d, J = 6.5 Hz, 9H), 2.11-2.06 (m, 4H), 1.64- |
| | 1.53 (m, 5H), 1.03 (t, J = 7.2 Hz, 3H), 0.80 (d, J = 6.9 Hz, 6H). |
| 54 | 10.46 (s, 1H), 9.74 (s, 1H), 9.57 (d, J = 6.4 Hz, 1H), 9.05 (s, 1H), 8.18-8.10 (m, 2H), 7.75-7.66 (m, 2H), 7.61-7.54 (m, 2H), 7.32 (q, |
| | J = 8.4 Hz, 4H), 6.60 (s, 1H), 6.33 (s, 1H), 4.56-4.48 (m, 1H), 4.36-4.22 (m, 3H), 4.01-3.89 (m, 2H), 3.68 (d, J = 8.8 Hz, 3H), 3.62-3.55 |
| | (m, 1H), 3.45 (s, 2H), 3.26-3.21 (m, 7H), 2.88 (p, J = 6.8 Hz, 1H), 2.37 (s, 3H), 2.35-2.22 (m, 2H), 1.72 (s, 4H), 0.80 (d, J = 6.8 |
| | Hz, 6H), 0.63 (s, 2H), 0.41 (s, 2H). |
| 55 | 10.44 (s, 1H), 9.73 (s, 1H), 9.54 (d, J = 9.0 Hz, 1H), 9.04 (s, 1H), 8.14 (ddd, J = 8.4, 7.0, 1.3 Hz, 2H), 7.74-7.67 (m, 2H), 7.60- |
| | 7.54 (m, 2H), 7.34 (d, J = 8.4 Hz, 2H), 7.28 (d, J = 8.5 Hz, 2H), 6.61 (s, 1H), 6.33 (s, 1H), 4.69-4.57 (m, 1H), 4.52-4.46 |
| | (m, 1H), 4.31-4.21 (m, 3H), 3.66 (s, 2H), 3.58-3.50 (m, 3H), 3.44 (s, 2H), 2.88 (p, J = 6.9 Hz, 1H), 2.42-2.34 (m, 5H), |
| | 2.35-2.24 (m, 2H), 1.67 (s, 4H), 1.31 (d, J = 7.0 Hz, 3H), 0.81 (d, J = 6.8 Hz, 6H), 0.63 (s, 2H), 0.40 (s, 2H). |
| 56 | 10.47 (s, 1H), 9.75 (s, 1H), 9.58 (t, J = 6.5 Hz, 1H), 8.02 (dd, J = 9.1, 5.8 Hz, 1H), 7.79 (dd, J = 8.0, 4.3 Hz, 1H), |
| | 7.65-7.48 (m, 2H), 7.46-7.22 (m, 5H), 6.85 (s, 1H), 6.61 (s, 1H), 6.34 (s, 1H), 6.22-6.17 (m, 1H), 5.78-5.74 (d, J = 10.4 Hz, |
| | 1H), 5.02-4.76 (m, 1H), 4.52-4.09 (m, 3H), 4.08-3.70 (m, 6H), 3.46 (s, 7H), 3.20-2.96 (m, 5H), 2.90 (h, J = 6.4, 5.8 Hz, 2H), 2.85- |
| | 2.76 (m, 2H), 2.71-2.67 (m, 1H), 2.61-2.51 (m, 2H), 2.48-2.25 (m, 10H), 2.24-2.16 (m, 1H), 1.99 (s, 2H), 1.84 (d, J = 7.4 |
| | Hz, 2H), 1.55 (s, 6H), 0.82 (d, J = 6.9 Hz, 6H). |
| 57 | 10.07 (s, 1H), 9.60 (s, 1H), 9.09 (s, 1H), 7.35-7.11 (m, 6H), 7.05 (s, 1H), 6.39 (s, 1H), 4.76 (s, 4H), 4.43-4.32 (m, 4H), 3.62-3.48 (m, |
| | 4H), 3.43 (s, 4H), 3.09 (p, J = 6.9 Hz, 1H), 3.04-2.90 (m, 2H), 2.79-2.77 (m, 2H), 2.60-2.56 (m, 2H), 2.37 (s, 2H), 2.32-2.38 (m, 9H), |
| | 2.10-2.06 (m, 4H), 1.98-1.92 (m, 2H), 1.67-1.44 (m, 9H), 1.13 (d, J = 6.9 Hz, 6H). |
| 58 | 10.06 (s, 1H), 9.62 (s, 1H), 9.15 (s, 1H), 7.94 (dd, J = 8.0, 1.4 Hz, 1H), 7.89-7.70 (m, 2H), 7.57 (d, J = 7.5 Hz, 1H), 7.21 (s, 3H), |
| | 7.04 (s, 1H), 6.39 (s, 1H), 4.76 (s, 4H), 4.57-4.36 (m, 2H), 4.46 (d, J = 4.8 Hz, 2H), 4.04 (s, 2H), 3.85-3.78 (m, 2H), 3.47 (s, 3H), |
| | 3.09 (p, J = 6.8 Hz, 2H), 2.81-2.64 (m, 1H), 2.47-2.28 (m, 12H), 2.11-2.02 (m, 2H), 1.86 (s, 4H), 1.66 (s, 1H), 1.13 (d, J = 6.9 Hz, 6H). |
| 59 | 10.59 (s, 1H), 9.74 (s, 1H), 9.09 (s, 1H), 8.95 (t, J = 5.9 Hz, 1H), 8.45 (dd, J = 8.5, 1.4 Hz, 1H), 8.33-8.24 (m, 1H), 8.13 (dd, J = 7.2, |
| | 1.3 Hz, 1H), 7.83-7.81 (m, 2H), 7.73 (dd, J = 8.3, 7.2 Hz, 1H), 7.36 (d, J = 8.4 Hz, 2H), 7.31-7.24 (m, 2H), 6.57 (s, 1H), 6.33 (s, 1H), |
| | 4.47-4.31 (m, 4H), 3.62 (s, 2H), 3.52 (s, 2H), 3.47 (s, 2H), 3.21-3.10 (m, 2H), 3.03-2.99 (m, 1H), 2.92-2.86 (m, 2H), 2.66-2.60 |
| | (m, 2H), 2.39 (s, 6H), 2.31 (s, 5H), 2.11-2.05 (m, 1H), 1.65-1.49 (m, 5H), 1.03 (t, J = 7.2 Hz, 3H), 0.80 (d, J = 6.9 Hz, 6H). |
| 60 | 9.10 (s, 1H), 8.95 (t, J = 5.9 Hz, 1H), 8.20 (s, 2H), 7.93 (dd, J = 8.0, 1.4 Hz, 1H), 7.86-7.79 (m, 1H), 7.75 (t, J = 7.6 Hz, 1H), 7.58 |
| | (d, J = 7.5 Hz, 1H), 7.36 (d, J = 8.3 Hz, 2H), 7.32-7.22 (m, 2H), 6.57 (s, 1H), 6.34 (s, 1H), 4.51-4.44 (m, 3H), 4.35-4.31 (m, 1H), 4.13 (s, |
| | 2H), 3.68-3.61 (m, 4H), 3.47 (s, 2H), 3.23-3.10 (m, 2H), 3.02-2.94 (m, 1H), 2.89 (h, J = 6.9, 6.3 Hz, 2H), 2.72-2.59 (m, 1H), |
| | 2.45-2.35 (m, 6H), 2.31 (s, 5H), 2.09-2.16 (m, 1H), 1.79-1.54 (m, 5H), 1.03 (t, J = 7.2 Hz, 3H), 0.80 (d, J = 6.9 Hz, 6H). |
| 61 | 10.09 (s, 1H), 9.60 (s, 1H), 9.10 (s, 1H), 7.43-7.33 (m, 1H), 7.32-7.15 (m, 5H), 7.04 (s, 1H), 6.39 (s, 1H), |
| | 4.76 (s, 4H), 4.45-4.27 (m, 4H), 3.57 (dd, J = 12.0, 7.1 Hz, 2H), 3.50 (s, 2H), 3.49-3.41 |
| | (m, 2H), 3.15-3.09 (m, 1H), 3.04-2.94 (m, 1H), 2.88 (s, 1H), 2.69-2.64 (m, 2H), 2.33 (d, J = 1.8 |
| | Hz, 7H), 2.29 (s, 5H), 2.12 (d, J = 2.2 Hz, 3H), 2.06 (dd, J = 12.8, 6.5 Hz, 1H), 1.59 (s, 5H), 1.13 (d, J = 6.9 Hz, 6H). |
| 62 | 10.59 (s, 1H), 9.74 (s, 1H), 9.14 (s, 1H), 8.95 (s, 1H), 7.35 (t, J = 8.8 Hz, 3H), 7.27 (dd, J = 10.9, 7.8 Hz, 3H), 6.57 (s, 1H), |
| | 6.33 (s, 1H), 4.64-4.28 (m, 4H), 3.67 (s, 4H), 3.48 (s, 2H), 3.23-3.06 (m, 2H), 3.02 (s, 1H), 2.96-2.75 (m, 2H), 2.60- |
| | 2.36 (m, 8H), 2.35-2.24 (m, 5H) 2.12 (s, 4H), 1.71 (s, 5H), 1.15 (s, 1H), 1.03 (t, J = 7.2 Hz, 3H), 0.80 (d, J = 6.9 Hz, 6H). |
| 63 | 10.06 (s, 1H), 9.60 (s, 1H), 9.09 (s, 1H), 8.45 (dd, J = 8.4, 1.3 Hz, 1H), 8.28 (dd, J = 7.1, 3.6 Hz, 1H), 8.13 (dd, J = 7.2, 1.3 |
| | Hz, 1H), 7.87-7.78 (m, 2H), 7.73 (dd, J = 8.3, 7.2 Hz, 1H), 7.35-7.14 (m, 3H), 7.04 (s, 1H), 6.39 (s, 1H), 4.75 (s, 4H), 4.52- |
| | 3.26 (m, 4H), 3.62 (s, 2H), 3.51 (s, 2H), 3.41 (s, 2H), 3.09 (p, J = 6.9 Hz, 1H), 3.01-2.95 (m, 1H), 2.91-2.85 (m, 1H), 2.67-2.57 (m, |
| | 1H), 2.43-2.32 (m, 4H), 2.31-2.20 (m, 8H), 2.12-2.01 (m, 1H), 1.71-1.42 (m, 5H), 1.13 (d, J = 6.9 Hz, 6H). |
| 64 | 10.07 (s, 1H), 9.96 (s, 1H), 9.60 (s, 1H), 9.15 (s, 1H), 7.80 (d, J = 8.2 Hz, 1H), 7.55 (d, J = 8.4 Hz, 1H), 7.43 (ddd, |
| | J = 8.1, 6.8, 1.2 Hz, 1H), 7.28 (d, J = 2.5 Hz, 1H), 7.27-7.23 (m, 2H), 7.22 (d, J = 2.4 Hz, 2H), 7.17 (d, J = 7.5 Hz, 1H), 7.04 (s, |
| | 1H), 6.39 (s, 1H), 4.76 (s, 4H), 4.48-4.42 (m, 2H), 4.38-4.33 (m, 1H), 4.19-4.15 (m, 1H), 3.62-3.55 (m, 2H), 3.52 (s, 2H), 3.41 (s, |
| | 2H), 3.32 (s, 2H), 3.12-3.07 (m, 1H), 2.74 (dd, J = 7.6, 4.9 Hz, 1H), 2.40 (s, 3H), 2.38-2.22 (m, 8H), 2.18-2.13 (m, 1H), 1.90- |
| | 1.80 (m, 2H), 1.69-1.53 (m, 5H), 1.52-1.40 (m, 1H), 1.13 (d, J = 6.9 Hz, 6H). |
| 65 | 10.60 (s, 1H), 9.96 (s, 1H), 9.74 (s, 1H), 9.16 (s, 1H), 8.94 (t, J = 5.9 Hz, 1H), 7.80 (d, J = 8.2 Hz, 1H), 7.56 (d, J = 8.4 Hz, 1H), |
| | 7.43 (ddd, J = 8.2, 6.8, 1.2 Hz, 1H), 7.36 (d, J = 8.4 Hz, 2H), 7.28 (dd, J = 5.6, 2.8 Hz, 3H), 7.26-7.20 (m, 2H), 6.57 (s, 1H), |
| | 6.34 (s, 1H), 4.48-4.42 (m, 2H), 4.40-4.35 (m, 1H), 4.20-4.15 (m, 1H), 3.65-3.58 (m, 2H), 3.54 (s, 2H), 3.47 (s, 2H), 3.21-3.10 (m, |
| | 2H), 2.92-2.88 (m, 1H), 2.74 (q, J = 6.3 Hz, 1H), 2.61-2.52 (m, 2H), 2.41 (s, 5H), 2.40-2.25 (m, 6H), 2.24-2.18 (m, 1H), 1.93-1.80 (m, |
| | 2H), 1.70-1.54 (m, 5H), 1.52-1.42 (m, 1H), 1.03 (t, J = 7.2 Hz, 3H), 0.80 (d, J = 6.9 Hz, 6H). |
| 66 | 10.60 (s, 1H), 9.74 (s, 1H), 9.18 (s, 1H), 8.94 (t, J = 5.9 Hz, 1H), 8.13-8.01 (m, 2H), 7.73 (d, J = 8.4 Hz, 1H), 7.70-7.62 (m, 2H), |
| | 7.61-7.54 (m, 1H), 7.50-7.46 (m, 1H), 7.36 (d, J = 8.1 Hz, 2H), 7.28 (d, J = 8.3 Hz, 2H), 6.57 (s, 1H), 6.34 (s, 1H), 4.46 (d, J = 11.9 Hz, |
| | 2H), 4.38 (dd, J = 10.7, 4.6 Hz, 1H), 4.17 (dd, J = 10.7, 6.7 Hz, 1H), 3.61 (d, J = 12.1 Hz, 2H), 3.56 (s, 2H), 3.47 (s, 2H), 3.22-3.10 |
| | (m, 2H), 2.89 (p, J = 6.9 Hz, 1H), 2.76 (t, J = 6.3 Hz, 1H), 2.54 (s, 2H), 2.40 (d, J = 7.5 Hz, 5H), 2.38-2.29 (m, 6H), 2.19 (dd, J = 12.3, |
| | 6.7 Hz, 1H), 1.87 (q, J = 11.2, 8.1 Hz, 2H), 1.64 (s, 4H), 1.58 (t, J = 7.5 Hz, 1H), 1.50 (s, 1H), 1.03 (t, J = 7.2 Hz, 3H), 0.80 (d, J = 6.9 |
| | Hz, 6H). |
| 67 | 10.07 (s, 1H), 9.58 (s, 1H), 9.16 (s, 1H), 8.12-8.01 (m, 2H), 7.73 (d, J = 8.4 Hz, 1H), 7.66 (d, J = 4.9 Hz, 2H), 7.61-7.53 (m, 1H), |
| | 7.50-7.41 (m, 1H), 7.32-7.12 (m, 3H), 7.05 (s, 1H), 6.39 (s, 1H), 4.76 (s, 4H), 4.44 (d, J = 12.2 Hz, 2H), 4.37 (dd, J = 11.2, 4.4 Hz, |
| | 1H), 4.18 (s, 1H), 3.61-3.53 (m, 2H), 3.54 (s, 2H), 3.42 (s, 2H), 3.13-3.05 (m, 1H), 2.74 (s, 1H), 2.40 (s, 5H), 2.37-2.22 (m, 8H), 2.17 |
| | (dd, J = 12.2, 6.7 Hz, 1H), 1.85 (s, 2H), 1.71-1.54 (m, 5H), 1.48 (s, 1H), 1.14 (d, J = 7.0 Hz, 6H). |
| 68 | 10.07 (s, 1H), 9.60 (s, 1H), 9.18-9.09 (m, 1H), 8.07 (dd, J = 8.2, 1.4 Hz, 1H), 7.91 (d, J = 8.1 Hz, 1H), 7.59 (t, J = 7.6 Hz, 1H), 7.47- |
| | 7.41 (m, 2H), 7.32 (t, J = 5.6 Hz, 1H), 7.27-7.17 (m, 3H), 7.04 (s, 1H), 6.39 (s, 1H), 4.79-4.55 (m, 5H), 4.47-4.29 (m, 2H), |
| | 4.22-4.12 (m, 1H), 3.74-3.71 (m, 1H), 3.51 (s, 1H), 3.41 (s, 5H), 3.12-3.05 (m, 1H), 3.02-2.93 (m, 1H), 2.90-2.86 (m, 2H), 2.70- |
| | 2.56 (m, 1H), 2.44-2.20 (m, 12H), 2.06 (s, 1H), 1.98-1.94 (m, 3H), 1.92-1.88 (m, 1H), 1.74-1.70 (m, 1H), 1.62-1.57 (m, 1H), |
| | 1.13 (d, J = 6.9 Hz, 6H). |
| 69 | 10.60 (s, 1H), 9.18-9.12 (m, 1H), 8.85 (t, J = 5.9 Hz, 1H), 8.07 (dd, J = 8.3, 1.5 Hz, 1H), 7.91 (d, J = 8.1 Hz, 1H), 7.60-7.57 (m, 1H), |
| | 7.50-7.39 (m, 2H), 7.38-7.29 (m, 3H), 7.29-7.22 (m, 2H), 6.57 (s, 1H), 6.34 (d, J = 2.8 Hz, 1H), 4.83-4.87 (m, 1H), 4.53-4.25 (m, 2H), |
| | 4.21-4.14 (m, 1H), 3.72-3.69 (m, 1H), 3.45 (s, 4H), 3.41-3.39 (m, 2H), 3.21-3.10 (m, 2H), 3.02-2.98 (m, 1H), 2.93-2.79 |
| | (m, 4H), 2.66-2.61 (m, 1H), 2.39 (s, 7H), 2.35-2.25 (m, 4H), 2.14-2.05 (m, 1H), 1.98-1.94 (m, 3H), 1.90-1.88 (m, 1H), 1.76- |
| | 1.66 (m, 1H), 1.64-1.57 (m, 1H), 1.03 (t, J = 7.2 Hz, 3H), 0.80 (d, J = 6.9 Hz, 6H). |
| 70 | 10.07 (s, 1H), 9.95 (s, 1H), 9.60 (s, 1H), 9.16 (s, 1H), 7.80 (d, J = 8.2 Hz, 1H), 7.55 (d, J = 8.1 Hz, 1H), 7.43 (ddd, J = 8.2, 6.7, |
| | 1.2 Hz, 1H), 7.36-7.12 (m, 6H), 7.05 (s, 1H), 6.39 (s, 1H), 4.76 (s, 4H), 4.48-4.43 (m, 2H), 4.42-4.36 (m, 1H), 4.22-4.15 (m, 1H), 3.63- |
| | 3.58 (m, 2H), 3.53 (s, 2H), 3.42 (s, 6H), 3.12-3.08 (m, 1H), 2.82-2.75 (m, 3H), 2.62-2.53 (m, 2H), 2.40 (s, 3H), 2.39-2.24 |
| | (m, 5H), 2.22-2.18 (m, 1H), 2.00-1.90 (m, 2H), 1.87 (d, J = 5.9 Hz, 2H), 1.63 (s, 5H), 1.59-1.49 (m, 5H), 1.14 (d, J = 6.9 Hz, 6H). |
| 71 | 10.05 (s, 1H), δ 9.60 (s, 1H), 9.08 (d, J = 8.0 Hz, 1H), 8.07 (dd, J = 8.3, 1.4 Hz, 1H), 7.91 (d, J = 8.1 Hz, 1H), 7.59 (td, J = 8.2, 7.6, |
| | 1.6 Hz, 1H), 7.49-7.42 (m, 2H), 7.36-7.13 (m, 4H), 7.04 (s, 1H), 6.39 (s, 1H), 4.76 (s, 4H), 4.53-4.47 (d, J = 12.1 Hz, 1H), 4.42-4.27 |
| | (m, 2H), 4.18-4.04 (m, 1H), 3.60-3.53 (m, 4H), 3.42 (s, 2H), 3.32 (s, 2H), 3.10-3.07 (m, 1H), 2.73 (s, 1H), 2.43-2.23 (m, 12H), |
| | 2.17 (s, 1H), 1.95 (s, 3H), 1.84 (s, 2H), 1.63 (s, 5H), 1.48 (s, 1H), 1.13-0.89 (d, J = 6.9 Hz, 6H). |
| 72 | 10.61 (s, 1H), 9.76 (s, 1H), 8.96 (t, J = 5.9 Hz, 1H), 8.30 (dt, J = 8.4, 1.5 Hz, 1H), 8.09 (dd, J = 7.3, 1.3 Hz, 1H), 7.91 (ddd, J = 7.2, 6.1, |
| | 1.0 Hz, 1H), 7.71-7.50 (m, 3H), 7.37 (d, J = 8.2 Hz, 2H), 7.29 (d, J = 8.3 Hz, 2H), 7.01-6.73 (m, 1H), 6.58 (s, 1H), 6.34 (s, 1H), |
| | 6.21-6.14 (m, 1H), 5.79-5.74 (m, 1H), 5.00-4.74 (m, 1H), 4.47-4.16 (m, 2H), 4.16-4.05 (m, 1H), 4.03-3.82 (m, 5H), 3.62 (s, 1H), |
| | 3.49-3.38 (m, 7H), 3.25 (d, J = 9.0 Hz, 3H), 3.15 (td, J = 7.3, 5.9 Hz, 3H), 3.08-3.01 (m, 2H), 2.91 (q, J = 6.9 Hz, 2H), 2.84-2.79 (m, 2H), |
| | 2.75-2.62 (m, 2H), 2.59-2.54 (m, 2H), 2.44-2.25 (m, 9H), 2.23-2.16 (m, 1H), 1.99 (s, 2H), 1.85 (s, 2H), 1.56 (s, 6H), 1.03 (t, J = 7.2 |
| | Hz, 3H), 0.80 (d, J = 6.9 Hz, 6H). |
| 73 | 10.24 (s, 1H), 9.95 (s, 1H), 9.69 (s, 1H), 9.58 (t, J = 6.5 Hz, 1H), 9.15 (s, 1H), 7.80 (d, J = 8.3 Hz, 1H), 7.55 (d, J = 8.1 Hz, 1H), 7.43 |
| | (ddd, J = 8.1, 6.8, 1.2 Hz, 1H), 7.37-7.30 (m, 2H), 7.28-7.15 (m, 5H), 6.59 (s, 1H), 6.31 (s, 1H), 4.49-4.45 (m, 2H), 4.42-4.35 (m, 1H), |
| | 4.20-4.16 (m, 1H), 4.03-3.89 (m, 2H), 3.65-3.58 (m, 2H), 3.54 (s, 2H), 3.46 (s, 2H), 2.80-2.71 (m, 1H), 2.52 (s, 2H), 2.45-2.28 |
| | (m, 11H), 2.26-2.18 (m, 3H), 1.86 (q, J = 8.7, 7.7 Hz, 2H), 1.63 (s, 4H), 1.60-1.41 (m, 2H), 0.84 (t, J = 7.5 Hz, 3H). |
| 74 | 9.98 (s, 2H), 9.61 (s, 1H), 9.21 (s, 1H), 7.80 (d, J = 8.3 Hz, 1H), 7.53 (d, J = 8.4 Hz, 1H), 7.48-7.35 (m, 1H), 7.32-7.12 |
| | (m, 6H), 7.04 (s, 1H), 6.39 (s, 1H), 4.81-4.75 (m, 5H), 4.50-4.29 (m, 2H), 4.26-4.23 (m, 1H), 3.73-3.67 (m, 1H), 3.48 (s, 1H), |
| | 3.42 (s, 6H), 3.09 (p, J = 6.8 Hz, 1H), 3.00-2.98 (m, 1H), 2.89-2.85 (m, 3H), 2.70-2.56 (m, 1H), 2.41-2.33 (m, 5H), 2.29 (s, 5H), 2.12- |
| | 2.00 (m, 1H), 1.99-1.83 (m, 1H), 1.74-1.69 (m, 1H), 1.61-1.59 (m, 1H), 1.13 (d, J = 7.0 Hz, 6H). |
| 75 | 9.35 (d, J = 15.7 Hz, 2H), 9.04 (s, 1H), 8.18-8.09 (m, 2H), 7.75-7.65 (m, 2H), 7.62-7.52 (m, 2H), 6.74 (s, 1H), 6.32 (s, 1H), 4.51- |
| | 4.39 (m, 3H), 4.31 (d, J = 12.1 Hz, 1H), 3.67-3.59 (m, 2H), 3.58-3.52 (m, 3H), 3.29-3.20 (m, 4H), 3.06 (p, J = 6.8 Hz, 1H), 2.67 |
| | (d, J = 11.5 Hz, 2H), 2.43-2.24 (m, 10H), 1.65 (s, 4H), 1.09 (d, J = 6.9 Hz, 6H), 1.03 (t, J = 7.0 Hz, 3H). |
| 76 | 10.50-9.82 (m, 1H), 9.61 (s, 1H), 9.12 (s, 1H), 8.38-8.02 (m, 3H), 8.00-7.88 (m, 1H), 7.82-7.70 (m, 1H), 7.62-7.60 (m, 1H), 7.58- |
| | 7.54 (m, 1H), 7.38-7.12 (m, 4H), 7.04 (s, 1H), 6.39 (s, 1H), 4.75 (s, 4H), 4.52-4.32 (m, 4H), 3.65 (s, 4H), 3.40 (s, 2H), 3.12-3.08 (m, |
| | 1H), 3.06-2.98 (m, 2H), 2.89 (s, 2H), 2.62 (s, 2H), 2.36 (s, 4H), 2.30 (s, 3H), 3.28-2.16 (m, 2H), 1.70 (s, 4H), 1.60 (s, 1H), 1.13 (d, J = |
| | 6.9 Hz, 6H). |
| 77 | 10.59 (s, 1H), 9.76 (s, 1H), 9.11 (s, 1H), 8.96 (t, J = 5.9 Hz, 1H), 8.17 (s, 2H), 7.96-7.90 (m, 1H), 7.78-7.70 (m, 1H), 7.65-7.63 (m, 1H), |
| | 7.62-7.56 (m, 1H), 7.40-7.22 (m, 5H), 6.57 (s, 1H), 6.33 (s, 1H), 4.54-4.30 (m, 4H), 3.72-3.50 (m, 5H), 3.47 (s, 3H), 3.21-3.10 (m, |
| | 2H), 3.06-3.00 (m, 1H), 2.96-2.84 (m, 2H), 2.66-2.60 (m, 1H), 2.39 (s, 5H), 2.30 (s, 5H), 2.12-2.10 (m 1H), 1.65 (s, 4H), 1.08- |
| | 1.00 (m, 3H), 0.90-0.70 (m, 6H). |
| 78 | 10.42 (s, 1H), 9.74 (s, 1H), 9.55 (d, J = 9.0 Hz, 1H), 9.10 (s, 1H), 7.35 (dd, J = 8.3, 2.3 Hz, 3H), 7.30-7.22 (m, 4H), 6.63 (s, 1H), 6.33 (s, |
| | 1H), 4.64 (h, J = 7.5 Hz, 1H), 4.42 (dt, J = 12.5, 6.5 Hz, 3H), 4.33 (dd, J = 10.9, 5.6 Hz, 1H), 3.58 (dd, J = 12.1, 6.0 Hz, 2H), 3.52 (s, |
| | 2H), 3.46 (s, 2H), 3.00 (dd, J = 10.1, 3.8 Hz, 1H), 2.96-2.86 (m, 1H), 2.62 (s, 1H), 2.38 (s, 6H), 2.35-2.25 (m, 6H), 2.12 (t, J = 1.7 |
| | Hz, 3H), 2.10-2.01 (m, 1H), 1.66-1.55 (m, 5H), 1.31 (d, J = 7.1 Hz, 3H), 0.86-0.79 (m, 6H). |
| 79 | 10.25 (s, 1H), 9.80-9.63 (s, 1H), 9.59 (t, J = 6.5 Hz, 1H), 9.10 (s, 1H), 7.37 (q, J = 7.5, 7.1 Hz, 1H), 7.34-7.28 (m, 3H), 7.25 (d, J = 8.0 Hz, |
| | 3H), 6.60 (s, 1H), 6.31 (s, 1H), 4.47-4.39 (m, 3H), 4.33 (dd, J = 10.9, 5.5 Hz, 1H), 3.95 (q, J = 9.5, 8.8 Hz, 2H), 3.58 (dd, J = 12.1, 6.4 |
| | Hz, 2H), 3.52 (s, 2H), 3.46 (s, 2H), 3.00 (d, J = 9.9 Hz, 1H), 2.89 (s, 1H), 2.67 (p, J = 1.9 Hz, 1H), 2.62 (s, 4H), 2.38 (s, 4H), 2.34-2.26 (m, |
| | 4H), 2.25-2.19 (m, 2H), 2.12 (s, 3H), 2.06 (d, J = 6.3 Hz, 1H), 1.60 (s, 5H), 0.84 (t, J = 7.5 Hz, 3H). |
| 80 | 9.38 (d, J = 15.0 Hz, 2H), 9.04 (s, 1H), 8.18-8.10 (m, 2H), 7.75-7.65 (m, 2H), 7.62-7.53 (m, 2H), 6.74 (s, 1H), 6.32 (s, 1H), 4.51-4.36 |
| | (m, 2H), 4.30 (s, 2H), 3.70-3.60 (m, 2H), 3.58-3.49 (m, 3H), 3.28-3.30 (m, 4H), 3.06 (p, J = 6.9 Hz, 1H), 2.97 (d, J = 9.3 Hz, |
| | 1H), 2.85 (s, 1H), 2.70-2.57 (m, 2H), 2.43-2.20 (m, 12H), 2.12-2.01 (m, 1H), 1.65 (s, 4H), 1.59-1.49 (m, 1H), 1.09 (d, J = 6.9 Hz, |
| | 6H), 1.03 (t, J = 6.9 Hz, 3H). |
| 81 | 10.25 (s, 1H), 9.71 (s, 1H), 9.60 (t, J = 6.5 Hz, 1H), 9.09 (s, 1H), 7.32 (d, J = 8.4 Hz, 2H), 7.30-7.22 (m, 3H), 7.22-7.15 (m, 2H), |
| | 6.60 (s, 1H), 6.31 (s, 1H), 4.44-4.38 (m, 3H), 4.32-4.30 (m, 1H), 3.98-3.94 (m, 2H), 3.59-3.55 (m, 2H), 3.52 (s, 2H), 3.46 (s, 2H), |
| | 3.01-2.98 (m, 1H), 2.91-2.87 (m, 1H), 2.63-2.59 (m, 1H), 2.45-2.34 (m, 6H), 2.33-2.30 (m, 9H), 2.22 (q, J = 7.5 Hz, 2H), 2.07 (s, |
| | 4H), 1.60 (s, 5H), 0.85 (t, J = 7.5 Hz, 3H). |
| 82 | 10.42 (s, 1H), 9.75 (s, 1H), 9.56 (d, J = 9.0 Hz, 1H), 9.10 (s, 1H), 7.35 (d, J = 8.2 Hz, 2H), 7.28 (d, J = 8.3 Hz, 3H), 7.24-7.15 |
| | (m, 2H), 6.62 (s, 1H), 6.33 (s, 1H), 4.64 (q, J = 7.7 Hz, 1H), 4.47-4.35 (m, 3H), 4.34-4.31 (m, 1H), 3.58 (s, 4H), 3.46 |
| | (s, 2H), 3.01-2.99 (m, 1H), 2.94 (s, 2H), 2.71-2.54 (m, 1H), 2.39 (s, 6H), 2.32 (s, 9H), 2.07 (s, 4H), 1.63 (s, 5H), 1.31 (d, J = 7.0 Hz, |
| | 3H), 0.83 (d, J = 6.8 Hz, 6H). |
| 83 | 10.62 (s, 1H), 9.75 (s, 1H), 9.11 (s, 1H), 8.95 (t, J = 5.9 Hz, 1H), 7.43-7.22 (m, 7H), 6.57 (s, 1H), 6.34 (s, 1H), 4.48-4.31 (m, 4H), |
| | 3.58 (d, J = 12.1 Hz, 2H), 3.47-3.39 (m, 8H), 3.15 (q, J = 6.8 Hz, 2H), 3.05-2.98 (m, 2H), 2.96 (s, 1H), 2.93-2.79 (m, 2H), 2.57 |
| | (s, 2H), 2.43-2.22 (m, 9H), 2.12 (s, 4H), 2.00 (s, 2H), 1.64-1.55 (m, 8H), 1.03 (t, J = 7.2 Hz, 3H), 0.80 (d, J = 6.9 Hz, 6H). |
| 84 | 10.06 (s, 1H), 9.60 (s, 1H), 9.10 (s, 1H), 7.41-7.31 (m, 1H), 7.30-7.15 (m, 5H), 7.05 (s, 1H), 6.39 (s, 1H), 4.76 (s, 4H), 4.47-4.30 (m, |
| | 4H), 3.58 (d, J = 12.3 Hz, 2H), 3.51 (s, 2H), 3.43 (s, 6H), 3.09 (p, J = 6.9 Hz, 1H), 2.99 (t, J = 9.3 Hz, 2H), 2.78 (d, J = 10.7 Hz, |
| | 2H), 2.67-2.55 (m, 2H), 2.30 (d, J = 10.0 Hz, 8H), 2.12 (t, J = 1.7 Hz, 3H), 2.10-2.03 (m, 1H), 1.95 (s, 2H), 1.65-1.49 |
| | (m, 9H), 1.13 (d, J = 6.9 Hz, 6H). |
| 85 | 10.60 (s, 1H), 9.95 (s, 1H), 9.23 (s, 1H), 8.94 (t, J = 5.8 Hz, 1H), 8.25 (s, 1H), 7.80 (d, J = 8.2 Hz, 1H), 7.53 (d, J = 8.4 Hz, 1H), |
| | 7.45-7.38 (m, 1H), 7.36 (d, J = 8.2 Hz, 2H), 7.31-7.14 (m, 5H), 6.57 (s, 1H), 6.33 (d, J = 0.8 Hz, 1H), 4.86-4.84 (m, 1H), |
| | 4.53-4.39 (m, 1H), 4.37-4.31 (m, 2H), 3.79-3.76 (m, 1H), 3.64 (s, 1H), 3.47 (s, 2H), 3.43 (s, 1H), 3.21-3.05 (m, 4H), 3.02- |
| | 2.99 (m, 1H), 2.95 (s, 2H), 2.90-2.87 (m, 2H), 2.70-2.55 (m, 2H), 2.40-2.36 (m, 5H), 2.32 (s, 5H), 2.10-2.06 (m, 1H), |
| | 1.97-1.85 (m, 1H), 1.78 (s, 1H), 1.60 (s, 1H), 1.03 (t, J = 7.2 Hz, 3H), 0.85-0.71 (m, 6H). |
| 86 | 10.22-9.97 (m, 2H), 9.61 (s, 1H), 9.02 (s, 1H), 7.88 (d, J = 8.4 Hz, 1H), 7.42 (dd, J = 13.8, 6.4 Hz, 2H), 7.35-7.15 (m, 4H), |
| | 7.11 (d, J = 2.4 Hz, 1H), 7.04 (s, 1H), 6.39 (s, 1H), 4.75 (s, 4H), 4.52-4.35 (m, 2H), 4.35-4.24 (m, 2H), 3.68-3.58 (m, 1H), |
| | 3.57 (d, J = 4.8 Hz, 1H), 3.45-3.36 (m, 3H), 3.15-3.03 (m, 2H), 3.03-2.95 (m, 2H), 2.90-2.75 (m, 1H), 2.71-2.55 (m, 1H), 2.43-2.21 |
| | (m, 12H), 2.12-2.00 (m, 1H), 1.69-1.52 (m, 5H), 1.23 (s, 1H), 1.13 (d, J = 6.8 Hz, 6H). |
| 87 | 10.57 (s, 1H), 10.13 (s, 1H), 9.75 (s, 1H), 9.03 (s, 1H), 8.76 (d, J = 8.2 Hz, 1H), 7.88 (dd, J = 8.2, 1.8 Hz, 1H), 7.48-7.30 (m, 5H), 7.28 |
| | (d, J = 8.4 Hz, 2H), 7.11 (d, J = 2.4 Hz, 1H), 6.59 (s, 1H), 6.33 (s, 1H), 4.53-4.36 (m, 2H), 4.30 (dt, J = 10.4, 5.2 Hz, 2H), |
| | 3.91 (q, J = 13.2 Hz, 1H), 3.63 (dd, J = 11.6, 4.9 Hz, 1H), 3.58 (d, J = 4.0 Hz, 1H), 3.53 (s, 2H), 3.47 (s, 2H), 3.01 (dd, J = |
| | 9.8, 3.6 Hz, 1H), 2.95-2.83 (m, 2H), 2.69-2.57 (m, 1H), 2.45-2.23 (m, 13H), 2.15-2.03 (m, 1H), 1.71-1.51 (m, 5H), 1.23 (s, 1H), |
| | 1.07 (d, J = 6.6 Hz, 6H), 0.81 (d, J = 6.8 Hz, 6H). |
| 88 | 10.56 (s, 1H), 9.76 (s, 1H), 9.10 (s, 1H), 8.76 (d, J = 8.2 Hz, 1H), 8.17 (s, 1H), 7.93 (dd, J = 7.8, 1.4 Hz, 1H), 7.87-7.79 (m, |
| | 1H), 7.75 (t, J = 7.6 Hz, 1H), 7.58 (d, J = 7.6 Hz, 1H), 7.36 (d, J = 8.4 Hz, 2H), 7.33-7.24 (m, 2H), 6.58 (s, 1H), 6.34 (s, |
| | 1H), 4.49-4.40 (m, 3H), 4.39-4.31 (m, 1H), 3.91 (dp, J = 8.4, 6.6 Hz, 1H), 3.78-3.59 (m, 5H), 3.29-3.18 (m, 1H), |
| | 3.05-2.98 (m, 1H), 2.97-2.83 (m, 2H), 2.70-2.58 (m, 1H), 2.45-2.28 (m, 11H), 2.17-2.00 (m, 1H), 1.70 (s, 4H), 1.65- |
| | 1.55 (m, 1H), 1.08 (d, J = 6.6 Hz, 6H), 0.81 (d, J = 6.8 Hz, 6H). |
| 89 | 10.62 (s, 1H), 9.75 (s, 1H), 9.08 (s, 1H), 8.94 (t, J = 6.0 Hz, 1H), 7.92 (dd, J = 7.8, 1.4 Hz, 1H), 7.88-7.79 (m, 1H), 7.75 (t, J = |
| | 7.8 Hz, 1H), 7.59 (d, J = 7.6 Hz, 1H), 7.37 (d, J = 8.4 Hz, 2H), 7.29 (d, J = 8.4 Hz, 2H), 6.57 (s, 1H), 6.34 (s, 1H), |
| | 4.47-4.32 (m, 4H), 3.66-3.57 (m, 2H), 3.54 (s, 2H), 3.48-3.39 (m, 6H), 3.22-3.11 (m, 2H), 3.07-2.99 (m, 1H), 2.95 |
| | (dd, J = 10.0, 6.8 Hz, 1H), 2.89 (q, J = 6.8 Hz, 1H), 2.85-2.79 (m, 2H), 2.63-2.53 (m, 3H), 2.44-2.25 (m, 8H), 2.13-2.04 |
| | (m, 1H), 2.03-1.95 (m, 2H), 1.69-1.52 (m, 8H), 1.03 (t, J = 7.2 Hz, 3H), 0.80 (d, J = 6.8 Hz, 6H). |
| 90 | 10.26-9.87 (s, 2H), 9.62 (s, 1H), 9.16 (s, 1H), 7.80 (d, J = 8.4 Hz, 1H), 7.55 (d, J = 8.4 Hz, 1H), 7.43 (dd, J = 8.2, 6.8 Hz, |
| | 1H), 7.32-7.12 (m, 6H), 7.04 (s, 1H), 6.39 (s, 1H), 4.78-4.73 (m, 4H), 4.50-4.35 (m, 3H), 4.27 (dd, J = 11.0, 5.6 Hz, 1H), |
| | 3.63-3.56 (m, 2H), 3.53 (s, 2H), 3.42 (s, 2H), 3.15-3.01 (m, 1H), 2.79 (d, J = 8.4 Hz, 1H), 2.71-2.54 (m, 1H), 2.44-1.99 |
| | (m, 16H), 1.65-1.60 (m, 4H), 1.44-1.34 (m, 1H), 1.13 (d, J = 6.8 Hz, 6H). |
| 91 | 9.18 (s, 1H), 7.95 (d, J = 7.8 Hz, 1H), 7.85 (t, J = 7.5 Hz, 1H), 7.79 (t, J = 7.6 Hz, 1H), 7.57 (d, J = 7.5 Hz, 1H), 7.53-7.34 |
| | (m, 3H), 7.04 (s, 1H), 6.42 (s, 1H), 4.81 (s, 4H), 4.69-4.59 (m, 4H), 4.31-4.15 (m, 4H), 3.89-3.83 (m, 2H), 3.55-3.28 (m, 10H), |
| | 3.10 (p, J = 6.9 Hz, 1H), 2.86 (s, 5H), 2.46-2.31 (m, 5H), 2.04-1.84 (m, 6H), 1.79 (s, 4H), 1.14 (d, J = 6.9 Hz, 6H). |
| 92 | 10.05 (s, 1H), 9.61 (s, 1H), 9.11 (s, 1H), 8.17 (d, J = 8.0 Hz, 1H), 7.93 (d, J = 8.4 Hz, 1H), 7.73 (t, J = 8.4 Hz, |
| | 1H), 7.68-7.55 (m, 2H), 7.32-7.10 (m, 4H), 7.04 (s, 1H), 6.54 (s, 1H), 6.39 (s, 1H), 4.76 (s, 4H), 4.47-4.30 (m, 4H), 3.68-3.52 |
| | (m, 4H), 3.50-3.38 (m, 6H), 3.30-3.22 (m, 2H), 3.10 (t, J = 10.8 Hz, 1H), 3.02-2.90 (m, 2H), 2.85-2.72 (m, 2H), 2.59 |
| | (s, 1H), 2.45-2.18 (m, 8H), 2.09 (t, J = 13.6, 6.8 Hz, 1H), 2.02-1.88 (m, 2H), 1.72-1.60 (m, 4H), 1.53 (s, 4H), 1.18-1.10 (m, 6H). |
| 93 | 10.42 (s, 1H), 9.73 (s, 1H), 9.54 (d, J = 9.0 Hz, 1H), 9.11 (s, 1H), 8.17 (d, J = 8.4 Hz, 1H), 7.92 (d, J = 8.2 Hz, 1H), |
| | 7.73 (t, J = 7.8 Hz, 1H), 7.62 (d, J = 8.2 Hz, 1H), 7.60-7.55 (m, 1H), 7.40-7.20 (m, 5H), 6.62 (s, 1H), 6.33 (s, 1H), 4.64 (q, J = 7.8 |
| | Hz, 1H), 4.43 (s, 3H), 4.34 (s, 1H), 3.68-3.55 (m, 2H), 3.52 (s, 2H), 3.46 (s, 2H), 3.01 (d, J = 8.2 Hz, 1H), 2.94-2.86 |
| | (m, 2H), 2.65 (s, 1H), 2.48-2.12 (m, 12H), 2.15-2.02 (m, 1H), 1.64 (s, 5H), 1.31 (d, J = 7.0 Hz, 3H), 0.82 (d, J = 6.8 Hz, 6H). |
| 94 | 10.40-9.42 (m, 2H), 9.10 (s, 1H), 8.24 (s, 3H), 8.07 (d, J = 8.4, 1.6 Hz, 1H), 7.91 (d, J = 8.2 Hz, 1H), 7.63-7.55 (m, 1H), |
| | 7.54-7.41 (m, 2H), 7.38-7.12 (m, 4H), 7.04 (s, 1H), 6.40 (s, 1H), 4.76 (s, 4H), 4.48 (d, J = 12.2 Hz, 1H), 4.43-4.32 (m, 2H), 4.25- |
| | 4.12 (m, 1H), 3.82-3.70 (m, 1H), 3.68-3.62 (m, 2H), 3.60-3.55 (m, 4H), 3.29 (s, 3H), 3.09 (p, J = 6.8 Hz, 2H), 2.76 (s, 3H), |
| | 2.58 (s, 2H), 2.40 (d, J = 3.6 Hz, 5H), 2.36-2.25 (m, 3H), 2.24-2.15 (m, 1H), 1.95 (s, 5H), 1.89-1.80 (m, 2H), 1.64 |
| | (s, 4H), 1.53 (s, 5H), 1.13 (d, J = 6.8 Hz, 6H), 1.05 (t, J = 7.2 Hz, 1H). |
| 95 | 10.60 (s, 1H), 10.12-9.62 (m, 2H), 9.16 (s, 1H), 8.96 (t, J = 5.8 Hz, 1H), 7.80 (d, J = 8.4 Hz, 1H), 7.56 (d, J = 8.4 Hz, 1H), 7.43 (dd, J = |
| | 8.4, 6.8 Hz, 1H), 7.39-7.32 (m, 2H), 7.28 (dd, J = 5.6, 2.8 Hz, 3H), 7.28-7.20 (m, 2H), 6.58 (s, 1H), 6.34 (s, |
| | 1H), 4.49-4.36 (m, 3H), 4.28 (dd, J = 10.8, 5.6 Hz, 1H), 3.65-3.56 (m, 2H), 3.54 (s, 2H), 3.47 (s, 2H), 3.22-3.10 (m, 2H), |
| | 2.89 (p, J = 6.8 Hz, 1H), 2.81 (d, J = 8.2 Hz, 1H), 2.56 (td, J = 8.0, 4.0 Hz, 1H), 2.42-2.14 (m, 14H), 2.10 (d, J = 9.0 Hz, |
| | 1H), 2.02-1.72 (m, 1H), 1.63 (s, 4H), 1.45-1.34 (m, 1H), 1.03 (t, J = 7.2 Hz, 3H), 0.80 (d, J = 6.8 Hz, 6H). |
| 96 | 10.53 (s, 1H), 10.36-9.57 (m, 2H), 8.96 (s, 1H), 8.88 (t, J = 5.8 Hz, 1H), 7.81 (dd, J = 8.2, 1.8 Hz, 1H), 7.43-7.15 (m, 7H), |
| | 7.05 (d, J = 2.5 Hz, 1H), 6.51 (s, 1H), 6.26 (s, 1H), 4.48-4.30 (m, 2H), 4.30-4.16 (m, 2H), 3.62-3.53 (m, 1H), 3.51 (d, |
| | J = 4.0 Hz, 1H), 3.47 (s, 2H), 3.40 (s, 2H), 3.09 (p, J = 7.0 Hz, 3H), 2.97-2.91 (m, 1H), 2.83 (dd, J = 13.4, 6.6 Hz, 2H), |
| | 2.63-2.49 (m, 1H), 2.33 (s, 6H), 2.31-2.19 (m, 6H), 2.01 (s, 1H), 1.62-1.45 (s, 5H), 0.96 (t, J = 7.2 Hz, 3H), 0.74 (d, |
| | J = 6.8 Hz, 6H). |
| 97 | 10.22-9.80 (m, 2H), 9.55 (s, 1H), 8.96 (s, 1H), 7.86-7.75 (m, 1H), 7.43-7.32 (m, 2H), 7.30-7.07 (m, 5H), 7.05 (d, J = 2.6 Hz, 1H), 6.97 |
| | (s, 1H), 6.32 (s, 1H), 4.69 (s, 4H), 4.47-4.30 (m, 2H), 4.30-4.16 (m, 2H), 3.56 (d, J = 12.2 Hz, 1H), 3.51-3.41 (m, 4H), 3.02 (p, J = |
| | 6.8 Hz, 1H), 2.97-2.81 (m, 3H), 2.70 (d, J = 10.4 Hz, 2H), 2.60 (s, 1H), 2.51 (s, 2H), 2.22 (s, 7H), 2.10-1.95 (m, 1H), 1.86 (s, |
| | 2H), 1.62-1.36 (m, 10H), 1.17 (s, 1H), 1.06 (d, J = 6.8 Hz, 6H), 0.83-0.69 (m, 1H). |
| 98 | 10.41 (s, 1H), 10.14 (s, 1H), 9.76 (s, 1H), 9.56 (d, J = 8.8 Hz, 1H), 9.03 (s, 1H), 8.01-7.79 (m, 1H), 7.51-7.19 (m, 7H), |
| | 7.11 (d, J = 2.6 Hz, 1H), 6.63 (s, 1H), 6.32 (s, 1H), 4.64 (q, J = 7.8 Hz, 1H), 4.55-4.36 (m, 2H), 4.29 (d, J = 11.2 Hz, 2H), |
| | 3.71-3.60 (m, 1H), 3.58 (d, J = 4.0 Hz, 1H), 3.54 (s, 3H), 3.46 (s, 2H), 3.06-2.81 (m, 4H), 2.70-2.57 (m, 2H), 2.45-2.26 |
| | (m, 11H), 2.13-2.03 (m, 1H), 1.70-1.52 (m, 5H), 1.31 (d, J = 6.8 Hz, 3H), 0.83 (d, J = 6.8 Hz, 6H). |
| 99 | 10.34-10.00 (m, 2H), 9.60 (t, J = 6.6 Hz, 1H), 9.03 (s, 1H), 7.88 (dd, J = 8.2, 1.8 Hz, 1H), 7.50-7.37 (m, 2H), 7.35-7.22 |
| | (m, 5H), 7.12 (d, J = 2.6 Hz, 1H), 6.60 (s, 1H), 6.31 (s, 1H), 4.52-4.38 (m, 2H), 4.30 (dt, J = 11.2, 6.0 Hz, 2H), 4.02-3.89 |
| | (m, 2H), 3.66 (s, 1H), 3.58 (d, J = 4.2 Hz, 1H), 3.56-3.51 (m, 3H), 3.46 (s, 2H), 3.17 (s, 1H), 3.00 (dd, J = 10.2, 3.8 |
| | Hz, 1H), 2.92-2.84 (m, 1H), 2.66-2.57 (m, 1H), 2.43-2.25 (m, 11H), 2.23 (q, J = 7.4 Hz, 3H), 2.12-2.03 (m, 1H), 1.71-1.51 |
| | (m, 5H), 0.85 (t, J = 7.4 Hz, 3H). |
| 100 | 10.42 (s, 1H), 9.95 (s, 1H), 9.74 (s, 1H), 9.54 (d, J = 9.0 Hz, 1H), 9.16 (s, 1H), 7.79 (d, J = 8.4 Hz, 1H), 7.56 (d, J = 8.4 Hz, 1H), |
| | 7.47-7.42 (m, 1H), 7.35 (d, J = 8.4 Hz, 2H), 7.32-7.26 (m, 3H), 7.26-7.18 (m, 2H), 6.62 (s, 1H), 6.33 (s, 1H), 4.64 (H, J = 7.6 |
| | Hz, 1H), 4.52-4.42 (m, 3H), 4.36-4.30 (m, 1H), 3.68-3.61 (m, 2H), 3.54 (s, 2H), 3.46 (s, 2H), 3.05-2.95 (m, 1H), 2.90 (p, |
| | J = 6.8 Hz, 2H), 2.70-2.57 (m, 1H), 2.46-2.33 (m, 6H), 2.31 (s, 6H), 2.12-2.05 (m, 1H), 1.63 (s, 5H), 1.31 (d, J = 7.0 Hz, |
| | 3H), 0.83 (d, J = 6.8 Hz, 6H). |
| 101 | 11.45 (s, 1H), 9.96 (s, 1H), 9.75 (s, 1H), 9.57 (t, J = 6.5 Hz, 1H), 9.16 (s, 1H), 7.79 (d, J = 8.4 Hz, 1H), 7.56 (d, J = 8.4 Hz, 1H), |
| | 7.48-7.42 (m, 1H), 7.35 (d, J = 8.4 Hz, 2H), 7.32-7.26 (m, 3H), 7.26-7.20 (m, 2H), 6.62 (s, 1H), 6.33 (s, 1H), 4.49-4.42 (m, 3H), 4.36-4.32 (m, |
| | 1H), 4.04-3.87 (m, 2H), 3.67-3.56 (m, 2H), 3.53 (s, 2H), 3.46 (s, 2H), 3.03-2.98 (m, 1H), 2.92-2.85 (m, 2H), 2.68-2.60 (m, 1H), |
| | 2.49-2.35 (m, 6H), 2.31 (s, 6H), 2.12-2.05 (m, 1H), 1.63 (s, 5H), 0.82 (d, J = 6.8 Hz, 6H). |
| 102 | 10.06 (s, 1H), 9.61 (s, 1H), 9.04 (s, 1H), 8.02 (dd, J = 8.2, 1.2 Hz, 1H), 7.61 (dt, J = 7.4, 3.6 Hz, 2H), 7.53-7.38 |
| | (m, 2H), 7.33-7.15 (m, 3H), 7.04 (s, 1H), 6.96 (d, J = 7.8 Hz, 1H), 6.39 (s, 1H), 4.75 (s, 4H), 4.55-4.22 (m, 4H), 3.71-3.48 (m, 4H), |
| | 3.46-3.37 (m, 5H), 3.09 (p, J = 6.8 Hz, 1H), 3.02-2.95 (m, 1H), 2.87 (s, 1H), 2.60 (s, 1H), 2.49-2.21 (m, 12H), 2.11-2.02 (m, 1H), |
| | 1.65-1.56 (m, 5H), 1.23 (s, 1H), 1.13 (d, J = 6.8 Hz, 6H), 0.84 (s, 1H). |
| 103 | 10.60 (s, 1H), 9.76 (s, 1H), 9.04 (s, 1H), 8.95 (t, J = 5.9 Hz, 1H), 8.05-7.99 (m, 1H), 7.61 (dt, J = 7.8, 3.6 Hz, 2H), 7.49 (t, J = 7.8 |
| | Hz, 1H), 7.42 (d, J = 7.0 Hz, 1H), 7.36 (d, J = 8.2 Hz, 2H), 7.28 (d, J = 8.2 Hz, 2H), 6.96 (d, J = 8.0 Hz, 1H), 6.57 (s, 1H), 6.33 (s, 1H), |
| | 4.43 (d, J = 11.2 Hz, 1H), 3.67-3.57 (m, 2H), 3.56-3.50 (m, 3H), 3.45 (d, J = 13.8 Hz, 5H), 3.21-3.10 (m, 2H), 3.00 (dd, J = 10.0, 3.6 |
| | Hz, 1H), 2.89 (p, J = 6.6 Hz, 2H), 2.70-2.66 (m, 1H), 2.61 (d, J = 6.8 Hz, 2H), 2.45-2.25 (m, 14H), 2.08 (dt, J = 13.6, 7.2 Hz, 1H), 1.63 (s, |
| | 5H), 1.03 (t, J = 7.2 Hz, 3H), 0.80 (d, J = 6.8 Hz, 7H). |
| 104 | 10.05 (s, 1H), 9.61 (s, 1H), 9.04 (s, 1H), 8.05-7.99 (m, 1H), 7.65-7.57 (m, 2H), 7.49 (t, J = 7.8 Hz, 1H), 7.42 (dd, J = 7.2, 1.2 Hz, 1H), |
| | 7.31-7.13 (m, 3H), 7.04 (s, 1H), 6.96 (d, J = 7.8 Hz, 1H), 6.39 (s, 1H), 4.76 (s, 4H), 4.50-4.28 (m, 4H), 3.66-3.51 (m, 4H), 3.48- |
| | 3.36 (m, 8H), 3.09 (p, J = 6.8 Hz, 1H), 3.03-2.89 (m, 2H), 2.77 (d, J = 10.4 Hz, 2H), 2.58 (s, 1H), 2.38-2.24 (m, 10H), 2.08 (s, 1H), |
| | 1.93 (s, 2H), 1.66-1.57 (m, 5H), 1.53 (s, 4H), 1.24 (s, 1H), 1.13 (d, J = 6.8 Hz, 6H). |
| 105 | 10.25 (s, 1H), 9.71 (s, 1H), 9.59 (t, J = 6.6 Hz, 1H), 9.04 (s, 1H), 8.02 (dd, J = 8.4, 1.2 Hz, 1H), 7.65-7.57 (m, 2H), 7.49 (t, J = 7.8 Hz, |
| | 1H), 7.42 (dt, J = 7.2, 1.2 Hz, 1H), 7.32 (d, J = 8.4 Hz, 2H), 7.25 (d, J = 8.2 Hz, 2H), 6.96 (d, J = 7.6 Hz, 1H), 6.60 (s, 1H), |
| | 6.31 (s, 1H), 4.56-4.24 (m, 4H), 4.00-3.89 (m, 2H), 3.66-3.50 (m, 4H), 3.48-3.41 (m, 4H), 3.32 (s, 2H), 3.00 (dd, J = 10.2, |
| | 3.8 Hz, 1H), 2.89 (s, 1H), 2.62 (d, J = 9.2 Hz, 1H), 2.39-2.15 (m, 13H), 2.07 (dt, J = 13.6, 7.2 Hz, 1H), 1.63 (s, 5H), 1.23 (s, 1H), |
| | 0.84 (t, J = 7.4 Hz, 3H). |
| 106 | 10.42 (s, 1H), 9.75 (s, 1H), 9.56 (d, J = 9.0 Hz, 1H), 9.04 (s, 1H), 8.02 (dd, J = 8.4, 1.2 Hz, 1H), 7.65-7.57 (m, 2H), 7.49 (t, J = 7.8 |
| | Hz, 1H), 7.42 (dt, J = 7.2, 1.2 Hz, 1H), 7.35 (d, J = 8.4 Hz, 2H), 7.28 (d, J = 8.2 Hz, 2H), 6.96 (dd, J = 7.8, 1.2 Hz, 1H), 6.63 (s, |
| | 1H), 6.33 (s, 1H), 4.64 (h, J = 7.8 Hz, 1H), 4.52-4.36 (m, 3H), 4.31 (dd, J = 11.2, 6.0 Hz, 1H), 3.67-3.51 (m, 4H), 3.48-3.41 (m, |
| | 5H), 3.00 (dd, J = 10.2, 3.8 Hz, 1H), 2.90 (p, J = 6.6 Hz, 2H), 2.62 (dd, J = 9.8, 5.6 Hz, 1H), 2.40-2.25 (m, 12H), 2.07 (dt, J = 13.4, |
| | 7.2 Hz, 1H), 1.61 (d, J = 17.4 Hz, 5H), 1.31 (d, J = 7.2 Hz, 3H), 0.83 (d, J = 6.8 Hz, 6H). |
| 107 | 10.41 (s, 1H), 9.75 (s, 1H), 9.57 (d, J = 9.0 Hz, 1H), 9.10 (s, 1H), 8.08 (d, J = 8.0 Hz, 1H), 7.91 (d, J = 8.2 Hz, 1H), 7.59 (t, J = 7.6 |
| | Hz, 1H), 7.46 (t, J = 7.0 Hz, 2H), 7.40-7.25 (m, 5H), 6.62 (s, 1H), 6.33 (s, 1H), 4.63 (p, J = 7.6 Hz, 1H), 4.50 (d, J = 12.4 |
| | Hz, 1H), 4.37 (d, J = 16.4 Hz, 2H), 4.18 (s, 1H), 3.66 (d, J = 12.6 Hz, 4H), 3.46 (s, 2H), 2.90 (p, J = 7.0 Hz, 1H), 2.75 (s, 1H), 2.41 (s, |
| | 12H), 2.19 (s, 2H), 1.95 (s, 3H), 1.86 (s, 2H), 1.67 (s, 5H), 1.49 (s, 1H), 1.31 (d, J = 7.2 Hz, 3H), 0.83 (d, J = 6.8 Hz, 6H). |
| 108 | 10.24 (s, 1H), 9.70 (s, 1H), 9.60 (t, J = 6.6 Hz, 1H), 9.09 (d, J = 4.2 Hz, 1H), 8.07 (d, J = 8.4 Hz, 1H), 7.91 (d, J = 8.2 Hz, 1H), 7.59 |
| | (t, J = 8.2 Hz, 1H), 7.50-7.41 (m, 2H), 7.32 (d, J = 8.4 Hz, 3H), 7.25 (d, J = 8.2 Hz, 2H), 6.59 (s, 1H), 6.31 (s, 1H), 4.47 (d, J = 12.2 |
| | Hz, 1H), 4.39-4.32 (m, 2H), 4.21-4.12 (m, 1H), 4.02-3.89 (m, 2H), 3.62 (d, J = 12.0 Hz, 1H), 3.54 (s, 3H), 3.46 (s, 2H), 2.74 (s, 1H), 2.43- |
| | 2.28 (m, 12H), 2.28-2.12 (m, 4H), 1.95 (s, 3H), 1.88-1.83 (m, 2H), 1.62 (s, 5H), 1.49 (s, 1H), 0.84 (t, J = 7.6 Hz, 3H). |
| 109 | 10.25 (s, 1H), 9.70 (s, 1H), 9.60 (t, J = 6.6 Hz, 1H), 9.11 (s, 1H), 8.17 (d, J = 8.2 Hz, 1H), 7.96-7.89 (m, 1H), 7.77-7.69 (m, 1H), 7.64- |
| | 7.55 (m, 2H), 7.35-7.21 (m, 5H), 6.60 (s, 1H), 6.31 (s, 1H), 4.43 (s, 3H), 4.33 (s, 1H), 4.00-3.89 (m, 2H), 3.61 (d, J = 6.8 Hz, 2H), |
| | 3.57 (s, 2H), 3.46 (s, 2H), 3.32 (s, 2H), 3.05-2.96 (m, 1H), 2.89 (s, 1H), 2.68-2.60 (m, 1H), 2.42-2.25 (m, 10H), 2.22 (q, J = 7.4 Hz, 2H), |
| | 2.12-2.01 (m, 1H), 1.62 (s, 5H), 0.84 (t, J = 7.4 Hz, 3H). |
| 110 | 10.42 (s, 1H), 9.73 (s, 1H), 9.54 (d, J = 9.0 Hz, 1H), 9.08 (s, 1H), 7.92 (d, J = 7.8 Hz, 1H), 7.82 (t, J = 7.6 Hz, 1H), 7.74 (t, J = 7.6 Hz, 1H), |
| | 7.58 (d, J = 7.6 Hz, 1H), 7.35 (d, J = 8.2 Hz, 2H), 7.28 (d, J = 8.2 Hz, 2H), 6.62 (s, 1H), 6.33 (s, 1H), 4.70-4.60 (m, 1H), |
| | 4.46-4.28 (m, 4H), 3.65-3.51 (m, 2H), 3.52 (s, 2H), 3.46 (s, 2H), 3.10-2.95 (m, 1H), 2.90 (p, J = 6.8 Hz, 2H), 2.69-2.57 (m, 1H), |
| | 2.38 (s, 6H), 2.35-2.29 (m, 6H), 2.11-2.02 (m, 1H), 1.60 (t, J = 8.6 Hz, 5H), 1.31 (d, J = 7.0 Hz, 3H), 0.83 (d, J = 6.8 Hz, 6H). |
| 111 | 10.70-9.70 (m 2H), 9.61 (t, J = 6.4 Hz, 1H), 9.13 (s, 1H), 8.16 (s, 2H), 7.93 (d, J = 7.8 Hz, 1H), 7.85-7.70 (m, 2H), 7.58 (d, J = 7.6 |
| | Hz, 1H), 7.36-7.20 (m, 4H), 6.60 (s, 1H), 6.31 (s, 1H), 4.59-4.42 (m, 3H), 4.41-4.33 (m, 1H), 4.02-3.84 (m, 4H), 3.79-3.69 (m, 2H), |
| | 3.48 (s, 2H), 3.09-3.01 (m, 1H), 2.99-2.90 (m, 1H), 2.74-2.65 (m, 1H), 2.55-2.54 (m, 2H), 2.47-2.35 (m, 6H), 2.34 (s, 4H), |
| | 2.23 (q, J = 7.6 Hz, 2H), 2.14-2.06 (m, 1H), 1.79 (s, 4H), 1.67-1.57 (m, 1H), 0.85 (t, J = 7.5 Hz, 3H). |
| 112 | 10.18-9.99 (m, 2H), 9.65 (s, 1H), 9.18 (s, 1H), 7.81 (d, J = 8.2 Hz, 1H), 7.57 (d, J = 8.4 Hz, 1H), 7.49-7.41 (m, 1H), 7.34-7.25 (m, 2H), |
| | 7.24-7.13 (m, 4H), 7.05 (s, 1H), 6.41 (s, 1H), 4.77 (s, 4H), 4.52-4.37 (m, 3H), 4.30 (dd, J = 10.8, 5.6 Hz, 1H), 3.66-3.58 (m, 2H), |
| | 3.55 (s, 2H), 3.44 (s, 5H), 3.10 (p, J = 6.8 Hz, 1H), 2.81 (t, J = 11.0 Hz, 3H), 2.70-2.52 (m, 2H), 2.39-2.06 (m, 12H), 1.93 (d, J = 13.0 Hz, |
| | 3H), 1.64 (s, 4H), 1.54 (s, 4H), 1.42 (t, J = 10.8 Hz, 1H), 1.14 (d, J = 6.8 Hz, 6H). |
| 113 | 10.20-10.05 (m, 2H), 9.62 (s, 1H), 9.03 (s, 1H), 7.88 (d, J = 8.0 Hz, 1H), 7.48-7.42 (m, 2H), 7.33 (d, J = 2.6 Hz, 1H), 7.25-7.15 |
| | (m, 3H), 7.11 (s, 1H), 7.04 (s, 1H), 6.39 (s, 1H), 4.75 (s, 4H), 4.48-4.42 (m, 1H), 4.37 (s, 1H), 4.28 (s, 2H), 3.71-3.49 (m, 4H), |
| | 3.42 (s, 2H), 3.09 (t, J = 6.8 Hz, 2H), 2.79 (s, 1H), 2.35-2.18 (m, 13H), 2.07 (s, 3H), 1.64 (s, 4H), 1.38 (s, 1H), 1.13 (d, J = 6.9 Hz, 6H). |
| 114 | 10.58 (s, 1H), 10.16 (s, 1H), 9.79 (s, 1H), 9.03 (s, 1H), 8.98 (t, J = 5.8 Hz, 1H), 7.88 (dd, J = 8.0, 1.6 Hz, 1H), 7.49-7.39 (m, 2H), 7.39- |
| | 7.32 (m, 3H), 7.28 (d, J = 8.2 Hz, 2H), 7.11 (d, J = 2.6 Hz, 1H), 6.58 (s, 1H), 6.33 (s, 1H), 4.50-4.42 (m, 1H), 4.44-4.34 (m, 1H), 4.34- |
| | 4.16 (m, 2H), 3.70-3.59 (m, 2H), 3.54 (s, 3H), 3.47 (s, 2H), 3.16 (p, J = 7.0 Hz, 2H), 2.89 (q, J = 6.8 Hz, 1H), 2.81 (d, J = 7.8 Hz, |
| | 1H), 2.38-2.10 (m, 15H), 1.65 (s, 4H), 1.39 (s, 1H), 1.03 (t, J = 7.2 Hz, 3H), 0.80 (d, J = 6.8 Hz, 6H) |
| 115 | 10.40 (s, 1H), 9.58 (s, 1H), 9.03 (s, 1H), 7.92-7.85 (m, 1H), 7.50-7.39 (m, 2H), 7.39-7.32 (m, 3H), 7.28 (d, J = 8.2 Hz, 2H), |
| | 7.11 (d, J = 2.6 Hz, 1H), 6.63 (d, J = 6.8 Hz, 1H), 6.32 (s, 1H), 4.64 (s, 1H), 4.50-4.40 (m, 1H), 4.39 (s, 1H), 4.34-4.12 (m, |
| | 2H), 3.65 (s, 1H), 3.62 (s, 1H), 3.53 (s, 3H), 3.46 (s, 2H), 2.90 (q, J = 6.8 Hz, 1H), 2.81 (d, J = 7.8 Hz, 1H), 2.67 (p, J = 1.8 Hz, |
| | 2H), 2.42-2.04 (m, 15H), 1.64 (s, 4H), 1.32 (d, J = 7.0 Hz, 4H), 0.83 (d, J = 6.8 Hz, 6H). |
| 116 | 10.22 (s, 1H), 10.16 (s, 1H), 9.72 (s, 1H), 9.62 (t, J = 6.4 Hz, 1H), 9.03 (s, 1H), 7.88 (d, J = 7.8 Hz, 1H), 7.49-7.39 |
| | (m, 2H), 7.39-7.29 (m, 3H), 7.26 (d, J = 8.2 Hz, 2H), 7.11 (d, J = 2.4 Hz, 1H), 6.61 (s, 1H), 6.30 (s, 1H), 4.52-4.42 (m, 1H), |
| | 4.44-4.35 (m, 1H), 4.35-4.16 (m, 2H), 3.95 (q, J = 9.2 Hz, 2H), 3.68-3.62 (m, 1H), 3.60 (d, J = 2.0 Hz, 1H), 3.57 (s, 2H), 3.46 (s, |
| | 2H), 3.34 (s, 2H), 2.81 (d, J = 8.2 Hz, 1H), 2.67 (p, J = 1.6 Hz, 2H), 2.45-2.00 (m, 16H), 1.66 (s, 4H), 1.43-1.35 (m, 1H), 0.85 (t, |
| | J = 7.2 Hz, 3H). |
| 117 | 10.19-9.99 (m, 2H), 9.63 (s, 1H), 9.02 (s, 1H), 8.28 (s, 1H), 7.88 (d, J = 7.8 Hz, 1H), 7.48-7.39 (m, 2H), 7.33 (d, J = 2.6 Hz, 1H), 7.30- |
| | 7.13 (m, 3H), 7.10 (d, J = 2.4 Hz, 1H), 7.03 (s, 1H), 6.39 (s, 1H), 4.83-4.65 (m, 4H), 4.49 (d, J = 13.2 Hz, 1H), 4.26 (d, J = 11.4 Hz, |
| | 3H), 3.68-3.47 (m, 5H), 3.41 (s, 2H), 3.13-3.02 (m, 1H), 2.79-2.64 (m, 2H), 2.34-2.22 (m, 10H), 2.08-2.01 (m, 2H), 1.91-1.80 (m, |
| | 2H), 1.70-1.54 (m, 5H), 1.47-1.35 (m, 1H), 1.23 (s, 2H), 1.13 (d, J = 6.8 Hz, 6H), 1.08-0.95 (m, 2H), 0.91-0.75 (m, 1H), 0.67-0.56 (m, 1H), |
| | 0.39 (s, 1H). |
| 118 | 10.42 (s, 1H), 9.99 (s, 1H), 9.81 (s, 1H), 9.57 (d, J = 9.0 Hz, 1H), 9.16 (s, 1H), 7.80 (d, J = 8.4 Hz, 1H), 7.56 (d, J = 8.4 Hz, 1H), |
| | 7.44 (ddd, J = 8.2, 6.6, 1.2 Hz, 1H), 7.35 (d, J = 8.2 Hz, 2H), 7.31-7.25 (m, 3H), 7.22 (d, J = 2.4 Hz, 2H), 6.63 (d, J = 1.8 Hz, |
| | 1H), 6.33 (s, 1H), 4.64 (h, J = 7.6 Hz, 1H), 4.49-4.36 (m, 3H), 4.28 (dd, J = 10.8, 5.6 Hz, 1H), 3.64-3.56 (m, 2H), 3.53 (s, 2H), |
| | 3.46 (s, 2H), 2.90 (q, J = 6.8 Hz, 1H), 2.81 (d, J = 8.0 Hz, 1H), 2.68-2.53 (m, 2H), 2.40-2.03 (m, 15H), 1.63 (s, 4H), 1.39 (t, J = 10.0 |
| | Hz, 1H), 1.32 (d, J = 7.0 Hz, 3H), 0.83 (d, J = 6.8 Hz, 6H). |
| 119 | 10.39 (s, 1H), 10.13 (s, 1H), 9.77 (s, 1H), 9.60 (t, J = 6.6 Hz, 1H), 9.02 (d, J = 2.2 Hz, 1H), 7.88 (dd, J = 8.2, 1.8 Hz, 1H), 7.48- |
| | 7.41 (m, 2H), 7.38 (dd, J = 8.4, 2.2 Hz, 2H), 7.35-7.29 (m, 3H), 7.13 (d, J = 2.4 Hz, 1H), 6.64 (d, J = 2.2 Hz, 1H), 6.33 (d, J = 2.2 Hz, |
| | 1H), 4.50 (d, J = 11.8 Hz, 1H), 4.25 (q, J = 8.4 Hz, 3H), 4.03-3.89 (m, 2H), 3.65 (d, J = 12.2 Hz, 1H), 3.62-3.50 (m, 5H), 3.49- |
| | 3.39 (m, 4H), 3.04-2.83 (m, 4H), 2.38 (s, 2H), 2.34-2.19 (m, 4H), 1.90 (s, 2H), 1.66 (s, 4H), 1.54 (s, 4H), 0.84 (dd, J = 6.8, 2.2 Hz, |
| | 6H), 0.64 (s, 2H), 0.39 (s, 2H). |
| 120 | 10.36 (s, 1H), 10.13 (s, 1H), 9.75 (s, 1H), 9.57 (d, J = 9.0 Hz, 1H), 9.02 (s, 1H), 7.88 (dd, J = 8.0, 1.8 Hz, 1H), 7.48-7.38 (m, 2H), |
| | 7.37-7.27 (m, 5H), 7.13 (d, J = 2.6 Hz, 1H), 6.64 (s, 1H), 6.33 (s, 1H), 4.63 (d, 9.8 Hz, 1H), 4.50 (d, J = 11.6 Hz, 1H), |
| | 4.32-4.20 (m, 3H), 3.64 (d, J = 11.8 Hz, 1H), 3.60 (s, 1H), 3.57-3.46 (m, 5H), 3.43 (s, 4H), 3.18-3.16 (m, 1H), 3.02-2.86 (m, 3H), |
| | 2.42-2.21 (m, 6H), 1.89 (s, 2H), 1.66 (s, 4H), 1.53 (s, 4H), 1.32 (d, J = 7.0 Hz, 3H), 0.88-0.81 (m, 6H), 0.63 (s, 2H), 0.39 (s, 2H). |
| 121 | 10.22-10.01 (m, 2H), 9.61 (s, 1H), 9.01 (s, 1H), 7.88 (dd, J = 8.2, 1.8 Hz, 1H), 7.48-7.37 (m, 2H), 7.33 (d, J = 2.5 Hz, 1H), 7.29- |
| | 7.17 (m, 3H), 7.12 (d, J = 2.6 Hz, 1H), 7.04 (s, 1H), 6.39 (s, 1H), 4.76 (s, 4H), 4.49 (d, J = 11.9 Hz, 1H), 4.24 (t, J = 10.0 |
| | Hz, 3H), 3.64 (d, J = 12.2 Hz, 1H), 3.60 (s, 1H), 3.54 (s, 2H), 3.50-3.37 (m, 9H), 3.09 (p, J = 6.8 Hz, 1H), 2.96 (d, |
| | J = 11.0 Hz, 2H), 2.80 (d, J = 10.4 Hz, 2H), 2.41-2.31 (m, 1H), 2.24 (d, J = 12.4 Hz, 1H), 2.04-1.83 (m, 5H), 1.65 (s, |
| | 4H), 1.55 (s, 9H), 1.13 (d, J = 6.8 Hz, 6H), 0.64 (s, 2H), 0.39 (s, 2H). |
| 122 | 10.08 (s, 1H), 9.62 (s, 1H), 9.04 (s, 1H), 8.14 (t, J = 7.8 Hz, 2H), 7.76-7.64 (m, 2H), 7.62-7.51 (m, 2H), 7.28-7.15 (m, 3H), 7.04 (s, |
| | 1H), 6.39 (s, 1H), 4.75 (s, 4H), 4.48-4.42 (m, 1H), 4.39-4.37 (m, 1H), 4.35-4.30 (m, 1H), 4.28-4.22 (m, 1H), 3.70-3.59 (m, 2H), 3.57 |
| | (s, 1H), 3.53 (s, 2H), 3.42 (s, 2H), 3.09 (p, J = 6.8 Hz, 1H), 2.79 (d, J = 8.2 Hz, 1H), 2.55 (s, 1H), 2.43-2.32 (m, 5H), 2.30 (s, 3H), |
| | 2.26-2.14 (m, 5H), 2.09-2.04 (m, 1H), 1.65 (s, 4H), 1.42-1.37 (m, 1H), 1.24 (s, 1H), 1.13 (d, J = 6.8 Hz, 6H). |
| 123 | 10.45 (s, 1H), 9.77 (s, 1H), 9.58 (t, J = 6.6 Hz, 1H), 9.04 (s, 1H), 8.14 (t, J = 7.6 Hz, 2H), 7.76-7.64 (m, 2H), 7.62-7.52 |
| | (m, 2H), 7.39-7.26 (m, 4H), 6.62 (s, 1H), 6.33 (s, 1H), 4.49-4.42 (m, 1H), 4.39 (p, J = 5.6 Hz, 1H), 4.32-4.28 (m, 1H), 4.27-4.18 |
| | (m, 1H), 4.03-3.87 (m, 2H), 3.70-3.60 (m, 2H), 3.58-3.49 (m, 3H), 3.47 (s, 2H), 2.90 (p, J = 6.8 Hz, 1H), 2.81 (d, J = 8.2 Hz, 1H), |
| | 2.55 (d, J = 9.4 Hz, 1H), 2.37 (s, 7H), 2.35-2.16 (m, 8H), 2.08 (d, J = 6.8 Hz, 1H), 1.65 (s, 4H), 1.42-1.30 (m, 1H), 0.82 (d, J = 6.8 Hz, 6H). |
| 124 | 10.83 (s, 1H), 10.15 (s, 1H), 9.77 (s, 1H), 9.02 (s, 1H), 8.92 (t, J = 5.8 Hz, 1H), 7.88 (dd, J = 8.2, 1.8 Hz, 1H), 7.49-7.38 |
| | (m, 2H), 7.34 (d, J = 2.6 Hz, 1H), 7.16-7.09 (m, 3H), 6.95 (d, J = 8.6 Hz, 2H), 6.59 (s, 1H), 6.34 (s, 1H), 4.50 (d, J = 11.4 Hz, |
| | 1H), 4.27 (d, J = 10.2 Hz, 3H), 3.76 (d, J = 12.4 Hz, 2H), 3.64 (d, J = 12.2 Hz, 5H), 3.59 (s, 2H), 3.22-3.11 (m, 2H), 2.92 (p, J = 7.0 |
| | Hz, 1H), 2.72 (d, J = 12.0 Hz, 2H), 2.33-2.23 (m, 11H), 2.10 (d, J = 6.8 Hz, 2H), 1.74 (d, J = 13.2 Hz, 7H), 1.21 (s, 2H), 1.14 (d, J = |
| | 11.8 Hz, 3H), 0.82 (d, J = 6.8 Hz, 6H), 0.63 (s, 2H), 0.40 (s, 2H). |
| 125 | 10.26-9.94 (m, 2H), 9.63 (s, 1H), 9.05 (s, 1H), 7.89 (dd, J = 8.0, 1.8 Hz, 1H), 7.49-7.38 (m, 2H), 7.34 (d, J = 2.6 |
| | Hz, 1H), 7.28-7.16 (m, 3H), 7.12 (d, J = 2.4 Hz, 1H), 7.03 (s, 1H), 6.40 (s, 1H), 4.75 (s, 4H), 4.58 (d, J = 13.2 Hz, 1H), |
| | 4.44-4.17 (m, 3H), 3.89 (s, 2H), 3.80-3.64 (m, 2H), 3.59 (s, 1H), 3.45 (s, 2H), 3.09 (p, J = 6.8 Hz, 1H), 2.79 (s, 2H), |
| | 2.64 (s, 2H), 2.07 (s, 4H), 1.91 (s, 1H), 1.85-1.66 (m, 9H), 1.38 (s, 4H), 1.24 (s, 1H), 1.13 (d, J = 6.8 Hz, 6H), 0.89-0.77 |
| | (m, 1H), 0.65 (s, 2H), 0.42 (s, 2H). |
| 126 | 10.43 (s, 1H), 10.15 (s, 1H), 9.77 (s, 1H), 9.58 (d, J = 9.0 Hz, 1H), 9.02 (s, 1H), 7.88 (dd, J = 8.0, 1.8 Hz, 1H), 7.49-7.38 |
| | (m, 2H), 7.37-7.23 (m, 5H), 7.11 (d, J = 2.6 Hz, 1H), 6.62 (s, 1H), 6.33 (s, 1H), 4.71-4.42 (m, 2H), 4.26 (d, J = 12.6 Hz, 3H), |
| | 3.64 (d, J = 12.0 Hz, 1H), 3.59-3.40 (m, 6H), 3.31 (s, 2H), 2.91 (p, J = 6.8 Hz, 1H), 2.78 (d, J = 10.8 Hz, 2H), 2.43-2.33 (m, 3H), |
| | 2.32 (d, J = 1.8 Hz, 5H), 2.06 (d, J = 7.0 Hz, 2H), 1.90 (t, J = 11.2 Hz, 2H), 1.71-1.56 (m, 6H), 1.44 (s, 1H), 1.32 (d, J = 7.0 |
| | Hz, 3H), 1.08 (dd, J = 15.2, 8.2 Hz, 2H), 0.88-0.80 (m, 6H), 0.63 (s, 2H), 0.40 (s, 2H). |
| 127 | 10.46 (s, 1H), 10.14 (s, 1H), 9.77 (s, 1H), 9.60 (t, J = 6.5 Hz, 1H), 9.02 (s, 1H), 7.88 (dd, J = 8.0, 1.8 Hz, 1H), 7.49-7.38 |
| | (m, 2H), 7.38-7.26 (m, 5H), 7.11 (d, J = 2.4 Hz, 1H), 6.61 (s, 1H), 6.34 (s, 1H), 4.49 (d, J = 11.2 Hz, 1H), |
| | 4.26 (d, J = 11.8 Hz, 3H), 3.94 (q, J = 9.6 Hz, 2H), 3.68-3.41 (m, 6H), 3.31 (s, 4H), 2.94-2.86 (m, 1H), 2.78 (d, J = |
| | 10.4 Hz, 1H), 2.28 (d, J = 8.6 Hz, 5H), 2.09-1.81 (m, 6H), 1.68-1.56 (m, 6H), 1.44 (s, 2H), 1.24 (s, 1H), 1.07 (d, J = 11.6 |
| | Hz, 2H), 0.82 (d, J = 6.8 Hz, 6H), 0.63 (s, 2H), 0.40 (s, 2H). |
| 128 | 10.07-9.99 (m, 1H), 9.62 (s, 1H), 9.17 (s, 1H), 8.13-8.02 (m, 2H), 7.73 (d, J = 8.4 Hz, 1H), 7.70-7.63 (m, 2H), 7.62- |
| | 7.54 (m, 1H), 7.54-7.46 (m, 1H), 7.31-7.11 (m, 3H), 7.04 (s, 1H), 6.39 (s, 1H), 4.75 (s, 5H), 4.46 (d, J = 12.2 |
| | Hz, 2H), 4.29 (s, 2H), 3.61 (d, J = 14.8 Hz, 4H), 3.41 (s, 2H), 3.31 (s, 2H), 3.09 (p, J = 7.0 Hz, 1H), 2.74 (s, 2H), |
| | 2.42-2.35 (m, 1H), 2.27 (s, 6H), 2.02 (d, J = 6.8 Hz, 2H), 1.86 (s, 2H), 1.65 (s, 4H), 1.58 (d, J = 12.4 Hz, 2H), 1.41 |
| | (s, 1H), 1.13 (d, J = 6.8 Hz, 6H), 1.03 (d, J = 11.7 Hz, 2H), 0.63 (s, 2H), 0.39 (s, 2H). |
| 129 | 10.12 (s, 2H), 9.59 (s, 1H), 9.17-8.90 (m, 1H), 7.88 (dd, J = 8.1, 1.6 Hz, 1H), 7.48-7.37 (m, 2H), 7.33 (d, J = 2.5 Hz, 1H), 7.25-7.18 |
| | (m, 3H), 7.13 (d, J = 2.6 Hz, 1H), 7.04 (s, 1H), 6.39 (s, 1H), 4.93-4.57 (m, 5H), 4.40-3.97 (m, 3H), 3.87-3.45 (m, 3H), 3.41 (s, 2H), |
| | 3.09 (p, J = 6.9 Hz, 1H), 2.93 (s, 1H), 2.80-2.60 (m, 2H), 2.61-2.51 (m, 1H), 2.42-2.36 (m, 4H), 2.34-2.16 (m, 7H), 2.05-2.02 |
| | (m, 2H), 1.94-1.92 (m, 2H), 1.88-1.83 (m, 2H), 1.75 (s, 1H), 1.61-1.58 (m, 2H), 1.42 (s, 1H), 1.13 (d, J = 6.9 Hz, 6H), 0.62 |
| | (s, 2H), 0.40 (s, 2H). |
| 130 | 10.46 (s, 1H), 10.12 (s, 1H), 9.75 (s, 1H), 9.58 (t, J = 6.5 Hz, 1H), 9.02 (s, 1H), 7.88 (dd, J = 8.1, 1.7 Hz, 1H), 7.49-7.38 |
| | (m, 2H), 7.38-7.23 (m, 5H), 7.11 (d, J = 2.6 Hz, 1H), 6.60 (s, 1H), 6.33 (s, 1H), 4.50 (d, J = 11.8 Hz, 1H), 4.31-4.21 (m, 3H), |
| | 3.95 (dd, J = 9.7, 6.7 Hz, 2H), 3.64 (d, J = 11.9 Hz, 1H), 3.56 (d, J = 11.8 Hz, 1H), 3.45 (s, 3H), 3.29 (s, 2H), 2.88 (p, J = 6.8 |
| | Hz, 10H), 1.66 (s, 4H), 0.80 (d, J = 6.9 Hz, 6H), 0.63 (s, 2H), 0.40 (s, 2H). |
| 131 | 10.13 (s, 2H), 9.60 (s, 1H), 9.02 (s, 1H), 7.88 (dd, J = 8.1, 1.7 Hz, 1H), 7.49-7.37 (m, 2H), 7.33 (d, J = 2.6 Hz, 1H), 7.16 (d, J = 7.6 |
| | Hz, 3H), 7.11 (d, J = 2.6 Hz, 1H), 7.04 (s, 1H), 6.39 (s, 1H), 4.75 (s, 4H), 4.49 (d, J = 12.0 Hz, 1H), 4.26 (d, J = 12.8 Hz, 3H), |
| | 3.64 (d, J = 12.0 Hz, 1H), 3.08 (q, J = 6.9 Hz, 1H), 2.41 (s, 10H), 1.65 (s, 4H), 1.13 (d, J = 6.9 Hz, 6H), 0.62 (s, 2H), 0.39 (s, 2H). |
| 132 | 10.46 (s, 1H), 10.13 (s, 1H), 9.76 (s, 1H), 9.59 (t, J = 6.5 Hz, 1H), 9.02 (s, 1H), 7.88 (dd, J = 8.1, 1.7 Hz, 1H), 7.49-7.39 |
| | (m, 2H), 7.38-7.33 (m, 3H), 7.30 (d, J = 8.5 Hz, 2H), 7.11 (d, J = 2.6 Hz, 1H), 6.61 (s, 1H), 6.34 (s, 1H), 4.50 (d, J = 12.1 Hz, |
| | 1H), 4.33-4.20 (m, 3H), 4.02-3.89 (m, 2H), 3.65 (d, J = 12.2 Hz, 1H), 3.58 (d, J = 4.4 Hz, 4H), 3.45 (s, 2H), 3.39 (s, 1H), |
| | 2.90 (p, J = 6.9 Hz, 1H), 2.76 (s, 2H), 2.69-2.59 (m, 2H), 2.28 (s, 2H), 2.06 (d, J = 10.1 Hz, 4H), 1.74-1.62 (m, 5H), 1.68 (s, |
| | 4H), 1.38 (s, 4H), 0.82 (d, J = 6.9 Hz, 6H), 0.63 (s, 2H), 0.39 (s, 2H). |
| 133 | 10.11 (d, J = 13.8 Hz, 2H), 9.59 (s, 1H), 9.02 (s, 1H), 7.88 (dd, J = 8.2, 1.6 Hz, 1H), 7.49-7.37 (m, 2H), 7.33 (d, J = 2.6 Hz, |
| | 1H), 7.11 (d, J = 2.6 Hz, 2H), 7.04 (s, 1H), 6.85 (d, J = 8.2 Hz, 2H), 6.38 (s, 1H), 4.69 (s, 4H), 4.50 (d, J = 11.8 Hz, 1H), 4.27 |
| | (d, J = 8.8 Hz, 3H), 3.72-3.47 (m, 7H), 3.09 (p, J = 6.8 Hz, 1H), 2.60 (t, J = 11.8 Hz, 2H), 2.45-2.18 (m, 10H), 2.09 (d, J = |
| | 7.2 Hz, 2H), 1.80-1.58 (m, 7H), 1.17 (s, 2H), 1.13 (d, J = 6.8 Hz, 6H), 0.63 (d, J = 4.6 Hz, 2H), 0.40 (s, 2H). |
| 134 | 10.44 (s, 1H), 10.12 (s, 1H), 9.75 (s, 1H), 9.55 (s, 1H), 9.11-9.00 (m, 1H), 7.88 (dd, J = 7.8, 1.6 Hz, 1H), 7.48-7.37 (m, 2H), 7.38-7.32 (m, |
| | 3H), 7.32-7.23 (m, 2H), 7.13 (d, J = 2.6 Hz, 1H), 7.09 (d, J = 2.4 Hz, 1H), 6.62 (s, 1H), 6.33 (s, 1H), 4.96-4.51 (m, 2H), 4.39-4.13 (m, |
| | 3H), 4.06-3.78 (m, 2H), 3.62-3.55 (m, 1H), 3.50-3.45 (m, 4H), 3.38 (s, 1H), 2.97-2.82 (m, 3H), 2.79-2.76 (m, 2H), |
| | 2.42-2.15 (m, 9H), 2.06 (d, J = 7.2 Hz, 2H), 1.93-1.90 (m, 3H), 1.75-1.73 (m, 1H), 1.62-1.59 (m, 2H), 1.44 (s, 1H), 1.31 (d, J = |
| | 7.0 Hz, 3H), 1.08-1.06 (m, 2H), 0.86-0.77 (m, 6H), 0.63 (s, 2H), 0.40 (s, 2H). |
| 135 | 10.47 (s, 1H), 10.13 (s, 1H), 9.76 (s, 1H), 9.58 (t, J = 6.6 Hz, 1H), 9.21-8.91 (m, 1H), 7.96-7.78 (m, 1H), 7.48-7.37 (m, 2H), |
| | 7.37-7.32 (m, 3H), 7.30 (d, J = 8.4 Hz, 2H), 7.13-7.09 (m, 1H), 6.61 (s, 1H), 6.34 (s, 1H), 4.89-4.61 (m, 1H), 4.41-4.14 (m, 3H), |
| | 4.11-3.86 (m, 3H), 3.79-3.56 (m, 2H), 3.46 (s, 3H), 2.92-2.86 (m, 3H), 2.83-2.65 (m, 2H), 2.43-2.12 (m, 9H), 2.06 (d, J = 7.2 |
| | Hz, 2H), 1.93-1.86 (m, 4H), 1.75-1.73 (m, 1H), 1.63-1.60 (m, 2H), 1.44 (s, 2H), 1.16-0.98 (m, 2H), 0.82 (d, J = 6.8 Hz, 6H), |
| | 0.63 (s, 2H), 0.40 (s, 2H). |
| 136 | 10.13 (s, 2H), 9.60 (s, 1H), 9.12-9.00 (m, 1H), 7.88 (d, J = 7.6 Hz, 1H), 7.47-7.35 (m, 2H), 7.33 (d, J = 2.4 Hz, 1H), 7.28-7.19 (m, 3H), 7.12- |
| | 7.19 (m, 1H), 7.04 (s, 1H), 6.39 (s, 1H), 4.96-4.53 (m, 5H), 4.31-4.19 (m, 3H), 4.13-3.72 (m, 3H), 3.66-3.50 (m, 1H), 3.47- |
| | 3.45 (m, 3H), 3.09 (p, J = 6.8 Hz, 1H), 2.99-2.80 (m, 2H), 2.79-2.77 (m, 2H), 2.43-2.10 (m, 2H), 2.05 (s, 1H), 2.00-1.82 |
| | (m, 3H), 1.79-1.58 (m, 4H), 1.53 (s, 4H), 1.13 (d, J = 6.8 Hz, 6H), 0.99-0.69 (m, 2H), 0.63 (s, 2H), 0.40 (s, 2H). |
| 137 | 10.43 (s, 1H), 9.74 (s, 1H), 9.55 (d, J = 9.0 Hz, 1H), 9.18 (s, 1H), 8.12-8.01 (m, 2H), 7.73 (d, J = 8.2 Hz, 1H), 7.66 (d, J = |
| | 5.2 Hz, 2H), 7.58 (t, J = 7.6 Hz, 1H), 7.50 (t, J = 7.2 Hz, 1H), 7.35 (d, J = 8.4 Hz, 2H), 7.28 (d, J = 8.2 Hz, 2H), 6.62 (s, 1H), 6.33 (s, |
| | 1H), 4.64 (d, J = 8.2 Hz, 1H), 4.47 (d, J = 12.2 Hz, 2H), 4.31 (s, 2H), 3.62 (d, J = 12.6 Hz, 4H), 3.45 (s, 2H), 2.90 (q, J = 6.8 Hz, 1H), 2.77 |
| | (d, J = 10.8 Hz, 2H), 2.38 (s, 3H), 2.28 (d, J = 6.8 Hz, 6H), 2.08 (s, 2H), 1.87 (s, 2H), 1.83 (s, 2H), 1.67 (s, 4H), 1.59 (d, J = 12.4 |
| | Hz, 2H), 1.43 (s, 3H), 1.31 (d, J = 7.0 Hz, 3H), 1.06 (d, J = 11.8 Hz, 2H), 0.88-0.79 (m, 6H), 0.63 (s, 2H), 0.40 (s, 2H). |
| 138 | NA |
| 139 | 11.92 (s, 1H), 9.59 (s, 1H), 9.41 (s, 1H), 7.30-7.23 (m, 2H), 10.12 (s, 2H), 9.60 (s, 1H), 9.02 (s, 1H), 7.87 (d, J = 7.8 Hz, 1H), |
| | 7.48-7.37 (m, 2H), 7.33 (d, J = 2.6 Hz, 1H), 7.26-7.18 (m, 3H), 7.11 (d, J = 2.6 Hz, 1H), 7.04 (s, 1H), 6.39 (s, 1H), 4.75 (s, |
| | 4H), 4.48 (d, J = 11.8 Hz, 1H), 4.27 (t, J = 10.2 Hz, 2H), 4.17 (d, J = 10.8 Hz, 1H), 3.63 (d, J = 12.0 Hz, 1H), 3.59 (d, J = 4.2 |
| | Hz, 1H), 3.52 (d, J = 12.2 Hz, 3H), 3.44 (d, J = 9.2 Hz, 6H), 3.15 (s, 1H), 3.12-3.05 (m, 1H), 2.82 (s, 1H), 2.67 (s, 1H), 2.38-2.24 |
| | (m, 8H), 1.87 (s, 2H), 1.65 (s, 4H), 1.24 (s, 2H), 1.13 (d, J = 6.8 Hz, 6H), 0.84 (d, J = 7.2 Hz, 1H), 0.60 (s, 2H), 0.42 (s, 2H). |
| 140 | 10.43 (s, 1H), 10.12 (s, 1H), 9.75 (s, 1H), 9.55 (d, J = 8.8 Hz, 1H), 9.02 (s, 1H), 7.88 (dd, J = 8.2, 1.6 Hz, 1H), 7.49-7.39 (m, 2H), |
| | 7.38-7.31 (m, 3H), 7.29 (d, J = 8.4 Hz, 2H), 7.24 (s, 1H), 7.11 (d, J = 2.6 Hz, 1H), 6.62 (s, 1H), 6.33 (s, 1H), 4.64 (dq, J = 15.4, |
| | 7.4 Hz, 1H), 4.49 (d, J = 11.8 Hz, 1H), 4.34-4.18 (m, 3H), 3.64 (d, J = 12.0 Hz, 1H), 3.57 (s, 1H), 3.54 (d, J = 12.2 Hz, 3H), |
| | 3.45 (s, 2H), 3.38 (s, 1H), 2.91 (p, J = 6.8 Hz, 1H), 2.75 (s, 2H), 2.67 (dt, J = 3.8, 1.8 Hz, 2H), 2.64 (s, 1H), 2.31-2.22 (m, |
| | 2H), 2.04 (s, 4H), 1.74 (d, J = 10.2 Hz, 4H), 1.66 (s, 4H), 1.39 (d, J = 11.6 Hz, 4H), 1.32 (d, J = 7.0 Hz, 3H), 0.88-0.79 |
| | (m, 6H), 0.63 (s, 2H), 0.39 (s, 2H). |
| 141 | 10.12 (s, 2H), 9.60 (s, 1H), 9.01 (s, 1H), 7.88 (dd, J = 8.0, 1.8 Hz, 1H), 7.49-7.37 (m, 2H), 7.33 (d, J = 2.6 Hz, 1H), 7.19 |
| | (d, J = 7.4 Hz, 2H), 7.11 (d, J = 2.4 Hz, 1H), 7.04 (s, 1H), 6.97 (s, 1H), 6.39 (s, 1H), 4.76 (s, 5H), 4.48 (d, J = 11.8 Hz, |
| | 1H), 4.31-4.20 (m, 4H), 3.84 (s, 1H), 3.67-3.55 (m, 3H), 3.54-3.49 (m, 2H), 3.09 (p, J = 6.8 Hz, 1H), 3.06-3.02 (m, 1H), |
| | 2.99 (s, 1H), 2.78 (d, J = 11.8 Hz, 4H), 2.28 (d, J = 6.0 Hz, 2H), 2.03 (s, 2H), 1.95 (s, 2H), 1.73 (d, J = 11.4 Hz, 4H), 1.65 (s, |
| | 5H), 1.53 (s, 4H), 1.23 (s, 2H), 1.13 (d, J = 6.8 Hz, 6H), 0.62 (s, 2H), 0.39 (s, 2H). |
| 142 | 10.14 (s, 1H), 10.05 (s, 1H), 9.61 (s, 1H), 9.05 (s, 1H), 7.89 (dd, J = 8.0, 1.8 Hz, 1H), 7.48-7.38 (m, 2H), 7.34-7.25 (m, 4H), |
| | 7.11 (t, J = 2.4 Hz, 1H), 7.04 (s, 1H), 6.39 (s, 1H), 4.78-4.72 (m, 6H), 4.64 (s, 1H), 4.51-4.49 (m, 1H), 4.29 (s, 2H), 3.76- |
| | 3.74 (m, 1H), 3.67-3.65 (m, 1H), 3.58 (d, J = 6.2 Hz, 1H), 3.09 (p, J = 6.8 Hz, 1H), 2.90 (s, 3H), 2.34-2.32 (m, 10H), 2.17-1.76 (m, |
| | 8H), 2.09-1.90 (m, 4H), 1.13 (d, J = 6.8 Hz, 8H), 0.65 (s, 2H), 0.43 (s, 2H). |
| 143 | 10.47 (s, 1H), 10.15 (s, 1H), 9.76 (s, 1H), 9.57 (t, J = 6.6 Hz, 1H), 9.05 (s, 1H), 7.89 (dd, J = 7.8, 1.8 Hz, 1H), 7.50-7.39 (m, 2H), |
| | 7.37-7.29 (m, 5H), 7.12 (t, J = 2.4 Hz, 1H), 6.61 (s, 1H), 6.34 (s, 1H), 4.80 (s, 1H), 4.75-4.71 (m, 1H), 4.64 (s, 1H), 4.53- |
| | 4.49 (m, 1H), 4.29 (s, 2H), 4.00-3.89 (m, 2H), 3.78-3.74 (m, 1H), 3.69-3.65 (m, 1H), 3.58 (d, J = 6.2 Hz, 1H), 3.46 (s, 2H), 2.94- |
| | 2.86 (m, 1H), 2.79 (s, 2H), 2.40-2.25 (m, 10H), 2.07-1.87 (m, 8H), 1.64-1.60 (m, 2H), 1.45 (s, 1H), 1.10-10.7 (m, 2H), 0.82 |
| | (d, J = 6.8 Hz, 6H), 0.64 (s, 2H), 0.41 (s, 2H). |
| 144 | 10.29-1.13 (m, 2H), 9.01 (s, 1H), 7.88 (dd, J = 8.2, 1.8 Hz, 1H), 7.48-7.36 (m, 2H), 7.33 (d, J = 2.6 Hz, 1H), 7.31-7.14 (m, 3H), |
| | 7.10 (d, J = 2.6 Hz, 2H), 6.49 (s, 1H), 4.78 (s, 4H), 4.49-4.47 (m, 1H), 4.26-4.24 (m, 3H), 3.78 (s, 3H), 3.64-3.62 (m, 1H), 3.58 (s, 1H), |
| | 3.54-3.50 (m, 3H), 3.41 (s, 2H), 3.13 (t, J = 6.9 Hz, 1H), 2.75-2.71 (m, 2H), 2.46-2.09 (m, 9H), 2.10-1.98 (m, 2H), 1.88-1.83 (m, 2H), |
| | 1.65 (s, 3H), 1.60-1.58 (m, 2H), 1.36 (s, 1H), 1.13 (d, J = 6.8 Hz, 6H), 1.04-1.01 (m, 2H), 0.63 (s, 2H), 0.39 (s, 2H). |
| 145 | 10.81 (d, J = 13.9 Hz, 1H), 10.20 (s, 1H), 9.76 (s, 1H), 9.39-8.83 (m, 4H), 7.99 (dd, J = 9.2, 5.8 Hz, 1H), 7.47 (t, J = 9.0 Hz, 1H), 7.41 |
| | (d, J = 2.6 Hz, 1H), 7.20-7.12 (m, 3H), 6.97 (s, 2H), 6.59 (s, 1H), 6.35 (s, 1H), 4.67 (s, 1H), 4.48 (d, J = 14.8 Hz, 1H), 4.32 (s, 2H), |
| | 4.21 (s, 2H), 3.99-3.69 (m, 5H), 3.21-3.13 (m, 2H), 3.05-2.85 (m, 4H), 2.72 (d, J = 12.2 Hz, 2H), 2.40-2.17 (m, 6H), 1.97 (s, 4H), 1.77 |
| | (s, 3H), 1.24 (s, 5H), 1.04 (t, J = 7.2 Hz, 3H), 0.82 (d, J = 6.8 Hz, 6H), 0.67 (s, 2H), 0.46 (s, 2H). |
| 146 | 10.17 (s, 1H), 10.05 (s, 1H), 9.72 (s, 1H), 9.63 (d, J = 9.0 Hz, 1H), 9.46 (s, 1H), 9.09 (s, 2H), 7.90 (dd, J = 8.2, 1.8 Hz, 1H), 7.55 (d, J = 8.2 |
| | Hz, 2H), 7.50-7.40 (m, 4H), 7.36 (d, J = 2.4 Hz, 1H), 7.11 (t, J = 2.4 Hz, 1H), 6.71 (s, 1H), 6.31 (s, 1H), 4.88-4.48 (m, 6H), 4.39-4.32 (m, |
| | 4H), 3.83-3.64 (m, 2H), 3.35-3.23 (m, 2H), 3.16-2.80 (m, 10H), 2.38-2.29 (m, 2H), 2.02 (s, 2H), 1.96-1.83 (m, 7H), 1.73 (s, 2H), 1.34 |
| | (d, J = 7.0 Hz, 3H), 1.31-1.25 (m, 1H), 0.89 (dd, J = 6.8, 2.4 Hz, 6H), 0.85-0.78 (m, 2H), 0.77-0.47 (m, 2H). |
| 147 | 10.62 (s, 1H), 10.14 (s, 1H), 9.75 (s, 1H), 9.04 (s, 1H), 8.93 (t, J = 5.8 Hz, 1H), 8.16 (s, 1H), 7.89 (dd, J = 8.2, 1.8 Hz, 1H), 7.49-7.39 |
| | (m, 2H), 7.39-7.32 (m, 3H), 7.32-7.25 (m, 2H), 7.12 (t, J = 2.4 Hz, 1H), 6.57 (s, 1H), 6.34 (s, 1H), 4.80 (s, 1H), 4.78-4.69 (m, 1H), |
| | 4.64 (s, 1H), 4.51 (d, J = 12.8 Hz, 1H), 4.29 (s, 2H), 3.76 (d, J = 12.8 Hz, 1H), 3.67 (d, J = 12.8 Hz, 1H), 3.58 (d, J = 6.6 |
| | Hz, 1H), 3.47 (s, 2H), 3.22-3.10 (m, 2H), 2.89 (p, J = 6.8 Hz, 1H), 2.79 (d, J = 10.6 Hz, 2H), 2.39 (s, 10H), 2.06 (d, J = 7.2 Hz, 2H), 1.97- |
| | 1.87 (m, 6H), 1.62 (d, J = 12.4 Hz, 2H), 1.45 (s, 1H), 1.15-0.99 (m, 5H), 0.80 (d, J = 6.8 Hz, 6H), 0.64 (s, 2H), 0.41 (s, 2H). |
| 148 | 10.62 (s, 1H), 10.13 (s, 1H), 9.74 (s, 1H), 9.02 (s, 1H), 8.93 (t, J = 5.8 Hz, 1H), 7.97 (dd, J = 9.4, 5.8 Hz, 1H), 7.46 (t, J = 9.0 Hz, 1H), 7.41- |
| | 7.33 (m, 3H), 7.28 (d, J = 8.2 Hz, 2H), 7.16 (d, J = 2.5 Hz, 1H), 6.57 (s, 1H), 6.34 (s, 1H), 4.48 (d, J = 12.2 Hz, 1H), 4.28 (d, J = |
| | 12.4 Hz, 3H), 3.92 (d, J = 1.2 Hz, 1H), 3.63 (d, J = 12.0 Hz, 1H), 3.54 (d, J = 11.4 Hz, 3H), 3.46 (s, 2H), 3.43-3.35 (m, 2H), 3.20-3.10 (m, |
| | 2H), 2.90 (p, J = 6.8 Hz, 1H), 2.79 (d, J = 10.6 Hz, 2H), 2.43-2.35 (m, 3H), 2.31-2.22 (m, 6H), 2.06 (d, J = 7.2 Hz, 2H), 1.97-1.87 |
| | (m, 2H), 1.69-1.58 (m, 6H), 1.45 (s, 1H), 1.11 (d, J = 11.2 Hz, 1H), 1.03 (t, J = 7.2 Hz, 3H), 0.80 (d, J = 6.8 Hz, 6H), |
| | 0.63 (s, 2H), 0.40 (s, 2H). |
| 149 | 10.41 (s, 1H), 10.13 (s, 1H), 9.69 (s, 1H), 9.02 (s, 1H), 8.93 (t, J = 5.8 Hz, 1H), 8.20 (s, 1H), 7.97 (dd, J = 9.2, 5.8 |
| | Hz, 1H), 7.46 (t, J = 9.0 Hz, 1H), 7.38 (d, J = 2.6 Hz, 1H), 7.33 (d, J = 8.2 Hz, 2H), 7.25 (d, J = 8.2 Hz, 2H), 7.16 (d, J = 2.6 Hz, |
| | 1H), 6.52 (d, J = 6.2 Hz, 1H), 6.32 (s, 1H), 4.48 (d, J = 12.0 Hz, 1H), 4.28 (d, J = 13.0 Hz, 3H), 3.92 (d, J = 1.2 |
| | Hz, 1H), 3.64 (d, J = 11.8 Hz, 1H), 3.54 (d, J = 12.2 Hz, 3H), 3.46 (s, 2H), 3.20-3.13 (m, 3H), 2.77 (d, J = 10.8 Hz, 2H), |
| | 2.44-2.34 (m, 4H), 2.35-2.26 (m, 4H), 2.20 (q, J = 7.4 Hz, 2H), 2.06 (d, J = 7.0 Hz, 2H), 1.95-1.85 (m, 2H), 1.73-1.54 |
| | (m, 6H), 1.43 (s, 1H), 1.09 (s, 1H), 1.04 (t, J = 7.2 Hz, 3H), 0.82 (t, J = 7.4 Hz, 3H), 0.62 (s, 2H), 0.40 (s, 2H). |
| 150 | 10.43 (s, 1H), 10.13 (s, 1H), 9.74 (s, 1H), 9.55 (d, J = 9.0 Hz, 1H), 9.03 (d, J = 3.2 Hz, 1H), 7.89 (dd, J = 8.2, 1.8 Hz, 1H), |
| | 7.49-7.38 (m, 2H), 7.38-7.32 (m, 3H), 7.29 (d, J = 8.2 Hz, 2H), 7.11 (t, J = 2.8 Hz, 1H), 6.62 (s, 1H), 6.33 (s, 1H), 4.69- |
| | 4.58 (m, 3H), 4.43 (s, 1H), 4.42-4.30 (m, 1H), 4.27 (s, 2H), 3.81-3.67 (m, 1H), 3.64-3.50 (m, 2H), 3.45 (s, 2H), |
| | 2.91 (p, J = 6.8 Hz, 1H), 2.80-2.76 (m, 2H), 2.44-2.20 (m, 10H), 2.09-2.05 (m, 5H), 1.94-1.87 (m, 3H), 1.82-1.75 (m, 3H), 1.63-1.59 |
| | (m, 2H), 1.44 (s, 1H), 1.31 (d, J = 7.0 Hz, 3H), 1.10-1.04 (m, 2H), 0.83 (d, J = 6.8 Hz, 6H), 0.63 (s, 2H), 0.41 (s, 2H). |
| 151 | 10.13 (s, 1H), 10.05 (s, 1H), 9.60 (s, 1H), 9.03 (d, J = 3.2 Hz, 1H), 7.88 (dd, J = 8.0, 1.8 Hz, 1H), 7.47-7.34 (m, 2H), 7.34 (d, J = |
| | 2.6 Hz, 1H), 7.28-7.21 (m, 3H), 7.11 (t, J = 2.8 Hz, 1H), 7.04 (s, 1H), 6.39 (s, 1H), 4.76 (s, 4H), 4.66-4.58 (m, 2H), 4.43 |
| | (s, 1H), 4.40-4.29 (m, 1H), 4.27 (s, 2H), 3.80-3.76 (m, 1H), 3.59 (d, J = 7.6 Hz, 2H), 3.42 (s, 1H), 3.09 (p, J = 6.8 Hz, 1H), 2.76 |
| | (s, 2H), 2.44-2.17 (m, 9H), 2.09 (d, J = 1.4 Hz, 3H), 2.05 (s, 2H), 1.94-1.90 (m, 2H), 1.80-1.76 (m, 4H), 1.61-1.59 (m, 2H), |
| | 1.43 (s, 1H), 1.13 (d, J = 6.8 Hz, 6H), 1.06-1.04 (m, 2H), 0.63 (s, 2H), 0.40 (s, 2H). |
| 152 | 10.12 (s, 1H), 9.66 (s, 1H), 9.01 (d, J = 1.0 Hz, 1H), 7.88 (dd, J = 8.0, 1.8 Hz, 1H), 7.51-7.35 (m, 2H), 7.36-7.24 (m, 2H), |
| | 7.24-7.06 (m, 3H), 6.97 (s, 1H), 6.51 (s, 1H), 4.74 (s, 2H), 4.49 (t, J = 5.8 Hz, 3H), 4.35-4.15 (m, 3H), 3.71 (s, 3H), 3.65-3.63 |
| | (m, 1H), 3.59-3.47 (m, 4H), 3.41-3.38 (m, 2H), 3.13 (p, J = 6.8 Hz, 1H), 2.81-2.64 (m, 2H), 2.44-2.14 (m, 10H), 2.04 (t, J = 7.1 |
| | Hz, 2H), 1.89-1.83 (m, 2H), 1.66 (s, 3H), 1.59 (s, 3H), 1.41 (s, 1H), 1.24 (s, 2H), 1.13 (d, J = 6.8 Hz, 6H), 1.05-1.01 |
| | (m, 2H), 0.90-0.75 (m, 1H), 0.62 (s, 2H), 0.39 (s, 2H). |
| 153 | 10.44 (s, 1H), 10.15 (s, 1H), 9.75 (s, 1H), 9.55 (d, J = 9.0 Hz, 1H), 9.02 (s, 1H), 7.97 (dd, J = 9.2, 5.8 Hz, 1H), 7.46 |
| | (t, J = 9.0 Hz, 1H), 7.39 (d, J = 2.4 Hz, 1H), 7.35 (d, J = 8.4 Hz, 2H), 7.32-7.23 (m, 2H), 7.17 (d, J = 2.6 Hz, 1H), |
| | 6.62 (s, 1H), 6.34 (s, 1H), 4.69-4.62 (m, 1H), 4.52-4.48 (m, 1H), 4.32-4.25 (m, 3H), 3.93 (d, J = 1.0 Hz, 1H), 3.68-3.62 (m, |
| | 1H), 3.58-3.52 (m, 3H), 3.45 (s, 2H), 2.90 (h, J = 6.8 Hz, 1H), 2.82-2.72 (m, 2H), 2.45-2.14 (m, 10H), 2.06 (d, J = 6.8 Hz, |
| | 2H), 1.93-1.88 (m, 2H), 1.67 (s, 4H), 1.65-1.61 (m, 2H), 1.48-1.42 (m, 1H), 1.32 (d, J = 7.0 Hz, 3H), 1.12-1.05 (m, 2H), |
| | 0.90-0.75 (m, 6H), 0.63 (d, J = 4.9 Hz, 2H), 0.40 (s, 2H). |
| 154 | 10.47 (s, 1H), 10.15 (s, 1H), 9.76 (s, 1H), 9.58 (t, J = 6.6 Hz, 1H), 9.03 (s, 1H), 7.97 (dd, J = 9.2, 5.8 Hz, 1H), 7.46 (t, J = 9.0 Hz, 1H), |
| | 7.42-7.22 (m, 5H), 7.17 (d, J = 2.6 Hz, 1H), 6.61 (s, 1H), 6.34 (s, 1H), 4.52-4.48 (m, 1H), 4.32-4.26 (m, 3H), 4.02-3.88 (m, 3H), 3.68- |
| | 3.64 (m, 1H), 3.62-3.50 (m, 3H), 3.46 (s, 2H), 2.90 (p, J = 6.8 Hz, 1H), 2.78 (m, 2H), 2.42-2.20 (m, 10H), 2.06 (d, J = 7.2 Hz, 2H), |
| | 1.92-1.82 (m, 2H), 1.72-1.65 (m, 4H), 1.62-1.55 (m, 2H), 1.46 (s, 1H), 1.12-1.07 (m, 2H), 0.82 (d, J = 6.8 Hz, 6H), 0.63 (s, |
| | 2H), 0.40 (s, 2H). |
| 155 | 10.43 (s, 1H), 10.13 (s, 1H), 9.74 (s, 1H), 9.55 (d, J = 8.8 Hz, 1H), 9.02 (s, 1H), 7.88 (dd, J = 8.2, 1.8 Hz, 1H), 7.53-7.37 |
| | (m, 2H), 7.37-7.21 (m, 5H), 7.11 (d, J = 2.4 Hz, 1H), 6.61 (s, 1H), 6.33 (s, 1H), 4.64 (q, J = 7.6 Hz, 1H), 4.50 (d, J = 11.8 Hz, |
| | 1H), 4.28 (d, J = 12.2 Hz, 3H), 3.72-3.36 (m, 6H), 2.89 (p, J = 6.8 Hz, 1H), 2.72-2.59 (m, 1H), 2.37 (s, 12H), 1.67 (s, 4H), |
| | 1.34-1.13 (m, 4H), 0.81 (d, J = 6.8 Hz, 6H), 0.63 (s, 2H), 0.40 (s, 2H). |
| 156 | 10.13 (s, 2H), 9.60 (s, 1H), 7.97 (dd, J = 9.2, 5.8 Hz, 1H), 7.46 (t, J = 9.0 Hz, 1H), 7.38 (d, J = 2.6 Hz, 1H), 7.31-7.11 (m, 4H), 7.04 |
| | (s, 1H), 6.39 (s, 1H), 4.76 (s, 4H), 4.25 (s, 2H), 4.12 (s, 1H), 3.87 (d, J = 2.2 Hz, 4H), 3.47 (s, 2H), 3.41 (s, 3H), 3.36 (s, 2H), 3.09 |
| | (p, J = 6.8 Hz, 1H), 2.75-2.73 (m, 2H), 2.44-2.20 (m, 10H), 2.04 (d, J = 7.2 Hz, 2H), 1.88-1.84 (m, 2H), 1.60-1.58 (m, 6H), |
| | 1.40-1.38 (m, 1H), 1.13 (d, J = 6.8 Hz, 6H), 1.05-1.03 (m, 2H), 0.61 (s, 2H), 0.38 (s, 2H). |
| 157 | 10.13 (s, 2H), 9.60 (s, 1H), 9.02 (s, 1H), 8.22 (s, 1H), 7.88 (dd, J = 8.2, 1.6 Hz, 2H), 7.47-7.38 (m, 1H), 7.33 (d, J = 2.6 Hz, 3H), 7.20 |
| | (s, 1H), 7.10 (d, J = 2.6 Hz, 1H), 7.04 (s, 1H), 6.39 (s, 1H), 4.76 (s, 4H), 4.48 (d, J = 12.6 Hz, 1H), 4.28 (s, 2H), 3.65 |
| | (s, 1H), 3.62 (s, 1H), 3.57 (s, 2H), 3.53 (d, J = 12.8 Hz, 1H), 3.43 (s, 1H), 3.10 (q, J = 6.8 Hz, 2H), 2.82-2.57 (m, 1H), 2.32 (d, |
| | J = 3.6 Hz, 4H), 1.92 (d, J = 13.2 Hz, 2H), 1.65 (s, 5H), 1.53 (s, 4H), 1.23 (s, 2H), 1.13 (d, J = 6.8 Hz, 4H), 0.84 (d, J = 7.2 |
| | Hz, 6H), 0.64 (s, 4H), 0.41 (s, 2H). |
| 158 | 10.44 (s, 1H), 10.14 (s, 1H), 9.75 (s, 1H), 9.55 (d, J = 9.0 Hz, 1H), 8.01-7.90 (m, 1H), 7.46 (s, 1H), 7.40-7.32 (m, 3H), 7.28 (d, J = |
| | 8.4 Hz, 2H), 7.21 (d, J = 2.5 Hz, 1H), 6.62 (s, 1H), 6.34 (s, 1H), 4.71-4.55 (m, 7.6 Hz, 1H), 4.25 (s, 2H), 4.12 (s, 1H), 3.88 |
| | (s, 5H), 3.53-3.43 (m, 4H), 3.39-3.22 (m, 4H), 2.91 (p, J = 6.8 Hz, 1H), 2.83-2.71 (m, 2H), 2.44-2.33 (m, 3H), 2.33- |
| | 2.24 (m, 4H), 2.05 (d, J = 7.2 Hz, 2H), 1.97-1.81 (m, 2H), 1.70-1.50 (m, 5H), 1.44 (s, 1H), 1.32 (d, J = 7.2 Hz, 3H), |
| | 1.17-0.98 (m, 2H), 0.90-0.74 (m, 6H), 0.61 (s, 2H), 0.39 (s, 2H). |
| 159 | 10.06 (s, 1H), 9.94 (s, 1H), 9.59 (s, 1H), 9.15 (s, 1H), 7.80 (s, 1H), 7.78 (s, 1H), 7.55 (d, J = 8.4 Hz, 1H), 7.43 (t, J = 7.5 Hz, |
| | 1H), 7.31-7.16 (m, 5H), 7.05 (s, 1H), 6.39 (s, 1H), 4.75 (s, 4H), 4.44 (d, J = 12.1 Hz, 2H), 4.30 (s, 2H), 3.61 (s, 2H), |
| | 3.56 (d, J = 15.9 Hz, 2H), 3.31 (s, 2H), 3.09 (p, J = 6.8 Hz, 1H), 2.74 (d, J = 10.8 Hz, 2H), 2.37 (s, 4H), 2.27 (s, 6H), |
| | 2.01 (d, J = 7.0 Hz, 2H), 1.84 (d, J = 11.4 Hz, 2H), 1.64 (s, 4H), 1.58 (d, J = 12.5 Hz, 2H), 1.40 (s, 1H), 1.24 (s, 1H), |
| | 1.13 (d, J = 6.9 Hz, 6H), 1.04 (d, J = 12.2 Hz, 2H), 0.63 (s, 2H), 0.39 (s, 2H). |
| 160 | 10.42 (s, 1H), 10.14 (s, 1H), 9.74 (s, 1H), 9.54 (d, J = 9.0 Hz, 1H), 9.04 (s, 1H), 7.97 (dd, J = 9.2, 5.9 Hz, 1H), 7.45 (t, J = 9.0 |
| | Hz, 1H), 7.39 (d, J = 2.6 Hz, 1H), 7.35 (d, J = 8.3 Hz, 2H), 7.32-7.23 (m, 2H), 7.17 (d, J = 2.6 Hz, 1H), 6.62 (s, 1H), 6.33 (s, |
| | 1H), 4.64 (h, J = 7.5 Hz, 1H), 4.52-4.50 (m, 1H), 4.35-4.25 (m, 3H), 3.92 (d, J = 1.1 Hz, 1H), 3.66 (d, J = 9.3 Hz, |
| | 3H), 3.61-3.58 (m, 1H), 3.45 (s, 2H), 2.91 (p, J = 6.9 Hz, 1H), 2.89-2.82 (m, 2H), 2.33 (p, J = 1.9 Hz, 8H), 2.28 (s, 2H), |
| | 2.19-2.15 (m, 1H), 1.95-1.88 (m, 2H), 1.72 (s, 4H), 1.68-1.62 (m, 2H), 1.42-1.35 (m, 2H), 1.31 (d, J = 7.1 Hz, 3H), 1.24 |
| | (s, 1H), 0.82 (d, J = 6.9 Hz, 6H), 0.63 (s, 2H), 0.40 (s, 2H). |
| 161 | 10.14 (s, 1H), 9.60 (s, 1H), 9.02 (s, 1H), 7.97 (dd, J = 9.2, 5.9 Hz, 1H), 7.45 (t, J = 9.0 Hz, 1H), 7.38 (d, J = 2.6 Hz, 1H), 7.33- |
| | 7.10 (m, 4H), 7.04 (s, 1H), 6.39 (s, 1H), 4.76 (s, 4H), 4.52-4.47 (m, 1H), 4.30-4.22 (m, 3H), 3.92 (d, J = 1.1 Hz, 1H), |
| | 3.68-3.62 (m, 1H), 3.53 (d, J = 8.5 Hz, 3H), 3.40 (s, 2H), 3.09 (p, J = 6.9 Hz, 1H), 2.82-2.78 (m, 2H), 2.46-2.29 |
| | (m, 8H), 2.27 (d, J = 4.1 Hz, 2H), 2.08 (s, 1H), 1.90-1.82 (m, 2H), 1.68-1.62 (m, 6H), 1.38-1.30 (m, 2H), 1.13 (d, J = 6.9 Hz, |
| | 6H), 0.62 (s, 2H), 0.39 (s, 2H). |
| 162 | 10.43 (s, 1H), 10.17 (s, 1H), 9.76 (s, 1H), 9.57 (d, J = 9.0 Hz, 1H), 9.04 (s, 1H), 7.97 (dd, J = 9.3, 5.9 Hz, 1H), 7.46 (t, J = 9.0 |
| | Hz, 1H), 7.39 (d, J = 2.6 Hz, 1H), 7.34 (d, J = 8.1 Hz, 2H), 7.28 (d, J = 8.1 Hz, 2H), 7.17 (d, J = 2.6 Hz, 1H), 6.62 (s, 1H), 6.33 |
| | (s, 1H), 4.64 (q, J = 7.9 Hz, 1H), 4.54-4.47 (m, 1H), 4.36-4.21 (m, 3H), 3.93 (s, 1H), 3.74-3.50 (m, 4H), 3.44 (s, 2H), |
| | 3.03-2.93 (m, 2H), 2.93-2.86 (m, 1H), 2.85-2.77 (m, 2H), 2.34-2.21 (m, 2H), 1.93-1.66 (m, 6H), 1.62-1.52 (m, 4H), |
| | 1.31 (d, J = 7.1 Hz, 3H), 1.12 (s, 4H), 0.99 (d, J = 7.0 Hz, 2H), 0.82 (d, J = 6.9 Hz, 6H), 0.63 (s, 2H), 0.39 (s, 2H). |
| 163 | 10.20 (s, 1H), 10.03 (s, 1H), 9.62 (s, 1H), 9.12 (s, 1H), 8.90-8.30 (m, 1H), 7.98 (dd, J = 9.2, 6.0 Hz, 1H), 7.46 (t, J = 9.0 Hz, |
| | Hz, 1H), 7.41 (d, J = 2.5 Hz, 1H), 7.30 (s, 2H), 7.17 (d, J = 2.5 Hz, 1H), 7.04 (s, 1H), 6.40 (s, 1H), 4.79 (s, 4H), 4.68-4.61 |
| | (m, 1H), 4.53-4.45 (m, 1H), 4.38-4.25 (m, 2H), 4.14 (s, 3H), 3.91 (s, 1H), 3.86-3.77 (m, 2H), 3.24-3.14 (m, 8H), 3.10 (t, J = |
| | 6.9 Hz, 1H), 2.43-2.35 (m, 5H), 1.92 (d, J = 9.8 Hz, 5H), 1.78-1.55 (m, 4H), 1.14 (d, J = 6.9 Hz, 9H). 0.91-0.51 (m, 4H). |
| 164 | 10.33-9.84 (m, 2H), 9.60 (s, 1H), 9.02 (s, 1H), 7.97 (dd, J = 9.2, 5.9 Hz, 1H), 7.46 (t, J = 9.0 Hz, 1H), 7.39 (d, J = 2.6 Hz, 1H), |
| | 7.17 (d, J = 2.5 Hz, 3H), 7.13 (d, J = 7.8 Hz, 1H), 7.04 (s, 1H), 6.39 (s, 1H), 4.74 (s, 4H), 4.48 (d, J = 12.0 Hz, 1H), 4.27 |
| | (d, J = 15.1 Hz, 3H), 3.92 (d, J = 1.1 Hz, 1H), 3.63 (d, J = 12.0 Hz, 1H), 3.54 (d, J = 11.6 Hz, 3H), 3.47 (s, 2H), 3.25 (d, J = 6.8 |
| | Hz, 4H), 3.10 (h, J = 7.0 Hz, 1H), 2.78-2.61 (m, 2H), 2.39 (d, J = 7.0 Hz, 3H), 2.36-2.07 (m, 8H), 1.66 (s, 4H), 1.13 (d, J = |
| | 6.9 Hz, 6H), 0.62 (d, J = 4.1 Hz, 2H), 0.39 (d, J = 4.6 Hz, 2H). |
| 165 | 10.43 (s, 1H), 10.15 (s, 1H), 9.75 (s, 1H), 9.54 (d, J = 9.0 Hz, 1H), 9.02 (s, 1H), 7.97 (dd, J = 9.2, 5.9 Hz, 1H), 7.46 (t, J = 9.0 |
| | Hz, 1H), 7.38 (d, J = 2.6 Hz, 1H), 7.28 (q, J = 8.5 Hz, 4H), 7.17 (d, J = 2.6 Hz, 1H), 6.59 (s, 1H), 6.33 (s, 1H), 4.62 (dt, J = 15.8, 7.8 Hz, |
| | 1H), 4.48 (d, J = 12.1 Hz, 1H), 4.27 (d, J = 14.4 Hz, 3H), 3.92 (d, J = 1.1 Hz, 1H), 3.63-3.45 (m, 1H), 3.54 (d, J = 6.6 Hz, 5H), |
| | 3.29 (s, 4H), 2.90 (p, J = 6.8 Hz, 1H), 2.75 (t, J = 6.6 Hz, 2H), 2.42 (d, J = 7.1 Hz, 2H), 2.34-2.17 (m, 9H), 1.66 (s, |
| | 4H), 1.31 (d, J = 7.1 Hz, 3H), 0.83 (d, J = 6.9 Hz, 6H), 0.62 (s, 2H), 0.39 (s, 2H). |
| 166 | 10.46 (s, 1H), 10.14 (s, 1H), 9.75 (s, 1H), 9.57 (t, J = 6.5 Hz, 1H), 9.02 (s, 1H), 7.97 (dd, J = 9.2, 5.9 Hz, 1H), 7.46 |
| | (t, J = 9.0 Hz, 1H), 7.39 (d, J = 2.5 Hz, 1H), 7.35-7.22 (m, 4H), 7.17 (d, J = 2.6 Hz, 1H), 6.58 (s, 1H), 6.33 (s, 1H), 4.48 (d, J = |
| | 12.0 Hz, 1H), 4.28 (d, J = 14.8 Hz, 3H), 4.05-3.86 (m, 3H), 3.63 (d, J = 12.0 Hz, 1H), 3.55 (d, J = 6.0 Hz, 5H), 2.89 |
| | (h, J = 6.7 Hz, 1H), 2.76 (t, J = 6.6 Hz, 2H), 2.55 (d, J = 13.8 Hz, 2H), 2.42 (d, J = 7.0 Hz, 2H), 2.36 (s, 2H), 2.35- |
| | 1.86 (m, 9H), 1.67 (s, 4H), 0.82 (d, J = 6.9 Hz, 6H), 0.63 (d, J = 4.9 Hz, 2H), 0.40 (d, J = 4.4 Hz, 2H). |
| 167 | 10.41 (s, 1H), 10.18 (s, 1H), 9.54 (d, J = 9.0 Hz, 1H), 9.03 (s, 1H), 7.97 (dd, J = 9.2, 5.9 Hz, 1H), 7.45 (t, J = 9.0 Hz, 1H), 7.39 |
| | (d, J = 2.6 Hz, 1H), 7.32 (d, J = 8.1 Hz, 2H), 7.26 (d, J = 8.1 Hz, 2H), 7.17 (d, J = 2.6 Hz, 1H), 6.60 (s, 1H), |
| | 6.32 (s, 1H), 4.63 (h, J = 7.6 Hz, 1H), 4.49 (d, J = 12.0 Hz, 1H), 4.27 (d, J = 7.9 Hz, 3H), 3.92 (s, 1H), 3.72-3.52 (m, 6H), 2.91 |
| | (p, J = 6.9 Hz, 2H), 2.82 (s, 3H), 2.40 (s, 4H), 2.35-2.28 (m, 3H), 2.28-1.91 (m, 4H), 1.68 (s, 4H), 1.32 (d, J = 7.0 Hz, 3H), 0.83 |
| | (d, J = 6.8 Hz, 6H), 0.63 (s, 2H), 0.41 (d, J = 4.4 Hz, 2H). |
| 168 | 10.47 (s, 1H), 10.13 (s, 1H), 9.75 (s, 1H), 9.57 (t, J = 6.5 Hz, 1H), 7.97 (dd, J = 9.2, 5.9 Hz, 1H), 7.46 (t, J = 9.0 Hz, 1H), 7.38 |
| | (d, J = 2.6 Hz, 1H), 7.35 (d, J = 8.4 Hz, 2H), 7.30 (d, J = 8.4 Hz, 2H), 7.21 (d, J = 2.6 Hz, 1H), 6.61 (s, 1H), 6.34 (s, 1H), 4.26 (s, |
| | 2H), 4.12 (s, 1H), 4.00-3.91 (m, 2H), 3.88 (s, 5H), 3.46 (s, 2H), 3.41 (s, 4H), 3.39-3.33 (m, 4H), 2.90 (p, J = 7.0 Hz, 1H), 2.78 |
| | (d, J = 10.3 Hz, 2H), 2.28 (d, J = 5.9 Hz, 2H), 2.06 (d, J = 7.1 Hz, 2H), 1.95-1.84 (m, 2H), 1.61 (d, J = 12.7 Hz, 6H), |
| | 1.34-1.51 (m, 2H), 1.24 (s, 1H), 1.07 (d, J = 11.9 Hz, 2H), 0.99 (d, J = 6.3 Hz, 2H), 0.82 (d, J = 6.9 Hz, 6H), 0.61 (s, 2H), 0.39 (s, 2H). |
| 169 | 10.62 (s, 1H), 10.13 (s, 1H), 9.75 (s, 1H), 8.93 (t, J = 5.9 Hz, 1H), 7.83-8.04 (m, J = 9.2, 5.9 Hz, 1H), 7.46 (t, J = 9.0 Hz, 1H), |
| | 7.39-7.33 (m, 3H), 7.31-7.26 (m, 2H), 7.21 (d, J = 2.6 Hz, 1H), 6.57 (s, 1H), 6.34 (s, 1H), 4.26 (s, 2H), 4.12 (s, 1H), 3.87 (d, |
| | J = 3.8 Hz, 5H), 3.46 (s, 4H), 3.41 (s, 2H), 3.20 (s, 1H), 3.16-3.18 (m, J = 7.3, 6.0 Hz, 2H), 2.90 (p, J = 6.8 Hz, 1H), 2.79 |
| | (d, J = 10.5 Hz, 2H), 2.35-2.14 (m, 12H), 2.06 (d, J = 7.0 Hz, 2H), 1.97-1.82 (m, 2H), 1.59 (t, J = 17.0 Hz, 6H), 1.45 (s, |
| | 1H), 1.03 (t, J = 7.2 Hz, 3H), 0.80 (d, J = 6.9 Hz, 6H), 0.61 (s, 2H), 0.40 (d, J = 5.1 Hz, 2H). |
| 170 | 10.13 (s, 2H), 9.60 (s, 1H), 7.96 (dd, J = 9.2, 5.9 Hz, 1H), 7.45 (t, J = 9.0 Hz, 1H), 7.37 (d, J = 2.6 Hz, 1 H), 7.33-7.08 (m, 4 H), |
| | 7.04 (s, 1H), 6.39 (s, 1H), 4.75 (s, 4H), 4.25 (s, 2H), 4.12 (s, 1H), 3.87 (s, 5H), 3.46 (s, 2 H), 3.39 (s, 2 H), 3.31 (s, |
| | 1H), 3.12-3.04 (m, 1H), 2.78 (s, 2H), 2.50 (p, J = 1.9 Hz, 6 H), 2.39 (s, 2 H), 2.06 (d, J = 10.4 Hz, 2H), 1.88 (d, J = 20.0 Hz, 2H), |
| | 1.61 (s, 8H), 1.33 (s, 2 H), 1.13 (d, J = 6.9 Hz, 6 H), 0.61 (s, 2H), 0.38 (s, 2 H). |
| 172 | 9.82 (m, 2H), 9.05 (s, 1H), 8.20 (s, 1H), 7.97 (dd, J = 9.2, 5.9 Hz, 1H), 7.46 (t, J = 9.0 Hz, 1H), 7.39 (d, J = 2.6 Hz, 1H), 7.31-7.13 (m, |
| | 4H), 7.04 (s, 1H), 6.39 (s, 1H), 4.75 (s, 4H), 4.56-4.40 (m, 3H), 4.35 (d, J = 12.3 Hz, 1H), 3.93 (s, 1H), 3.66-3.63 (m, 3H), 3.59 (s, |
| | 2H), 3.33 (t, J = 7.3 Hz, 2H), 3.09 (p, J = 6.9 Hz, 1H), 3.03 (t, J = 6.5 Hz, 2H), 2.84-2.82 (m, 1H), 1.70 (s, 4H), 1.13 (d, J = 6.9 Hz, 6H). |
| 173 | 10.10 (d, J = 37.1 Hz, 2H), 9.60 (s, 1H), 9.04 (s, 1H), 7.98 (dd, J = 9.2, 5.9 Hz, 1H), 7.54-7.23 (m, 5H), 7.18 (d, J = 2.6 Hz, 1H), 7.04 |
| | (s, 1H), 6.39 (s, 1H), 5.42 (s, 2H), 4.79 (s, 4H), 4.47 (d, J = 12.1 Hz, 1H), 4.31 (d, J = 12.1 Hz, 1H), 3.94 (d, J = 1.1 Hz, 1H), |
| | 3.63 (d, J = 12.1 Hz, 1H), 3.55 (d, J = 16.4 Hz, 3H), 3.09 (p, J = 6.9 Hz, 1H), 1.60 (s, 4H), 1.13 (d, J = 6.9 Hz, 6H). |
| 174 | 10.41 (s, 1H), 9.81 (s, 1H), 9.55 (d, J = 9.0 Hz, 1H), 9.05 (s, 1H), 8.20 (s, 1H), 7.97 (dd, J = 9.2, 5.9 Hz, 1H), 7.51-7.40 |
| | (m, 1H), 7.39 (d, J = 2.6 Hz, 1H), 7.33 (s, 2H), 7.29-7.23 (m, 2H), 7.17 (d, J = 2.6 Hz, 1H), 6.60 (s, 1H), 6.33 (s, 1H), 4.66-4.63 (m, |
| | 1H), 4.52 (t, J = 6.8 Hz, 1H), 4.47 (s, 1H), 4.36-4.34 (m, 1H), 3.93 (s, 1H), 3.63-3.58 (m, 6H), 3.51-3.49 (m, 1H), |
| | 3.35 (t, J = 7.2 Hz, 2H), 3.06 (t, J = 6.3 Hz, 2H), 2.94-2.79 (m, 2H), 1.69 (s, 4H), 1.31 (d, J = 7.1 Hz, 3H), 0.82 (d, J = 6.9 Hz, 6H). |
| 175 | 10.44 (s, 1H), 10.14 (s, 1H), 9.75 (s, 1H), 9.57 (t, J = 6.5 Hz, 1H), 9.04 (s, 1H), 7.97 (dd, J = 9.2, 5.9 Hz, 1H), 7.46 (t, J = 9.0 Hz, 1H), |
| | 7.39 (d, J = 2.5 Hz, 1H), 7.36 (s, 2H), 7.28 (d, J = 8.5 Hz, 2H), 7.17 (d, J = 2.5 Hz, 1H), 6.59 (s, 1H), 6.33 (s, 1H), 4.52 (d, J = 7.1 Hz, 2H), |
| | 4.48-4.46 (m, 1H), 4.32-4.30 (m, 1H), 4.02-3.88 (m, 3H), 3.63 (s, 3H), 3.56 (s, 3H), 3.37-3.35 (m, 2H), 3.05 (t, J = 6.3 Hz, |
| | 2H), 2.89-2.84 (m, 2H), 1.65 (s, 4H), 0.81 (d, J = 6.9 Hz, 6H). |
| 176 | 10.39 (s, 1H), 10.11 (s, 1H), 9.80 (s, 1H), 9.53 (d, J = 9.0 Hz, 1H), 9.04 (s, 1H), 7.97 (dd, J = 9.2, 5.9 Hz, 1H), 7.45 (t, J = 9.0 |
| | Hz, 1H), 7.40-7.33 (m, 3H), 7.33-7.25 (m, 2H), 7.16 (d, J = 2.6 Hz, 1H), 6.61 (s, 1H), 6.32 (s, 1H), 5.24 (p, J = 5.8 Hz, 1H), |
| | 4.66-4.63 (m, 1H), 4.48-4.48 (m, 1H), 4.304.28 (m, 1H), 3.91 (s, 1H), 3.75 (s, 2H), 3.70 (s, 2H), 3.63-3.61 (m, |
| | 1H), 3.56 (m, 2H), 3.153.13 (m, 2H), 2.89 (p, J = 6.9 Hz, 1H), 1.65 (s, 4H), 1.31 (d, J = 7.0 Hz, 3H), 0.82 (d, J = 6.9 Hz, 6H). |
| 177 | 10.25-9.91 (m, 2H), 9.59 (s, 1H), 9.02 (s, 1H), 7.97 (dd, J = 9.2, 5.9 Hz, 1H), 7.45 (t, J = 9.0 Hz, 1H), 7.38 (d, J = 2.5 Hz, 1H), 7.32- |
| | 7.08 (m, 4H), 7.04 (s, 1H), 6.39 (s, 1H), 4.74 (s, 4H), 4.47 (d, J = 12.1 Hz, 1H), 4.27 (d, J = 16.0 Hz, 3H), 3.92 (d, J = 1.1 Hz, |
| | 1H), 3.63 (d, J = 12.0 Hz, 1H), 3.53 (d, J = 10.3 Hz, 5H), 3.09 (p, J = 6.9 Hz, 1H), 2.77 (d, J = 5.8 Hz, 3H), 2.45-2.31 (m, 7H), |
| | 2.29 (d, J = 5.0 Hz, 2H), 2.26-2.05 (m, 4H), 1.66 (s, 4H), 1.13 (d, J = 6.9 Hz, 6H), 0.63 (d, J = 5.3 Hz, 2H), 0.39 (s, 2H). |
| 178 | 10.42 (s, 1H), 10.12 (s, 1H), 9.75 (s, 1H), 9.53 (d, J = 9.0 Hz, 1 H), 7.96 (dd, J = 9.2, 5.9 Hz, 1 H), 7.45 (t, J = 9.0 |
| | Hz, 1H), 7.38 (d, J = 2.5 Hz, 1H), 7.34 (d, J = 8.4 Hz, 2H), 7.28 (d, J = 8.4 Hz, 2H), 7.21 (d, J = 2.6 Hz, 1 H), 6.62 (s, 1H), 6.33 |
| | (s, 1H), 4.64 (dq, J = 15.4, 7.6 Hz, 1H), 4.26 (s, 2H), 4.12 (s, 1H), 3.87 (d, J = 3.8 Hz, 3H), 3.48 (s, 2H), 3.44 (s, 2H), 3.41 (s, 2 H), |
| | 3.38 (s, 1 H), 3.31 (s, 1 H), 2.91 (p, J = 6.9 Hz, 1 H), 2.81 (d, J = 10.7 Hz, 2 H), 2.67 (p, J = 1.8 Hz, 8 H), 2.50 (p, J = 1.9 Hz, 2H), |
| | 2.45-2.25 (m, 1H), 2.09 (d, J = 11.6 Hz, 2 H), 1.90 (t, J = 11.3 Hz, 6 H), 1.61 (q, J = 2.15 Hz, 17.0 Hz, 2 H), 1.38 (t, J = 11.5 |
| | Hz, 3 H), 0.88-0.77 (m, 6H), 0.62 (d, J = 5.6 Hz, 2H), 0.39 (d, J = 5.3 Hz, 2H). |
| 179 | 10.42 (s, 1H), 9.98 (s, 2H), 9.56 (t, J = 6.5 Hz, 1H), 9.04 (s, 1H), 7.96 (dd, J = 9.2, 5.9 Hz, 1H), 7.45 (t, J = 9.0 Hz, 1H), |
| | 7.39-7.33 (m, 3H), 7.33-7.22 (m, 2H), 7.17 (d, J = 2.6 Hz, 1H), 6.59 (s, 1H), 6.32 (s, 1H), 5.24 (p, J = 5.8 Hz, 1H), 4.48-4.46 |
| | (m, 1H), 4.30-4.28 (m, 1H), 3.97-3.93 (m, 2H), 3.91 (d, J = 1.0 Hz, 1H), 3.76 (d, J = 5.8 Hz, 2H), 3.71 (d, J = 2.7 Hz, 2H), 3.68- |
| | 3.59 (m, 1H), 3.56 (s, 3H), 3.20-3.07 (m, 2H), 2.88 (p, J = 6.9 Hz, 1H), 1.65 (s, 4H), 0.80 (dd, J = 6.9, 1.9 Hz, 6H). |
| 180 | 10.14 (s, 1H), 9.60 (s, 1H), 9.02 (s, 1H), 7.96 (dd, J = 9.2, 5.9 Hz, 1H), 7.45 (t, J = 9.0 Hz, 1H), 7.38 (d, J = 2.6 Hz, 1H), 7.35- |
| | 7.11 (m, 5H), 7.04 (s, 1H), 6.39 (s, 1H), 4.76 (s, 4H), 4.47 (d, J = 12.1 Hz, 1H), 4.28 (d, J = 13.6 Hz, 3H), 3.92 (d, J = 1.1 |
| | Hz, 1H), 3.62 (d, J = 12.0 Hz, 1H), 3.53 (d, J = 9.1 Hz, 3H), 3.40 (s, 2H), 3.09 (p, J = 6.9 Hz, 1H), 2.96 (d, J = 10.5 |
| | Hz, 2H), 2.40 (d, J = 5.8 Hz, 4H), 2.33 (dt, J = 5.2, 2.6 Hz, 2H), 2.26 (s, 4H), 2.09 (s, 1H), 1.81 (t, J = 11.5 Hz, 2H), |
| | 1.66 (s, 6H), 1.41-1.25 (m, 2H), 1.13 (d, J = 6.9 Hz, 6H), 0.62 (s, 2H), 0.38 (s, 2H). |
| 181 | 10.40 (s, 1H), 10.13 (s, 1H), 9.74 (s, 1H), 9.54 (d, J = 9.0 Hz, 1H), 9.02 (s, 1H), 7.97 (dd, J = 9.2, 5.9 Hz, 1H), 7.45 (t, J = 9.0 |
| | Hz, 1H), 7.40-7.24 (m, 5H), 7.17 (d, J = 2.6 Hz, 1H), 6.62 (s, 1H), 6.33 (s, 1H), 4.75-4.55 (m, 1H), 4.48 (d, J = 11.9 Hz, |
| | 1H), 4.29 (d, J = 17.4 Hz, 3H), 3.92 (d, J = 1.1 Hz, 1H), 3.69-3.50 (m, 5H), 3.45 (s, 2H), 2.97 (d, J = 10.7 Hz, 3H), 2.90 (p, J = |
| | 6.9 Hz, 1H), 2.44 (s, 1H), 2.33 (p, J = 1.9 Hz, 1H), 2.27 (s, 2H), 2.27-2.03 (m, 1H), 1.82 (s, 2H), 1.67 (s, 7H), 1.31 (d, |
| | J = 7.0 Hz, 4H), 1.24 (s, 2H), 0.89-0.75 (m, 7H), 0.63 (s, 3H), 0.39 (s, 3H). |
| 182 | 10.43 (s, 1H), 10.11 (s, 1H), 9.81 (s, 1H), 9.54 (d, J = 9.0 Hz, 1H), 8.20 (s, 1H), 7.97 (dd, J = 9.2, 5.9 Hz, 1H), 7.46 (t, |
| | J = 9.0 Hz, 1H), 7.38 (d, J = 2.5 Hz, 1H), 7.34 (d, J = 8.4 Hz, 2H), 7.31-7.25 (m, 2H), 7.21 (d, J = 2.5 Hz, 1H), 6.62 (s, 1H), |
| | 6.33 (s, 1H), 4.68-4.62 (m, 1H), 4.27 (s, 2H), 4.13 (s, 1H), 3.87 (s, 5H), 3.57 (s, 2H), 3.43 (s, 2H), 3.34 (s, 1H), 2.98-2.88 |
| | (m, 4H), 2.81 (d, J = 10.6 Hz, 2H), 2.27 (s, 2H), 1.86-1.82 (m, 2H), 1.80-1.71 (m, 2H), 1.68-1.62 (m, 4H), 1.57 (d, J = |
| | 12.2 Hz, 4H), 1.31 (d, J = 7.0 Hz, 3H), 1.21-1.02 (m, 4H), 0.98 (d, J = 9.8 Hz, 2H), 0.88-0.75 (m, 6H), 0.62 (s, 2H), 0.40 |
| | (d, J = 5.1 Hz, 2H). |
| 183 | 10.42 (s, 1H), 10.15 (s, 1H), 9.75 (s, 1H), 9.56 (d, J = 8.9 Hz, 1H), 9.02 (s, 1H), 7.97 (dd, J = 9.2, 5.9 Hz, 1H), 7.46 (t, J = 9.0 |
| | Hz, 1H), 7.39 (d, J = 2.6 Hz, 1H), 7.34 (d, J = 8.4 Hz, 2H), 7.31-7.24 (m, 2H), 7.16 (d, J = 2.6 Hz, 1H), 6.62 (s, 1H), 6.33 (s, |
| | 1H), 4.63 (dt, J = 15.5, 7.8 Hz, 1H), 4.48 (d, J = 12.1 Hz, 1H), 4.27 (d, J = 17.1 Hz, 3H), 3.93 (d, J = 1.1 Hz, 1H), 3.63 (d, J = 12.0 |
| | Hz, 1H), 3.54 (d, J = 8.6 Hz, 3H), 3.45 (s, 2H), 2.90 (p, J = 6.9 Hz, 1H), 2.32 (d, J = 17.9 Hz, 10H), 1.66 (s, 4H), 1.37 (s, 7H), |
| | 1.31 (d, J = 7.0 Hz, 4H), 1.24 (s, 1H), 0.92-0.79 (m, 6H), 0.62 (s, 2H), 0.40 (s, 2H). |
| 184 | 10.15 (s, 1H), 10.04 (s, 1H), 9.61 (s, 1H), 9.02 (s, 1H), 7.97 (dd, J = 9.2, 6.0 Hz, 1H), 7.46 (t, J = 9.0 Hz, 1H), 7.38 (d, J = |
| | 2.6 Hz, 1H), 7.25 (s, 1H), 7.20-7.13 (m, 3H), 7.04 (s, 1H), 6.39 (s, 1H), 4.75 (s, 4H), 4.48 (d, J = 12.1 Hz, 1H), 4.27 (d, J = 15.6 |
| | Hz, 3H), 3.93 (d, J = 1.1 Hz, 1H), 3.63 (d, J = 12.0 Hz, 1H), 3.53 (d, J = 9.8 Hz, 3H), 3.45 (s, 2H), 3.09 (p, J = 6.9 Hz, 1H), 2.32 |
| | (d, J = 17.9 Hz, 10H), 1.65 (s, 4H), 1.35 (s, 8H), 1.13 (d, J = 6.9 Hz, 6H), 0.62 (s, 2H), 0.39 (s, 2H). |
| 185 | 10.45 (s, 1H), 10.10 (s, 2H), 9.54 (d, J = 9.0 Hz, 1H), 9.08 (d, J = 42.7 Hz, 1H), 7.97 (dd, J = 9.3, 5.8 Hz, 1H), 7.45 |
| | (t, J = 9.0 Hz, 1H), 7.41-7.25 (m, 5H), 7.17 (dd, J = 17.2, 2.6 Hz, 1H), 6.62 (s, 1H), 6.33 (d, J = 2.6 Hz, 1H), 4.95-4.59 (m, |
| | 2H), 4.57-4.04 (m, 3H), 4.02-3.54 (m, 3H), 3.45 (d, J = 11.2 Hz, 3H), 3.39 (s, 2H), 2.97 (t, J = 12.3 Hz, 2H), 2.93-2.85 |
| | (m, 2H), 2.81 (d, J = 10.5 Hz, 2H), 2.36-2.16 (m, 2H), 1.86 (q, J = 11.6, 11.2 Hz, 2H), 1.75 (d, J = 11.2 Hz, 4H), 1.57 |
| | (d, J = 12.0 Hz, 4H), 1.31 (d, J = 7.0 Hz, 3H), 1.12 (s, 4H), 0.98 (s, 6H), 0.82 (d, J = 6.9 Hz, 6H), 0.63 (d, J = 4.7 Hz, |
| | 2H), 0.39 (s, 2H). |
| 186 | 10.06 (s, 2H), 9.62 (s, 1H), 8.19 (s, 2H), 7.97 (dd, J = 9.2, 6.0 Hz, 1H), 7.46 (t, J = 9.0 Hz, 1H), 7.38 (d, J = 2.6 Hz, 1H), 7.35-7.13 |
| | (m, 4H), 7.04 (s, 1H), 6.39 (s, 1H), 4.75 (s, 4H), 4.27 (s, 2H), 4.14 (s, 1H), 3.88 (d, J = 4.8 Hz, 5H), 3.61 (s, 2H), |
| | 3.44 (s, 4H), 3.38 (s, 2H), 3.35 (s, 2H), 3.09 (p, J = 6.9 Hz, 1H), 2.95 (s, 2H), 2.78 (d, J = 10.5 Hz, 2H), 2.27 (s, 2H), 1.88- |
| | 1.73 (m, 4H), 1.72-1.60 (m, 4H), 1.58-1.52 (m, 4H), 1.13 (d, J = 7.0 Hz, 6H), 1.09-1.07 (m, 1H), 0.95 (s, 1H), 0.66- |
| | 0.62 (m, 2H), 0.41-0.37 (m, 2H). |
| 187 | 10.14 (s, 2H), 9.61 (s, 1H), 9.19-8.92 (m, 1H), 7.97 (dd, J = 9.3, 5.9 Hz, 1H), 7.45 (dd, J = 9.6, 8.4 Hz, 1H), 7.39 (d, J = 2.6 Hz, |
| | 1H), 7.31-7.12 (m, 4H), 7.04 (s, 1H), 6.39 (s, 1H), 4.75 (s, 5H), 4.47-4.18 (m, 3H), 4.17-3.57 (m, 3H), 3.54-3.43 (m, 2H), |
| | 3.38 (s, 2H), 3.30 (s, 2H), 3.09 (p, J = 6.8 Hz, 1H), 2.98-2.87 (m, 4H), 2.77 (d, J = 10.4 Hz, 2H), 2.32-2.17 (m, 2H), 1.91 |
| | (d, J = 10.8 Hz, 1H), 1.85-1.75 (m, 4H), 1.55 (s, 4H), 1.14 (s, 6H), 1.12 (s, 2H), 0.97 (s, 2H), 0.62 (s, 2H), 0.39 (s, 2H). |
| 188 | 10.43 (s, 1H), 10.10 (s, 1H), 9.80 (s, 1H), 9.54 (d, J = 9.0 Hz, 1H), 9.20-8.93 (m, 1H), 7.97 (dd, J = 9.2, 6.0 Hz, 1H), 7.45 |
| | (t, J = 9.0 Hz, 1H), 7.40-7.32 (m, 3H), 7.28 (d, J = 8.2 Hz, 2H), 7.17 (dd, J = 17.4, 2.5 Hz, 1H), 6.62 (s, 1H), 6.33 (d, |
| | J = 2.4 Hz, 1H), 4.95-4.59 (m, 2H), 4.39-4.04 (m, 3H), 3.97-3.89 (m, 1H), 3.81-3.57 (m, 1H), 3.51-3.44 (m, 5H), |
| | 2.96-2.85 (m, 3H), 2.81 (d, J = 10.7 Hz, 2H), 2.45-2.22 (m, 10H), 2.11 (s, 1H), 1.90 (s, 3H), 1.79-1.62 (m, 3H), |
| | 1.38 (t, J = 11.7 Hz, 2H), 1.31 (d, J = 7.1 Hz, 3H), 0.83 (d, J = 6.9 Hz, 6H), 0.63 (s, 2H), 0.40 (s, 2H). |
| 189 | 10.61 (s, 1H), 10.14 (s, 1H), 9.75 (s, 1H), 8.94 (t, J = 5.9 Hz, 1H), 7.97 (dd, J = 9.2, 5.9 Hz, 1H), 7.46 (t, J = 9.0 Hz, |
| | 1H), 7.38 (d, J = 2.6 Hz, 1H), 7.35 (d, J = 8.2 Hz, 2H), 7.28 (d, J = 8.1 Hz, 2H), 7.22 (d, J = 2.5 Hz, 1H), 6.56 (s, |
| | 1H), 6.33 (s, 1H), 4.26 (s, 2H), 4.13 (s, 1H), 3.88 (d, J = 2.3 Hz, 5H), 3.51 (s, 2H), 3.45 (s, 2H), 3.42 (s, 1H), |
| | 3.16 (p, J = 7.0 Hz, 1H), 2.88 (p, J = 6.9 Hz, 2H), 2.67 (t, J = 2.0 Hz, 1H), 2.45-2.32 (m, 6H), 2.32-2.18 (m, 4H), 1.80-1.49 |
| | (m, 4H), 1.03 (t, J = 7.2 Hz, 3H), 0.79 (d, J = 6.9 Hz, 6H), 0.62 (s, 2H), 0.40 (s, 2H). |
| 190 | 10.28-9.92 (m, 2H), 9.60 (s, 1H), 7.97 (dd, J = 9.2, 5.9 Hz, 1H), 7.46 (t, J = 9.0 Hz, 1H), 7.38 (d, J = 2.6 Hz, 1H), 7.33- |
| | 7.08 (m, 4H), 7.04 (s, 1H), 6.39 (s, 1H), 4.75 (s, 4H), 4.24 (s, 2H), 4.11 (s, 1H), 3.88 (d, J = 2.5 Hz, 5H), 3.47 (s, 2H), |
| | 3.41 (s, 2H), 3.37 (s, 1H), 3.30 (s, 1H), 3.09 (p, J = 6.9 Hz, 1H), 2.44-2.15 (m, 10H), 1.71-1.42 (m, 4H), 1.13 (d, J = |
| | 6.9 Hz, 6H), 0.61 (d, J = 5.3 Hz, 2H), 0.38 (s, 2H). |
| 191 | 10.12 (s, 2H), 9.60 (s, 1H), 8.00-7.92 (m, 1H), 7.45 (dd, J = 9.6, 8.4 Hz, 1H), 7.39-7.34 (m, 1H), 7.30-7.10 (m, 4H), 7.03 (s, 1H), |
| | 6.39 (s, 1H), 4.74 (s, 4H), 4.34-4.15 (m, 2H), 4.08 (s, 1H), 3.86 (d, J = 1.9 Hz, 5H), 3.70 (d, J = 10.9 Hz, 1H), 3.45 (s, 4H), 3.38 (s, |
| | 2H), 3.36 (s, 2H), 3.32 (s, 4H), 3.08 (p, J = 7.0 Hz, 1H), 2.70 (d, J = 10.9 Hz, 1H), 2.57-2.54 (m, 1H), 1.96 (s, 1H), 1.77 (s, 1H), |
| | 1.59 (s, 4H), 1.12 (d, J = 6.9 Hz, 6H), 0.52 (s, 2H), 0.42 (s, 2H). |
| 192 | 10.09 (s, 2H), 9.59 (s, 1H), 7.96 (dd, J = 9.2, 5.9 Hz, 1H), 7.45 (t, J = 9.0 Hz, 1H), 7.36 (d, J = 2.6 Hz, 1H), 7.25- |
| | 7.16 (m, 4H), 7.04 (d, J = 8.8 Hz, 1H), 6.40 (s, 1H), 4.96 (s, 1H), 4.80-4.65 (m, 4H), 4.03-3.88 (m, 3H), |
| | 3.84 (d, J = 12.3 Hz, 3H), 3.79-3.67 (m, 4H), 3.56 (d, J = 13.8 Hz, 2H), 3.50-3.37 (m, 4H), 3.26-3.22 (m, 1H), 3.21- |
| | 3.15 (m, 2H), 3.14 (dd, J = 7.2 Hz, 1H), 2.96 (d, J = 8.0 Hz, 1H), 2.67 (dd, J = 3.8, 2.0 Hz, 2H), 2.12 (s, 1H), 1.83 |
| | (s, 1H), 1.58 (s, 4H), 1.13 (d, J = 6.9 Hz, 6H), 0.51-0.41 (m, 2H), 0.34 (s, 2H). |
| 193 | 10.10 (s, 2H), 9.61 (s, 1H), 9.04 (s, 1H), 7.97 (dd, J = 9.3, 5.9 Hz, 1H), 7.45 (t, J = 9.0 Hz, 1H), 7.38 (d, J = 2.6 Hz, 1H), 7.21- |
| | 7.14 (m, 4H), 7.04 (s, 1H), 6.39 (s, 1H), 4.74 (s, 4H), 4.55-4.38 (m, 3H), 4.31 (d, J = 12.1 Hz, 1H), 3.93 (d, J = 1.1 |
| | Hz, 1H), 3.67-3.55 (m, 2H), 3.52-3.50 (m, 4H), 3.27 (d, J = 7.2 Hz, 2H), 3.20-3.17 (m, 3H), 3.09 (p, J = 6.9 Hz, 1H), 3.01 (d, J = |
| | 5.7 Hz, 2H), 2.88 (t, J = 5.9 Hz, 2H), 2.77 (p, J = 6.7 Hz, 1H), 1.64 (s, 4H), 1.13 (d, J = 6.9 Hz, 6H). |
| 194 | 10.14 (s, 2H), 9.59 (s, 1H), 9.04 (s, 1H), 7.96 (dd, J = 9.2, 5.9 Hz, 1H), 7.45 (t, J = 9.0 Hz, 1H), 7.38 (d, J = 2.6 Hz, |
| | 1H), 7.17 (d, J = 2.6 Hz, 1H), 7.04 (s, 1H), 6.93 (s, 1H), 6.54 (d, J = 8.5 Hz, 2H), 6.37 (s, 1H), 5.64 (s, 1H), 4.65 (s, 2H), |
| | 4.59 (s, 2H), 4.50-4.47 (m, 3H), 4.32-4.29 (m, 1H), 3.92 (d, J = 1.1 Hz, 1H), 3.66-3.56 (m, 1H), 3.53 (d, J = 5.9 Hz, 3H), 3.34 |
| | (s, 2H), 3.08 (p, J = 6.9 Hz, 1H), 3.04-2.89 (m, 4H), 2.83 (q, J = 6.7 Hz, 1H), 2.40-2.29 (m, 2H), 1.64 (s, 4H), 1.12 (d, J = 6.9 |
| | Hz, 6H), 0.40-0.21 (m, 4H). |
| 195 | 10.12 (s, 1H), 9.01 (s, 1H), 8.95 (t, J = 5.9 Hz, 1H), 7.88 (dd, J = 8.0, 1.7 Hz, 1H), 7.47-7.39 (m, 2H), 7.33 (d, J = 2.5 Hz, |
| | 1H), 7.21 (d, J = 8.4 Hz, 2H), 7.11 (d, J = 2.6 Hz, 1H), 7.08 (s, 2H), 7.06 (d, J = 1.8 Hz, 1H), 6.48 (s, 1H), 4.49 (d, J = 11.8 Hz, |
| | 1H), 4.27 (d, J = 10.0 Hz, 3H), 3.79 (s, 3H), 3.67-3.60 (m, 1H), 3.55 (d, J = 12.8 Hz, 4H), 3.45 (s, 3H), 3.39 (s, 2H), 3.23- |
| | 3.14 (m, 2H), 3.08 (p, J = 6.8 Hz, 1H), 2.69 (d, J = 10.5 Hz, 2H), 2.43-2.30 (m, 5H), 2.28 (d, J = 9.0 Hz, 6H), 2.04 (d, J = |
| | 7.0 Hz, 2H), 1.85 (t, J = 11.2 Hz, 2H), 1.65 (s, 4H), 1.59 (d, J = 12.8 Hz, 2H), 1.48-1.32 (m, 2H), 1.23 (s, 3H), 1.08-1.05 |
| | (m, 6H), 1.04 (d, J = 2.1 Hz 3H), 0.62 (s, 2H), 0.39 (s, 2H). |
| 196 | 10.13 (s, 1H), 9.02 (d, J = 1.6 Hz, 1H), 8.02-7.90 (m, 1H), 7.45 (t, J = 9.0 Hz, 1H), 7.38 (dd, J = 2.7, 1.1 Hz, 1 H), 7.33-7.25 |
| | (m, 1 H), 7.23-7.12 (m, 3 H), 7.04 (s, 1 H), 6.69 (d, J = 1.3 Hz, 1 H), 4.75 (s, 2 H), 4.53-4.42 (m, 3 H), 4.33-4.21 (m, 3H), 3.91 |
| | (t, J = 1.4 Hz, 1H), 3.88 (s, 3 H), 3.82 (s, 3 H), 3.62 (d, J = 12.2 Hz, 1H), 3.53 (s, 3H), 3.39 (d, J = 13.0 Hz, 2H), 3.16 (p, J = 6.9 |
| | Hz, 1 H), 2.96 (d, J = 10.5 Hz, 2H), 2.67 (q, J = 1.9 Hz, 1 H), 2.41 (s, 4H), 2.34-2.23 (m, 6H), 2.07 (s, 1H), 1.89-1.74 |
| | (m, 2H), 1.65 (s, 6H), 1.31 (d, J = 12.3 Hz, 2 H), 1.19-1.08 (m, 6 H), 0.62 (s, 2H), 0.38 (s, 2H). |
| 197 | 10.62 (s, 1H), 10.12 (s, 1H), 9.75 (s, 1H), 9.13-9.01 (m, 1H), 8.94 (t, J = 5.9 Hz, 1H), 7.88 (d, J = 8.1 Hz, 1H), 7.48- |
| | 7.38 (m, 2H), 7.38-7.32 (m, 3H), 7.28 (d, J = 8.4 Hz, 2H), 7.14-7.07 (m, 1H), 6.57 (s, 1H), 6.34 (s, 1H), 4.98-4.57 |
| | (m, 1H), 4.31 (d, J = 10.5 Hz, 2H), 4.22 (d, J = 10.8, 4.1 Hz, 1H), 4.06 (d, J = 12.7 Hz, 1H), 3.79 (d, J = 12.2 Hz, 1H), |
| | 3.65-3.49 (m, 2H), 3.46 (s, 3H), 3.35-3.27 (m, 2H), 3.16 (dd, J = 7.4, 6.0 Hz, 2H), 2.90 (dd, J = 13.8, 6.9 Hz, 3H), |
| | 2.79 (d, J = 10.6 Hz, 2H), 2.39-2.20 (m, 10H), 2.07 (d, J = 6.8 Hz, 2H), 1.92 (t, J = 10.9 Hz, 3H), 1.78-1.67 (m, 1H), |
| | 1.62 (d, J = 12.3 Hz, 2H), 1.45 (s, 1H), 1.04 (q, J = 7.0 Hz, 3H), 0.80 (d, J = 6.9 Hz, 6H), 0.63 (s, 2H), 0.40 (s, 2H). |
| 198 | 10.15 (s, 1 H), 9.02 (s, 1H), 8.97 (t, J = 5.9 Hz, 1H), 7.97 (dd, J = 9.2, 5.9 Hz, 1 H), 7.46 (t, J = 9.0 Hz, 1H), 7.38 |
| | (d, J = 2.5 Hz, 1H), 7.21 (d, J = 8.4 Hz, 2H), 7.16 (d, J = 2.5 Hz, 1 H), 7.10-7.04 (m, 3 H), 6.48 (s, 1H), 4.48 (d, J = 12.1 |
| | Hz, 1H), 4.27 (d, J = 13.0 Hz, 3H), 3.93 (d, J = 1.0 Hz, 1H), 3.79 (s, 3 H), 3.63 (d, J = 11.9 Hz, 1H), 3.53 (d, |
| | J = 7.4 Hz, 3H), 3.45 (s, 3 H), 3.39 (s, 2 H), 3.23-3.14 (m, 2 H), 3.08 (p, J = 6.9 Hz, 1 H), 2.68 (t, J = 7.0 Hz, 2 |
| | H), 2.45-2.20 (m, 12H), 2.04 (d, J = 7.1 Hz, 2 H), 1.89-1.79 (m, 2 H), 1.65 (s, 4H), 1.59 (d, J = 12.7 Hz, |
| | 2H), 1.41 (s, 1H), 1.11-0.96 (m, 9H), 0.62 (s, 2 H), 0.39 (s, 2 H). |
| 199 | 9.03 (d, J = 1.7 Hz, 1H), 8.21 (s, 2H), 7.97 (ddd, J = 9.3, 5.9, 1.3 Hz, 1H), 7.51-7.42 (m, 1H), 7.39 (dd, J = 2.7, |
| | 1.3 Hz, 1H), 7.33-7.23 (m, 1H), 7.22-7.09 (m, 3H), 7.04 (s, 1H), 6.69 (d, J = 1.7 Hz, 1H), 4.75 (s, 2H), 4.48 |
| | (d, J = 5.4 Hz, 3H), 4.32 (d, J = 12.4 Hz, 1H), 4.28-4.20 (m, 2H), 3.93 (t, J = 1.4 Hz, 1H), 3.88 (s, 3H), 3.85 (s, 3H), |
| | 3.67 (s, 4H), 3.60 (d, J = 12.8 Hz, 2H), 3.39 (d, J = 13.7 Hz, 2H), 3.23-3.10 (m, 1H), 2.99 (d, J = 10.5 Hz, 2H), 2.78 (t, |
| | J = 10.4 Hz, 2H), 2.36-2.21 (m, 2H), 1.80 (t, J = 11.0 Hz, 4H), 1.72 (s, 4H), 1.56 (d, J = 9.9 Hz, 5H), 1.18-1.12 (m, |
| | 5H), 1.12 (s, 2H), 0.97 (s, 2H), 0.63 (s, 2H), 0.40 (s, 2H). |
| 200 | 10.10 (s, 1H), 9.50 (d, J = 9.0 Hz, 1H), 8.95 (s, 1H), 7.89 (dd, J = 9.2, 5.9 Hz, 1H), 7.38 (t, J = 9.0 Hz, 1H), 7.32 (d, J = 2.6 |
| | Hz, 1H), 7.16 (d, J = 8.1 Hz, 2H), 7.10 (d, J = 2.5 Hz, 1H), 7.03 (d, J = 8.1 Hz, 3H), 6.41 (s, 1H), 4.59 (h, J = 7.5 Hz, 1H), |
| | 4.40 (d, J = 12.1 Hz, 1H), 4.21 (d, J = 15.3 Hz, 3H), 3.85 (s, 1H), 3.71 (s, 3H), 3.56 (d, J = 12.0 Hz, 1H), 3.47 (t, J = 5.3 |
| | Hz, 3H), 3.38 (s, 3H), 3.37-3.36 (m, 2H), 3.01 (hept, J = 7.0 Hz, 1H), 2.95-2.81 (m, 2H), 2.34 (s, 4H), 2.20 (s, 6H), 2.07-1.94 |
| | (m, 1H), 1.74 (t, J = 11.3 Hz, 2H), 1.58 (d, J = 11.0 Hz, 6H), 1.25 (t, J = 7.8 Hz, 5H), 0.98 (dd, J = 6.9, 1.5 Hz, 6H), |
| | 0.56 (d, J = 5.2 Hz, 2H), 0.32 (d, J = 4.8 Hz, 2H). |
| 201 | 10.62 (s, 1H), 10.18-9.68 (m, 1H), 9.16-9.02 (m, 1H), 8.95 (t, J = 5.9 Hz, 1H), 7.97 (dd, J = 9.2, 6.0 Hz, 1H), 7.46 |
| | (t, J = 9.0 Hz, 1H), 7.39 (d, J = 2.5 Hz, 1H), 7.36 (d, J = 8.1 Hz, 2H), 7.28 (d, J = 8.1 Hz, 2H), 7.22-7.12 (m, 1H), 6.57 (s, |
| | 1H), 6.34 (s, 1H), 5.76 (s, 1H), 4.99-4.57 (m, 1H), 4.34-4.17 (m, 2H), 3.99 (s, 1H), 3.92 (s, 1H), 3.83-3.59 (m, 1H), 3.46 |
| | (s, 4H), 3.19-3.15 (m, 2H), 3.14-3.11 (m, 2H), 2.93-2.85 (m, 2H), 2.79 (d, J = 10.5 Hz, 2H), 2.45-2.18 |
| | (m, 12H), 2.06 (d, J = 7.0 Hz, 2H), 1.91 (t, J = 10.5 Hz, 2H), 1.73 (s, 1H), 1.62 (d, J = 12.4 Hz, 2H), 1.45 (s, 1H), 1.09 |
| | (s, 1H), 1.03 (t, J = 7.2 Hz, 3H), 0.80 (d, J = 6.9 Hz, 6H), 0.63 (s, 2H), 0.40 (s, 2H). |
| 202 | 10.15 (s, 2H), 9.60 (s, 1H), 9.02 (s, 1H), 8.19 (s, 1H), 7.97 (dd, J = 9.2, 5.9 Hz, 1H), 7.45 (t, J = 9.0 Hz, 1H), 7.38 (d, J = |
| | 2.6 Hz, 1H), 7.24 (s, 2H), 7.21 (m, 2H), 7.04 (s, 1H), 6.39 (s, 1H), 4.75 (s, 4H), 4.46 (d, J = 12.1 Hz, 1H), 4.30 (d, J = |
| | 12.0 Hz, 1H), 4.18 (s, 2H), 3.93 (s, 1H), 3.62 (d, J = 12.0 Hz, 1H), 3.55 (d, J = 10.9 Hz, 3H), 3.43 (s, 2H), 3.37 (s, |
| | 2H), 3.13 (m, 1H), 2.87 (m, 1H), 2.76 (s, 2H), 2.40 (s, 2H), 2.30 (s, 2H), 2.20 (s, 6H), 1.66 (s, 4H), 1.13 (d, J = 6.8 |
| | Hz, 6H), 0.51 (s, 2H), 0.43 (s, 2H). |
| 203 | 10.41 (s, 1H), 10.18 (s, 1H), 9.60 (s, 1H), 9.54 (d, J = 9.0 Hz, 1H), 9.03 (s, 1H), 8.22 (s, 1H), 7.97 (dd, J = 9.2, 5.9 Hz, 1H), 7.50 |
| | (s, 1H), 7.45 (t, J = 9.0 Hz, 1H), 7.41 (m, 2H), 7.31 (m, 2H), 7.17 (d, J = 2.6 Hz, 1H), 6.62 (s, 1H), 6.33 (s, 1H), 4.63 (dt, J = |
| | 15.4, 7.6 Hz, 1H), 4.47 (d, J = 12.0 Hz, 1H), 4.30 (d, J = 12.1 Hz, 1H), 4.19 (s, 2H), 3.93 |
| | (d, J = 1.1 Hz, 1H), 3.63 (d, J = 11.9 Hz, 1H), 3.56 (d, J = 11.6 Hz, 3H), 3.47 (s, 2H), 3.35 (, 2H), 2.96 (m 1H), 2.84 |
| | (s, 1H), 2.71 (s, 2H), 2.41 (d, J = 2.7 Hz, 2H), 2.37 (s, 3H), 1.97 (s, 6H), 1.67 (s, 4H), 1.31 (d, J = 7.1 Hz, 3H), |
| | 0.82 (d, J = 6.8 Hz, 6H), 0.51 (s, 2H), 0.44 (s, 2H). |
| 204 | 10.07 (s, 2H), 9.01 (d, J = 4.8 Hz, 1H), 7.96 (ddd, J = 8.8, 5.8, 2.6 Hz, 1H), 7.45 (td, J = 9.0, 2.0 Hz, 1H), 7.37 (t, J = |
| | 3.0 Hz, 1H), 7.30-7.08 (m, 4H), 7.03 (s, 1H), 6.38 (s, 1H), 4.72 (s, 4H), 4.50-4.30 (m, 2H), 4.29-4.09 (m, 3H), 3.94 |
| | (s, 1H), 3.60 (m, 1H), 3.53 (d, J = 10.6 Hz, 3H), 3.45 (m, 1H), 3.08 (p, J = 6.8 Hz, 1H), 2.95 (d, J = 8.2 Hz, 1H), |
| | 2.81 (s, 1H), 2.21 (m, 1H), 1.96 (s, 1H), 1.65 (m, 7H), 1.12 (d, J = 6.8 Hz, 6H). |
| 205 | 10.17 (s, 1H), 9.58 (d, J = 9.0 Hz, 1H), 9.02 (s, 1H), 7.97 (dd, J = 9.2, 6.0 Hz, 1H), 7.45 (t, J = 9.0 Hz, 1H), 7.39 |
| | (d, J = 2.5 Hz, 1H), 7.27-7.19 (m, 2H), 7.16 (d, J = 2.5 Hz, 1H), 7.13-7.01 (m, 3H), 6.48 (s, 1H), 4.66 (h, J = |
| | 7.6 Hz, 1H), 4.48 (d, J = 12.0 Hz, 1H), 4.34-4.20 (m, 3H), 3.92 (d, J = 1.1 Hz, 1H), 3.78 (s, 3H), 3.63 (d, J = 11.9 Hz, 1H), |
| | 3.55 (d, J = 12.1 Hz, 3H), 3.46 (s, 3H), 3.41 (s, 4H), 3.08 (p, J = 6.8 Hz, 1H), 2.97 (d, J = 10.6 Hz, 2H), 2.78-2.62 (m, 2H), 2.27 |
| | (s, 2H), 1.77 (q, J = 10.7 Hz, 4H), 1.67 (s, 4H), 1.55 (t, J = 10.8 Hz, 4H), 1.33 (d, J = 7.0 Hz, 3H), 1.09 (d, J = 11.6 |
| | Hz, 2H), 1.04 (dd, J = 6.9, 1.6 Hz, 6H), 0.97 (d, J = 11.0 Hz, 2H), 0.63 (d, J = 4.9 Hz, 2H), 0.39 (d, J = 4.9 Hz, 2H). |
| 206 | 10.44 (s, 1H), 9.87 (s, 2H), 9.57 (d, J = 8.9 Hz, 1H), 9.03 (s, 1H), 7.96 (dd, J = 9.2, 5.9 Hz, 1H), 7.45 (t, J = 9.0 Hz, 1H), |
| | 7.40-7.32 (m, 3H), 7.26 (d, J = 8.1 Hz, 2H), 7.17 (t, J = 2.6 Hz, 1H), 6.61 (d, J = 2.9 Hz, 1H), 6.32 (s, 1H), |
| | 4.63 (h, J = 7.5 Hz, 1H), 4.48 (dt, J = 9.2, 4.0 Hz, 2H), 4.28 (m, 3H), 3.98-3.84 (m, 1H), 3.63 (d, J = 12.0 Hz, 1H), 3.52 (s, 2H), |
| | 3.48-3.42 (m, 1H), 3.02 (q, J = 6.8, 6.2 Hz, 1H), 2.86 (m, 2H), 2.74-2.57 (m, 1H), 2.19 (td, J = 9.1, 4.5 Hz, 1H), |
| | 2.04-1.91 (m, 1H), 1.88-1.45 (m, 7H), 1.30 (d, J = 7.0 Hz, 3H), 0.80 (dt, J = 5.5, 2.7 Hz, 6H). |
| 207 | 10.13 (s, 2H), 9.61 (s, 1H), 9.15-8.97 (m, 1H), 7.97 (dd, J = 9.3, 5.8 Hz, 1H), 7.45 (t, J = 9.0 Hz, 1H), 7.40-7.33 (m, 1H), |
| | 7.31-7.12 (m, 4H), 7.04 (s, 1H), 6.39 (s, 1H), 5.08-4.56 (m, 5H), 4.33-4.16 (m, 2H), 4.11-4.00 (m, 1H), 3.91 (d, J = 1.1 |
| | Hz, 1H), 3.82-3.57 (m, 1H), 3.47 (d, J = 12.8 Hz, 4H), 3.09 (p, J = 6.9 Hz, 1H), 2.96 (s, 2H), 2.89 (d, J = 10.0 |
| | Hz, 1H), 2.50 (q, J = 1.8 Hz, 2H), 2.44-2.39 (m, 4H), 2.32-2.18 (m, 5H), 2.14-1.97 (m, 2H), 1.90 (d, J = 10.8 |
| | Hz, 1H), 1.83-1.72 (m, 3H), 1.65 (d, J = 11.5 Hz, 2H), 1.33 (s, 2H), 1.13 (d, J = 6.9 Hz, 6H), 0.63 (s, 2H), 0.39 (s, 2H). |
| 208 | 10.10 (m, 2H), 9.62 (s, 1H), 9.03 (s, 1H), 7.97 (dd, J = 9.2, 5.8 Hz, 1H), 7.46 (t, J = 9.0 Hz, 1H), 7.38 (d, J = 2.6 |
| | Hz, 1H), 7.32-7.11 (m, 4H), 7.03 (s, 1H), 6.39 (s, 1H), 4.75 (s, 4H), 4.50 (t, J = 9.4 Hz, 1H), 4.33 (d, J = 12.1 |
| | Hz, 1H), 4.26 (s, 2H), 3.94 (d, J = 1.0 Hz, 1H), 3.71-3.53 (m, 4H), 3.47 (d, J = 4.4 Hz, 2H), 3.40 (s, 2H), 3.08 |
| | (h, J = 6.8 Hz, 1H), 2.96 (d, J = 10.4 Hz, 2H), 2.40 (s, 6H), 2.34-2.28 (m, 2H), 2.27-2.21 (m, 2H), 2.08 (s, |
| | 1H), 1.90 (s, 2H), 1.79 (t, J = 11.6 Hz, 2H), 1.64 (d, J = 10.4 Hz, 4H), 1.30 (m, 2H), 1.13 (d, J = 6.8 Hz, 6H), |
| | 0.62 (s, 2H), 0.38 (s, 2H). |
| 209 | 10.33-9.89 (m, 2H), 9.62 (s, 1H), 9.03 (s, 1H), 8.18 (s, 1H), 7.97 (dd, J = 9.2, 5.9 Hz, 1H), 7.46 (t, J = 9.0 Hz, 1H), 7.38 |
| | (d, J = 2.6 Hz, 1H), 7.36-7.24 (m, 1H), 7.16 (d, J = 2.5 Hz, 2H), 7.02 (s, 1H), 6.39 (s, 1H), 4.72 (s, 4H), |
| | 4.49 (d, J = 12.1 Hz, 1H), 4.34-4.20 (m, 3H), 3.93 (d, J = 1.1 Hz, 1H), 3.69-3.52 (m, 4H), 3.44 (s, 2H), 3.08 (p, J = 6.9 |
| | Hz, 1H), 2.97 (d, J = 10.4 Hz, 2H), 2.48-2.46 (m, 3H), 2.42 (s, 3H), 2.33 (p, J = 2.7, 1.9 Hz, 3H), 2.27 (s, 2H), 2.09 (d, J = 8.0 Hz, |
| | 1H), 1.81 (t, J = 11.5 Hz, 2H), 1.72-1.59 (m, 6H), 1.31 (d, J = 12.3 Hz, 2H), 1.13 (d, J = 6.9 Hz, 6H), 0.63 (s, 2H), 0.39 (s, 2H). |
| 210 | 10.12 (m, 2H), 9.62 (s, 1H), 7.97 (dd, J = 9.2, 5.9 Hz, 1 H), 7.46 (t, J = 9.0 Hz, 1H), 7.38 (d, J = 2.5 Hz, 1 H), |
| | 7.22 (q, J = 13.1, 11.0 Hz, 4 H), 7.03 (s, 1 H), 6.39 (s, 1 H), 5.17 (p, J = 5.8 Hz, 1H), 4.75 (s, 4H), 4.04 (s, 1 H), |
| | 3.88 (d, J = 5.9 Hz, 5H), 3.78 (t, J = 7.2 Hz, 2 H), 3.62 (s, 2H), 3.47 (s, 2 H), 3.33 (s, 2H), 3.18 (t, J = 6.8 Hz, 2H), |
| | 3.13-3.08 (m, 2H), 3.05 (t, J = 6.9 Hz, 3 H), 2.87 (m, 2 H), 1.58 (s, 4H), 1.13 (d, J = 6.9 Hz, 6H). |
| 211 | 9.02 (s, 1H), 8.19 (s, 1H), 8.03 (s, 1H), 7.97 (dd, J = 9.2, 5.9 Hz, 1H), 7.45 (t, J = 9.0 Hz, 1H), 7.38 (d, J = 2.6 |
| | Hz, 1H), 7.31 (s, 1H), 7.16 (d, J = 2.6 Hz, 1H), 6.69 (s, 2H), 5.26 (s, 2H), 4.51-4.48 (m, 1H), 4.34-4.18 (m, |
| | 3H), 3.92 (d, J = 1.1 Hz, 1H), 3.71 (s, 3H), 3.68-3.50 (m, 6H), 3.45 (s, 2H), 2.51 (s, 2H), 2.40 (s, 4H), 2.38-2.25 |
| | (m, 2H), 2.24 (s, 3H), 2.13 (s, 3H), 1.67 (s, 4H), 0.64 (d, J = 4.8 Hz, 2H), 0.41 (s, 2H). |
| 213 | 10.07 (s, 2H), 9.63 (s, 1H), 9.04 (s, 1H), 8.19 (s, 1H), 7.97 (dd, J = 9.2, 5.9 Hz, 1H), 7.46 (t, J = 9.0 Hz, 1H), |
| | 7.39 (d, J = 2.6 Hz, 1H), 7.18 (q, J = 14.2, 8.0 Hz, 4H), 7.02 (s, 1H), 6.39 (s, 1H), 4.73 (s, 4H), 4.48 (d, J = 12.2 |
| | Hz, 1H), 4.31 (d, J = 11.9 Hz, 1H), 4.18 (s, 2H), 3.95 (d, J = 1.1 Hz, 1H), 3.71-3.54 (m, 4H), 3.50 (s, 2H), |
| | 3.35 (s, 2H), 3.21 (s, 2H), 3.08 (p, J = 6.9 Hz, 1H), 2.90-2.73 (m, 4H), 2.67 (p, J = 1.9 Hz, 2H), 2.41 (s, 2H), 1.68 |
| | (s, 4H), 1.13 (d, J = 6.9 Hz, 6H), 0.52 (s, 2H), 0.43 (s, 2H). |
| 214 | 10.13-10.07 (m, 2H), 9.61 (s, 1H), 9.01 (s, 1H), 7.88 (dd, J = 8.1, 1.7 Hz, 1H), 7.47-7.36 (m, 2H), 7.33 (d, J = 2.6 Hz, 1H), |
| | 7.27-7.19 (m, 3H), 7.11 (d, J = 2.6 Hz, 1H), 7.04 (s, 1H), 6.39 (s, 1H), 4.76 (s, 4H), 4.48 (d, J = 12.0 Hz, 1H), 4.27-4.25 |
| | (m, 3H), 3.63 (d, J = 12.0 Hz, 1H), 3.58 (s, 1H), 3.53-3.51 (m, 3H), 3.40 (s, 2H), 3.09 (hept, J = 6.9 Hz, 1H), 2.96 (d, J = 10.6 |
| | Hz, 2H), 2.41 (s, 4H), 2.30-2.26 (m, 6H), 2.14-2.00 (m, 1H), 1.80 (t, J = 11.4 Hz, 2H), 1.64 (s, 6H), 1.34-1.29 (m, 2H), 1.13 |
| | (d, J = 6.9 Hz, 6H), 0.63 (s, 2H), 0.39 (s, 2H). |
| 215 | 10.07 (s, 2H), 9.62 (s, 1H), 9.04 (s, 1H), 8.18 (s, 1H), 7.97 (dd, J = 9.2, 5.9 Hz, 1H), 7.46 (t, J = 9.0 Hz, 1H), 7.39 (d, J = 2.6 Hz, |
| | 1H), 7.32-7.15 (m, 4H), 7.04 (s, 1H), 6.39 (s, 1H), 4.75 (s, 4H), 4.48 (d, J = 12.2 Hz, 1H), 4.33 (d, J = 12.1 Hz, 1H), |
| | 4.25-4.14 (m, 2H), 3.93 (s, 1H), 3.62 (q, J = 12.4 Hz, 4H), 3.20 (s, 4H), 3.13-3.05 (m, 1H), 2.87 (s, 2H), 2.74 (d, J = 10.7 Hz, 2H), |
| | 2.45 (s, 2H), 2.14-2.05 (m, 1H), 1.83 (t, J = 11.2 Hz, 2H), 1.69 (s, 6H), 1.50 (d, J = 12.3 Hz, 2H), 1.32 (d, J = 11.3 Hz, 1H), |
| | 1.13 (d, J = 6.9 Hz, 6H), 1.04-0.94 (m, 2H), 0.53 (d, J = 5.5 Hz, 2H), 0.45 (s, 2H). |
| 216 | 10.06 (d, J = 58.5 Hz, 2H), 9.60 (s, 1H), 9.02 (s, 1H), 7.96 (dd, J = 9.2, 5.9 Hz, 1H), 7.64 (d, J = 26.5 Hz, 1H), 7.45 |
| | (t, J = 9.0 Hz, 1H), 7.38 (d, J = 2.6 Hz, 1H), 7.16 (d, J = 2.6 Hz, 1H), 7.02 (s, 1H), 6.67 (d, J = 8.4 Hz, 1H), |
| | 6.40 (s, 1H), 4.88 (s, 1H), 4.63 (s, 4H), 4.47 (d, J = 12.1 Hz, 1H), 4.28 (d, J = 11.9 Hz, 3H), 3.93 (d, J = 1.1 Hz, |
| | 1H), 3.62 (d, J = 12.0 Hz, 1H), 3.54 (s, 3H), 3.09 (p, J = 6.9 Hz, 1H), 2.99 (s, 2H), 2.80-2.63 (m, 2H), 2.37- |
| | 2.22 (m, 4H), 2.18 (s, 1H), 1.92 (s, 2H), 1.81 (t, J = 11.2 Hz, 2H), 1.66 (s, 6H), 1.56 (s, 2H), 1.36 (s, 2H), 1.13 (d, |
| | J = 6.9 Hz, 6H), 0.63 (s, 2H), 0.39 (s, 2H). |
| 218 | 10.13 (s, 1H), 10.05 (s, 1H), 9.58 (s, 1H), 9.03 (s, 1H), 7.97 (dd, J = 9.2, 5.9 Hz, 1H), 7.45 (t, J = 9.0 Hz, 1H), |
| | 7.38 (d, J = 2.5 Hz, 1H), 7.17 (d, J = 2.6 Hz, 2H), 7.03 (s, 1H), 6.91 (s, 1H), 6.86-6.75 (m, 1H), 6.38 (s, 1H), 4.70 |
| | (d, J = 16.2 Hz, 4H), 4.47 (d, J = 12.1 Hz, 1H), 4.30 (d, J = 12.1 Hz, 1H), 4.19 (s, 2H), 3.98 (s, 2H), |
| | 3.92 (d, J = 1.1 Hz, 1H), 3.67-3.51 (m, 4H), 3.34-3.31 (m, 2H), 3.09 (p, J = 6.8 Hz, 1H), 2.77 (s, 2H), 2.70-2.60 (m, 2H), |
| | 2.55-2.53 (m, 2H), 2.42-2.29 (m, 2H), 2.16 (s, 3H), 1.67 (s, 4H), 1.13 (d, J = 6.9 Hz, 6H), 0.51 (s, 2H), 0.42 (s, 2H). |

### Results

### Cell Lines

The following cancer cell lines were employed: AGS gastric carcinoma [heterozygous G12D](ATCC, CRL-1739); A-427 lung carcinoma [heterozygous G12D](ATCC, HTB-53); ASPC1 pancreatic adenocarcinoma [homozygous G12D](ATCC, CRL-1682) and SW1990 pancreatic adenocarcinoma [homozygous G12D](ATCC CRL-2172). Cell lines were cultured essentially according to ATCC recommendations.

### KRAS(G12D)/SOS1 Homogeneous Time-Resolved Fluorescence (HTRF) Assay

Binding of test compounds to KRAS(G12D) target protein, which in turn blocks KRAS(G12D) interaction with the SOS1 protein, was measured in the absences of GTP by homogeneous time-resolved fluorescence using the KRAS-G12D/SOS1 Binding Assay Kit (Cisbio, 63ADK000CB17PEH), following the manufacturer's instructions, except as noted. 3-fold serial dilutions of each test compound were prepared ranging from 20 M to 1.02 nM. The test compound was mixed and incubated with reaction components, incubated in a sealed plate at 4° C. for 3 hr and fluorescence was measured using a PerkinElmer Envision plate reader. The KRAS(G12D)-SOS1 IC50 values (the concentration of 50% of the maximal inhibition) were calculated using GraphPad Prism 7 software. Results are listed in Table 2.

### Cancer Cell Line Proliferation (CellTiter-Glo® Assays)

AGS, A-427, ASPC1, SW1990, and GP2D cells were plated in 96-well tissue culture plates at 4,000 cells/well and incubated at 37° C./5% CO2 for 72 hr in 100 μl of media. 3-fold serial dilutions of each test compound were prepared ranging from 20 μM to 1.02 nM. Each cell line was then treated with test compounds at various concentrations with a final concentration of 0.5% DMSO/well, and then incubated at 37° C./5% CO2 for 72 hr. 100 l of CellTiter-Glo® Reagent (Promega Corporation, Madison, WI) was added to each well and processed according manufacturer's protocol. Results were analyzed and EC50 values were calculated in GraphPad 7 software. Results are listed in Table 2.

TABLE 2
Biochemical and Cell-based Assays of Compounds

| | HTRF of | | | | |
|---|---|---|---|---|---|
| | KRAS^{G12D}- | AGS | A427 | ASPC-1 | SW1990 |
| Cmp | SOS1 | CTG | CTG | CTG | CTG |
| 1 | B | B | B | B | B |
| 2 | — | A | A | A | A |
| 3 | A | A | B | | |
| 4 | B | | | C | |
| 5 | A | | C | C | |
| 6 | B | B | B | C | |
| 7 | B | A | B | B | |
| 8 | A | C | C | C | |
| 9 | B | B | C | C | |
| 10 | A | B | B | C | |
| 11 | A | B | B | B | |
| 12 | A | A | A | B | |
| 13 | B | B | B | C | |
| 14 | B | B | B | B | |
| 15 | A | B | B | C | |
| 16 | A | A | A | B | |
| 17 | A | A | A | B | |
| 18 | A | B | B | C | |
| 19 | A | A | B | B | |
| 20 | B | B | B | C | |
| 21 | B | A | B | C | |
| 22 | B | B | B | C | |
| 23 | B | B | B | C | |
| 24 | B | A | A | B | |
| 25 | A | A | B | C | A |
| 26 | A | B | B | C | |
| 27 | A | A | A | B | |
| 28 | A | A | A | B | |
| 29 | B | A | A | B | |
| 30 | C | B | B | C | |
| 31 | A | B | B | C | |
| 32 | A | B | B | C | |
| 33 | A | B | C | C | |
| 34 | C | A | B | B | |
| 35 | C | B | B | B | B |
| 36 | A | A | B | B | |
| 37 | A | B | B | B | |
| 38 | A | B | B | B | |
| 39 | A | B | B | C | |
| 40 | A | B | B | C | |
| 41 | A | B | C | B | |
| 42 | A | C | C | C | |
| 43 | A | C | C | C | |
| 44 | A | B | B | C | |
| 45 | B | B | B | C | |
| 46 | A | — | — | — | — |
| 47 | A | B | | | |
| 48 | | B | | | |
| 49 | A | B | B | C | |
| 50 | A | B | B | B | |
| 51 | B | B | B | B | |
| 52 | C | B | B | C | |
| 53 | A | A | B | B | |
| 54 | A | A | B | B | A |
| 55 | B | B | B | B | B |
| 56 | C | A | B | B | A |
| 57 | C | B | B | C | |
| 58 | C | A | A | B | B |
| 59 | C | A | B | B | |
| 60 | C | A | B | B | |
| 61 | C | B | B | B | |
| 62 | C | A | A | B | |
| 63 | B | B | A | B | |
| 64 | A | B | B | B | |
| 65 | A | A | A | B | |
| 66 | B | A | A | B | |
| 67 | | — | — | — | — |
| 68 | C | B | B | C | |
| 69 | C | B | B | C | |
| 70 | A | B | B | C | |
| 71 | B | B | B | | |
| 72 | B | A | A | | |
| 73 | A | B | B | | |
| 74 | C | B | B | | |
| 75 | B | C | C | | |
| 76 | B | B | B | | |
| 77 | B | A | B | | |
| 78 | C | B | B | | |
| 79 | C | B | B | | |
| 80 | A | C | C | | |
| 81 | C | B | B | | |
| 82 | C | B | B | | |
| 83 | C | A | A | | A |
| 84 | | B | B | | |
| 85 | C | B | B | | |
| 86 | A | B | B | | |
| 87 | A | B | B | | |
| 88 | C | A | B | | |
| 89 | C | A | A | | |
| 90 | | B | B | | |
| 91 | C | B | B | | |
| 92 | B | B | B | | |
| 93 | B | A | B | | |
| 94 | B | B | B | | |
| 95 | A | A | B | | |
| 96 | A | B | B | | |
| 97 | A | B | B | | |
| 98 | A | B | B | | |
| 99 | A | B | B | | |
| 100 | A | A | B | | |
| 101 | A | A | B | | |
| 102 | B | B | B | B | B |
| 103 | C | A | B | B | B |
| 104 | C | B | B | | |
| 105 | C | B | B | | |
| 106 | C | A | B | | |
| 107 | B | B | B | B | B |
| 108 | B | B | B | | |
| 109 | B | A | B | | |
| 110 | | B | B | | |
| 111 | | B | B | | |
| 112 | | C | C | | |
| 113 | | B | B | | |
| 114 | | B | B | | |
| 115 | | B | B | | |
| 116 | | B | C | | |
| 117 | | A | B | B | A |
| 118 | A | A | B | | |
| 119 | A | A | B | | |
| 120 | A | A | B | B | A |
| 121 | A | A | C | C | B |
| 122 | A | B | B | B | B |
| 123 | A | A | B | B | A |
| 124 | A | A | A | B | A |
| 125 | A | A | B | B | A |
| 126 | A | A | A | A | A |
| 127 | A | A | A | A | A |
| 128 | A | B | B | B | B |
| 129 | A | B | B | C | B |
| 130 | A | A | A | B | A |
| 131 | A | A | B | B | A |
| 132 | A | A | A | A | A |
| 133 | A | B | B | B | |
| 134 | A | B | B | B | |
| 135 | A | B | B | B | |
| 136 | B | B | B | C | |
| 137 | B | B | B | B | |
| 138 | | — | — | — | — |
| 139 | A | B | B | C | |
| 140 | A | A | A | A | |
| 141 | A | A | A | B | |
| 142 | B | B | B | B | B |
| 143 | B | A | A | B | A |
| 144 | A | A | B | B | B |
| 145 | A | A | A | B | A |
| 146 | B | A | A | B | A |
| 147 | A | A | A | B | A |
| 148 | A | A | A | A | A |
| 149 | A | A | A | A | A |
| 150 | B | A | A | B | A |
| 151 | A | B | B | B | B |
| 152 | A | A | B | B | A |
| 153 | A | A | A | A | A |
| 154 | A | A | A | A | A |
| 155 | A | B | B | B | B |
| 156 | A | A | B | A | A |
| 157 | A | A | B | B | B |
| 158 | A | A | A | A | A |
| 159 | A | B | B | B | B |
| 160 | | A | A | A | A |
| 161 | A | A | B | B | A |
| 162 | A | A | A | A | A |
| 163 | A | A | B | B | A |
| 164 | A | A | | B | A |
| 165 | A | A | | B | A |
| 166 | A | A | | A | A |
| 167 | A | A | | | A |
| 168 | A | A | | | A |
| 169 | A | A | | | A |
| 170 | A | A | | | A |
| 172 | A | B | | | B |
| 173 | B | B | | | C |
| 174 | A | B | | | C |
| 175 | A | B | | | B |
| 176 | B | B | | | C |
| 177 | A | A | | | A |
| 178 | A | A | | | A |
| 179 | B | B | | | C |
| 180 | A | A | | | A |
| 181 | A | A | | | A |
| 182 | A | A | | | A |
| 183 | A | A | | | A |
| 184 | A | A | | | A |
| 185 | B | B | | | B |
| 186 | A | A | | | A |
| 187 | B | B | | | C |
| 188 | B | B | | | B |
| 189 | A | A | | | A |
| 190 | A | A | | | A |
| 191 | A | A | | | B |
| 192 | C | B | | | B |
| 193 | A | B | | | B |
| 194 | A | B | | | B |
| 195 | | B | | | B |
| 196 | | A | | | |
| 197 | | A | | | B |
| 198 | | | | | |
| 199 | | B | | | B |
| 200 | | B | | | C |
| 201 | | | | | |
| 202 | | B | | | B |
| 203 | | | | | A |
| 204 | | B | | | C |
| 205 | | | | | |
| 206 | | B | | | |
| 207 | | B | | | |
| 208 | | A | | | |
| 209 | | A | | | |
| 210 | | B | | | |
| 211 | | A | | | |
| 213 | | A | | | |
| 214 | | A | | | |
| 215 | | A | | | |
| 216 | | A | | | |
| 218 | | B | | | |

KRAS(G12D)/SOS1 HTRF assay: A. IC50 < 100 nM; B. IC50 = 100-1000 nM; C. IC50 > 1000 nM;
AGS proliferation assay: A. EC50 < 100 nM; B. EC50 = 100-1000 nM; C. EC50 > 1000 nM;
A427 proliferation assay: A. EC50 < 100 nM; B. EC50 = 100-1000 nM; C. EC50 > 1000 nM;
ASPC proliferation assay: A. EC50 < 100 nM; B. EC50 = 100-1000 nM; C. EC50 > 1000 nM;
SW 1990 proliferation assay: A. EC50 < 100 nM; B. EC50 = 100-1000 nM; C. EC50 > 1000 nM

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound having the Formula I or II: 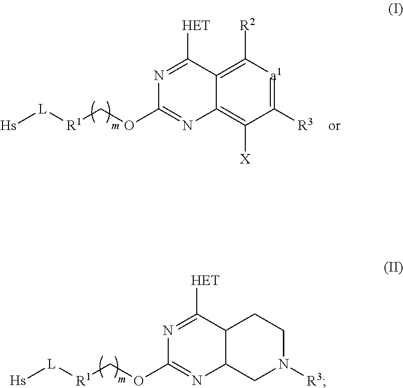
or a pharmaceutically acceptable salt thereof, wherein
HET is an optionally substituted heterocyclyl;
Hs is a chemical moiety that binds HSP90;
X is hydrogen or halo;
L is a linker;
m is 0, 1, 2, or 3;
R¹ is selected from (C₁-C₄)alkylO(C₁-C₄)alkyl, —(C₁-C₄)alkylNH(C₁-C₄)alkyl, —(C₁-C₄)alkylN[(C₁-C₄)alkyl]₂, heterocyclyl, and cycloalkyl, wherein said heterocyclyl and cycloalkyl are each optionally and independently substituted;
R² is selected from hydrogen, (C₁-C₄)alkyl, (C₁-C₄)haloalkyl, (C₁-C₄)alkoxy, (C₁-C₄)haloalkoxy, (C₁-C₄)alkynyl, (C₁-C₄)alkenyl, halo, (C₃-C₆)cycloalkyl, —O(C₃-C₆)cycloalkyl, cyano, NH₂, —NH(C₁-C₄)alkyl, —N[(C₁-C₄)alkyl]₂, —P(O)[(C₁-C₄)alkyl]₂, and —S(C₁-C₄)alkyl;
R³ is selected from 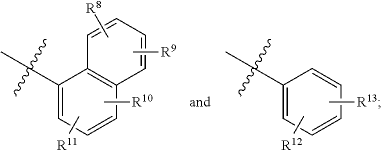
a¹ is N or CHZ¹;
Z¹ is selected from hydrogen, halo, (C₁-C₄)alkyl, (C₂-C₄)alkenyl, cyano, cyano(C₁-C₄)alkyl, —S[halo(C₁-C₄)alkyl], and (C₃-C₆)cycloalkyl;
R⁸, R⁹, R¹⁰, R¹¹, R¹², and R¹³ are each independently selected from hydrogen, (C₁-C₄)alkyl, (C₁-C₄)haloalkyl, (C₁-C₄)cyanoalkyl, (C₁-C₄)hydroxyalkyl, —(C₁-C₄)alkylNR^{a}R^{b}, —(C₁-C₄)alkylC(O)NR^{a}R^{b}, (C₁-C₄)alkylO(C₁-C₄)alkyl, —(C₁-C₄)alkylC(O)OR^{a}, —(C₁-C₄)alkylNR^{a}C(O)OR^{b}, —(C₁-C₄)alkylC(O)R^{a}, —(C₁-C₄)alkylheterocyclyl, —(C₁-C₄)alkylaryl, —(C₁-C₄)alkylheteroaryl, (C₂-C₄)alkenyl, (C₂-C₄)haloalkenyl, (C₂-C₄)cyanoalkenyl, (C₂-C₄)hydroxyalkenyl, —(C₂-C₄)alkenylNR^{a}R^{b}, (C₂-C₄)alkynyl, (C₂-C₄)haloalkynyl, (C₂-C₄)cyanoalkynyl, (C₂-C₄)hydroxyalkynyl, —(C₂-C₄)alkynylNR^{a}R^{b}, (C₁-C₄)alkoxy, (C₁-C₄)haloalkoxy, halo, cyano, oxo, hydroxy, —S(C₁-C₄)alkyl, —S(C₁-C₄)haloalkyl, —NR^{a}R^{b}, —NR^{a}C(O)R^{b}, —C(O)R^{a}, —C(O)OR^{a}, —SO₂R^{a}, —S(O)R^{a}, —SO₂NR^{a}R^{b}, —NR^{a}SO₂R^{b}, (C₃-C₆)cycloalkyl, 5- or 6-membered heteroaryl, and 4- to 6-membered heterocyclyl, wherein said heterocyclyl, aryl, and heteroaryl of —(C₁-C₄)alkylheterocyclyl, —(C₁-C₄)alkylaryl, and —(C₁-C₄)alkylheteroaryl, and said (C₃-C₆)cycloalkyl, 5- or 6-membered heteroaryl, and 4-to 6-membered heterocyclyl are each optionally and independently substituted with 1 to 3 groups selected from R^{c};
R^{a} and R^{b} are each independently selected from hydrogen, (C₁-C₄)alkyl, and (C₁-C₄)haloalkyl, or R^{a} and R^{b}, when on the same nitrogen atom, may be taken together to form a heterocyclyl; and
R^{c} is selected from halo, (C₁-C₄)alkyl, (C₁-C₄)haloalkyl, (C₁-C₄)alkoxy, (C₁-C₄)haloalkoxy, cyano, hydroxyl, oxo, —C(O)OR^{a}, —C(O)R^{a}, —SO₂R^{a}, —S(O)R^{a}, —SO₂NR^{a}R^{b}, —NR^{a}C(O)R^{b}, —NRaSO₂R^{b}, —NR^{a}R^{b}, and NO₂.

2. The compound of claim 1, wherein the compound is of the Formula I: 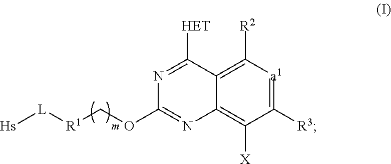
or a pharmaceutically acceptable salt thereof.

3. The compound of claim 1 or 2, or a pharmaceutically acceptable salt thereof, wherein HET is optionally substituted 3,8-diazabicyclo[3.2.1]octanyl or 3,6-diazabicyclo[3.2.1]octanyl.

4. The compound of claim 1 or 2, or a pharmaceutically acceptable salt thereof, wherein HET is optionally substituted 3,8-diazabicyclo[3.2.1]octanyl.

5. The compound of any one of claims 1 to 4, wherein the compound is of the Formula Ia or IIa: 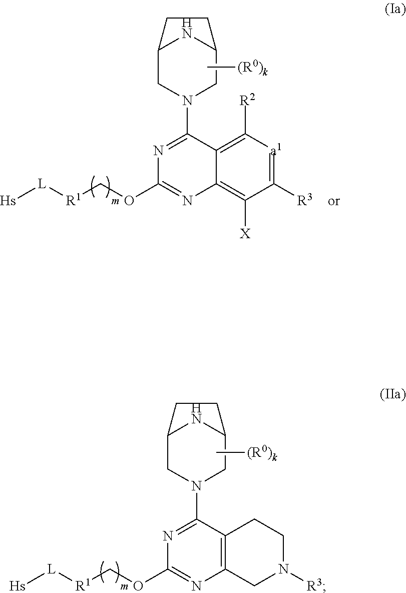
or a pharmaceutically acceptable salt thereof, wherein
R⁰ is halo, (C₁-C₄)alkyl, hydroxy(C₁-C₄)alkyl, cyano(C₁-C₄)alkyl, —C(O)H, —C(O)₂H, —C(O)₂(C₁-C₄)alkyl, —C(O)(C₁-C₄)alkyl, —C(O)(C₁-C₄)haloalkyl, —C(O)₂(C₁-C₄)haloalkyl, C(O)₂NH₂, —C(O)₂NH(C₁-C₄)alkyl, —C(O)₂N[(C₁-C₄)alkyl]₂, —S(O)₂(C₁-C₄)alkyl and —S(O)₂(C₁-C₄)haloalkyl, or a 5- to 6-membered optionally substituted heteroaryl; and
k is0, 1, 2, or 3.

6. The compound of any one of claims 1 to 5, wherein the compound is of the Formula Ia: 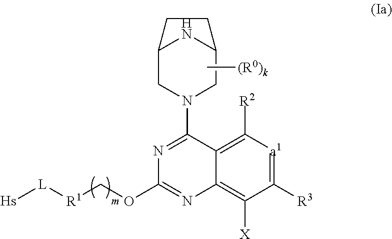
or a pharmaceutically acceptable salt thereof.

7. The compound of claim 5, wherein the compound is of the Formula Ib or IIb: 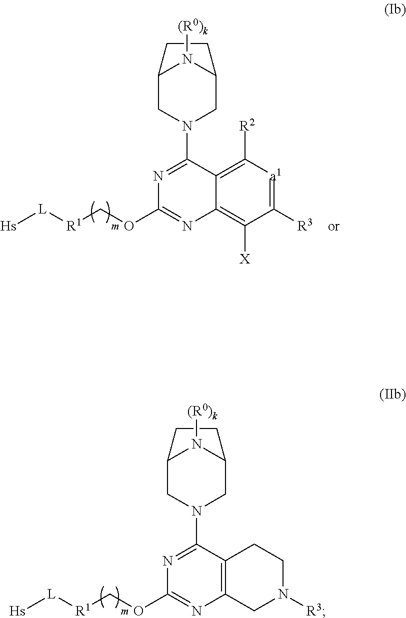
or a pharmaceutically acceptable salt thereof, wherein k is 0 or 1.

8. The compound of claim 5 or 6, wherein the compound is of the Formula Ib: 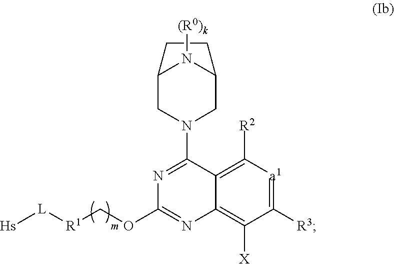
or a pharmaceutically acceptable salt thereof, wherein k is 0 or 1.

9. The compound of any one of claims 5 to 8, or a pharmaceutically acceptable salt thereof, wherein R⁰ is cyano(C₁-C₄)alkyl, —C(O)(C₁-C₄)alkyl, or —C(O)(C₁-C₄)haloalkyl.

10. The compound of any one of claims 5 to 8, or a pharmaceutically acceptable salt thereof, wherein R⁰ is —C(O)(C₁-C₄)alkyl or —C(O)(C₁-C₄)haloalkyl.

11. The compound of any one of claims 5 to 10, or a pharmaceutically acceptable salt thereof, wherein k is 0.

12. The compound of any one of claims 1 to 11, or a pharmaceutically acceptable salt thereof, wherein a¹ is N.

13. The compound of any one of claims 1 to 12, or a pharmaceutically acceptable salt thereof, wherein X is halo.

14. The compound of any one of claims 1 to 13, or a pharmaceutically acceptable salt thereof, wherein X is fluoro.

15. The compound of any one of claims 1 to 14, or a pharmaceutically acceptable salt thereof, wherein R² is hydrogen or (C₁-C₄)alkoxy.

16. The compound of any one of claims 1 to 15, or a pharmaceutically acceptable salt thereof, wherein R² is hydrogen.

17. The compound of any one of claims 1 to 16, or a pharmaceutically acceptable salt thereof, wherein R⁸, R⁹, R¹⁰, R¹¹, R¹², and R¹³ are each independently selected from hydrogen, halo, (C₂-C₄)alkynyl, halo(C₁-C₄)alkyl, (C₁-C₄)alkyl, cyano, (C₁-C₄)alkoxy, and hydroxy.

18. The compound of any one of claims 1 to 17, or a pharmaceutically acceptable salt thereof, wherein
R⁸, R⁹, R¹⁰, and R¹¹ are each hydrogen; or
R⁹, R¹⁰ and R¹¹ are each hydrogen and R⁸ is selected from halo, cyano, hydroxy, (C₁-C₄)alkoxy, and (C₂-C₄)alkynyl; or
R⁸, R⁹ and R¹⁰ are each hydrogen and R¹¹ is hydroxy; or
R¹⁰ is hydrogen and R⁸, R⁹, and R¹¹ are each independently selected from halo, (C₂-C₄)alkynyl, (C₁-C₄)alkyl, cyano, (C₁-C₄)alkoxy, and hydroxy; or
R¹² and R¹³ are each hydrogen; or
R¹² is halo(C₁-C₄)alkyl and R¹³ is hydrogen; or
R¹² and R¹³ are each independently selected from halo(C₁-C₄)alkyl, (C₁-C₄)alkyl, and halo.

19. The compound of any one of claims 1 to 18, or a pharmaceutically acceptable salt thereof, wherein R³ is selected from

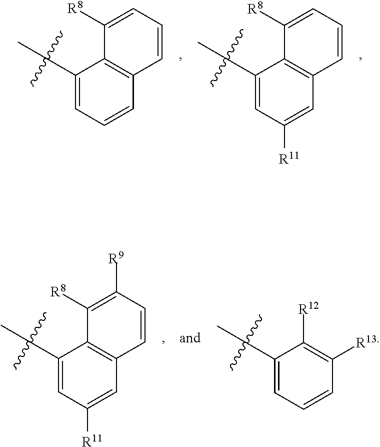

20. The compound of any one of claims 1 to 19, or a pharmaceutically acceptable salt thereof, wherein R³ is selected from

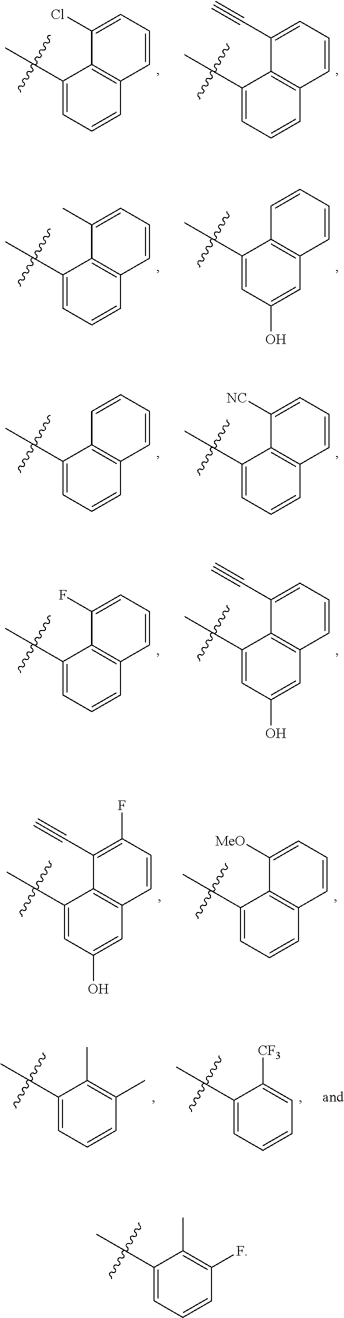

21. The compound of any one of claims 1 to 20, or a pharmaceutically acceptable salt thereof, wherein R¹ is selected from an optionally substituted heterocyclyl and an optionally substituted cycloalkyl.

22. The compound of any one of claims 1 to 21, or a pharmaceutically acceptable salt thereof, wherein R¹ is selected from an optionally substituted 4- to 6-membered nitrogen containing heterocyclyl, an optionally substituted 8- to 10-membered fused bicyclic heterocyclyl, and an optionally substituted (C₃-C₄)cycloalkyl.

23. The compound of any one of claims 1 to 22, or a pharmaceutically acceptable salt thereof, wherein R¹ is selected from azetidinyl, pyrrolidinyl, cyclopropyl, piperazinyl, and hexahydro-1H-pyrrolizinyl, each of which are optionally substituted with 1 to 3 groups selected from (C₁-C₄)alkyl, (C₁-C₄)haloalkyl, (C₁-C₄)alkoxy, C₁-C₄)haloalkoxy, and halo.

24. The compound of any one of claims 1 to 22, or a pharmaceutically acceptable salt thereof, wherein R¹ is selected from pyrrolidinyl, cyclopropyl, piperazinyl, and hexahydro-1H-pyrrolizinyl, each of which are optionally substituted with 1 to 3 groups selected from (C₁-C₄)alkyl, (C₁-C₄)haloalkyl, (C₁-C₄)alkoxy, C₁-C₄)haloalkoxy, and halo.

25. The compound of any one of claims 1 to 23, or a pharmaceutically acceptable salt thereof, wherein R¹ is selected from azetidinyl, pyrrolidinyl, cyclopropyl, piperazinyl, and hexahydro-1H-pyrrolizinyl, each of which are optionally substituted with (C₁-C₄)alkyl.

26. The compound of any one of claims 1 to 24, or a pharmaceutically acceptable salt thereof, wherein R¹ is selected from pyrrolidinyl, cyclopropyl, piperazinyl, and hexahydro-1H-pyrrolizinyl, each of which are optionally substituted with (C₁-C₄)alkyl.

27. The compound of any one of claims 1 to 25, or a pharmaceutically acceptable salt thereof, wherein R¹ is selected from 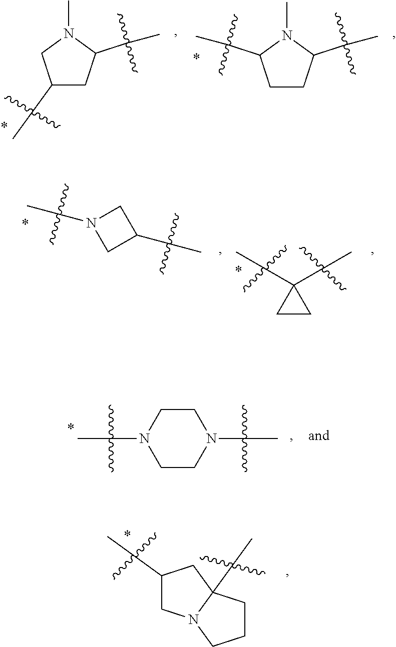
wherein the * indicates the point of attachment to L.

28. The compound of any one of claims 1 to 26, or a pharmaceutically acceptable salt thereof, wherein R¹ is selected from

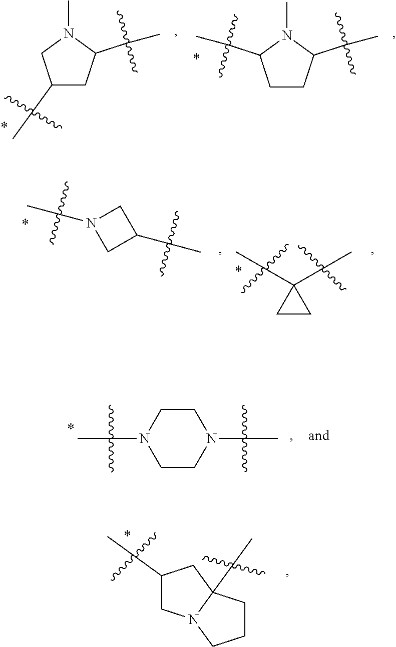

29. The compound of any one of claims 1 to 28, or a pharmaceutically acceptable salt thereof, wherein m is 0, 1 or 2.

30. The compound of any one of claims 1 to 29, or a pharmaceutically acceptable salt thereof, wherein Hs is selected from 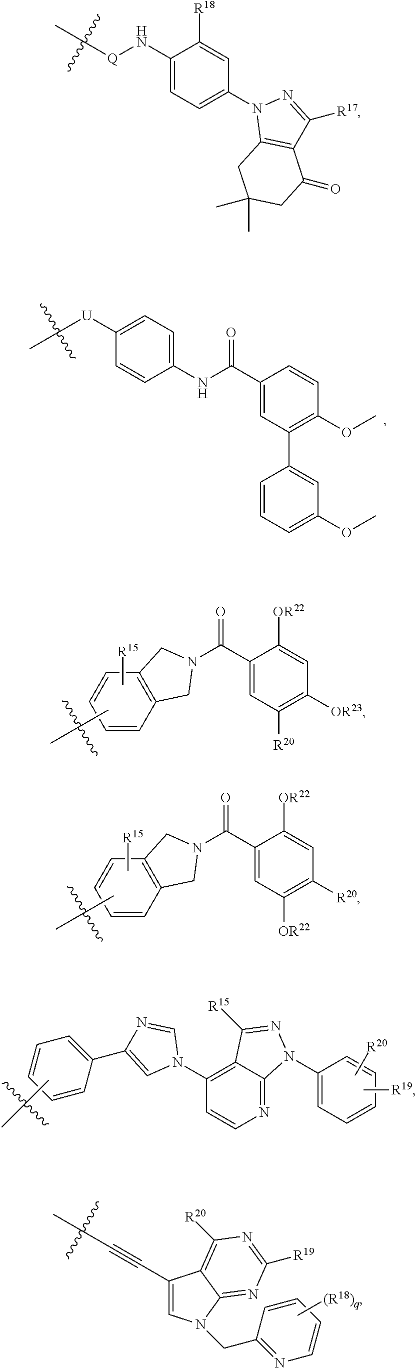 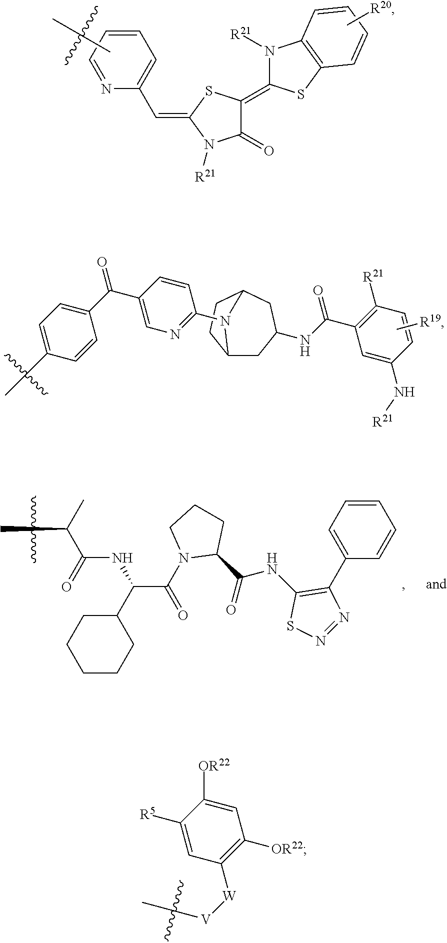
wherein
Q and U are each independently selected from phenyl, heteroaryl, heterocyclyl, and cycloalkyl, each of which being optionally substituted with 1 to 3 groups selected from R¹⁴;
R¹⁷, R¹⁸, R¹⁹ and R²⁰ are each independently selected from hydrogen, halo, CN, (C₁-C₄)alkyl, halo(C₁-C₄)alkyl, —NR^{a}R^{b}, —OR^{e}, and —C(O)NR^{a}R^{b};
q is 0, 1, 2, or 3;
R²¹, R²², and R²³ are each independently hydrogen, (C₁-C₄)alkyl, or halo(C₁-C₄)alkyl;
W is 5- or 6-membered heteroaryl optionally substituted with 1 to 3 groups selected from R¹⁴;
V is phenyl or 5- to 9-membered heteroaryl optionally substituted with 1 to 3 groups selected from R¹⁵;
R⁵ is halo, (C₁-C₄)alkyl, halo(C₁-C₄)alkyl, (C₁-C₄)alkoxy, or halo(C₁-C₄)alkoxy;
R¹⁴ is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{e}, —OR^{e}, —C(O)R^{e}, —C(O)OR^{e}, —C(O)NR^{e}R^{f}, —C(O)NR^{e}(C₁₋₄alkylene)OR^{e}, —C(O)NR^{e}(C₁₋₄alkylene)NR^{e}R^{f}, —C(O)NR^{e}(C₁₋₄alkylene)OR, —NR^{e}R^{f}, —O(C₁₋₄alkylene)NR^{e}R^{f}, —SH, —S(C₁₋₄alkyl), —C₁₋₄alkylNR^{e}R^{f}, —SR^{e}, —S(O)R^{e}, —S(O)₂R^{e}, —S(O)NR^{e}R^{f}, —SO₂NR^{e}R^{f}, —NR^{e}(C₁₋₄alkyl)OR^{e}, —NR^{e}(C₁₋₄alkyl)NR^{e}R^{f}, —C₁₋₆alkylC(O)NR^{e}R^{f}, phenyl or 5- to 7-membered heteroaryl, wherein said phenyl and 5- to 7-membered heteroaryl are each optionally and independently substituted with 1 to 3 groups selected from R¹⁶;
R^{e} and R^{f} are each independently selected from hydrogen and (C₁-C₄)alkyl, wherein said (C₁-C₄)alkyl is optionally substituted with one or more halo or a 3- to 7-membered heterocyclyl, or both; and
R¹⁵ and R¹⁶ are each independently halo, —NR^{e}R^{f}, (C₁-C₄)alkyl, halo(C₁-C₄)alkyl, (C₁-C₄)alkoxy, or halo(C₁-C₄)alkoxy.

31. The compound of any one of claims 1 to 29, or a pharmaceutically acceptable salt thereof, wherein Hs is selected from 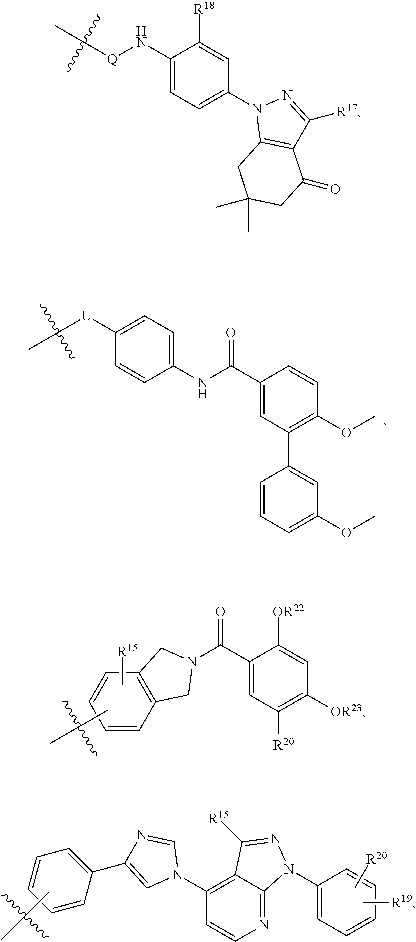 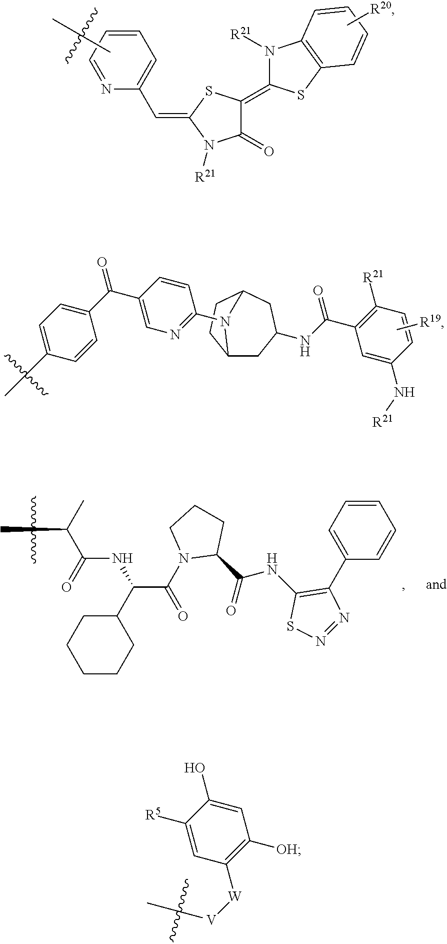
wherein
Q and U are each independently selected from phenyl, heteroaryl, heterocyclyl, and cycloalkyl, each of which being optionally substituted with 1 to 3 groups selected from R¹⁴;
R¹⁷, R¹⁸, R¹⁹ and R²⁰ are each independently selected from hydrogen, halo, CN, (C₁-C₄)alkyl, halo(C₁-C₄)alkyl, and —C(O)NR^{a}R^{b};
R²¹, R²², and R²³ are each independently hydrogen, (C₁-C₄)alkyl, or halo(C₁-C₄)alkyl;
W is 5- or 6-membered heteroaryl optionally substituted with 1 to 3 groups selected from R¹⁴;
V is phenyl or 5- to 9-membered heteroaryl optionally substituted with 1 to 3 groups selected from R¹⁵;
R⁵ is halo, (C₁-C₄)alkyl, halo(C₁-C₄)alkyl, (C₁-C₄)alkoxy, or halo(C₁-C₄)alkoxy;
R¹⁴ is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{e}, —OR^{e}, —C(O)R^{e}, —C(O)OR^{e}, —C(O)NR^{e}R^{f}, —C(O)NR^{e}(C₁₋₄alkylene)OR^{e}, —C(O)NR^{e}(C₁₋₄alkylene)NR^{e}R^{f}, —C(O)NR^{e}(C₁₋₄alkylene)OR, —NR^{e}R^{f}, —O(C₁₋₄alkylene)NR^{e}R^{f}, —SH, —S(C₁₋₄alkyl), —C₁₋₄alkylNR^{e}R^{f}, —SR^{e}, —S(O)R^{e}, —S(O)₂R^{e}, —S(O)NR^{e}R^{f}, —SO₂NR^{e}R^{f}, —NR^{e}(C₁₋₄alkyl)OR^{e}, —NR^{e}(C₁₋₄alkyl)NR^{e}R^{f}, —C₁₋₆alkylC(O)NR^{e}R^{f}, phenyl or 5- to 7-membered heteroaryl, wherein said phenyl and 5- to 7-membered heteroaryl are each optionally and independently substituted with 1 to 3 groups selected from R¹⁶;
R^{e} and R^{f} are each independently selected from hydrogen and (C₁-C₄)alkyl, wherein said (C₁-C₄)alkyl is optionally substituted with one or more halo or a 3- to 7-membered heterocyclyl, or both; and
R¹⁵ and R¹⁶ are each independently halo, —NR^{e}R^{f}, (C₁-C₄)alkyl, halo(C₁-C₄)alkyl, (C₁-C₄)alkoxy, or halo(C₁-C₄)alkoxy.

32. The compound of any one of claims 1 to 31, or a pharmaceutically acceptable salt thereof, wherein Hs is

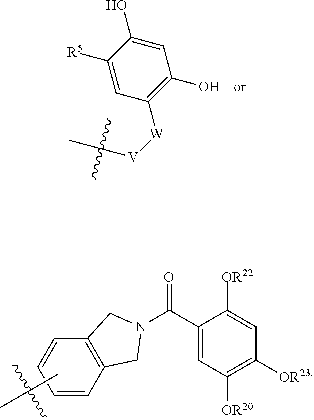

33. The compound of any one of claims 30 to 32, or a pharmaceutically acceptable salt thereof, wherein R²⁰ is (C₁-C₄)alkyl.

34. The compound of any one of claims 30 to 33, or a pharmaceutically acceptable salt thereof, wherein R²² and R²³ are each hydrogen, R²² and R²³ are each (C₁-C₄)alkyl, R²² is hydrogen and R²³ is (C₁-C₄)alkyl, or R²³ is hydrogen and R²² is (C₁-C₄)alkyl.

35. The compound of any one of claims 30 to 33, or a pharmaceutically acceptable salt thereof, wherein R²² and R²³ are each hydrogen, R²² is hydrogen and R²³ is (C₁-C₄)alkyl, or R²³ is hydrogen and R²² is (C₁-C₄)alkyl.

36. The compound of any one of claims 1 to 32, or a pharmaceutically acceptable salt thereof, wherein Hs is selected from 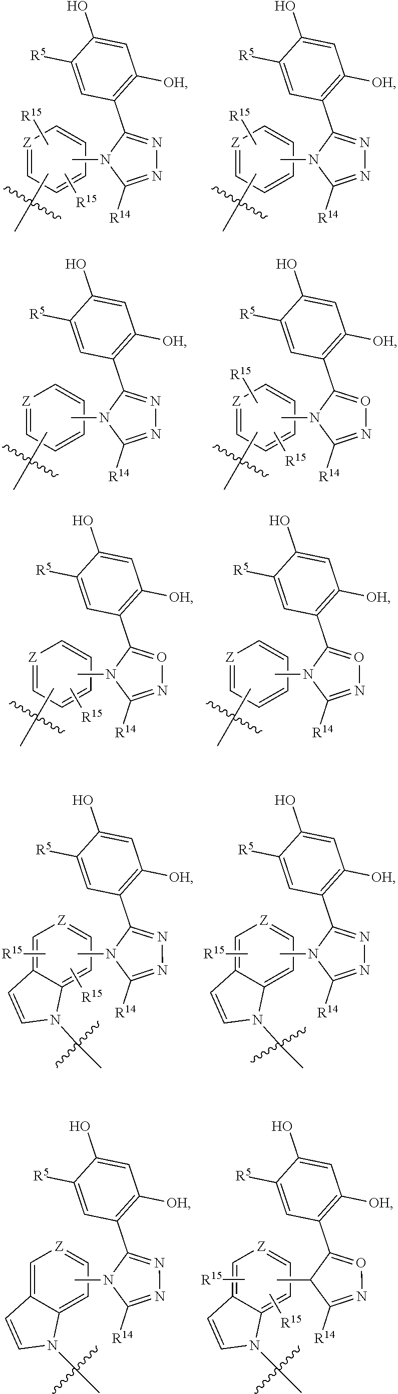 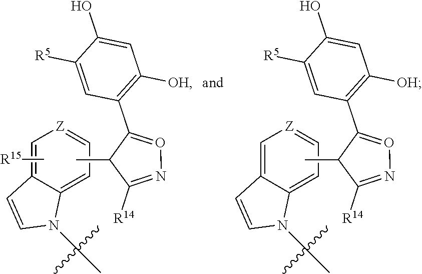
and
Z is N or CH.

37. The compound of claim 36, or a pharmaceutically acceptable salt thereof, wherein Z is CH.

38. The compound of claim 36 or 37, wherein each R¹⁵ is independently (C₁-C₄)alkyl or halo.

39. The compound of any one of claims 1 to 32, or a pharmaceutically acceptable salt thereof, wherein Hs is

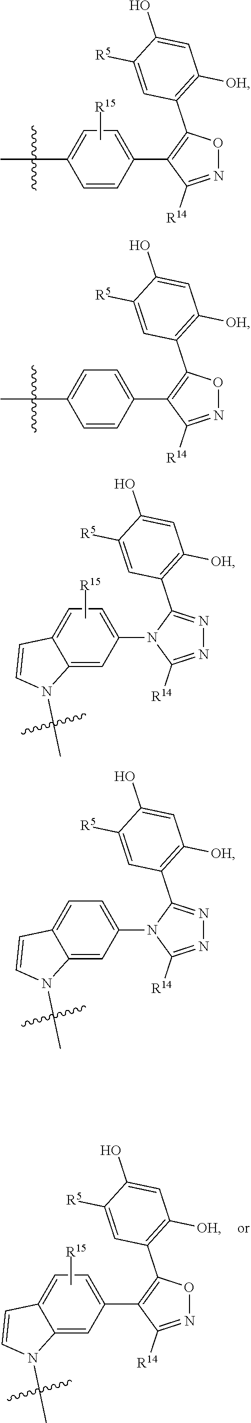

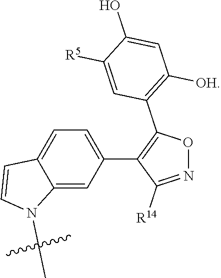

40. The compound of any one of claims 1 to 32, or a pharmaceutically acceptable salt thereof, wherein Hs is

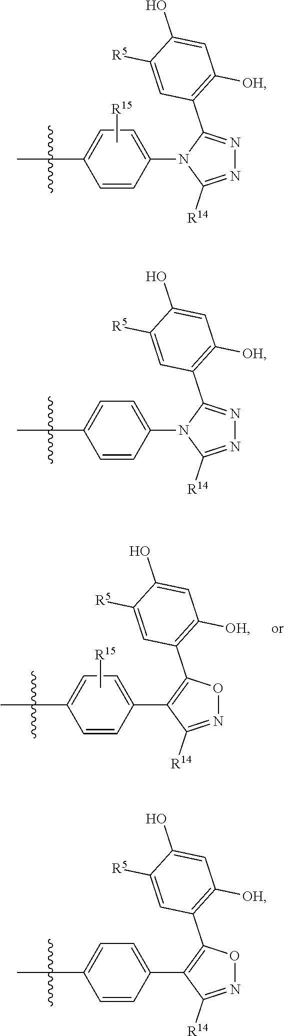

41. The compound of any one of claims 1 to 32, or a pharmaceutically acceptable salt thereof, wherein Hs is

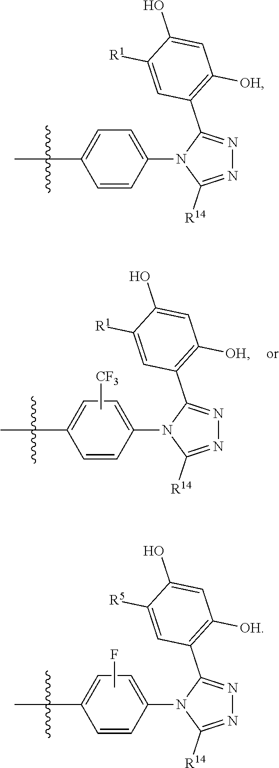

42. The compound of any one of claims 1 to 32, or a pharmaceutically acceptable salt thereof, wherein Hs is

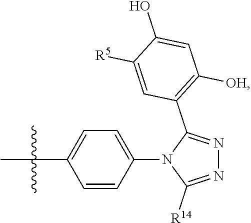

43. The compound of any one of claims 30 to 42, or a pharmaceutically acceptable salt thereof, wherein R⁵ is halo or (C₁-C₄)alkyl.

44. The compound of any one of claims 30 to 43, or a pharmaceutically acceptable salt thereof, wherein R⁵ is chloro, isopropyl, methyl, propyl, or ethyl.

45. The compound of any one of claims 30 to 44, or a pharmaceutically acceptable salt thereof, wherein R⁵ is isopropyl or ethyl.

46. The compound of any one of claims 30 to 45, or a pharmaceutically acceptable salt thereof, wherein R¹⁴ is —OR^{e}, —SR^{e}, —C(O)NR^{e}R^{f}, or —C(O)NR^{e}(C₁₋₄alkylene)NR^{e}R^{f}.

47. The compound of any one of claims 30 to 46, or a pharmaceutically acceptable salt thereof, wherein R^{e} and R^{f} are each independently selected from hydrogen and (C₁-C₄)alkyl, wherein said (C₁-C₄)alkyl is optionally substituted with 1 to 3 halo or a 6-membered heterocyclyl.

48. The compound of any one of claims 30 to 47, or a pharmaceutically acceptable salt thereof, wherein R¹⁴ is OH, —C(O)NHCH₂CF₃, —C(O)NHCH₂CH₃, —C(O)NHCH(CH₃)₂, —C(O)NH(CH₂CH₃)₂, —C(O)NHCH(CH₃)CF₃, —C(O)NHcyclopropyl, —C(O)NHmethylcyclopropyl, —C(O)NH₂, or —C(O)NH(CH₂)₂piperidinyl.

49. The compound of any one of claims 30 to 48, or a pharmaceutically acceptable salt thereof, wherein R¹⁴ is —C(O)NHCH₂CF₃ or OH.

50. The compound of any one of claims 30 to 49, or a pharmaceutically acceptable salt thereof, wherein R¹⁴ is OH.

51. The compound of any one of claims 1 to 30, or a pharmaceutically acceptable salt thereof, wherein Hs is selected from

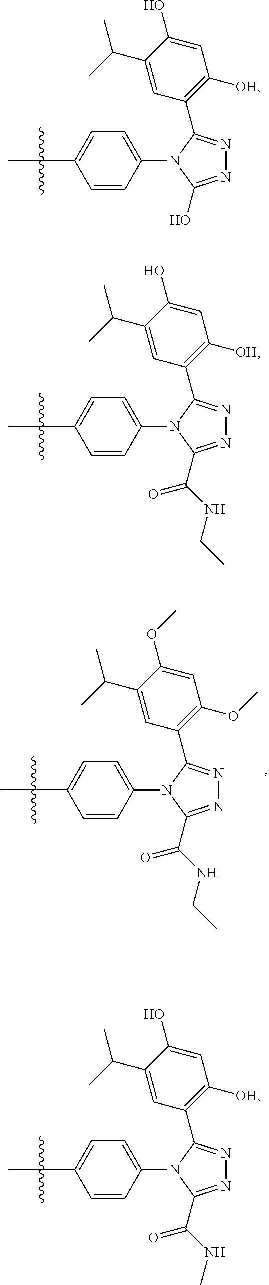 [SMILES: *c1ccc(-n2c(O)nnc2-c2cc(C(C)C)c(O)cc2O)cc1 ; *c1ccc(-n2c(C(=O)NCC)nnc2-c2cc(C(C)C)c(O)cc2O)cc1 ; *c1ccc(-n2c(C(=O)NCC)nnc2-c2cc(C(C)C)c(OC)cc2OC)cc1 ; *c1ccc(-n2c(C(=O)NC)nnc2-c2cc(C(C)C)c(O)cc2O)cc1]

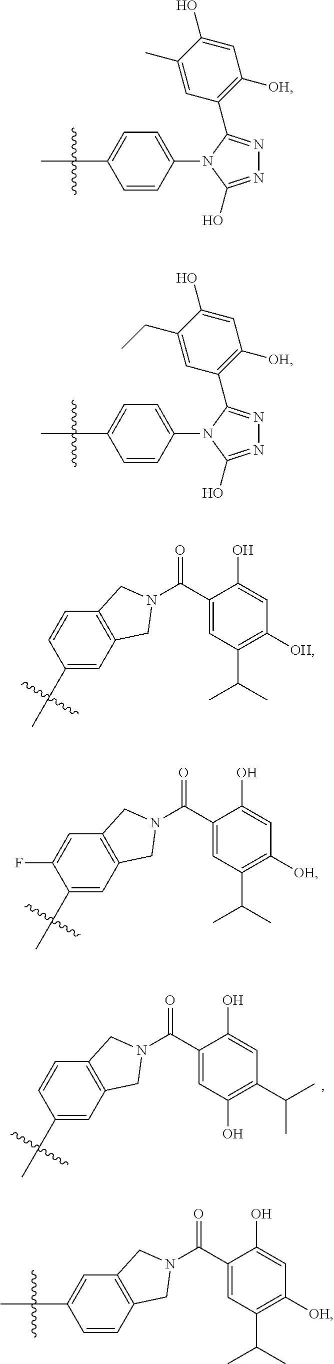 [SMILES: *c1ccc(-n2c(O)nnc2-c2cc(C)c(O)cc2O)cc1 ; *c1ccc(-n2c(O)nnc2-c2cc(CC)c(O)cc2O)cc1 ; *c1ccc2c(c1)CN(C(=O)c1cc(C(C)C)c(O)cc1O)C2 ; *c1cc2c(cc1F)CN(C(=O)c1cc(C(C)C)c(O)cc1O)C2 ; *c1ccc2c(c1)CN(C(=O)c1cc(O)c(C(C)C)cc1O)C2 ; *c1ccc2c(c1)CN(C(=O)c1cc(C(C)C)c(O)cc1O)C2]

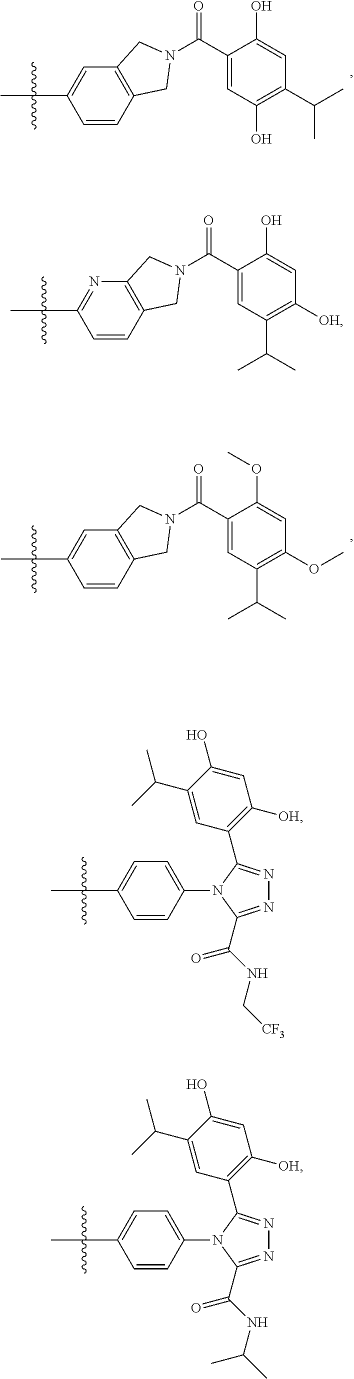 [SMILES: *c1ccc2c(c1)CN(C(=O)c1cc(O)c(C(C)C)cc1O)C2 ; *c1ccc2c(n1)CN(C(=O)c1cc(C(C)C)c(O)cc1O)C2 ; *c1ccc2c(c1)CN(C(=O)c1cc(C(C)C)c(OC)cc1OC)C2 ; *c1ccc(-n2c(C(=O)NCC(F)(F)F)nnc2-c2cc(C(C)C)c(O)cc2O)cc1 ; *c1ccc(-n2c(C(=O)NC(C)C)nnc2-c2cc(C(C)C)c(O)cc2O)cc1]

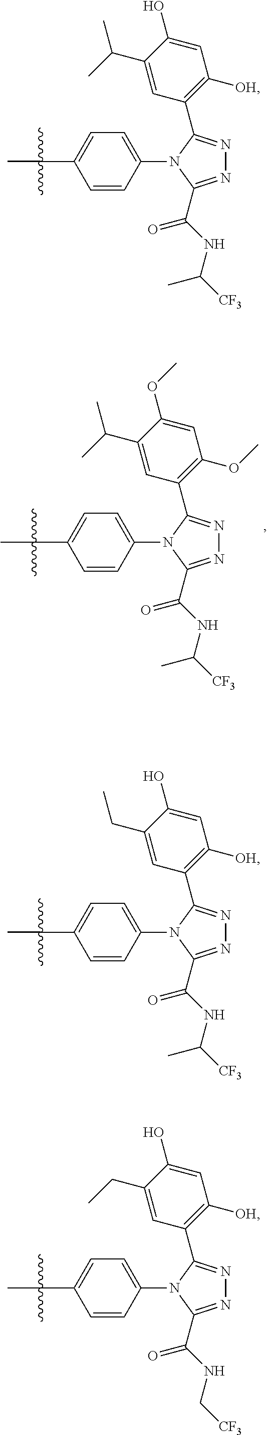 [SMILES: *c1ccc(-n2c(C(=O)NC(C)C(F)(F)F)nnc2-c2cc(C(C)C)c(O)cc2O)cc1 ; *c1ccc(-n2c(C(=O)NC(C)C(F)(F)F)nnc2-c2cc(C(C)C)c(OC)cc2OC)cc1 ; *c1ccc(-n2c(C(=O)NC(C)C(F)(F)F)nnc2-c2cc(CC)c(O)cc2O)cc1 ; *c1ccc(-n2c(C(=O)NCC(F)(F)F)nnc2-c2cc(CC)c(O)cc2O)cc1]

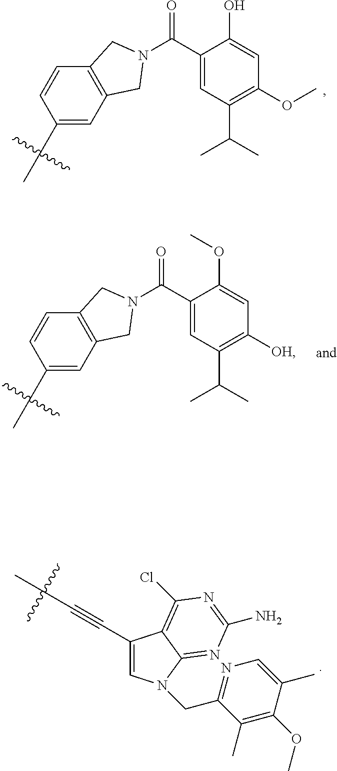 [SMILES: *c1ccc2c(c1)CN(C(=O)c1cc(C(C)C)c(OC)cc1O)C2 ; *c1ccc2c(c1)CN(C(=O)c1cc(C(C)C)c(O)cc1OC)C2 ; *C#Cc1cn(Cc2ncc(C)c(OC)c2C)c2nc(N)nc(Cl)c12]

52. The compound of any one of claims 1 to 31, or a pharmaceutically acceptable salt thereof, wherein Hs is selected from

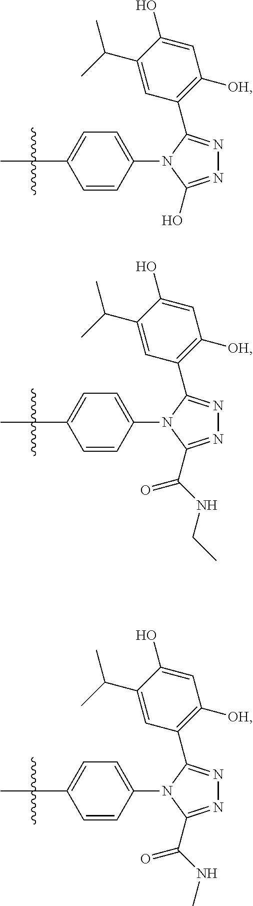 [SMILES: *c1ccc(-n2c(O)nnc2-c2cc(C(C)C)c(O)cc2O)cc1 ; *c1ccc(-n2c(C(=O)NCC)nnc2-c2cc(C(C)C)c(O)cc2O)cc1 ; *c1ccc(-n2c(C(=O)NC)nnc2-c2cc(C(C)C)c(O)cc2O)cc1]

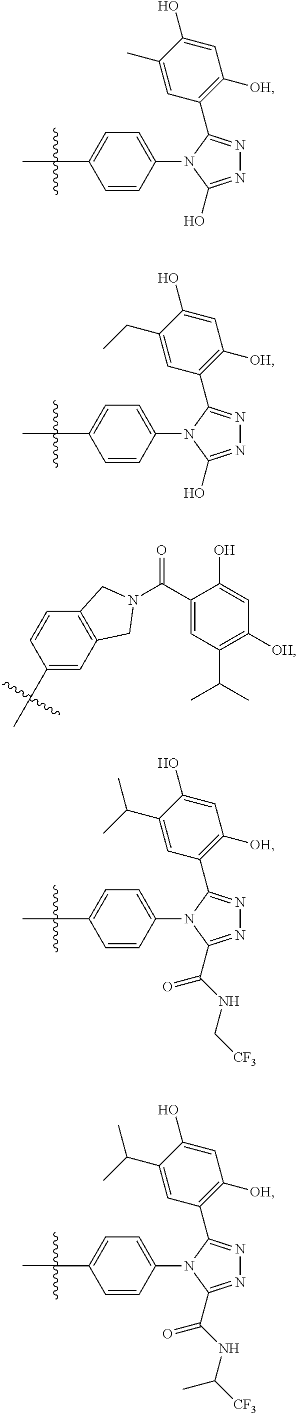 [SMILES: *c1ccc(-n2c(O)nnc2-c2cc(C)c(O)cc2O)cc1 ; *c1ccc(-n2c(O)nnc2-c2cc(CC)c(O)cc2O)cc1 ; *c1ccc2c(c1)CN(C(=O)c1cc(C(C)C)c(O)cc1O)C2 ; *c1ccc(-n2c(C(=O)NCC(F)(F)F)nnc2-c2cc(C(C)C)c(O)cc2O)cc1 ; *c1ccc(-n2c(C(=O)NC(C)C(F)(F)F)nnc2-c2cc(C(C)C)c(O)cc2O)cc1]

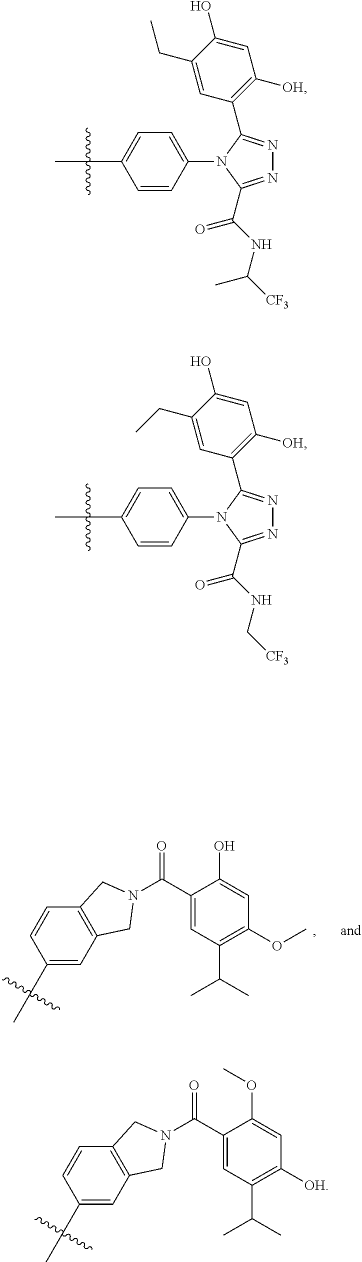 [SMILES: *c1ccc(-n2c(C(=O)NC(C)C(F)(F)F)nnc2-c2cc(CC)c(O)cc2O)cc1 ; *c1ccc(-n2c(C(=O)NCC(F)(F)F)nnc2-c2cc(CC)c(O)cc2O)cc1 ; *c1ccc2c(c1)CN(C(=O)c1cc(C(C)C)c(OC)cc1O)C2 ; *c1ccc2c(c1)CN(C(=O)c1cc(C(C)C)c(O)cc1OC)C2]

53. The compound of any one of claims 1 to 52, or a pharmaceutically acceptable salt thereof, wherein
L is selected from (C₁-C₆)alkyl, (CH₂CH₂O)ᵥ, (C₁-C₆)alkylNR^{q}, (C₁-C₆)alkylC(O), (C₁-C₆)alkylC(O)O, (C₁-C₆)alkylOC(O), (C₁-C₆)alkylC(O)NR^{q}, (C₁-C₆)alkylNR^{q}C(O), X¹-Het¹-X², X¹-Het¹-X²—X³, X¹-Het¹-X²—X³—X⁴, X¹-Het¹-X²—X³—X⁴—X⁵, X¹-Het¹-X²-Het²-X³—, X¹-Het¹-X²-Het²-X³-Het³-X⁴—, (CH₂CH₂O), —X¹-Het¹-X², (CH₂CH₂O), —X¹-Het¹-X²—X³, (CH₂CH₂O), —X¹-Het¹-X²-Het²-X³—, (CH₂CH₂O), —X¹-Het¹-X²-Het²-X³-Het³-X⁴—, (C₁-C₆)alkylNR^{q}—X¹-Het¹-X², (C₁-C₆)alkylNR^{q}—X¹-Het¹-X²—X³, (C₁-C₆)alkylNR^{q}—X¹-Het¹-X²-Het²-X³—, (C₁-C₆)alkylNR^{q}—X¹-Het¹-X²-Het²-X³-Het³-X⁴—, (C₁-C₆)alkylC(O)—X¹-Het¹-X², (C₁-C₆)alkylC(O)—X¹-Het-X²—X³, (C₁-C₆)alkylC(O)—X¹-Het¹-X²-Het²-X³—, (C₁-C₆)alkylC(O)—X¹-Het¹-X²-Het²-X³-Het³-X⁴—, (C₁-C₆)alkylC(O)NR^{q}—X¹-Het¹-X², (C₁-C₆)alkylC(O)NR^{q}—X¹-Het¹-X²—X³, (C₁-C₆)alkylC(O)NR^{q}—X¹-Het¹-X²-Het²-X³—, and (C₁-C₆)alkylC(O)NR^{q}—X¹-Het¹-X²-Het²-X³-Het³-X⁴—;
each X¹, X², X³, X⁴, and X⁵ are independently absent or selected from (C₁-C₄)alkylene, O, NR^{q}, and C(O);
v is 1, 2, 3, 4, 5, or 6;
R^{q} is hydrogen or (C₁-C₄)alkyl; and
each Het¹, Het², and Het³ are independently selected from a 4- to 6-membered heterocyclyl and (C₃-C₆)cycloalkyl, each optionally substituted with 1 to 3 groups selected from halo, (C₁-C₄)alkyl, (C₁-C₄)haloalkyl, (C₁-C₄)alkoxy, and (C₁-C₄)haloalkoxy.

54. The compound of any one of claims 1 to 52, or a pharmaceutically acceptable salt thereof, wherein
L is selected from (C₁-C₆)alkyl, (CH₂CH₂O)ᵥ, (C₁-C₆)alkylNR^{q}, (C₁-C₆)alkylC(O), (C₁-C₆)alkylC(O)O, (C₁-C₆)alkylOC(O), (C₁-C₆)alkylC(O)NR^{q}, (C₁-C₆)alkylNR^{q}C(O), X¹-Het²-X², X¹-Het¹-X²—X³, X¹-Het¹-X²-Het²-X³—, X¹-Het¹-X²-Het²-X³-Het³-X⁴—, (CH₂CH₂O)ᵥ—X¹-Het¹-X², (CH₂CH₂O)ᵥ—X¹-Het¹-X²—X³, (CH₂CH₂O)ᵥ—X¹-Het¹-X²-Het²-X³—, (CH₂CH₂O)ᵥ—X¹-Het¹-X²-Het²-X³-Het³-X⁴—, (C₁-C₆)alkylNR^{q}—X¹-Het¹-X², (C₁-C₆)alkylNR^{q}—X¹-Het¹-X²—X³, (C₁-C₆)alkylNR^{q}—X¹-Het¹-X²-Het²-X³—, (C₁-C₆)alkylNR^{q}—X¹-Het¹-X²-Het²-X³-Het³-X⁴—, (C₁-C₆)alkylC(O)—X¹-Het¹-X², (C₁-C₆)alkylC(O)—X¹-Het-X²—X³, (C₁-C₆)alkylC(O)—X¹-Het-X²-Het²-X³—, (C₁-C₆)alkylC(O)—X¹-Het-X²-Het²-X³-Het³-X⁴—, (C₁-C₆)alkylC(O)NR^{q}—X¹-Het¹-X², (C₁-C₆)alkylC(O)NR^{q}—X¹-Het¹-X²—X³, (C₁-C₆)alkylC(O)NR^{q}—X¹-Het¹-X²-Het²-X³—, and (C₁-C₆)alkylC(O)NR^{q}—X¹-Het¹-X²-Het²-X³-Het³-X⁴—;
each X¹, X², X³, and X⁴ are independently absent or selected from (C₁-C₄)alkylene, O, NR^{q}, and C(O);
v is 1, 2, 3, 4, 5, or 6
R^{q} is hydrogen or (C₁-C₄)alkyl; and
each Het¹, Het², and Het³ are independently selected from a 4- to 6-membered heterocyclyl and (C₃-C₆)cycloalkyl, each optionally substituted with 1 to 3 groups selected from halo, (C₁-C₄)alkyl, (C₁-C₄)haloalkyl, (C₁-C₄)alkoxy, and (C₁-C₄)haloalkoxy.

55. The compound of any one of claims 1 to 53, or a pharmaceutically acceptable salt thereof, wherein L is selected from (C₁-C₆)alkyl, (C₁-C₆)alkylNR^{q}, X¹-Het²-X², X¹-Het¹-X²—X³, X¹-Het¹-X²—X³—X⁴, X¹-Het¹-X²—X³—X⁴—X⁵, X¹-Het²-X²-Het²-X³—, and X¹-Het¹-X²-Het²-X³-Het³-X⁴—.

56. The compound of any one of claims 1 to 54, or a pharmaceutically acceptable salt thereof, wherein L is selected from (C₁-C₆)alkyl, (C₁-C₆)alkylNR^{q}, X¹-Het²-X², X¹-Het¹-X²—X³, X¹-Het¹-X²-Het²-X³—, and X¹-Het²-X²-Het²-X³-Het³-X⁴—.

57. The compound of any one of claims 53 to 56, or a pharmaceutically acceptable salt thereof, wherein each Hetⁱ, Het², and Het³ are independently absent or selected from piperidinyl, piperazinyl, cyclohexyl, cyclopropyl, cyclobutyl, azetidinyl, and pyrrolidinyl, each optionally substituted with 1 to 3 groups selected from halo, (C₁-C₄)alkyl, (C₁-C₄)haloalkyl, (C₁-C₄)alkoxy, and (C₁-C₄)haloalkoxy.

58. The compound of any one of claims 53 to 57, or a pharmaceutically acceptable salt thereof, wherein each Het¹, Het², and Het³ are independently absent or selected from piperidinyl, piperazinyl, and pyrrolidinyl.

59. The compound of any one of claims 1 to 58, or a pharmaceutically acceptable salt thereof, wherein L is selected from —CH₂—, *CH₂N(CH₃), 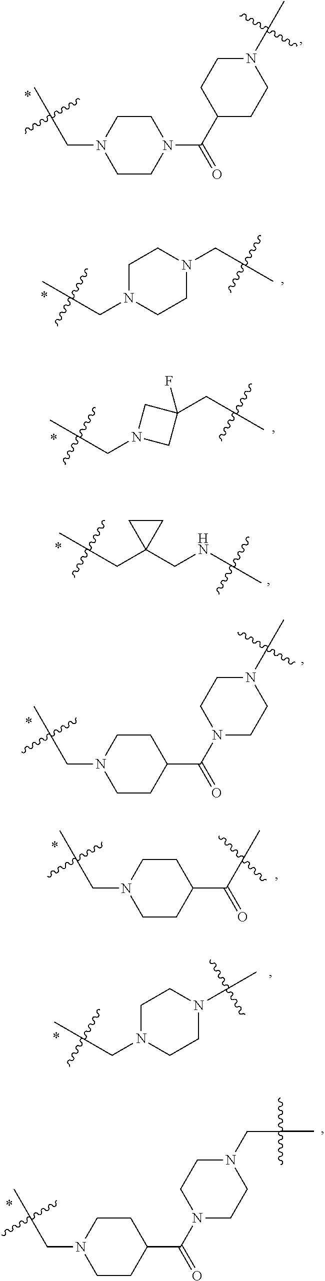 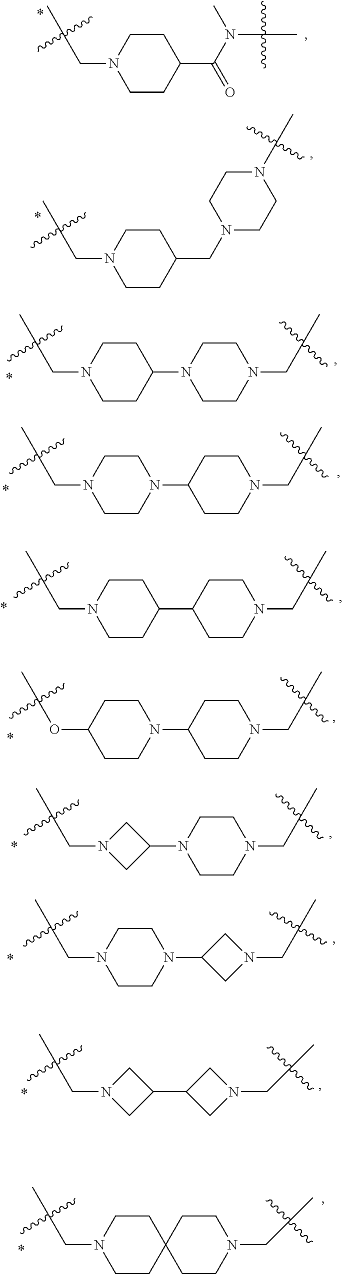 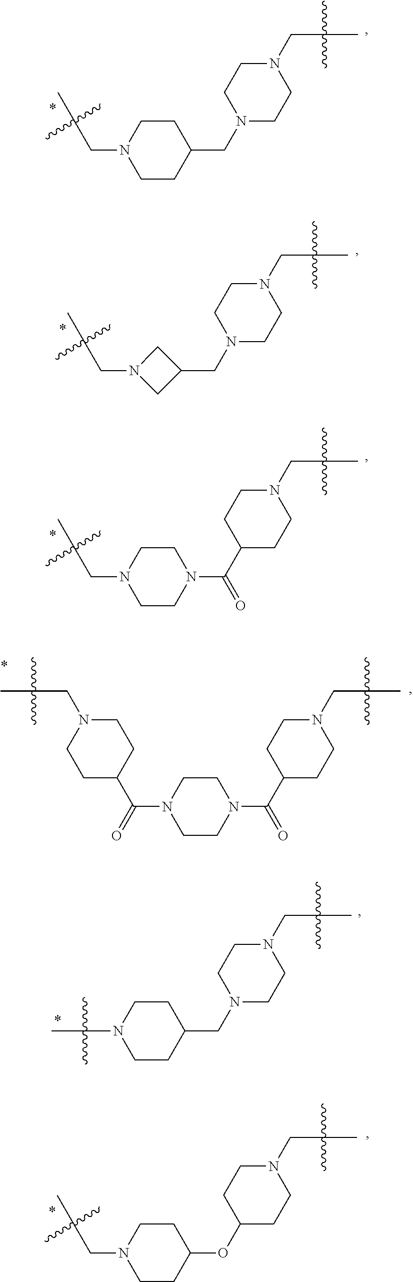 [SMILES: *CN1CCC(CN2CCN(C*)CC2)CC1 ; *CN1CCN(CC2CN(C*)C2)CC1 ; *CN1CCC(C(=O)N2CCN(C*)CC2)CC1 ; *CN1CCC(C(=O)N2CCN(C(=O)C3CCN(C*)CC3)CC2)CC1 ; *CN1CCN(CC2CCN(*)CC2)CC1 ; *CN1CCC(OC2CCN(C*)CC2)CC1] 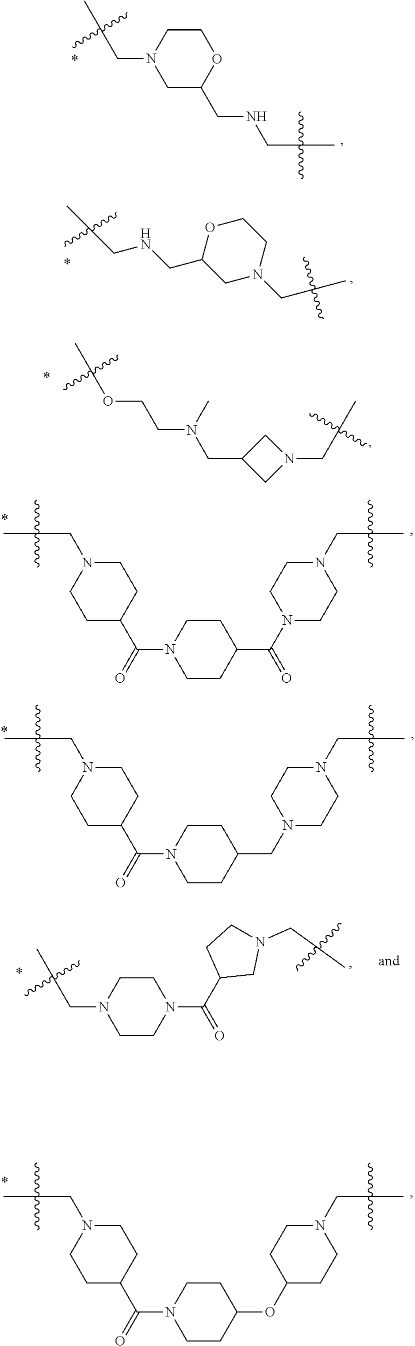
wherein the * indicates the point of attachment to Hs.

60. The compound of claim 1, wherein the compound is selected from Examples 1 to 3 and any of those in Table 1; or a pharmaceutically acceptable salt thereof.

61. A pharmaceutical composition comprising a compound of any one of claims 1 to 60, or a pharmaceutically acceptable salt thereof; and a pharmaceutically acceptable carrier.

62. A method for treating cancer in a subject comprising administering to the subject and effective amount of a compound of any one of claims 1 to 60, or a pharmaceutically acceptable salt thereof, or the pharmaceutically acceptable composition of claim 61.
